(19) United States
(12) Patent Application Publication
(10) Pub. No.: US 2024/0153578 A1
(43) Pub. Date: May 9, 2024

(54) METHOD OF CHARACTERISING A CANCER

(71) Applicant: Cambridge Enterprise Limited, Cambridge (GB)

(72) Inventors: Serena Nik-Zainal, Cambridge (GB); Andrea Degasperi, Cambridge (GB); Xueqing Zou, Cambridge (GB)

(73) Assignee: Cambridge Enterprise Limited, Cambridge (GB)

(21) Appl. No.: 18/283,540

(22) PCT Filed: Mar. 21, 2022
(86) PCT No.: PCT/EP2022/057387

§ 371 Date: Sep. 22, 2023

(30) Foreign Application Priority Data
Mar. 26, 2021 (GB) 2104308.8

(51) Int. Cl. G16B 20/20; G16B 40/20; G16H 20/10; G16H 50/20

(52) U.S. Cl. CPC G16B 20/20; G16B 40/20; G16H 20/10; G16H 50/20

(57) ABSTRACT

The invention provides a method of characterising a DNA sample obtained from a tumour, the method including the steps of: determining the value of one or more mutational signature metrics for the sample, wherein the mutational signature metrics are selected from: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts; and based on said values of said one or more mutational signature metrics, classifying said sample between a class associated with a high likelihood of being mismatch repair (MMR)-deficient and a class associated with a low likelihood of being MMR-deficient. Identification of a tumour as MMR-deficient may be used to inform treatment choices, for example treatment with an immune therapy such as a checkpoint inhibitor, and for providing a prognosis.

### FIELD OF INVENTION

The present invention relates to a method for characterising the properties of cancer based on a DNA sample from a tumour. It is particularly, but not exclusively, concerned with a method for identifying whether the tumour is deficient in mismatch repair (MMR), and methods for identifying a treatment accordingly.

### BACKGROUND TO THE INVENTION

Somatic mutations are a hallmark of cancer and can arise through both endogenous and exogenous processes. Endogenous processes that have been shown to give rise to DNA lesions include endogenous biochemical activities such as hydrolysis and oxidation (Lindhal et al., 1972), and errors at replication. Fortuitously, our cells are equipped with DNA repair pathways that constantly mitigate this endogenous damage (Mardis et al., 2019; Berger & Mardis, 2018). One such pathway is the DNA mismatch repair (MMR) pathway. This pathway is highly conserved and plays a key role in maintaining genomic stability (Li, 2007). In eukaryotes, the pathway is mediated by key proteins collectively referred to as “Mut homologue” proteins. These include MSH2 and MSH6 (together forming the heterodimer MutSα), MSH2 and MSH3 (together forming the heterodimer MSHβ), MLH1 and PMS2 (together forming the heterodimer MutLα), MLH1 and PMS1 (together forming the heterodimer MutLβ), and MLH1 and MLH3 (together forming the heterodimer MutLγ).

Mutations in the Mut homologue proteins affect genomic stability, and are known to be associated with genetic conditions such as Lynch syndrome (also known as Hereditary nonpolyposis colorectal cancer (HNPCC)), an autosomal dominant genetic condition that is associated with a high risk of colon cancer as well as endometrial, ovary, stomach, small intestine, hepatobiliary tract, upper urinary tract, brain, and skin cancer. MMR deficiency can result in microsatellite instability (MSI), a condition that manifests in the creation of novel microsatellite fragments (repeated sequences of DNA, with repeats often a few base pairs long). MSI has been associated with many cancers, and is most prevalent in association with colon cancer. Studies have found that patients stratified on the basis of whether they were MSI-High (MSI-H), MSI-low (MSI-L) or microsatellite stable (MSS) had different prognosis, with the MSI-H status associated with better survival (Popat et al., 2005). This relationship with cancer prognosis has led to the development of multiple commercial diagnostic assays for the detection of microsatellite instability. However, MSI is only one possible manifestation of impaired DNA mismatch repair. Therefore, testing for MSI is not equivalent to testing for MMR deficiency, which is the true biological difference underlying differences in prognosis and response to therapy. Sequence data (such as e.g. whole exome sequencing or whole genome sequencing data) is increasingly commonly acquired in the context of cancer therapy. This data can potentially be leveraged to acquire a wealth of information about a patient's tumour, including their MMR status. Algorithms to classify MMR-deficiency tumors have been developed using massively-parallel sequencing data (Ni Huang et al., 2013; Wang & Liang, 2018; Cortes-Ciriano, 2017; Salipante et al., 2014; Hause et al., 2016). These classifiers depend on detecting elevated tumor mutational burdens (TMB) or microsatellite instability (MSI). Thus, they also rely on relatively crude metrics of genomic instability that common manifestations of MMR deficiency.

Therefore, there is still a need for improved methods for identifying MMR-deficient tumours using sequence data.

Statements of Invention

The present inventors postulated that improved prediction of the MMR status of tumours could be obtained through the use of mutational signatures. Somatic mutations arising through endogenous and exogenous processes mark the genome with distinctive patterns, termed mutational signatures (Helleday et al., 2014; Alexandrov et al., 2013; Nik-Zainal et al., 2012; Nik-Zainal et al., 2012). While there have been advancements in analytical aspects of deriving mutational signatures from human cancers (Alexandrov et al., 2020; Haradhvala et al., 2018; Kim et al., 2016), etiologies and mechanisms underpinning these mutational patterns (Nik-Zainal, S. et al., 2015; Zou, X. et al., 2018; Christensen, S. et al., 2019; Kucab, J. E. et al., 2019) are often still unclear. The present inventors used an experimental approach to create biallelic gene knockouts that produce mutational signatures in the absence of administered DNA damage, and are thus indicative of genes that are important at maintaining the genome from intrinsic sources of DNA perturbations. They identified signatures of substitutions and/or indels in a plurality of genes including 5 genes in the MMR pathway: ΔMLH1, ΔMSH2, ΔMSH6, ΔPMS2, and ΔPMS1, suggesting that proteins of these genes are critical guardians of the genome in non-transformed cells, and supporting the hypothesis that mutational signatures could provide a useful indication of the presence of a deficiency in this pathway. These insights led them to develop a more sensitive and specific mutational-signature-based assay to detect MMR deficiency, MMRDetect. Current TMB-based assays have reduced sensitivity to detect MMR deficiency because many tissues do not have high proliferative rates and may not meet the detection criteria of such assays. They may also falsely call MMR-deficient cases as MMR-proficient, because single components were used for measurement (e.g., indel burden or substitution count only). High mutational burdens can be due to different biological processes (Campbell et al., 2017). Consequently, assays based on burden alone are unlikely to be adequately specific. By contrast, the new approach was shown to have excellent specificity and sensitivity, and was able to correctly classify cases that were misclassified with previous approaches.

Thus, according to a first aspect, there is provided a method of characterising a DNA sample obtained from a tumour, the method including the steps of: determining the value of one or more mutational signature metrics for the sample, wherein the mutational signature metrics are selected from: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts; and based on said values of said one or more mutational signature metrics, determining whether said sample has a high or low likelihood of being mismatch repair (MMR)-deficient. Determining the value of one or more mutational signature metrics for the sample may comprise determining the similarity between the substitution profile of the sample and that of one or more MMR gene knockouts.

The present inventors have identified the similarity between the substitution profile of the sample and that of one or more MMR gene knockouts to have high predictive value in relation to the sample's MMR status. Prior to the present invention, prediction of MMR status was based primarily on the observation of signs of microsatellite instability. The inventors postulated that mutational profiles that can be identified in samples known to have an MMR deficiency may provide a good indicator of MMR status in test samples. They found that this was indeed the case, but only for some mutational profiles and metrics derived therefrom. The similarity between substitution profiles of a test and MMR gene knockouts was surprisingly found to be a particularly good predictor of MMR status. By contrast, the similarity between the profile of repeat-mediated insertion of a sample and that of knockout generated indel signatures was found to be a poor predictor of MMR status.

Determining the value of one or more mutational signature metrics for the sample may comprise determining the exposure of one or more mutational signatures of MMR. The present inventors have identified the exposure of mutational signatures that have been associated with MMR as having high predictive value in relation to the sample's MMR status. Importantly, associations between mutational signatures and possible underlying biological mechanisms are typically proposed aetiologies that are not underlined by direct mechanistic evidence. Thus, the observation that exposure of MMR signatures is actually predictive of MMR status could not have been predicted from the mere fact that these signatures have been postulated to be associated with MMR deficiency. For example, patterns of mutations that are similar to those caused by MMR deficiency may also result from other mutational processes or combinations thereof, such that the observation of the presence of such patterns may in practice not correlate or not sufficiently correlate with MMR status.

Determining the value of one or more mutational signature metrics for the sample may further comprise determining the number of repeat mediated indels in the mutational profile of the sample. Determining the value of one or more mutational signature metrics for the sample may further comprise determining the similarity between the repeat mediated deletion profile of the sample and that of one or more MMR gene knockouts. The present inventors have identified the number of repeat mediated indels in the mutational profile of a sample, and the similarity between the repeat mediated deletion profile of the sample and that of one or more MMR gene knockouts to improve the MMR status prediction obtained using MMR signature exposure and/or similarity between substitution profiles of the sample and that of one or more MMR gene knockouts, at least in the training cohort used. By contrast, the similarity between the repeat mediated insertion profile of the sample and that of one or more MMR gene knockouts was not found to improve the prediction of MMR status in the training cohort used.

Determining the value of one or more mutational signature metrics for the sample may comprise determining the value of all of: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts.

Determining whether said sample has a high or low likelihood of being MMR-deficient comprises using said values of said one or more mutational signature metrics to classify said sample between a class associated with a high likelihood of being mismatch repair (MMR)-deficient and a class associated with a low likelihood of being MMR-deficient. Classifying said sample may comprise classifying the sample between a class associated with a high likelihood of being mismatch repair (MMR)-deficient, a class associated with a low likelihood of being MMR-deficient, and one or more additional classes. The one or more additional classes may comprise one or more classes associated with different likelihood of being MMR deficient, and/or one or more classes associated with unknown status (e.g. a class associated with a medium likelihood of being MMR deficient in addition to classes associated with high and low likelihoods of being MMR deficient, respectively). In other words, the classification may be binary or may be a multi-class classification. Determining whether said sample has a high or low likelihood of being mismatch repair (MMR)-deficient may be performed based on the values of one or more further metrics in addition to the values of the one or more mutational signature metrics.

The step of classifying the sample may be performed using one or more machine learning models selected from: a decision tree, a logistic regression classifier, a support vector machine, a naïve Bayes classifier, and a k-nearest neighbour classifier. The machine learning model is preferably a logistic regression classifier. The present inventors have found that logistic regression classifiers were particularly robust, and in particular performed best when applied to data sets that are different from those on which the classifier was trained (such as e.g. when applied to samples from a different type of tumour from those represented in the data that was used to train the classifier).

Determining whether said sample has a high or low likelihood of being MMR-deficient may comprise: generating, using said values of said one or more mutational signature metrics, a probabilistic score; and based on said probabilistic score, determining whether said sample has a high or low likelihood of being MMR-deficient. Determining, based on said probabilistic score, whether said sample has a high or low likelihood of being MMR-deficient may comprise comparing said probabilistic score with one or more predetermined thresholds, and determining that the sample has a high likelihood of being MMR-deficient if the probabilistic score is below a first predetermined threshold, and a low likelihood of being MMR-deficient if the probabilistic score is at or above a second predetermined threshold. The first and second predetermined threshold may be the same or different.

The method may further comprise receiving (e.g. from a user through a user interface, or from a database) or determining a first and or second predetermined threshold. The first and/or second predetermined thresholds may be determined (or may have been determined) using test data comprising the values of said probabilistic score for a plurality of samples that have a known MMR deficiency status. For example, the predetermined threshold(s) may be chosen so as to optimise (maximise or minimise, as the case may be) one or more performance metrics such as accuracy, specificity or sensitivity of detection of samples from MMR-deficient tumours.

The first and second predetermined thresholds may be the same, and may be between about 0.5 and about 0.9, between about 0.6 and about 0.8, such as about 0.7. The present inventors have found a threshold of 0.7 to be associated with a particularly high accuracy, at least based on the test data used (comprising colorectal tumour samples).

In embodiments, determining, based on said probabilistic score, whether said sample has a high or low likelihood of being MMR-deficient comprises comparing said probabilistic score with one or more predetermined thresholds, and determining that the sample has a high likelihood of being MMR-deficient if the probabilistic score is above a first predetermined threshold, and a low likelihood of being MMR-deficient if the probabilistic score is at or below a second predetermined threshold, optionally wherein the first and second predetermined threshold are the same.

The probabilistic score may be obtained using a logistic regression model, optionally wherein the probabilistic score is generated using the formula:

$$log ( \frac{p}{1 - p} ) = {β}_{0} + {\sum }_{i = 1}^{k} {β}_{i} {x}_{i}$$

where p is the probability that a sample has a particular MMR deficiency status, so is an intercept weight, β is a vector of weights for each of k variables, and x is a vector of variables associated with the sample, wherein the variables comprise said one or more mutational signature metrics or variables derived therefrom. For example, variables derived from the one or more mutational signature metrics may be obtained by scaling each of the mutational signature metrics. The value of the weights β and intercept weight β₀ may be determined using a suitable training cohort.

Determining the value of one or more mutational signature metrics for the sample may comprise scaling the value of each mutational signature metric. Scaling the mutational signature metrics may advantageously increase the comparability of the values of the respective variables and reduce the risk that metrics that are on different scales disproportionately affect the probabilistic score obtained. Scaling may be performed using any method known in the art, such as e.g. by normalisation (also known as min-max scaling, i.e. transforming a variable such that the range of possible values for the variable ranges between 0 and 1), or by standardisation (where values are centred around the mean with a unit standard deviation by, for each observation, subtracting the mean and dividing by the standard deviation for the variable). The present inventors have found simple normalisation, for example dividing each value by the maximum observed or expected value for the variable to strike a good balance between simplicity and improving the comparability of the variables thus improving the performance of the MMR deficiency identification process. The scaling may be performed using one or more parameters for each mutational signature metric, such as e.g. a value by which every value for a particular metric should be divided in order to obtain the corresponding derived (i.e. normalised) value. Thus, the method may further comprise receiving or determining the value of said one or more parameters.

Determining whether said sample has a high or low likelihood of being mismatch repair (MMR)-deficient based on the value of said mutational signature metrics for the sample may comprise weighting each of said values by a predetermined weighting factor. The predetermined weighting factors may represent the relative importance of the mutational signature metrics in the determination of the likelihood of the sample being MMR-deficient. The predetermined weighting factors may be such that the exposure of one or more mutational signatures of mismatch repair (MMR) has a higher weight than any of: the similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts. Instead or in addition to this, the predetermined weighting factors may be such that the similarity between the substitution profile of the sample and that of one or more MMR gene knockouts has a higher weight than any of: the number of repeat mediated indels in the mutational profile of the sample, and the similarity between the repeat mediated deletion profile of the sample and that of one or more MMR gene knockouts Instead or in addition to this, the predetermined weighting factors may be such that the exposure of one or more mutational signatures of mismatch repair (MMR) and the similarity between the substitution profile of the sample and that of one or more MMR gene knockouts both have a higher respective weight than any of: the number of repeat mediated indels in the mutational profile of the sample, and the similarity between the repeat mediated deletion profile of the sample and that of one or more MMR gene knockouts Instead or in addition to this, the predetermined weighting factors may be such that the exposure of one or more mutational signatures of mismatch repair (MMR) has a higher weight than the similarity between the substitution profile of the sample and that of one or more MMR gene knockouts, the similarity between the substitution profile of the sample and that of one or more MMR gene knockouts has a higher weight than the similarity between the repeat mediated deletion profile of the sample and that of one or more MMR gene knockouts, and the similarity between the repeat mediated deletion profile of the sample and that of one or more MMR gene knockouts has a higher weight than the number of repeat mediated indels in the mutational profile of the sample.

For example, the exposure of one or more mutational signatures of mismatch repair (MMR) may have a weight between about −60 and about −20, between about −50 and about −30, between about −40 and −45, such as about −43, e.g. −42.95. As another example, the similarity between the substitution profile of the sample and that of one or more MMR gene knockouts may have a weight between about −20 and about 0, between about −20 and about −10, about −15, such as e.g. −14.53. As another example, the similarity between the repeat mediated deletion profile of the sample and that of one or more MMR gene knockouts may have a weight between about −15 and about 0, between about −10 and about 0, about −5, such as e.g. −4.62. As another example, the number of repeat mediated indels in the mutational profile of the sample may have a weight between about −20 and about 0, between about −15 and 0, between about −10 and 0, between about −5 and 0, about −3, such as e.g. −2.96. When a linear model is used (such as e.g. a logistic regression model), an intercept weight so may additionally be used. The intercept weight may have a value between 10 and 20, such as e.g. 16.043. The precise value of the intercept is not critical as it is identical for every sample and hence samples can still be compared to each other regardless of the value used for the intercept weight. However, when using models such as a logistic regression model, an intercept value fitted using a suitable training dataset is preferably used as this enables the interpretation of the resulting score in a more straightforward manner as indicative of the likelihood of samples being MMR deficient.

All of the variables are preferably normalised prior to weighting. Alternatively, the respective weights may be adjusted so as to obtain equivalent weights for un-normalised values. As the skilled person understands, the exact values of the weights used are likely to depend on the training data used. For example, the examples herein demonstrate how to obtain suitable values using training data comprising colorectal cancer samples. Using a different training data set (comprising additional samples and/or different samples such as e.g. samples from other types of tumours) may result in different weights. However, the relative importance of the variables may remain similar.

Determining whether said sample has a high or low likelihood of being mismatch repair (MMR)-deficient based on said values of said one or more mutational signature metrics may comprise using a machine learning model that has been trained using training data comprising the values of said mutational signature metrics for a plurality of samples that have a known MMR deficiency status. In embodiments, the machine learning model is able to provide a prediction of whether a sample has a high or low likelihood of being mismatch repair (MMR)-deficient with above 99% accuracy (as evaluated using the AUC metric), such as e.g. AUC=1, on at least one test set of samples. The test set of samples preferably comprises at least 50 samples, at least 60 samples, at least 70 samples, at least 80 samples, at least 90 samples, or about 100 samples. The test set of samples may comprise samples from one or more types of tumours. The one or more types of tumours in the test set of samples may be represented in the training set used to train the machine learning algorithm. The test set of samples may comprise colorectal cancer samples. The training set of samples may comprise colorectal cancer samples. The test set of samples and the training set of samples preferably comprise samples that are known to be MMR deficient and samples that are known to be MMR proficient. The test set of samples and/or the trainings et of samples preferably comprise a plurality of samples that are known to be MMR deficient and a plurality of samples that are known to be MMR proficient. The training set of samples and/or the training set of samples preferably comprise between about 5% and about 50%, between about 10% and about 40%, between about 10% and about 30% of samples that are known to be MMR deficient. In embodiments, the proportion of samples that are known to be MMR deficient in the training set of samples is similar to that in the test set of samples. The proportion of samples that are known to be MMR deficient in the training set of samples and/or in the test set of samples may be similar to the expected proportion of tumours that are MMR deficient in the tumour samples represented in the data set.

Determining the value of one or more mutational signature metrics for the sample may comprise cataloguing the somatic mutations in said sample to produce a mutational catalogue for that sample, wherein the value of said mutational signature metrics is derived from said mutational catalogue. A mutational catalogue may also be referred to herein as a mutation profile. A mutational catalogue may be separated into sub-catalogues that catalogue mutations of a particular type such as e.g. substitutions, deletions, insertions, indels, etc. These may be referred to as a “substitution profile/catalogue”, “deletion profile/catalogue”, etc. A catalogue may comprise the number of mutations in each of a plurality of classes considered as part of a catalogue or subcatalogue.

A mutational profile may refer to a somatic mutational profile. A somatic mutational profile may comprise exclusively mutations that are not present (or assumed not to be present) in a corresponding germline genome. Thus, cataloguing the somatic mutations in a sample may comprise identifying all mutations present in a sample and removing or otherwise excluding mutations that are present or assumed to be present in a corresponding germline genome. Mutations that are present in a corresponding germline genome may be identified by identifying the mutations present in a germline sample obtained from the same subject. In other words, mutations that are present in a corresponding germline genome may be defined as mutations that have been identified by analysing genomic material from a matched normal (e.g. non-tumour and/or non-modified) sample. For example, a somatic mutational profile for a tumour may be obtained by comparison with a germline sample from the same subject (i.e. a sample of normal/non-tumour cells or genomic material derived therefrom). In the case of a mutational profile that has been obtained from a sample that has been engineered or selected to contain a particular modification, a somatic mutational profile may be obtained using a sample obtained prior to the engineering or selection step that resulted in the particular modification. For example, in the case of MMR gene knockout samples, a corresponding “germline” profile may be obtained from the parent sample, prior to introducing the MMR gene knockout modification. Mutations that are assumed to be present in a corresponding germline genome may be defined as mutations that are present in a reference genome or set of reference genomes. A reference genome or set of reference genomes may be obtained from one or more reference samples that are not strictly matched normal samples. For example, the reference sample(s) may be process matched, or may comprise a plurality of normal (i.e. non-tumour/non-modified) samples not all of which are matched to the sample for which a somatic mutational profile is determined (e.g. pooled normal samples may be used as references for a plurality of tumour samples). A reference genome or set of reference genomes may be obtained from one or more databases. For example, a reference genome may be used and all mutations compared to this reference genome may be assumed to be somatic mutations. Alternatively, a set of reference genomes may be obtained from a database as a catalogue of known germline mutations in one or more populations (e.g. a genetic variation database such as dbSNP https://www.ncbi.nlm.nih.gov/snp/, 1000 genomes https://www.internationalgenome.org/, etc.). The use of a matched normal sample advantageously provides greatest certainty that the mutations identified in the DNA from the tumour sample are somatic mutations. The use of pooled normal samples comprising a matched normal sample may provide similar (though less precise information) and may be useful e.g. when sequencing resources are limited. Compared to the use of a matched normal sample, this may risk excluding more somatic mutations are seemingly germline mutations. The use of a reference genome or set of reference genome advantageously does not require the acquisition and analysis of a separate normal sample. However, the reference genome or set of reference genome is unlikely to capture all germline mutations present in the subject, and to include mutations that are in fact somatic in the subject. This is particularly true if a single reference genome is used rather than a collection capturing common sequence variation. Thus, this may result in a less accurate identification of somatic mutations.

Cataloguing the somatic mutations in said sample may comprise determining the number of mutations in the mutational catalogue which are attributable to each of a plurality of base substitution classes and/or indel classes which are determined to be present, optionally wherein the base substitution classes include all possible trinucleotide substitution classes and/or wherein the indel classes include classes for multiple combinations of indel type, e.g. selected from insertion, deletion and complex, indel size, e.g. selected from 1-bp or longer, and flanking sequence, such as e.g. repeat-mediated, microhomology-mediated or other. The base substitution classes may be described according to the “96 channels convention” known in the art, i.e. the product of 6 types of substitution multiplied by 4 types of 5′ base (A,C,G,T) and 4 types of 3′ base (A,C,G,T). Trinucleotide substitution classes are listed in Table 3 (column “mutation type”). The indel classes may include the following 15 channels: 1 bp C/T insertion at short repetitive sequence (<5 bp), 1 bp C/T insertion at long repetitive sequence (>=5 bp), long insertions (>1 bp) at repetitive sequences, microhomology-mediated insertions, 1 bp C/T deletions at short repetitive sequence (<5 bp), 1 bp C/T deletions at long repetitive sequence (>=5 bp), long deletions (>lbp) at repetitive sequences, microhomology-mediated deletions, other deletion and complex indels. Alternatively, the indel classes may include 45 channels including the preceding 15 channels but where the 1 bp C/T indels at repetitive sequences are further expanded according to the exact length of the repetitive sequences (from 0 to 9).

Determining the value of the exposure of one or more mutational signatures of MMR for the sample may comprise determining the value of the exposure to a plurality of mutational signatures of MMR and summing the values of the exposure to each of the plurality of mutational signatures of MMR. Determining the value of the exposure of one or more mutational signatures of MMR for the sample may be performed as described in Degasperi et al. Determining the value of the exposure of one or more mutational signatures of MMR for the sample may be performed by identifying the matrix E that satisfies C≈PE where C is a mutational catalogue for the sample, P is a signature matrix comprising the one or more mutational signatures of MMR, and E is an exposure matrix. The one or more mutational signatures of MMR may be selected from RefSig MMR1 and RefSig MMR2. The one or more mutational signatures of MMR may be selected from known mutational signatures that have been derived from mutational catalogues associated with a plurality of cancer samples. Known mutational signatures that have been derived from mutational catalogues associated with a plurality of cancer samples include COSMIC signatures (e.g. as described in Alexandrov et al., 2020) or RefSig signatures (as described in e.g. Degasperi et al., 2020). The one or more mutational signatures of MMR may be signatures selected from such sets of signatures that have MMR deficiency as a postulated aetiology.

RefSig MMR1 (also referred to as “MMR1”) and RefSig MMR2 (also referred to as MMR2) are described in Degasperi et al., 2020 and available at https://signal.mutationalsignatures.com/explore/study/1 (see https://signal.mutationalsignatures.com/explore/referenceCancerSignature/52 for RefSig MMR1 and https://signal.mutationalsignatures.com/explore/referenceCancerSignature/56 for RefSiq MMR2).

The signature matrix P typically comprises the one or more mutational signatures of MMR and additional signatures that have been identified together with the one or more mutational signatures of MMR. The coefficients of the E matrix corresponding to the MMR signatures of interest in the sample under investigation may then be used as the exposure value(s) for the one or more signatures of MMR. The signature matrix P may comprise all of the reference signatures (RefSig) described in Degasperi et al., 2020 (and available at https://signal.mutationalsignatures.com/explore/study/1), or organ specific equivalents thereof. When organ-specific signatures equivalent to RefSig signatures are used, the values of the exposure RefSig MMR1 and/or RefSig MM2 may be obtained using a conversion matrix, such as described in Degasperi et al., 2020, and available at https://signal.mutationalsignatures.com/explore/study/1.

Determining the value of the similarity between a substitution or repeat mediated deletion profile of the sample and that of one or more MMR gene knockouts may comprise determine the cosine similarity between pairs of profiles. Determining the value of similarity between a substitution or repeat mediated deletion profile of the sample and that of one or more MMR gene knockouts may comprise determining the value of similarity between a substitution or repeat mediated deletion profile of the sample and that of each of a plurality of MMR gene knockouts to obtain a plurality of similarity values, and obtaining a summarised similarity value for the plurality of similarity values, optionally wherein the summarised similarity value is the maximum or the mean similarity value. Determining the value of similarity between a substitution profile of the sample and that of one or more MMR gene knockouts may comprise determining the value of similarity between a substitution profile of the sample and that of each of a plurality of MMR gene knockouts to obtain a plurality of similarity values, and obtaining a summarised similarity value for the plurality of similarity values, wherein the summarised similarity value is the maximum similarity value. Determining the value of similarity between a repeat mediated deletion profile of the sample and that of one or more MMR gene knockouts may comprise determining the value of similarity between a repeat mediated deletion profile of the sample and that of each of a plurality of MMR gene knockouts to obtain a plurality of similarity values, and obtaining a summarised similarity value for the plurality of similarity values, wherein the summarised similarity value is the mean similarity value.

The one or more MMR gene knockouts may be selected from: MSH2, MSH3, MSH6, MLH1, PMS2, and PMS1. The one or more MMR gene knockouts may be selected from: MSH2, MSH6, MLH1, PMS2, and PMS1. The one or more MMR gene knockouts may be selected from PMS2, MLH1, MSH2 and MSH6. The one or more MMR gene knockouts may include a plurality of gene knockouts, such as all of the gene knockouts, selected from: MSH2, MSH6, MLH1, PMS2, and PMS1. The one or more MMR gene knockouts include a plurality of gene knockouts selected from: PMS2, MLH1, MSH2 and MSH6. The one or more gene knockouts may include (all of) PMS2, MLH1, MSH2 and MSH6.

The substitution and/or repeat mediated deletion profile (collectively referred to as mutational profile) of an MMR gene knockout may have been derived from one or more MMR gene knockout samples as described herein. The term “MMR gene knockout sample” refers to any sample of cells or genetic material derived therefrom, in which the function of one or more genes of the MMR pathway is impaired. These one or more genes are the one referred to as “gene knockouts”, i.e. a MMR gene knockout sample which is MSH2 is a sample of cells or genetic material derived therefrom, in which the function of MSH2 is impaired.

A mutational profile for an MMR gene knockout may have been derived from a plurality of MMR gene knockout samples. Using a plurality of MMR gene knockout samples to generate each MMR gene knockout mutational profile may advantageously reduce the effect of variability between different gene knockout samples. For example, the plurality of MMR gene knockout samples may comprise a plurality (e.g. between 2 and 4) of samples of cells or material genetic derived therefrom in which the same MMR gene has been impaired. The samples may be technical and/or biological replicates, for examples samples of cells or material genetic derived therefrom where the same gene has been impaired using the same technical means. The function of a gene in the MMR pathway may have been impaired through a knockout, through silencing, through one or more mutations (e.g. coding or truncating mutations), or through downregulation. Preferably, the function of a gene in the MMR pathway has been impaired through knockout, such as e.g. using CRISPR-Cas9.

A mutational profile for an MMR gene knockout may have been derived from one or more MMR gene knockout samples and one or more background mutational profiles. The background mutational profiles may have been obtained from one or more control samples.

A mutational profile for an MMR gene knockout may have been derived from a MMR gene knockout sample by: obtaining a plurality of mutational profiles for respective bootstrap samples for the MMR gene knockout, obtaining a plurality of mutational profiles for respective bootstrap background samples, and subtracting a summarised value for the bootstrap background mutational profiles from a summarised value for the bootstrap MMR knockout mutational profiles. A summarised value may be the centroid of a plurality of mutational profiles. Mutational profiles for bootstrap samples (whether for MMR gene knockouts or background) may be obtained using a plurality of mutational profiles each obtained from a respective sample (MMR knockout sample or background sample). A background sample may be a sample in which no gene in the MMR pathway has had its function impaired. A background sample may be a sample in which the function of a control gene has been impaired. A control gene may be chosen as a gene not involved in the MMR pathway or a gene which, if impaired, does not result in a functional impairment of the MMR pathway. A control gene may be chosen as a gene that is not involved in a DNA repair pathway, or a gene which, if impaired, does not result in functional impairment in a DNA repair pathway.

A mutational profile for an MMR gene knockout may have been derived from a plurality of MMR gene knockout samples by obtaining a mutational profile for each MMR gene knockout sample and deriving a summarised mutational profile for the plurality of MMR gene knockout samples from the mutational profiles of the respective samples. Similarly, a background mutational profile may have been derived from a plurality of control samples by obtaining a mutational profile for each control sample and deriving a summarised mutational profile for the plurality of control samples from the mutational profiles of the respective samples. Alternatively, mutational profiles derived from a plurality of MMR gene knockout samples may each be used individually. For example, when determining the similarity between a mutational profile of a sample and that of a plurality of gene knockout samples, each of the profiles of the respective gene knockout samples may be compared individually with the profile of the sample, and a summarised value for the similarity (such as e.g. the maximum or average) may be used as the value of the corresponding mutational signature metric. Thus, the step of determining the value of a mutational signature metric that uses a mutational profile may comprise obtaining the mutational profile using any of the steps described above.

The similarity between two mutation profiles may be obtained as the cosine similarity. The cosine similarity is a measure of similarity between two non-zero vectors of an inner product space. It is equal to the cosine of the angle between the two vectors. It is also equal to the inner products of the two vectors, normalised to each have length 1. Alternatively, the similarity between two mutation profiles may be obtained as the angular distance or angular similarity between the two vectors encoding the mutation profiles. As another alternative, the similarity between two mutation profiles may be obtained as the Euclidian distance between L₂ normalised version of the two vectors encoding the mutation profiles. As another alternative, the similarity between two mutation profiles may be obtained s the correlation between the two vectors encoding the mutation profiles.

Determining the number of repeat mediated indels in the mutational profile of the sample may comprise obtaining a mutational catalogue for the sample and determining the number of insertions and deletions in the mutational profile that occur within repetitive regions. Repetitive regions may be regions comprising multiple repeats of the same sequence motif, optional wherein a sequence motif is a sequence of between 1 and 9 bases in length. A repetitive region may be defined as a region of a reference genome (e.g. the reference genome used to call mutational profiles, such as a defined release of the human reference genome, if human genetic material is being analysed) comprise multiple (i.e. 2 or more) repeats of the same sequence motif. A sequence motif may be defined as a sequence of one or more specific bases. For example, AA, AAA, AAAA, AAAAA, ATAT, ATATAT, ATATATAT, CAGCAG, CAGCAGCAG, CAGCAGCAGCAGCAG are all repetitive regions.

The method may further comprise obtaining the sample from a tumour of a subject. The method may further comprise obtaining sequence data from a sample from a tumour. The method may further comprise providing to a user one or more of: the value of the one or more mutational signature metrics, a value derived therefrom (such as e.g. a probabilistic score), and a determination of whether the sample has a high likelihood or a low likelihood of being MMR-deficient. The method may further comprise obtaining a germline sample from the subject and/or obtaining sequence data from a germline sample from the subject. The tumour sample may be a sample comprising tumour cells or genetic material derived therefrom. The tumour sample may be a sample of cells or tissue that has been obtained directly from a tumour (e.g. a tumour biopsy). The tumour sample may be a sample comprising cells or genetic material derived from a tumour, such as e.g. a liquid biopsy sample comprising circulating tumour cells or circulating tumour DNA.

According to a further aspect, there is provided a method of predicting whether a subject with cancer is likely to respond to an immunotherapy, the method comprising characterising a sample obtained from a tumour in the subject as having a high or low likelihood of being MMR-deficient using a method of any embodiment of the first aspect, wherein if the sample is characterised as having a high likelihood of being MMR-deficient, the subject is likely to respond to immunotherapy. The method may further comprise administering the immunotherapy, to a subject that has been diagnosed as likely to respond to immunotherapy. The method may comprise recommending a subject that has been diagnosed as likely to respond to the immunotherapy for treatment with the immunotherapy. The method may comprise administering an alternative therapy (e.g. a conventional chemotherapy, radiotherapy, etc.) and/or recommending a subject for treatment with an alternative therapy, where the subject has been diagnosed as not likely to respond to immunotherapy.

According to a further aspect, there is provided a method of selecting a subject having cancer for treatment with an immunotherapy, the method comprising characterising a sample obtained from a tumour in the subject as having a high or low likelihood of being MMR-deficient using a method of any embodiment of the first aspect, and selecting the subject for treatment with an immunotherapy if the sample is characterised as having a high likelihood of being MMR-deficient.

According to a further aspect, there is provided an immunotherapy for use in a method of treatment of cancer in a subject from whom a DNA sample has been obtained and the DNA sample has been characterised by a method according to any one of claims x to x as having a high likelihood of being MMR-deficient.

According to a further aspect, there is provided a method of treating cancer in a subject determined to have a tumour with a high likelihood of being MMR-deficient, wherein the likelihood of the tumour being MMR-deficient is determined by characterising a DNA sample obtained from the tumour using a method according to any embodiment of the first aspect.

According to any of these aspects, the immunotherapy may be administered (or recommended for administration) in combination with one or more therapies, such as one or more chemotherapies, one or more courses of radiotherapy and/or one or more surgical interventions.

According to any of these aspects, the immunotherapy may be administered (or recommended for administration) in combination with a PARP inhibitor or platinum-based therapy if the subject has been determined as having a high likelihood of being HR-deficient and/or having a high-likelihood of responding to a PARP inhibitor or platinum-based therapy. Thus, any such method may further comprise determining whether the subject is likely to respond to a PARP inhibitor or platinum-based therapy and/or characterising a sample obtained from a tumour in the subject as having a high or low likelihood of being HR-deficient. Methods suitable for this purpose are described in WO 2018/115452, WO 2017/191074, and WO 2017/191073, all of which are incorporated herein by reference.

According to a further aspect, there is provided an immunotherapy for use in a method of treatment of cancer in a subject, the method comprising: (i) determining whether a DNA sample obtained from said subject has a high or low likelihood of being MMR-deficient using a method according to any embodiment of the first aspect; and (ii) administering the immunotherapy to said subject if the DNA sample is determined to have a high likelihood of being MMR-deficient. An immunotherapy may be a checkpoint inhibitor drug, such as a PD-1 or PD-L1 inhibitor.

According to a further aspect, there is provided a method of predicting whether a subject with cancer is likely to respond to a non-fluorouracil-based chemotherapy, the method comprising characterising a sample obtained from a tumour in the subject as having a high or low likelihood of being MMR-deficient using a method of any preceding claim, wherein if the sample is characterised as having a high likelihood of being MMR-deficient, the subject is likely to respond to the non-fluorouracil-based chemotherapy.

According to a further aspect, there is provided a method of predicting whether a subject with cancer is likely to respond to a fluorouracil-based chemotherapy, the method comprising characterising a sample obtained from a tumour in the subject as having a high or low likelihood of being MMR-deficient using a method of any preceding claim, wherein if the sample is characterised as having a high likelihood of being MMR-deficient, the subject is unlikely to respond to the fluorouracil-based chemotherapy.

According to any of these aspects, the fluorouracil-based therapy or non-fluorouracil based therapy may be administered (or recommended for administration) in combination with one or more therapies, such as one or more chemotherapies, one or more courses of radiotherapy and/or one or more surgical interventions.

According to any of these aspects, the fluorouracil-based therapy or non-fluorouracil based therapy may be administered (or recommended for administration) in combination with a PARP inhibitor or platinum-based therapy if the subject has been determined as having a high likelihood of being HR-deficient and/or having a high-likelihood of responding to a PARP inhibitor or platinum-based therapy. Thus, any such method may further comprise determining whether the subject is likely to respond to a PARP inhibitor or platinum-based therapy and/or characterising a sample obtained from a tumour in the subject as having a high or low likelihood of being HR-deficient. Methods suitable for this purpose are described in WO 2018/115452, WO 2017/191074, and WO 2017/191073.

According to a further aspect, there is provided a method of providing a prognosis for a subject who has been diagnosed with cancer, the method comprising characterising a sample obtained from a tumour in the subject as having a high or low likelihood of being MMR-deficient using a method of any preceding claim, wherein if the sample is characterised as having a high likelihood of being MMR-deficient, the subject is likely to have a better prognosis than a subject characterised as having a low likelihood of being MMR-deficient.

According to a further aspect there is provided a chemotherapy for use in a method of treatment of cancer in a subject, the method comprising: (i) determining whether a DNA sample obtained from said subject has a high or low likelihood of being MMR-deficient using a method according to any embodiment of the first aspect; and (ii) administering the chemotherapy to said subject if the DNA sample is determined to have a high likelihood of being MMR-deficient, preferably wherein the chemotherapy is a non-fluorouracil-based therapy. Alternatively, the method may comprise administering the chemotherapy to said subject if the DNA sample is determined to have a low likelihood of being MMR-deficient, preferably wherein the chemotherapy is a fluorouracil-based therapy.

According to a further aspect, there is provided a method of providing a tool for characterising a DNA sample obtained from a tumour, the method including the steps of: obtaining mutational signature profiles for a plurality of training samples associated with known MMR-deficiency status; determining the value of one or more mutational signature metrics for the training samples, wherein the mutational signature metrics are selected from: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts; and training a machine learning model to predict, based on said values of said one or more mutational signature metrics, whether each training sample has a high or low likelihood of being mismatch repair (MMR)-deficient. The method of the present aspect may have any of the features described in relation to the first aspect.

According to a further aspect, there is provided a system comprising: a processor; and

a computer readable medium comprising instructions that, when executed by the processor, cause the processor to perform the (computer-implemented) steps of the method of any preceding aspect. According to a further aspect, there is provided a non-transitory computer readable medium or media comprising instructions that, when executed by at least one processor, cause the at least one processor to perform the method of any embodiment of any aspect described herein. According to a further aspect, there is provided a computer program comprising code which, when the code is executed on a computer, causes the computer to perform the method of any embodiment of any aspect described herein.

### BRIEF DESCRIPTION OF THE FIGURES

FIG. 1 is a flow diagram showing, in schematic form, a method of characterising a DNA sample according to the disclosure.

FIG. 2 shows an embodiment of a system for characterising a DNA sample.

FIG. 3 is a flow diagram illustrating schematically a method of providing a prognosis, identifying a therapy or treating a subject according to an embodiment of the present disclosure.

FIG. 4 shows the results of experiments to dissect the mutational consequences of DNA repair gene knockouts. (A) Experimental workflow from isolation of gene knockouts to generating subclones for WGS. (B) Forty-three genes were knocked out, including 42 DNA repair/replication genes and one control gene (ATP2B4). (C) Distinguishing substitution profiles of control subclones and knockout subclones. Green line shows the cosine similarities between bootstrapped profiles of controls against aggregated control substitution profile. X-axis shows the aggregated substitution number of each genotype of a knockout. (D) Distinguishing indel profile of control subclones and knockout subclones. Light blue line shows the cosine similarities between bootstrapped indel profiles of controls against aggregated control indel profile. X-axis shows the aggregated indel number of each genotype of a knockout. (E) De novo mutation number of knockout subclones cultured for 15 days. Bars and error bars represent mean±SD (standard deviation) of subclone observations.

FIG. 5 shows the substitution (A), indel (B) and double substitution (C) counts of whole-genome-sequenced subclones of gene knockout. In all comparative analyses, all gene knockouts were cultured for fifteen days and only daughter subclones that were fully clonal (i.e. clearly derived from a single cell) were included.

FIG. 6 schematically depicts the principle of detecting mutational consequences of knockouts in the absence of added external DNA damage. (A) Potential components of background signature. (B) Possible mutational consequences of the DNA repair gene knockouts for proteins that are critical mitigators of mutagenesis.

FIG. 7 shows the results of contrastive principal component analysis and t-SNE applied to the mutation profile data illustrated in FIGS. 4 and 5. (A) Contrastive principal component analysis (cPCA) was employed to discriminate knockout profiles from control profiles (ΔATP2B4). Each figure contains six different genes. Nine gene knockouts separate from the controls. Using this method, ΔADH5 did not separate clearly from ΔATP2B4, indicative of either having no signature or a weak signature. Dot colour indicate the repair/replicative pathway that each gene is involved in: black—control; green—MMR; orange—BER; dark purple—HR and HR regulation; light purple—checkpoint. (B) The t-SNE algorithm was applied to discriminate the mutational profiles of gene knockouts from those of control knockouts. Gene knockouts that produce mutational signatures separate clearly from control subclones and other knockouts which do not have signatures. Subclones of the gene knockouts which produce signatures are clustered together, indicating consistency between subclones.

FIG. 8 shows the results of investigation of the endogenous sources of DNA damage managed by mismatch repair. (A) Substitution and (B) indel signatures for five mismatch repair gene knockouts. The indel signature of ΔPMS1 is shown in panel J. (C) Dissection of DNA mismatch repair mutational signatures: C>A mutations believed to be due to unrepaired oxidative damage of guanine, and proposed mechanism of how DNA polymerase errors cause mis-incorporated bases that result in C>T and T>C. All other mismatch possibilities and their outcomes are demonstrated in Figure S10 The red and black strands represent lagging and leading strands, respectively. The arrowed strand is the nascent strand. (D) Replicative strand asymmetry observed for mutational signatures generated by four MMR gene knockouts. Data are represented as calculated odds ratio with 95% confidence interval. (E) The relative frequency of occurrence of G>T/C>A in polyG tracts for ΔMSH6. The count and relative frequency of occurrence of G>T/C>A in polyG tracts for ΔMSH2 and ΔMLH1 are shown in Figure S12. (F) T>A mutation frequency is highest at junctions of poly(A)poly(T) or poly(T)poly(A). (G) Odds for T>A mutations to occur at poly(A)poly(T) or poly(T)poly(A) are higher than AT sequences flanked by other nucleotides, corrected for sequence context through whole genome. Data are represented as mean±SEM. (H) Putative models of T>A substitutions at poly(A)poly(T) or poly(T)poly(A) junctions due to template strand slippage and slippage reversal. (1) Indel signatures in 186 channels. (J) Indel signature of MMR gene knockouts in 15 channels.

FIG. 9 illustrates the putative outcomes of all possible base-base mismatches. Outcomes from 12 possible base-base mismatches. The red and black strands represent lagging and leading strands, respectively. The arrowed strand is the nascent strand. The highlighted pathways are the ones that generate C>A (blue), C>T (red) and T>C mutations (green) in the ΔMSH2 mutational signature.

FIG. 10 shows a comparison of trinucleotide context of C>A mutations generated by ΔOGG1 and ΔMSH6.

FIG. 11 shows the observed distribution of G>T/C>A mutations in polyG tracts of MSH2, MSH6 and MLH1. (A) Relative frequency of occurrence of G>T/C>A in polyG tracts for ΔMSH2, ΔMSH6 and ΔMLH1. (B) Occurrence of G>T/C>A in polyG tracts for ΔMSH2, ΔMSH6 and ΔMLH1.

FIG. 12 shows the proportion of different mutation types of substitution (A) and indel (B) signatures for 4 MMR gene knockouts. (C) The ratio of substitution and indel burden. (D) Schematic interpretation of the relative mutation burdens of ΔMSH2 and ΔMSH6.

FIG. 13 shows results illustrating gene-specific characteristics of mutational signatures of MMR-deficiency. (A) MMR knockouts demonstrate consistent gene-specificity regardless of model system, e.g., cancer (in vivo) and CMMRD patient-derived hiPSCs (in vitro). Whole-genome plots are shown for two patient-derived hiPSCs and two cancer samples. CMMRD77 is a PMS2-mutant patient. CMMRD89 is an MSH6-mutant patient. PD11365a and PD23564a are breast tumors with PMS2 deficiency and MSH2/MSH6 deficiency, respectively. Genome plots show somatic mutations including substitutions (outermost, dots represent six mutation types: C>A, blue; C>G, black; C>T, red; T>A, grey; T>C, green; T>G, pink), indels (the second outer circle, colour bars represent five types of indels: complex, grey; insertion, green; deletion other, red; repeat-mediated deletion, light red; microhomology-mediated deletion, dark red) and rearrangements (innermost, lines representing different types of rearrangements: tandem duplications, green; deletions, orange; inversions, blue; translocations, grey). (B) Hierarchical clustering of cancer-derived tissue-specific MMR signature and MMR knockout signatures. 96-bar plots of ΔPMS2-related tissue-specific signatures can be viewed here: https://signal.mutationalsignatures.com/explore/cancer/consensusSubstitutionSignatures/6.

FIG. 14 shows mutational profiles of hIPSCs derived from patients with Constitutional MisMatch Repair Deficiency (CMMRD). (A) Experimental workflow used to generate hiPSCs from CMMRD patients, subcloning of hiPSCs and whole-genome sequencing. (B) Genome plots. Top: genome plots of four iPS cells from two PMS2 mutant patients. Bottom: genome plots of three iPS cells derived from two MSH6 mutant patients. Genome plots show somatic mutations including substitutions (outermost, dots represent six mutation types: C>A, blue; C>G, black; C>T, red; T>A, grey; T>C, green; T>G, pink), indels (the second outer circle, colour bars represent five types of indels: complex, grey; insertion, green; deletion other, red; repeat-mediated deletion, light red; microhomology-mediated deletion, dark red) and rearrangements (innermost, lines representing different types of rearrangements: tandem duplications, green; deletions, orange; inversions, blue; translocations, grey). (C) Substitution profiles. (D) Indel profiles.

FIG. 15 shows the distribution of the five parameters across IHC-determined MMR gene abnormal (orange) and MMR gene normal (green) samples. (A) Exposure of MMR signatures. (B) Cosine similarity between the substitution profile of cancer samples and that of MMR gene knockouts. (C) Number of indels in repetitive regions. (D) Cosine similarity between the profile of repeat-mediated deletions of cancer sample and that of knockout generated indel signatures, (E) the cosine similarity between the profile of repeat-mediated insertion of cancer sample and that of knockout generated indel signatures. P-values were calculated through Mann-Whitney test.

FIG. 16 shows the distribution of coefficients from 10-fold cross validation using training data set.

FIG. 17 shows MMRDetect-calculated probabilities for 336 colorectal cancers. With cutoff of 0.7, 77 out of 336 were predicted to be MMR-deficient samples (probability <0.7). Color bars represent the MSI status determined by IHC staining: red—abnormal; blue—normal. 4 samples with abnormal IHC staining have probabilities >0.7, whilst 2 samples with normal IHC staining have probabilities <0.7. The 4 samples were revealed to be false positive cases and the 2 samples were false negative ones for IHC staining through validation using MSIseq and seeking coding mutations in MMR genes.

FIG. 18 shows the distribution of the mutation number of repeat-mediated indels, MMR-deficiency signatures and non-MMR-deficiency signatures across four groups of samples: MMR-deficient samples determined by only MMRDetect, MMR-deficient samples determined by only MSIseq, MMR-deficient samples determined by both MMRDetect and MSIseq and non-MMR-deficient samples determined by both MMRDetect and MSIseq. P-values were calculated through Mann-Whitney test.

FIG. 19 shows the results of a mutational signature-based mismatch repair(MMR) deficiency classifier, MMRDetect disclosed herein. (A) Concordance of three MMR-deficiency detection methods—immunohistochemistry (IHC) staining, MSIseq and MMRDetect—on 336 colorectal cancers is illustrated in the Venn diagram. Details of the eight samples with discordant outcomes from the three methods are provided in the table. Four samples classified as MMR-proficient by MMRDetect and MSIseq have abnormal IHC staining (highlighted in dark yellow). However, no functional mutations in MMR genes were found. Two samples classified as MMR-proficient by MMRDetect and IHC staining were identified as MMR-deficient by MSIseq (highlighted in pink) and did not have MMR gene mutations but had POLE mutations and signatures instead. Two samples classified as MMR-deficient by MMRDetect and MSIseq have normal IHC staining (highlighted in orange). Both have mutations in MMR genes. (B) Receiver operating characteristic (ROC) curves of IHC staining, MMRDetect and MSIseq classification. (C) Concordance between MSIseq and MMRDetect on 2012 GEL colorectal cancers, 713 GEL uterine cancers, 2024 Hartwig metastatic cancers and 2610 cancers from PCAWG & SCANB projects. The bars show the numbers of samples that were identified as MMR deficient by only MSIseq (pink), only MMRDetect (blue), both (yellow) and none (purple). (D) The distribution of three variables amongst samples that were discordantly (blue, pink) and concordantly (yellow and purple) detected by MSIseq and MMRDetect: the number of repeat-mediated indels, number of mutations associated with MMRD signatures and non MMRD mutations.

FIG. 20 illustrates schematically the impact of experimental validation of cancer-derived mutational signatures on biological understanding and development of clinical applications. Some genes (often involved in DNA repair pathways) which are important guardians against endogenous DNA damage under non-malignant circumstances, have been identified in this work. They help to validate and to understand the etiologies of cancer-derived mutational signatures. The biological insights help to drive the development of new genomic clinical tools to detect these abnormalities with greater accuracy and sensitivity across tumor types.

FIG. 21 shows the results of a pilot study performed using three genes for knockout (Δ): MSH6, UNG and ATP2B4 (negative control). (A) Substitution burden for knockouts of ATP2B4, UNG and MSH6 under hypoxic and normoxic conditions as well as different culturing time. (B) The cosine similarities between the mutational profile of each subclone and background signature of culture. (C) Indel burden for knockouts of ATP2B4, UNG and MSH6 under hypoxic and normoxic conditions as well as different culturing time. (D) The cosine similarities between the mutational profile of each subclone with background signature of culture.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a cell or tissue sample (e.g. a biopsy), a biological fluid, an extract (e.g. a protein or DNA extract obtained from the subject), from which genomic material can be obtained for genomic analysis, such as genomic sequencing (whole genome sequencing, whole exome sequencing, targeted (also referred to as “panel”) sequencing). In particular, the sample may be a blood sample, or a tumour sample. The sample may be one which has been freshly obtained from a subject or may be one which has been processed and/or stored prior to making a determination (e.g. frozen, fixed or subjected to one or more purification, enrichment or extractions steps). In particular, the sample may be a cell or tissue culture sample. As such, a sample as described herein may refer to any type of sample comprising cells or genomic material derived therefrom, whether from a biological sample obtained from a subject, or from a sample obtained from e.g. a cell line. The sample is preferably from a mammalian (such as e.g. a mammalian cell sample or a sample from a mammalian subject, including in particular a model animal such as mouse, rat, etc.), preferably from a human (such as e.g. a human cell sample or a sample from a human subject). Further, the sample may be transported ad/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider).

A “tumour sample” refers to a sample that contains tumour cells or genetic material derived therefrom. The tumour sample may be a cell or tissue sample (e.g. a biopsy) obtained directly from a tumour. A tumour sample may be a sample that comprises tumour cell or genetic material derived therefrom, that has not be obtained directly from a tumour. For example, a tumour sample may be a sample comprising circulating tumour cells or circulating tumour DNA. Thus, a tumour sample may also be a biological fluid (e.g. a liquid biopsy such as a blood, urine, or cerebrospinal fluid biopsy). A sample comprising a mixture of tumour cells and other cells (or material genetic derived therefrom) may be subject to one or more processing steps, whether prior to or subsequent to the acquisition of sequence data, in order to identify sequence data that is representative of the genetic material from the tumour. For example, a sample comprising cells may be subject to one or more cell purification steps which selectively enrich the sample for tumour cells. Similarly, a sample comprising modified and non-modified cells can be subject to one or more purification or selection steps to enrich the sample for modified cells. Protocols for doing this are known in the art. As another example, a sample of genetic material may be subject to one or more capture and/or size selection steps to selectively enrich the sample for tumour-derived genetic material. Protocols for doing this are known in the art. As another example, sequence data may be subject to one or more filtering steps (e.g. based on fragment length) to enrich the data for information that relates to tumour-derived genetic material. Protocols for doing this are known in the art.

A “normal sample” (also referred to as “germline sample” or “parent sample”) refers to a sample that contains non-tumour or non-modified cells or genetic material derived therefrom. A normal sample may be matched to a particular tumour or modified sample in the sense that it is obtained from the same biological source (subject or cell line) as the tumour or modified sample. A normal sample may be a cell or tissue sample obtained from a subject, or a sample of biological fluid. A sample comprising a mixture of normal cells and other cells (or material genetic derived therefrom) may be subject to one or more processing steps, whether prior to or subsequent to the acquisition of sequence data, in order to identify sequence data that is representative of the genetic material from the normal cells (as already described above). For example, a sample comprising modified and non-modified cells can be subject to one or more purification or selection steps to enrich the sample for non-modified cells. Similarly, a sample comprising normal and tumour-derived cells can be subject to one or more purification steps which selectively enrich the sample for normal cells.

The term “sequence data” refers to information that is indicative of the presence and/or amount of genomic material in a sample that has a particular sequence. Such information may be obtained using sequencing technologies, such as e.g. next generation sequencing (NGS, such as e.g. whole exome sequencing (WES), whole genome sequencing (WGS), or sequencing of captured genomic loci (targeted or panel sequencing)), or using array technologies, such as e.g. SNP arrays, or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location. Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling”, and can be performed using methods known in the art (such as e.g. the GATK HaplotypeCaller, https://gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

The term “mutation” refers to a difference in a nucleotide sequence (e.g. DNA or RNA) in a sample compared to a reference. For example, a mutation may be a single nucleotide variant (SNV), multiple nucleotide variants, a deletion mutation, an insertion mutation, a translocation, a missense mutation, a translocation, a fusion, etc. Mutations may be identified using sequence data. An “indel mutation” (or simply “indel”) refers to an insertion and/or deletion of bases in a nucleotide sequence (e.g. DNA or RNA) of an organism.

Within the context of the present invention, a mutation is typically a somatic mutation, unless the context indicates otherwise. A “somatic mutation” is a mutation that is present in a tumour or modified cell (or genetic material derived therefrom), but not in a corresponding (matched) normal or non-modified cell.

The present invention relates broadly to the identification of MMR deficiencies. A cell (or by extension, a tissue, tumour or subject comprising such a cell) may be referred to as “MMR-deficient” if it has one or more alterations that impair the function of the mismatch repair pathway. The alteration may be genetic (e.g. a mutation of any kind in one or more genes of the MMR pathway) or epigenetic (e.g. direct or indirect epigenetic silencing of one or more genes of the MMR pathway) or post-translational through complex interactions between multiple proteins. The alteration may directly affect a gene in the MMR pathway, or may indirectly affect a gene in the MMR pathway (for example by directly affecting a gene that is not in the MMR pathway but which, if impaired, affects the function of the MMR pathway, by physical or functional interaction). For example, alteration of the function of a gene in DNA repair pathway different from the MMR pathway may alter the function of the MMR pathway as a knock-on effect.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

As used herein “treatment” refers to reducing, alleviating or eliminating one or more symptoms of the disease which is being treated, relative to the symptoms prior to treatment.

The systems and methods described herein may be implemented in a computer system, in addition to the structural components and user interactions described. As used herein, the term “computer system” includes the hardware, software and data storage devices for embodying a system or carrying out a method according to the above described embodiments. For example, a computer system may comprise a central processing unit (CPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

Prediction of DNA from a Tumour Sample as MMR Deficient or Proficient

In embodiments of the present invention, a prediction of whether a DNA sample from a tumour of a patient is MMR proficient or deficient is performed. In these embodiments, this prediction is performed by a computer-implemented method or tool that takes as its inputs sequence data from the sample or the values of one or more mutational signature metrics derived therefrom, and produces as output a probabilistic score indicative of whether the sample is MMR proficient or deficient, or information derived therefrom such as a classification of the sample as likely MMR deficient/unlikely MMR deficient.

In a development of this embodiment, the computer-implemented method or tool may take as its inputs a list of somatic mutations generated from sequence data associated with a tumour sample (such as e.g. sequencing data obtained from genomic material from fresh-frozen derived DNA, circulating tumour DNA or formalin-fixed paraffin-embedded (FFPE) DNA representative of a suspected or known tumour from a patient). These somatic mutations can then be analysed to determine the value(s) of the one or more mutational signature metrics.

In a development of this embodiment, the computer-implemented method or tool may take as its inputs sequence data associated with a tumour sample, and may use this data to generate a list of somatic mutations. These somatic mutations can then be analysed to determine the value(s) of the one or more mutational signature metrics. A list of somatic mutation may be obtained by identifying mutations present in sequence data associated with a tumour sample, and removing or otherwise excluding mutations that are present or assumed to be present in a corresponding germline genome. Mutations that are present in a corresponding germline genome may be identified by identifying the mutations present in a germline sample obtained from the same subject (also referred to as a “matched germline” or “matched normal” sample). Thus, the computer-implemented method or tool may further take as input sequence data associated with a matched germline sample. Mutations that are assumed to be present in a corresponding germline genome may be identified by identifying mutations that are present in a reference genome or set of reference genomes. A reference genome or set of reference genomes may be obtained from one or more reference samples that are not (or not all) matched normal samples. For example, the reference sample(s) may be process matched, or may comprise a plurality of normal (i.e. non-tumour/non-modified) samples not all of which are matched to the sample for which a somatic mutational profile is determined (e.g. pooled normal samples may be used as references for a plurality of tumour samples). A reference genome or set of reference genomes may be obtained from one or more databases.

A list of somatic mutations may comprise mutations of one or more types selected from: substitutions, deletions, and insertions. A list of somatic substitutions associated with a sample or a group of samples may be referred to as a “substitution profile”. A list of somatic deletions associated with a sample or a group of samples may be referred to as a “deletion profile”. A list of somatic insertions associated with a sample or a group of samples may be referred to as a “insertion profile”. A list comprising both somatic insertions and deletions associated with a sample or group of samples may be referred to as an “indel profile”. An insertion or deletion may be referred to as “repeat mediated” if it occurs in a repetitive region. A repetitive region may be defined as a region that includes a plurality (e.g. 2 or more) of repeats of a sequence motif. A sequence motif may be defined as a sequence of between 1 and n bases, where n may be selected as 3, 4, 5, 6, 7, 8, 9, 10, 11, or 12. For example n=9 may be convenient. The use of higher values of n requires more extensive cataloguing of such regions, which may be associated with diminishing returns as repeats of longer motifs are less likely. A repetitive region may be defined by reference to a reference genome. In other words, a repetitive region may be defined as a particular locus (defined by its genomic coordinates) in a reference genome. Thus, any mutation identified within such a locus may be considered to be “repeat mediated”.

In some embodiments, the present invention provides methods for classifying samples from tumours between classes that are associated with different likelihoods of MMR deficiency. In particular, mutational signature metrics may be evaluated using one or more pattern recognition algorithms. Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from samples of known subgroup (e.g., from subjects known to have a MMR deficient or MMR proficient tumour), and second to classify an unknown sample (e.g., “test sample”) according to subgroup. Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyse data, and hence to classify samples and to predict the value of some dependent variable based on a range of observed measurements. In the context of the present invention, “supervised” approaches are suitably used, whereby a training set of samples with known class or outcome is used to produce a mathematical model which is then evaluated with independent validation data sets. Here, a “training set” of gene expression data is used to construct a statistical model that predicts correctly the “subgroup” of each sample. This training set is then tested with independent data (referred to as a test or validation set) to determine the robustness of the computer-based model. These models may be based on a range of different mathematical procedures such as logistic regression models, support vector machine, decision trees, k-nearest neighbour and naïve Bayes classifiers. The robustness of the predictive models can for example be checked using cross-validation, by leaving out selected samples from the analysis.

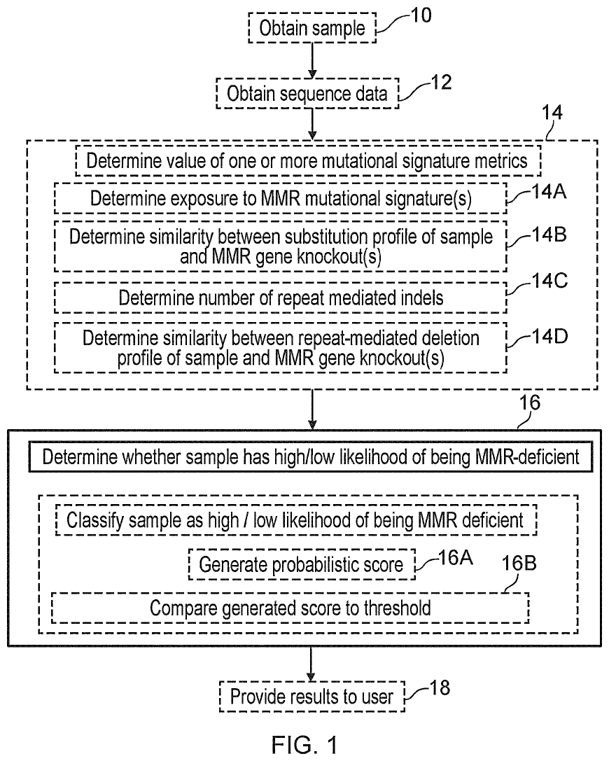

FIG. 1 is a flow diagram showing, in schematic form, a method of characterising a DNA sample according to the disclosure. At optional step 10, a DNA sample is obtained from a tumour of a subject. Optionally, a matched normal sample may also be obtained from the subject. At optional step 12, sequence data is obtained from the tumour (and optionally the matched normal) DNA sample(s). At optional step 14, the value of one or more mutational signature metrics for the tumour DNA sample is/are obtained. This may comprise obtaining a catalogue of somatic mutations in the tumour DNA, for example by identifying somatic mutations in the tumour DNA and counting the number of mutations of a plurality of types (also referred to as “mutation channels”. The types of mutations catalogued may comprise substitutions, deletions, insertions, and subsets (e.g. different trinucleotide substitutions, different lengths of indels, different indel contexts, etc.)/supersets (e.g. indels) thereof. The mutational catalogue is also referred to herein as “mutational profile”. The mutational profile may then be used to determine the exposure to one or more MMR mutational signatures at step 14A, to determine the similarity between the substitution profile of the sample and that of one or more MMR gene knockouts at step 14B, to determine the number of repeat mediated indels in the sample at step 14C, and/or to determine the similarity between the repeat-mediated deletion profile of the sample and that of one or more MMR gene knockouts at step 14D. Steps 10-14 are optional because the method may start from sequence data, from a mutational profile associated with the sample, or directly from the (previously determined) value of the one or more mutational signature metrics described above.

The one or more mutational signature metrics may be selected from: the exposure to one or more MMR mutational signatures (E_{MMRD}), the similarity between the substitution profile of the sample and that of one or more MMR gene knockout(s) (S_{sub}), the number of repeat mediated indels (N_{rep.indel}), and the similarity between the repeat-mediated deletion profile of the sample and that of one or more MMR gene knockout(s) (S_{rep.del}).

Methods for determining the exposure to a mutational signature are known in the art (see e.g. Alexandrov et al., 2020; Degasperi et al., 2020; Fantini et al., 2020; Gehring et al., 2015). In particular, the determination of the exposure to one or more mutational signatures may be performed by identifying the matrix E that satisfies C≈PE where C is a mutational catalogue for one or more samples for which exposure is to be determined, P is a signature matrix comprising the one or more mutational signatures for which exposure is to be determined, and E is an exposure matrix The determination of the exposure to one or more mutational signatures may be performed as described in Degasperi et al., 2020.

The one or more MMR mutational signatures may be selected from MMR1, MMR2, or any corresponding tissue specific signatures as described in Degasperi et al., 2020 (and available at https://signal.mutationalsignatures.com/explore/study/1), SBS6, SBS14, SBS15, SBS20, SBS21, SBS26, or ID7 as described in Alexandrov et al., 2020 (and available at https://cancer.sanger.ac.uk/cosmic/signatures/). In general, any mutational signature that has been mechanistically or phenotypically associated with MMR deficiency may be used as an MMR mutational signature. A mutational signature may have been mechanistically associated with MMR if it has been identified in cells that are known to have one or more impairment (e.g. one or more natural or engineered molecular impairment) that lead to MMR deficiency, or if it is more similar than expected by chance to a signature that has been derived from cells that are known to have one or more impairments that lead to MMR deficiency (e.g. a signature that is more similar than expected by chance to a mutational signature derived from a MMR knockout sample). For example, a mutational signature that is enriched (e.g. associated with comparatively strong exposure values) in cells that are known to be MMR deficient (e.g. cancer cells that are known to be MMR deficient) may be a suitable MMR mutational signature. A mutational signature may have been phenotypically associated with MMR deficiency if it is enriched in mutation types that are known hallmarks of MMR deficiency (e.g. small (e.g. 1 bp) insertions and deletions of T at mononucleotide T repeats, C>T substitutions, T>C substitutions) and/or if it is frequently identified in cells that have a phenotype indicative of MMR deficiency, such as e.g. cells that are microsatellite unstable. For example, mutational signatures that are often found (more often than expected by chance and/or more often than other signatures) in samples that are microsatellite unstable may be phenotypically associated with MMR deficiency and may be used as MMR mutational signatures.

The determination of the similarity between two mutation profiles may be performed by calculating the cosine similarity between the two mutation profiles. The cosine similarity between two mutation profiles can be calculated as:

$$sim ( S , M ) = \frac{S . M}{‖ S ‖ ‖ M ‖}$$

where S and M are equally-sized vectors with nonnegative components being the respective mutation profiles (e.g. S being that of a sample and M that of a reference knockout profile).

The method may further comprise receiving (for example from a user, through a user interface, or from one or more databases) one or more of: one or more mutational signature(s) of MMR, and a mutation profile (e.g. substitution profile and/or repeat mediated deletion profile) of one or more MMR gene knockouts or gene knockout samples.

The mutational profile of an MMR gene knockout is a mutational profile derived from one or more MMR gene knockout samples. The term “MMR gene knockout sample” refers to any sample of cells or genetic material derived therefrom, in which the function of one or more genes of the MMR pathway is impaired. Any manipulation that impairs the function of at least one MMR gene may therefore result in an MMR gene knockout cell. Such a manipulation may directly affect a gene in the MMR pathway, or may affect a gene in another pathway, indirectly affecting the function of the MMR pathway. In embodiments, an MMR gene knockout sample has one or more alterations that directly affect the function of a gene in the MMR pathway. Such an alteration may be genetic or epigenetic. In embodiments, an MMR gene knockout has one or more alterations that indirectly affect the function of a gene in the MMR pathway. For example, the function of a gene in the MMR pathway may be affect post-translationally through complex interactions with multiple proteins, at least one of these interactions having been impaired by directly impairing the gene coding for a protein involved in the interaction. For example, an MMR gene knockout cell (or cell line) may be a cell in which one or more genes of the MMR pathway has been silenced, mutated, downregulated or knocked out. Techniques for performing such manipulations are known in the art. In embodiments, an MMR gene knockout sample is a sample of cells or genetic material derived therefrom, in which one or more genes in the MMR pathway has been knocked out, for example using CRISPR-Cas9. An MMR gene may be selected from MSH2 (Homo sapiens Gene ID: 4436, or a homologue thereof), MSH6 (Homo sapiens Gene ID:2956, or a homologue thereof), MSH3 (Homo sapiens Gene ID: 4437, or a homologue thereof), MLH1 (Homo sapiens Gene ID:4292, or a homologue thereof), PMS1 (Homo sapiens Gene ID:5378, or a homologue thereof) or PMS2 (Homo sapiens Gene ID:5395, or a homologue thereof). In embodiments, the one or more MMR genes are selected from MSH2, MSH6, MLH1, PMS2, and PMS1. In embodiments, an MMR gene knockout sample is a sample of cells or genetic material derived therefrom, in which the function of a single gene in the MMR pathway is impaired. A gene knockout sample may be a sample of mammalian cells, suitably human cells, or genetic material derived therefrom.

At step 16, it is determined whether the sample has a high or low likelihood of being MMR deficient, based on the value of the one or more signature metrics received or determined at step 14. This may optionally be performed by classifying the sample between at least two classes, a first class associated with a high likelihood of being MMR deficient, and a second associated with a low likelihood of being MMR deficient. Such as classification may be performed by generating a probabilistic score at step 16A using the value(s) of the one or more mutational signature metrics or values derived therefrom (such as e.g. by normalisation), and comparing the score thus obtained at step 16B to one or more predetermined thresholds that define the boundary(ies) of the first and second classes. At step 18, one or more results of this analysis may optionally be provided to a user through a user interface.

Uses of Predictor Outcome

A prediction of whether a tumour is likely to be MMR deficient can be used in the treatment of cancer. Thus, the invention also provides a method of treating cancer in a subject, wherein the method comprises administering or recommending a subject for administration of a particular therapy, depending on whether a tumour of the subject is identified as likely to be MMR deficient. FIG. 3 illustrates a method of providing a prognosis and/or treating a subject that has been diagnosed with cancer, according to embodiments described herein. The method may comprise optional step 30 of obtaining a DNA sample from a tumour of a subject. Optionally, a matched normal sample may also be obtained from the subject. The step of obtaining a sample from a subject may comprise physically obtaining the sample from the subject. Alternatively, the sample may have been previously obtained and no interaction with the subject may be required. In other words, obtaining a DNA sample may comprise receiving a previously acquired DNA sample. At optional step 32, sequence data is obtained from the tumour (and optionally the matched normal) DNA sample(s). The step of obtaining sequence data from a DNA sample may comprise sequencing the DNA sample. Alternatively, sequence data may have been previously obtained. Thus, obtaining sequence data may comprise receiving the data from one or more databases, or from a user through a user interface. At step 34, it is determined whether the tumour sample has a low or high likelihood of being MMR deficient, using methods described herein such as e.g. by reference to FIG. 1. Based on this determination, the subject may be classified as having a good or poor prognosis at step 36A (as will be explained further below). Instead or in addition to this, the subject may be classified at step 36B as being likely to respond or unlikely to respond to a particular course of treatment, where responder/non-responder status is known to be associated with MMR-deficiency (i.e. tumours that are MMR-deficient are known to be more or less likely to respond to the particular course of treatment, compared to tumours that are not MMR deficient). At optional step 38, a particular course of treatment (which may comprise one or more different individual therapies) may be identified based on the results of step 36B. For example, a subject that has been identified at step 36B as unlikely to respond to the particular course of therapy may be identified as likely to benefit from a therapy that is different from the particular course of therapy. Alternatively, a subject that has been identified at step 36B as likely to respond to the particular course of therapy may be identified as likely to benefit from a therapy that includes the particular course of therapy. At optional step 40, the subject may be treated with the therapy identified at step 40.

In particular, MMR deficient cancers have been identified as having an increased likelihood of response to immunotherapy, and particularly checkpoint inhibitors (CPI) (see e.g. Zhao, Jiang & Li, 2019). CPI therapy includes for example treatment with an anti-CTL4 or anti-PD(L)1 drug. Thus, also described herein are methods of determining whether a subject that has been diagnosed as having a cancer is likely to benefit from treatment with an immunotherapy, preferably a CPI therapy, the method comprising determining the MMR status of a tumour from the subject using the methods described herein. The method may further comprise classifying the subject between a group that is likely to respond to CPI therapy, and a group that is not likely to respond to CPI therapy. For example, the method may comprise determining whether a sample from a tumour of the subject has a high or low likelihood of being MMR deficient (as explained above). A subject may then be classified in the group that is not likely to respond to CPI therapy if the sample is determined to have a low likelihood of being MMR deficient, and in a group that is likely to respond to CPI therapy otherwise. Alternatively, a subject may be classified in the group that is not likely to respond to CPI therapy if the likelihood of MMR deficiency (e.g. as captured in a probabilistic score as described above) is below a threshold, and in the group that is likely to respond to CPI therapy otherwise.

In some cases CPI therapy may comprise CTLA-4 blockade (cytotoxic T-lymphocyte associated protein 4, Gene ID:1493), PD-1 inhibition (PDCD1, programmed cell death 1, Gene ID:5133), PD-L1 inhibition (CD274, CD274 molecule, Gene ID: 29126), Lag-3 (Lymphocyte activating 3; Gene ID: 3902) inhibition, Tim-3 (T cell immunoglobulin and mucin domain 3; Gene ID: 84868) inhibition, TIGIT (T cell immunoreceptor with Ig and ITIM domains; Gene ID: 201633) inhibition and/or BTLA (B and T lymphocyte associated; Gene ID: 151888) inhibition. The CPI therapy may be an anti-PD1 or anti-PDL1 therapy (also referred to as anti-PD(L)1 inhibitor). The inhibitor may be a therapeutic antibody. For example, the CPI therapy may be a PD-1 inhibitor such as pembrolizumab, nivolumab, or tislelizumab. Pembrolizumab is a therapeutic antibody that has been approved by the FDA (U.s>Food and Drug Administration) for patients with unresectable or metastatic microsatellite instability-high (MSI-H) or mismatch repair deficient (dMMR) solid tumors that have progressed following prior treatment. This indication is independent of PD-L1 expression assessment, tissue type and tumor location. Nivolumab is a therapeutic antibody used to treat various cancers including melanoma, lung cancer, renal cell carcinoma, Hodgkin lymphoma, head and neck cancer, colon cancer, and liver cancer. Tislelizumab is a therapeutic antibody under investigation for the treatment of advanced solid tumours. The CPI therapy may be a PDL-1 (also referred to as “PD-L1”) inhibitor such as atezolizumab, avelumab, or durvalumab. Atezolizumab is a therapeutic antibody used to treat urothelial carcinoma, non-small cell lung cancer (NSCLC), triple-negative breast cancer (TNBC), small cell lung cancer (SCLC), and hepatocellular carcinoma (HCC). It was the first PD-L1 inhibitor approved by the FDA. Avelumab is a therapeutic antibody used for the treatment of Merkel cell carcinoma, urothelial carcinoma, and renal cell carcinoma. Durvalumab is a therapeutic antibody that has been approved by the FDA for the treatment of certain types of bladder and lung cancer. As another example, the CPI therapy may be a CTLA-4 inhibitor, such as ipilimumab or tremelimumab. Ipilimumab is a therapeutic antibody approved by the FDA for the treatment of melanoma, and under investigation for the treatment of non-small cell lung cancer, small cell lung cancer, bladder cancer and metastatic hormone-refractory prostate cancer. Tremelimumab is a therapeutic antibody under investigation for the treatment of melanoma, mesothelioma and non-small cell lung cancer.

Further, MMR deficient cancers have been identified as having a decreased likelihood of response to fluorouracil based treatment (e.g. adjuvant 5-fluorouracil chemotherapy) and/or an increased likelihood of response to non-fluorouracil based treatments (Devaud & Gallinger, 2013; Jover et al., 2009). Thus, also described herein are methods of determining whether a subject that has been diagnosed as having a cancer is likely to benefit from treatment with chemotherapy, preferably a fluorouracil based therapy or a non-fluorouracil based therapy, the method comprising determining the MMR status of a tumour from the subject using the methods described herein. Such a method may further comprise classifying the subject between a group that is likely to respond to fluorouracil based therapy, and a group that is not likely to respond to fluorouracil-based therapy. For example, the method may comprise determining whether a sample from a tumour of the subject has a high or low likelihood of being MMR deficient (as explained above). A subject may then be classified in the group that is likely to respond to fluorouracil-based therapy if the tumour is determined to have a low likelihood of being MMR deficient, and in a group that is not likely to respond to fluorouracil-based therapy otherwise. Alternatively, a subject may be classified in the group that is not likely to respond to fluorouracil-based therapy if the likelihood of MMR deficiency (e.g. as captured in a probabilistic score as described above) is above a threshold, and in the group that is likely to respond to fluorouracil-based therapy otherwise.

Alternatively, such a method may comprise classifying the subject between a group that is likely to respond to non-fluorouracil based therapy, and a group that is not likely to respond to no-fluorouracil-based therapy. For example, the method may comprise determining whether a sample from a tumour of the subject has a high or low likelihood of being MMR deficient (as explained above). A subject may then be classified in the group that is likely to respond to non-fluorouracil-based therapy if the tumour is determined to have a high likelihood of being MMR deficient, and in a group that is not likely to respond to non-fluorouracil-based therapy otherwise. Alternatively, a subject may be classified in the group that is not likely to respond to non-fluorouracil-based therapy if the likelihood of MMR deficiency (e.g. as captured in a probabilistic score as described above) is below a threshold, and in the group that is likely to respond to fluorouracil-based therapy otherwise.

Any treatment described herein may be used alone or in combination with another treatment. For example, any treatment with a drug may be used in combination with one or more chemotherapies, one or more course of radiation therapy, and/or one or more surgical interventions. In particular, any treatment described herein may be used in combination with a treatment for which the subject has been identified as likely to be responsive. For example, a subject may be identified as likely to be deficient for homologous recombination (HRdeficient) using one or more methods known in the art. Such a subject may be treated or identified as likely to benefit from treatment with a PARP inhibitor or platinum-based drug. For example, a subject may be identified as likely to be HR-deficient using the methods described in WO 2018/115452 or WO 2017/191074, or likely to respond to a PARP inhibitor or a platinum-based drug using the methods described in WO 2017/191073. As a particular example, a method of treating a subject that has been diagnosed as having cancer may comprise: determining whether the subject is likely to benefit from treatment with an immunotherapy, preferably a CPI therapy, the method comprising determining the MMR status of a tumour from the subject using the methods described herein; and determining whether the subject is likely to benefit from treatment with a PARP inhibitor or platinum based therapy, the method comprising determining the HR status of a tumour from the subject, for example using the methods described in WO 2018/115452 or WO 2017/191074. Such a method may further comprise treating the subject with an immunotherapy (e.g. a CPI therapy, such as a PD1/PDL1 inhibitor) if the subject has been identified as likely to be MMR deficient, and/or treating the subject with a PARP inhibitor or platinum-based therapy if the subject has been identified as likely to be HR deficient.

Additionally, the MMR status of a tumour has been shown to be associated with different prognosis in cancer (see e.g. Sinicrope, 2009). For example, MMR deficient tumours have been associated with improved prognosis compared to non-MMR deficient tumours, for example in terms of disease free survival and overall survival. Thus, also described herein are methods of providing a prognosis for a subject that has been diagnosed as having a cancer, the method comprising determining the MMR status of a tumour from the subject. The method may further comprise classifying the subject between a group that has good prognosis, and a group that has poor prognosis. For example, the method may comprise determining whether a sample from a tumour of the subject has a high or low likelihood of being MMR deficient (as explained above). A subject may then be classified in the group that has poor prognosis if the sample is determined to have a low likelihood of being MMR deficient, and in a group that has good prognosis otherwise. Alternatively, a subject may be classified in the group that has poor prognosis if the likelihood of MMR deficiency (e.g. as captured in a probabilistic score as described above) is below a threshold, and in the group that has good prognosis otherwise.

Whether a prognosis is considered good or poor may vary between cancers and stage of disease. In general terms a good prognosis is one where the overall survival (OS), disease free survival (DFS) and/or progression-free survival (PFS) is longer than that of a comparative group or value, such as e.g. the average for that stage and cancer type. A prognosis may be considered poor if OS, DFS and/or PFS is lower than that of a comparative group or value, such as e.g. the average for that stage and type of cancer. Thus, in general terms, a “good prognosis” is one where survival (OS, DFS and/or PFS) and/or disease stage of an individual patient can be favourably compared to what is expected in a population of patients within a comparable disease setting. Similarly, a “poor prognosis” is one where survival (OS, DFS and/or PFS) of an individual patient is lower (or disease stage worse) than what is expected in a population of patients within a comparable disease setting.

The subject is preferably a human patient.

The cancer may be any cancer that may be MMR deficient. In particular, the methods described herein may be used to characterise any type of cancer that is known to have MMR deficient subpopulations or in which MMR deficiencies have been reported in at least some patients. The cancer may be ovarian cancer, breast cancer, endometrial cancer (uterus/womb cancer), kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, gastrointestinal cancer (e.g. colorectal cancer), small bowel cancers (duodenal and jejunal), leukemia, pancreatic cancer, hepatobiliary tumours, germ cell cancers, prostate cancer, head and neck cancers, bladder cancer, thyroid cancer and sarcomas. For example, the cancer may be colorectal cancer, breast cancer, endometrial cancer, breast cancer, prostate cancer, bladder cancer or thyroid cancer, all of which are known to have MMR deficient subpopulations. As another example, the cancer may be colorectal cancer, endometrial/uterus cancer, biliary caner, bone/soft tissue cancer, breast cancer, central nervous system cancer, choroid melanoma, carcinoma of unknown primary (CUP), esophagus cancer, head and neck cancer, kidney cancer, liver cancer, lung cancer, lymphoid cancer, neuroendocrine tumour (NET), ovarian cancer, pancreatic cancer, prostate cancer, skin cancer, stomach cancer, urinary tract cancer. All of these have been tested with the methods described herein. In embodiments, the cancer is colorectal cancer. The links between MMR deficiency and prognosis as well as therapy response in colorectal cancer has been extensively studied and as such there is strong evidence that treatment and prognosis in such caners can be adjusted using information regarding the MMR status of such cancers. Such information is more accurately obtained using the methods described herein, compared to the prior art. As such, the treatment strategy designed for a subject and/or the prognosis provided for a subject having colorectal cancer can be improved using the methods of the present invention.

Systems

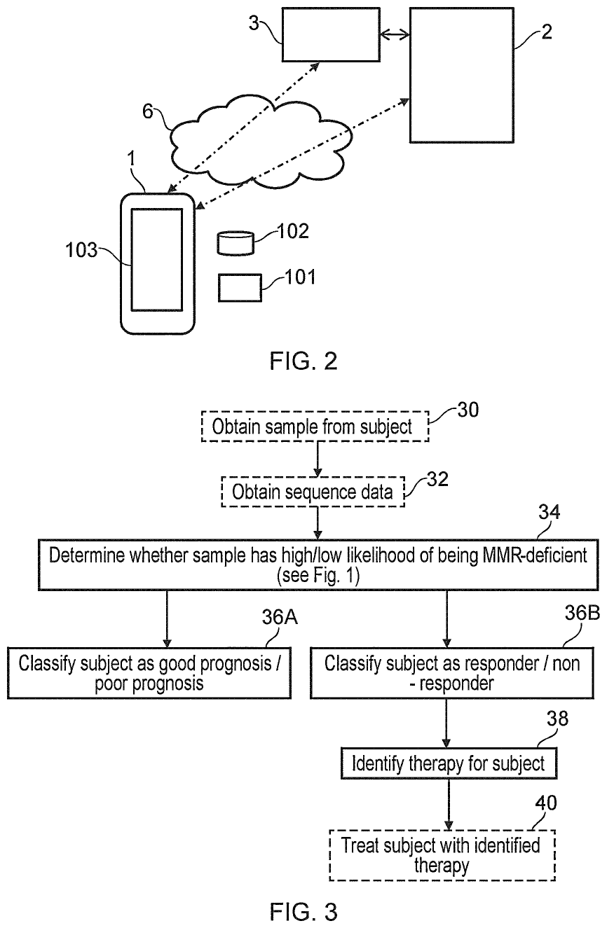

FIG. 2 shows an embodiment of a system for characterising a DNA sample and/or for providing a prognosis or treatment recommendation, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network, to sequence data acquisition means 3, such as a sequencing machine, and/or to one or more databases 2 storing sequence data. The one or more databases 2 may further store one or more of: mutational signatures information, training data, parameters (such as e.g. parameters of a machine learning model used to predict whether a tumour is MMR-deficient, e.g. weights of a logistic regression model, architecture and parameters of a decision tree model, etc.), clinical and/or sample related information, etc. The computing device may be a smartphone, tablet, personal computer or other computing device. The computing device is configured to implement a method for characterising a DNA sample, as described herein. In alternative embodiments, the computing device 1 is configured to communicate with a remote computing device (not shown), which is itself configured to implement a method of characterising a sample, as described herein. In such cases, the remote computing device may also be configured to send the result of the method of characterising a DNA sample to the computing device. Communication between the computing device 1 and the remote computing device may be through a wired or wireless connection, and may occur over a local or public network 6 such as e.g. over the public internet. The sequence data acquisition means may be in wired connection with the computing device 1, or may be able to communicate through a wireless connection, such as e.g. through WiFi and/or over the public internet, as illustrated. The connection between the computing device 1 and the sequence data acquisition means 3 may be direct or indirect (such as e.g. through a remote computer). The sequence data acquisition means 3 are configured to acquire sequence data from nucleic acid samples, for example genomic DNA samples extracted from cells and/or tissue samples. In some embodiments, the sample may have been subject to one or more preprocessing steps such as DNA purification, fragmentation, library preparation, target sequence capture (such as e.g. exon capture and/or panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a genomic alteration profile (whether whole genome or sequence specific) may be used within the context of the present invention. The sequence data acquisition means is preferably a next generation sequencer.

The following is presented by way of example and is not to be construed as a limitation to the scope of the claims.

### EXAMPLES

While there have been advancements in analytical aspects of deriving mutational signatures from human cancers (Haradhvala, N.J. et al., 2018; Alexandrov, L. B. et al., 2020; Kim, J. et al., 2016), there is an emerging need for experimental substantiation, elucidating etiologies and mechanisms underpinning these mutational patterns (Nik-Zainal, S. et al., 2015; Zou, X. et al., 2018; Christensen, S. et al., 2019; Kucab, J. E. et al., 2019). In these examples, the inventors combine CRISPR-Cas9-based biallelic knockouts of a selection of DNA replicative/repair genes in human induced Pluripotent Stem Cells (hiPSCs), whole-genome sequencing (WGS), and in-depth analysis of experimentally-generated data, to obtain mechanistic insights into mutation formation. This work focuses on directly mapping whole-genome mutational outcomes associated with human DNA repair defects, critically, in the absence of any applied, external damage. The insights derived from this are then used to develop a classifier, MMRDetect, for improved clinical detection of MMR-deficient tumors

### Example 1—Biallelic Knockouts of DNA Repair Genes

Methods

Cell lines and culture. The human iPSC line used in this study is previously described (Kucab et al., 2019). The line was derived at the Wellcome Trust Sanger Institute (Hinxton, UK). The use of this cell line model was approved by Proportionate Review Sub-committee of the National Research Ethics (NRES) Committee North West—Liver-pool Central under the project “Exploring the biological processes underlying mutational signatures identified in induced pluripotent stem cell lines (iPSCs) that have been genetically modified or exposed to mutagens” (ref: 14.NW.0129). It is a long-standing iPSC line that is diploid and does not have any known driver mutations. It does carry a balanced translocation between chromosomes 6 and 8. It grows stably in culture and does not acquire a vast number of karyotypic abnormalities. This is confirmed through mutational and copy number assessment of the WGS data reviewed of all subclones.

Cell culture reagents were obtained from Stem Cell Technologies unless otherwise indicated. Cells were routinely maintained on Vitronectin XF-coated plates (10-15 ug/mL) in TeSR-E8 medium. The medium was changed daily, and cells were passaged every 4-8 days depending on the confluence of the plates using Gentle Cell Dissociation Reagent.

All cell lines were grown at 37° C., with 20% oxygen and 5% carbon dioxide in a humidified incubator, except for the pilot study in which the iPSCs knockouts were also grown under hypoxic condition (3% oxygen) as one of the experimental conditions (see “Pilot study” below). Cells were cultivated as monolayers in their respective growth medium and passaged every 3-4 days to maintain sub-confluence during the mutation accumulation step. All cell lines were tested negative for mycoplasma contamination using MycoAlert™ Mycoplasma Detection Kit and LookOut® Mycoplasma PCR Detection Kit according to the manufacturers' protocol.

Generation of DNA repair gene knockouts in human iPSCs. Biallelic DNA repair gene knockouts in human iPSCs were performed by the High Throughput Gene Editing team of Cellular Operations at the Sanger Institute, Hinxton, UK. These knockouts were generated based on the principles of CRISPR/Cas9-mediated HRD and NHEJ as described in Bressan, R. B. et al., 2017.

Generation of donor plasmids for precise gene targeting via HDR. All knockouts were generated using an established protocol that was found to minimize potential off-target effects (Bressan, R. B. et al., 2017). Briefly, the intermediate targeting vectors were generated for each gene using GIBSON assembly of the four fragments: pUC19 vector, 5′ homology arm, R1-pheS/zeo-R2 cassette and 3′ homology arm. Gene-specific homology arms were amplified by PCR from the iPSC gDNA and were either gel-purified or column-purified (QIAquick, QIAGEN). pUC19 vector and R1-pheS/zeo-R2 cassette were prepared as gel-purified blunt fragments (EcoRV digested). Fragments were assembled via GIBSON assembly reactions (Gibson Assembly Master Mix, NEB, E2611) according to the manufacturer's instructions. Assembly reaction mix was transformed into NEB 5-alpha competent cells and clones resistant to carbenicillin (50 μg/mL) and zeocin (10 μg/mL) were analysed by Sanger sequencing to select for correctly-assembled constructs. Sequence-verified intermediate targeting vectors were converted into donor plasmids via a Gateway exchange reaction. LR Clonase II Plus enzyme mix (Invitrogen, 12538120) was used to perform a two-way reaction exchanging only the R1-pheSzeo-R2 cassette with the pL1-EF1αPuro-L2 cassette as previously described⁷⁸. The latter was generated by cloning synthetic DNA fragments of the EF1a promoter and puromycin resistance cassette into one of pL1/L2 vector (Tate, P. H. & Skarnes, W. C., 2011). Following Gateway reaction and selection on yeast extract glucose (YEG)+carbenicillin agar (50 μg/mL) plates, correct donor plasmids were verified by capillary sequencing across all junctions.

Guide RNA design & cloning. For every gene knockout, two separate gRNAs targeting within the same critical exon of a gene were also selected. The gRNAs were selected using the WGE CRISPR tool (Hodgkins, A. et al., 2015) based on their off-target scores. Selected gRNAs were suitably positioned to ensure DNA cleavage within the exonic region, excluding any sequence within the homology arms of the targeting vector. To generate individual gene targeting plasmids, gene-specific forward and reverse oligos were annealed and cloned into BsaI site of either U6_BsaI_gRNA (unpublished). The guide RNA (gRNA) sequences used are listed in Table 1.

Delivery of KO-targeting plasmids, donor templates and Cas9, selection and genotyping. Human iPSCs were dissociated to single cells and nucleofected with Cas9-coding plasmid (hCas9, Addgene 41815), sgRNA plasmid and donor plasmid on Amaxa 4D-Nucleofactor program CA-137 (Lonza). Following nucleofection, cells were selected for up to 11 days with 0.25 μg/mL puromycin. Edited cells were expanded to ˜70% confluency before subcloning. Approximately 1000 cells were subcloned onto 10 cm tissue culture dishes precoated with SyntheMAX substrate (Corning) at a concentration of 5 μg/cm² to allow colony formation for 8-10 days until colonies are approximately 1-2 mm in diameter. Individual colonies were picked into U-bottom 96-well plates using a dissection microscope and a p20 pipette, grown to confluence and then replica plated. Once confluent, the replica plates were either frozen as single cells in 96-well vials or the wells were lysed for genotyping.

To genotype individual clones from a 96-well replica plate, cells were lysed and used for PCR amplification with LongAmp Taq DNA Polymerase (NEB, M0323). Insertion of the cassette into the correct locus was confirmed by visualizing on 1% E-gel (Invitrogen, G700801) PCR products generated by gene-specific (GF1 and GR1) and cassette specific primers ((ER: TGATATCGTGGTATCGTTATGCGCCT and PF: CATGTCTGGATCCGGGGGTACCGCGTCGAG) for both 5′ and 3′ ends. We also confirmed single integration of the cassette by performing a qPCR copy number assay. To check the CRISPR site on the non-targeted allele, PCR products were generated from across the locus, using the same 5′ and the 3′ gene-specific genotyping primers. The PCR products were treated with exonuclease I and alkaline phosphatase (NEB, M0293; M0371) and Sanger sequenced to verify successful knockouts. Sequence reads and their traces were analysed and visualised on a laboratory information management system (LIMS)-2. For each targeted gene, two independently-derived clones with different specific mutations were isolated and studied further.

Genomic DNA extraction and WGS. Samples were quantified with Biotium Accuclear Ultra high sensitivity dsDNA Quantitative kit using Mosquito LV liquid platform, Bravo WS and BMG FLUOstar Omega plate reader and cherry picked to 500 ng/120 μl using Tecan liquid handling platform. Cherry picked plates were sheared to 450 bp using a Covaris LE220 instrument. Post-sheared samples were purified using Agencourt AMPure XP SPRI beads on Agilent Bravo WS. Libraries were constructed (ER, A-tailing and ligation) using ‘Agilent Sureselect kit’ on an Agilent Bravo WS automation system. KapaHiFi Hot start mix and IDT 96 iPCR tag barcodes were used for PCR set-up on Agilent Bravo WS automation system. PCR cycles include 6 standard cycles: 1) Incubate 95° C. 5 mins; 2) Incubate 98° C. 30 secs; 3) Incubate 65° C. 30 secs; 4) Incubate 72° C. 1 min; 5) Cycle from 2, 5 more times; 6) Incubate 72° C. 10 mins. Post PCR plate was purified using Agencourt AMPure XP SPRI beads on Beckman BioMek NX96 liquid handling platform. Libraries were quantified with Biotium Accuclear Ultra high sensitivity dsDNA Quantitative kit using Mosquito LV liquid handling platform, Bravo WS and BMG FLUOstar Omega plate reader, then pooled in equimolar amounts on a Beckman BioMek NX-8 liquid handling platform and finally normalized to 2.8 nM ready for cluster generation on a c-BOT and loading on requested Illumina sequencing platform. Pooled samples were loaded on the X10 using 150 PE run length, sequenced to ˜25× coverage. The details of sequence coverage for all clones and subclones are provided in Table 2.

Alignment and somatic variant-calling. Short reads were aligned to human reference genome GRCh37/hg19 assembly using the BWA-MEM algorithm (Li, H. 2013). Three algorithms, CaVEMan (http://cancerit.github.io/CaVEMan/) (Jones, D. et al., 2016), Pindel (http://cancerit.github.io/cgpPindel) (Raine, K. M. et al., 2015) and BRASS (https://github.com/cancerit/BRASS) were used to call somatic substitutions, indels and rearrangements in all subclones, respectively.

Assurance of knockout state using WGS data. First, we examined whether there were CRISPR-Cas9 off-target effects by seeking relevant mutations in other DNA repair genes besides the genes of interest. We also searched for potential off-target sites based on gRNA target sequences using COSMID (Cradick, T. J. et al., 2014) and confirmed that there were no off-target hits in knockouts that generated mutational signatures. We confirmed chromosome copy number in all subclones remained stable and unchanged from their parent. Second, we confirmed that there are frameshift indels near the gRNA targeted sequence in the genes of interest for all knockout subclones. One UNG knockout was found to be heterozygous and was excluded in the downstream analysis. Third, we checked mislabeled samples by examining the shared mutations between subclones. Subclones originally derived from the same parental knockout clone would share some mutations, in contrast to subclones from different knockouts. Consequently, one ΔPRKDC, one ΔTP53 and two ΔNBN subclones were removed from downstream analysis. Fourth, variant allele fraction (VAF) distribution for each knockout subclone was examined. VAF>=0.4 was used as a cut-off for determination of whether the subclone was derived from a single-cell. When contrasting mutation burden between subclones, we only selected subclones that were derived from single-cells, cultured for 15 days. Shared mutations among subclones were removed to obtain de novo somatic mutations accumulated after knocking out the gene of interest. Table 2 summarizes the number of de novo mutations (substitutions and indels) for all subclones.

Proteomics analysis. Cell pellets were dissolved in 150 μL buffer containing 1% sodium deoxycholate (SDC), 100 mM triethylammonium bicarbonate (TEAB), 10% isopropanol, 50 mM NaCl and Halt protease and phosphatase inhibitor cocktail (100×) (Thermo, #78442) using pulsed probe sonication followed by boiling at 90° C. for 5 min. Aliquots containing 50 μg of total protein, measured with the Coomassie Plus Bradford Protein Assay (Pierce), were reduced with 5 mM tris-2-carboxyethyl phosphine (TCEP) for 1 h at 60° C. and alkylated with 10 mM lodoacetamide (IAA) for 30 min in dark. Proteins were then digested with 75 ng/μL trypsin (Pierce) overnight. The tryptic digests from the ATP2B4, EXO1, OGG1, PMS1, PMS2, RNF168 and UNG knock-out clones as well as three biological replicates of the parental cell line were labelled with the TMTpro 16plex reagents (Thermo) according to manufacturer's instructions. The digests from MLH1, MSH2, MSH6 clones were subjected to label-free single-shot analysis. The TMTpro labelled peptides were fractionated with offline high-pH Reversed-Phase (RP) chromatography (XBridge C18, 2.1×150 mm, 3.5 μm, Waters) on a Dionex Ultimate 3000 HPLC system with 1% gradient. Mobile phase A was 0.1% ammonium hydroxide and mobile phase B was acetonitrile, 0.1% ammonium hydroxide. LC-MS analysis was performed on the Dionex Ultimate 3000 system coupled with the Orbitrap Lumos Mass Spectrometer (Thermo Scientific). Selected TMTpro peptide fractions were loaded to the Acclaim PepMap 100, 100 μm×2 cm C18, 5 μm, 100 Å trapping column and were analyzed with the EASY-Spray C18 capillary column (75 μm×50 cm, 2 μm). Mobile phase A was 0.1% formic acid and mobile phase B was 80% acetonitrile, 0.1% formic acid. The TMTpro peptide fractions were analyzed with a 90 min gradient from 5%-38% B. MS spectral were acquired with mass resolution of 120 k and precursors were isolated for CID fragmentation with collision energy 35%. MS3 quantification was obtained with HCD fragmentation of the top 5 most abundant CID fragments isolated with Synchronous Precursor Selection (SPS) and collision energy 55% at 50k resolution. For the label-free experiments, peptides were analyzed with a 240 min gradient and HCD fragmentation with collision energy 35% and ion trap detection. Database search was performed in Proteome Discoverer 2.4 (Thermo Scientific) using the SequestHT search engine with precursor mass tolerance 20 ppm and fragment ion mass tolerance 0.5 Da. TMTpro at N-terminus/K (for the labelled samples only) and Carbamidomethyl at C were defined as static modifications. Dynamic modifications included oxidation of M and Deamidation of N/Q. The Percolator node was used for peptide confidence estimation and peptides were filtered for q-value <0.01. All spectra were searched against reviewed UniProt human protein entries. Only unique peptides were used for quantification.

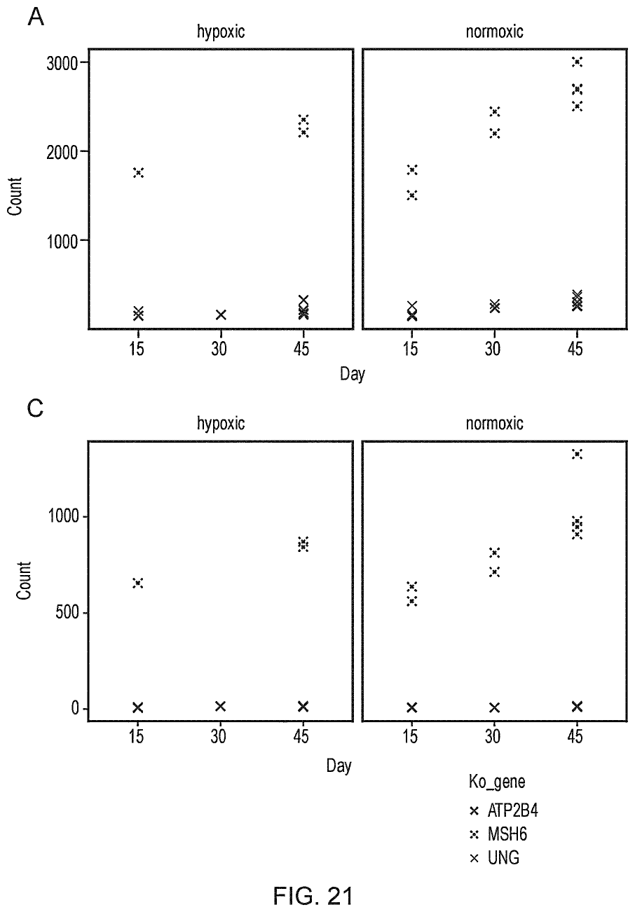

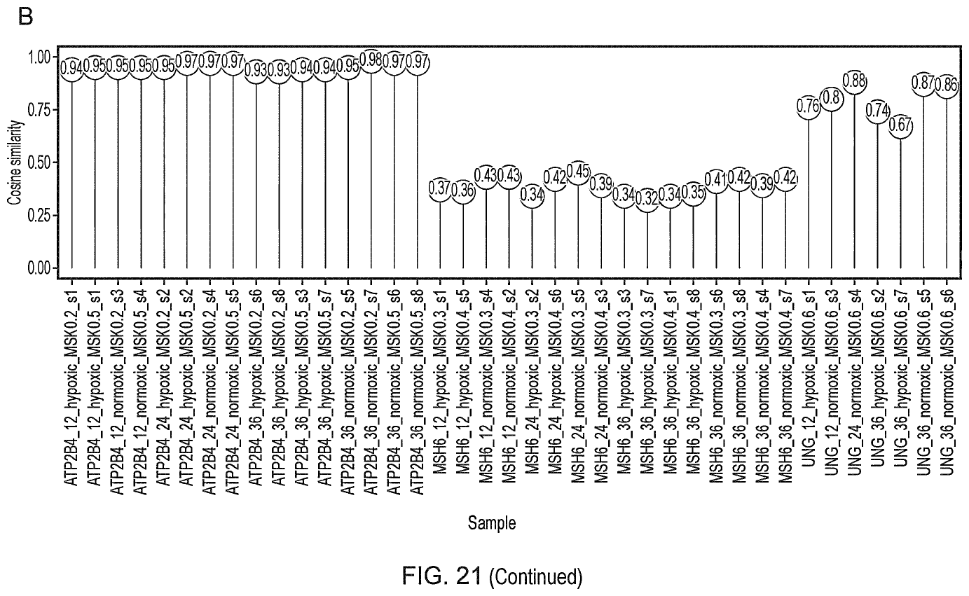

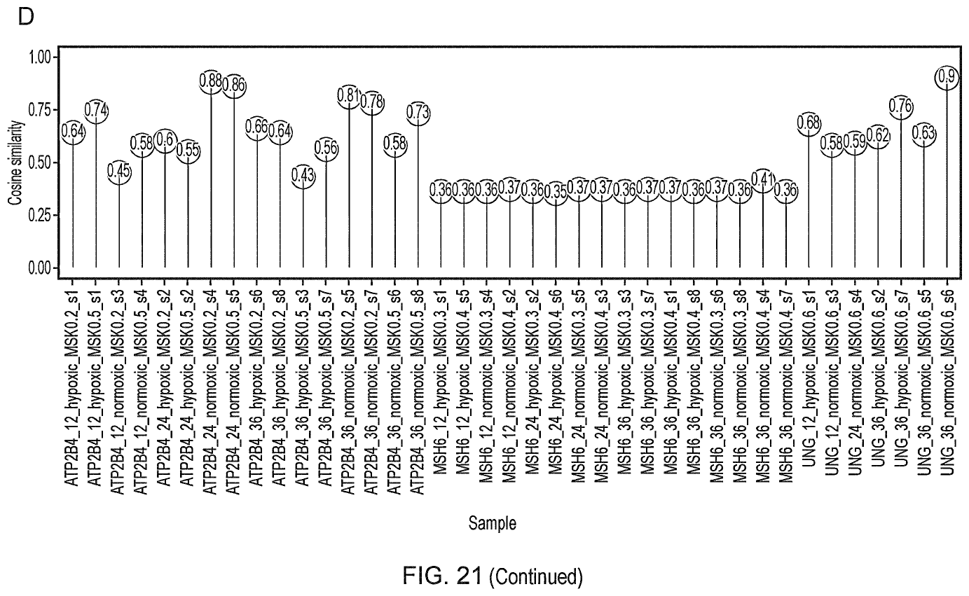

Pilot Study. Prior to generating the full set of knockouts described above, a pilot study was conducted to evaluate the effects of culture conditions and time on mutational signatures. Three genes were selected for knockout (Δ): MSH6, UNG and ATP2B4 (negative control). Two genotypes per gene were obtained and grown in culture to gauge reproducibility of signatures between different genotypes of a gene-knockout. These lines were cultured under normoxic (20%) and hypoxic (3%) states, for defined culture times of ˜15, 30 or 45 days. Two single-cell subclones were derived for whole genome sequencing for each parental line (equivalent to four subclones per gene edit). One of the UNG genotypes appeared to be heterozygous, which was excluded in downstream analysis. All classes of somatic mutations were called, subtracting variation of the primary hiPSC parental clone (see methods in Example 2), and the cosine similarity between mutational profiles of the subclones and the background signature were obtained. The results of this analysis are shown on FIG. 21. Overall, the differences between normoxic and hypoxic conditions were not marked, although normoxic conditions produced slightly more mutations. Time in culture made only a marginal, non-linear difference to burden of mutagenesis. Given the results of the pilot, weighing up the costs and risks associated with prolonged culture time (risk of infection, risk of selection, marked increase in cost of experimental reagents) with the minimal return in terms of mutation number, and also intending to minimize transitions between hypoxic to normoxic conditions while handling cell cultures, we opted to proceed with the full-scale study under normoxic conditions and for 15 days for the rest of study.

Results

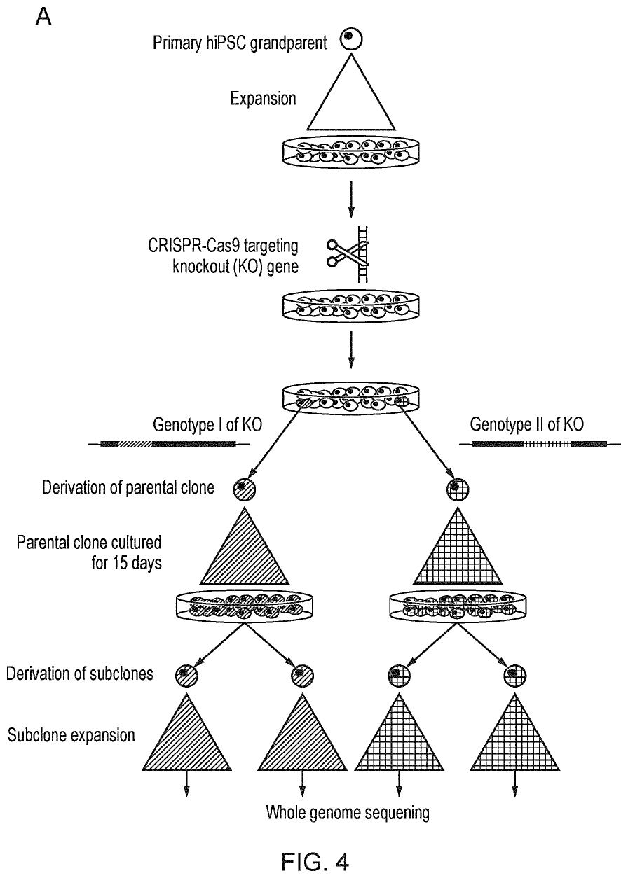

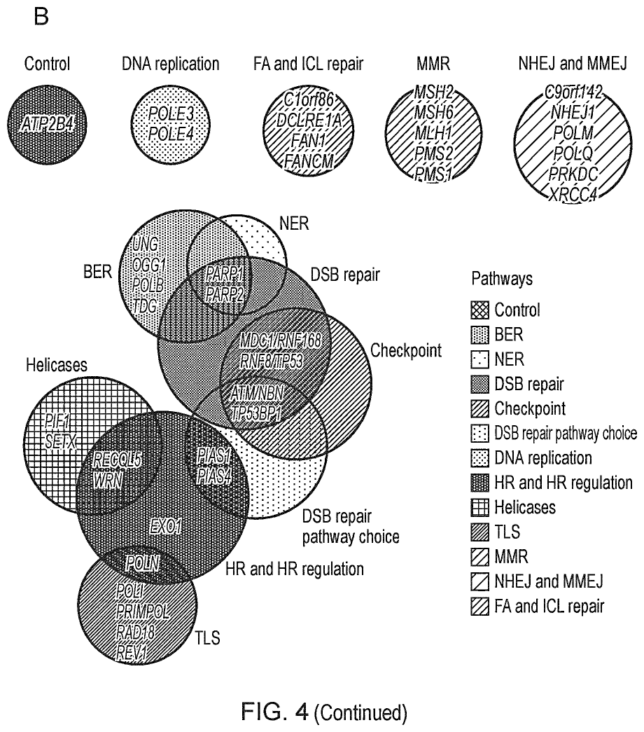

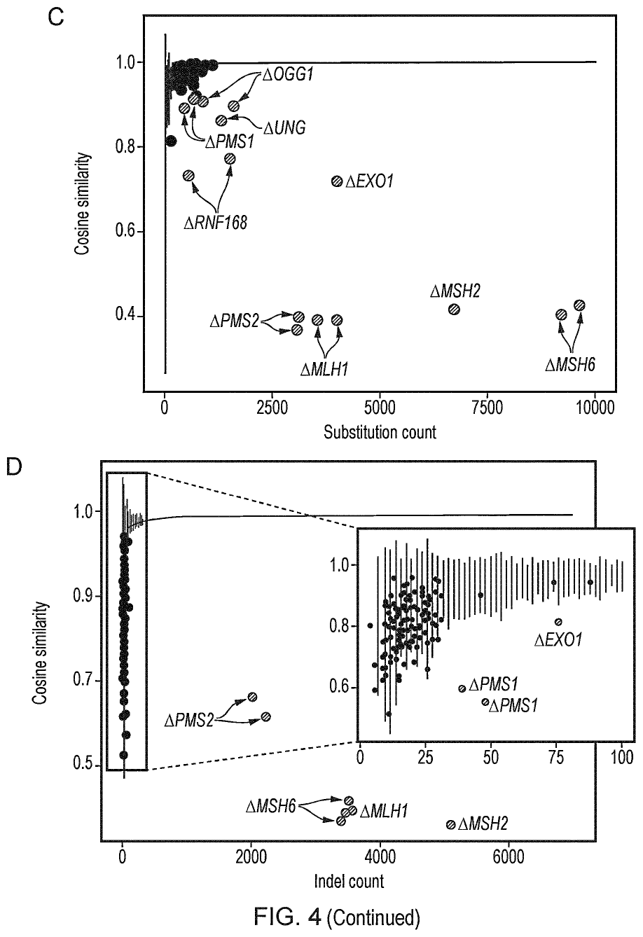

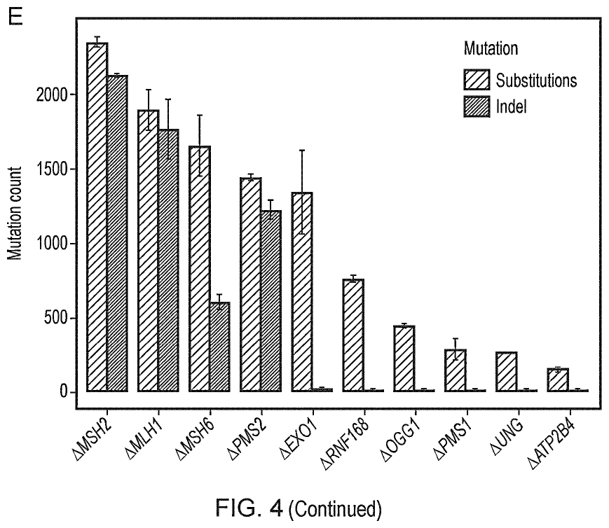

We knocked out (Δ) 42 genes involved in DNA repair/replicative pathways and an unrelated control gene, ATP2B4 (FIGS. 4A and 4B, Table 1). Two knockout genotypes were generated per gene except for EXO1, MSH2, TDG, MDC1, and REV1, where only one knockout genotype was obtained. All parental knockout lines analysed below were grown over 15 days under normoxic conditions (˜20% oxygen). For each genotype, two single-cell subclones were derived for whole-genome sequencing (WGS), aiming for four sequenced subclones per edited gene (FIG. 4A). For single genotype genes, three subclones were derived for ΔEXO1 and ΔMSH2, and four for ΔTDG, ΔMDC1, and ΔREV1.

TABLE 1
List of genes knocked out (KO). CP = checkpoint, DSB = double strand break,
BER = base excision repair, NER = nucleotide excision repair, HR = homologous recombination,
FA = Fanconi Anemia, ICL = interstrand DNA crosslinks, MMR = mismatch repair, NHEJ = non-
homologous end joining, TLS = translesion synthesis.

| Gene KO | Protein KO | Sub-pathway KO |
|---|---|---|
| UNG | Uracil-DNA glycosylase | BER |
| OGG1 | 8-Oxoguanine glycosylase | BER |
| POLB | DNA polymerase beta | BER |
| TDG | Thymine-DNA glycosylase | BER |
| PARP1 | Poly [ADP-ribose] polymerase 1 | BER/DSB repair/NER |
| PARP2 | Poly [ADP-ribose] polymerase 2 | BER/DSB repair/NER |
| MDC1 | Mediator of DNA damage checkpoint | CP/DSB repair |
| | protein 1 | |
| RNF168 | Ring finger protein 168 | CP/DSB repair |
| RNF8 | Ring finger protein 8 | CP/DSB repair |
| TP53 | Tumor protein p53 | CP/DSB repair |
| ATM | ATM serine/threonine kinase | CP/DSB repair/DSB repair pathway |
| | | choice |
| NBN | Nibrin | CP/DSB repair/DSB repair pathway |
| | | choice |
| TP53BP1 | Tumor suppressor p53-binding protein 1 | CP/DSB repair/DSB repair pathway |
| | | choice |
| ATP2B4 | Plasma membrane calcium-transporting | Control |
| | ATPase 4 | |
| POLE3 | DNA polymerase epsilon subunit 3 | DNA replication |
| POLE4 | DNA polymerase epsilon subunit 4 | DNA replication |
| PIAS1 | Protein inhibitor of activated STAT 1 | DSB repair pathway choice/HR and HR |
| | | regulation |
| PIAS4 | protein inhibitor of activated STAT | DSB repair pathway choice/HR and HR |
| | protein gamma | regulation |
| C1orf86 | Fanconi anemia core complex- | FA and ICL repair |
| | associated protein 20 | |
| DCLRE1A | DNA cross-link repair 1A | FA and ICL repair |
| FAN1 | Fanconi-associated nuclease 1 | FA and ICL repair |
| FANCM | Fanconi anemia, complementation group | FA and ICL repair |
| | M | |
| PIF1 | PIF1 5-To-3 DNA Helicase | Helicases |
| SETX | Probable helicase senataxin | Helicases |
| RECQL5 | ATP-dependent DNA helicase Q5 | Helicases/HR and HR regulation |
| WRN | Werner syndrome ATP-dependent | Helicases/HR and HR regulation |
| | helicase | |
| EXO1 | Exonuclease 1 | HR and HR regulation |
| POLN | DNA polymerase nu | HR and HR regulation/TLS |
| MSH6 | MutS protein homolog 6 | MMR |
| MLH1 | MutL homolog 1 | MMR |
| MSH2 | MutS protein homolog 2 | MMR |
| PMS1 | PMS1 protein homolog 1 | MMR |
| PMS2 | protein homolog 2 | MMR |
| C9orf142 | Non-homologous end joining factor | NHEJ and MMEJ |
| NHEJ1 | Non-Homologous End Joining Factor 1 | NHEJ and MMEJ |
| POLM | DNA polymerase mu | NHEJ and MMEJ |
| POLQ | DNA polymerase theta | NHEJ and MMEJ |
| PRKDC | DNA-dependent protein kinase, catalytic | NHEJ and MMEJ |
| | subunit | |
| XRCC4 | X-ray repair cross-complementing | NHEJ and MMEJ |
| | protein 4 | |
| POLI | DNA polymerase iota | TLS |
| PRIMPOL | PrimPol | TLS |
| RAD18 | E3 ubiquitin-protein ligase RAD18 | TLS |
| REV1 | DNA repair protein REV1 | TLS |

List of genes knocked out (KO).

| Gene KO | gRNA1 | gRNA2 |
|---|---|---|
| UNG | ATTGCTAATAGCAGAGTTGC TGG | |
| OGG1 | CGTGGACTCCCACTTCCAAG AGG | GAGCCAGGGTAACATCTAGC TGG |
| POLB | TCAGCCCAATTCGCTGATGA TGG | TGAACCATCATCAGCGAATT GGG |
| TDG | TTGTAAGCAGCCATTAGTCC CGG | |
| PARP1 | CTTTATCCTCTGTAGCAAGG AGG | TCCCAGGAGTCAAGAGTGAA GGG |
| PARP2 | GTGTACAGCCAAGGTGGGGA AGG | AGCTTTGCCCTTTAACAGCA AGG |
| MDC1 | AAAATCTGTCAAGAACAGAA AGG | GGCGTATGGTAAAAAAATCA AGG |
| RNF168 | GAGTCCACGACGATACCCGG CGG | CTCTCGTCAACGTGGAACTG TGG |
| RNF8 | TGAGGGCCAATGGACAATTA TGG | AGTGGTTTCGAGAAATCATC AGG |
| TP53 | GATGGCCATGGCGCGGACGC GGG | GAGCGCTGCTCAGATAGCGA TGG |
| ATM | CGAATTCGAGTGTGTGAATT AGG | AGTTGACAGCCAAAGTCTTG AGG |
| NBN | CAAGAAGAGCATGCAACCAA AGG | AATCAAGCTATATTGCAACT TGG |
| TP53BP1 | GTTGTCTGCACAAGAACTTA TGG | CATAGCAGCAACAGATGCTT TGG |
| ATP2B4 | GGCTTCCGTATGTACAGCAA GGG | ACCGTTGGGATTCCTGATGA CGG |
| POLE3 | GCTAACAACTTTGCAATGAA AGG | TCAATGGGGTAACGAACCGC TGG |
| POLE4 | TGCCTACTGTTGCGCTCAGC AGG | GCCTACTGTTGCGCTCAGCA GGG |
| PIAS1 | ATTCCACAACTCACTTACGA TGG | CATAGGACTTGAATGTACGT TGG |
| PIAS4 | AGTACTTAAACGGACTGGGA CGG | TCAATATTGGGGCCTGCCAG CGG |
| C1orf86 | AGTGGGCTCCGGGCCGCACC TGG | TCGGACCCAAGACCTTTTCC TGG |
| DCLRE1A | CGTCCTGTTTTGCAGATAAC TGG | ATATATTCACCCATTGCCAC TGG |
| FAN1 | GGTGGACGCCTTTCTCAAAT TGG | ATTGGGATTCACCAAGTGGA AGG |
| FANCM | GATGAAGCTCATAAAGCTCT CGG | CGGGACAAGCTCCTCTAGAA AGG |
| PIF1 | GACTTCCCTGTTCCTGGACA GGG | CTGGGCTCACTGCCCCCCAC AGG |
| SETX | TGTTGAAGCACTTTGTCGGA TGG | TGTTGAAGCACTTTGTCGGA TGG |
| RECQL5 | ATGCCTTTGGCCAACAGAGC AGG | TCATTGCTTTGATTCAGGTG AGG |
| WRN | TTAAAAATGGAAAGAAATCT GGG | GTTCTACCGTGCCACTATTG AGG |
| EXO1 | TTGGCTTGTCGTCTTCTGCA AGG | TCTTCGTGAGGGGAAAGTCT CGG |
| POLN | ACAGAAGAAACGGGGGTCTG TGG | GTAAAACGCCAAGCAGAGGG TGG |
| MSH6 | GGAACATTCATCCGCGAGAA AGG | AAACCAGACAAGGCCACCAG GGG |
| MLH1 | GGCTGCATACACTGTTTCTA TGG | |
| MSH2 | TCAAACTGAGAGAGATTGCC AGG | AATGATATGTCAGCTTCCAT TGG |
| PMS1 | GTATCCTTAAACCTGACTTA AGG | GCAGTTACAGTTGTACCTGT TGG |
| PMS2 | ATGCTGTCTTCTAGCACTTC AGG | |
| C9orf142 | AGGCTGCGGGCGCTGACACT GGG | GGGCCCCCCTGAAAGCGTCA GGG |
| NHEJ1 | TCACCAATGCTGCATGCCTC TGG | GAATCTGCAGGATCTGTATA TGG |
| POLM | AACATGTGTCGCTTCGGAGC TGG | AGAGGAGGCCGTCAGCTGGC AGG |
| POLQ | AATAAAAGTAGACGGTTATA TGG | TCTGATCAATCGCCTCATAG AGG |
| PRKDC | CCTGGAATCCTTTCTGAAAC AGG | TTTTCAATTCTACATTTGTG TGG |
| XRCC4 | AGAACTTATTTGTTATTGCT TGG | TTCAACTTTCTCTAGGTTGA AGG |
| POLI | TACTTGCTAGTCTTTTAAAC AGG | |
| PRIMPOL | TTTAACAAACCTGCCAACCC AGG | AGCTTGCACACAGCATTTTC AGG |
| RAD18 | CGCTTAGCCTCTGAGGGATC TGG | CTCCAGACAGTCTTTAAAGC AGG |
| REV1 | CCATTTGCTTGCGCAGAATC TGG | AATTGCATCTTGTAGTTATG AGG |

A total of 173 subclones were obtained from 78 genotyped knockouts of 43 genes (Table 2).

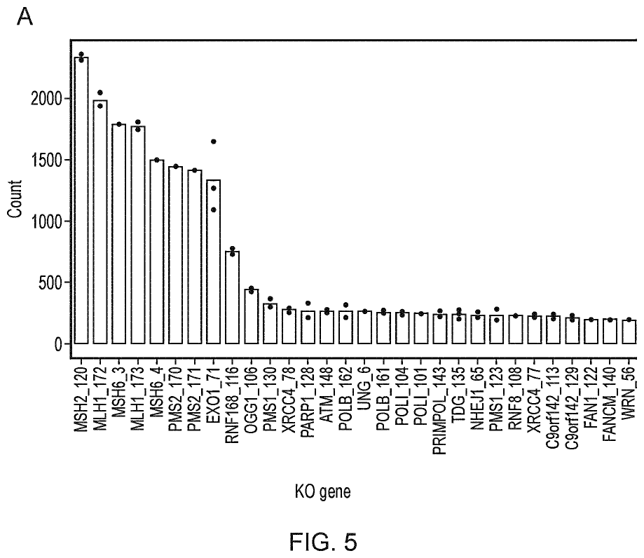

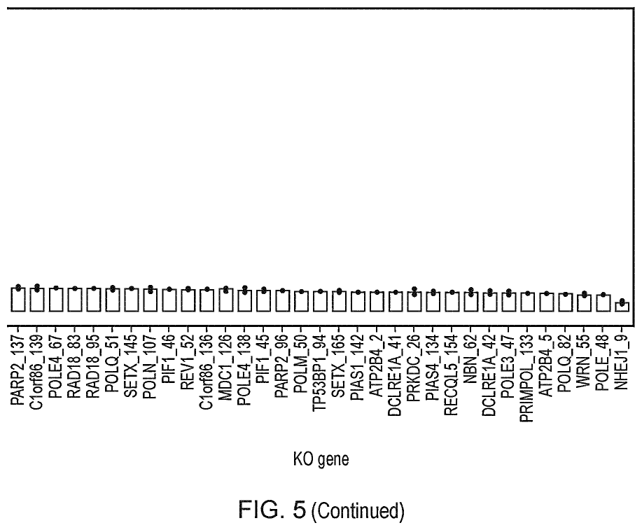

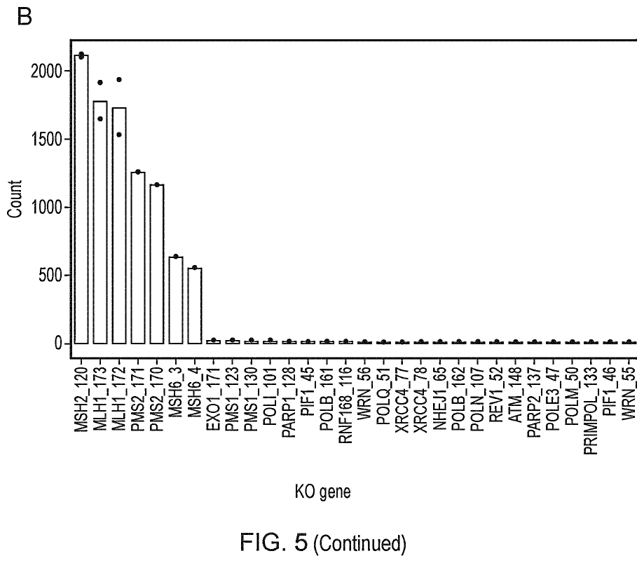

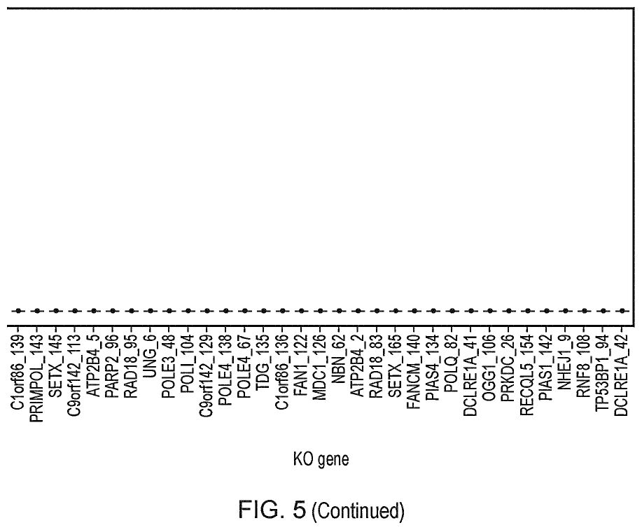

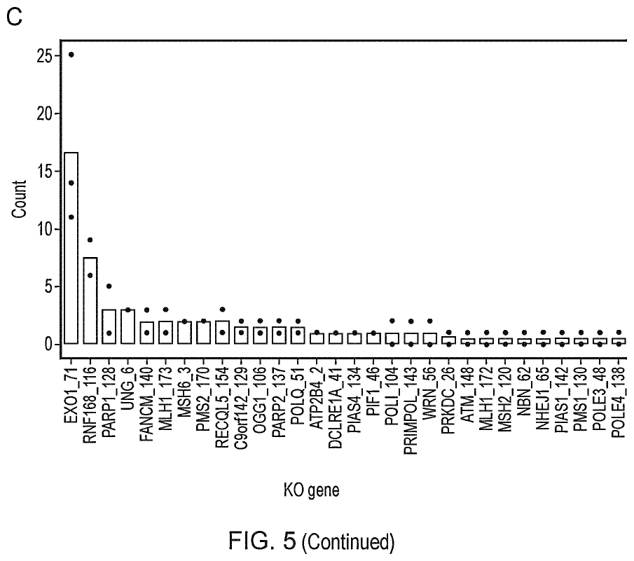

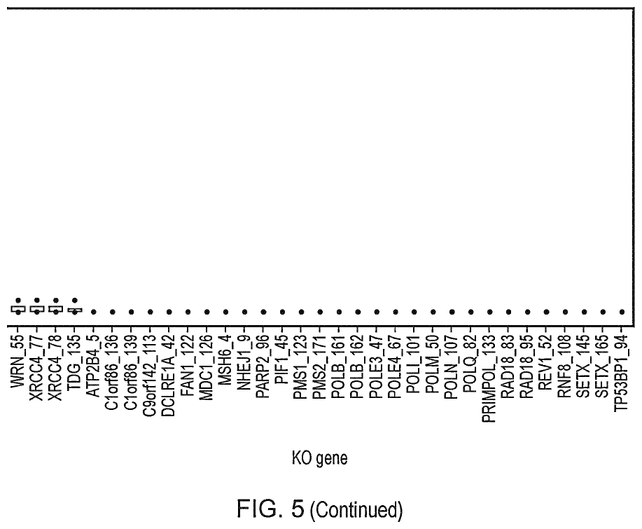

All subclones were sequenced to an average depth of ˜25-fold. Short-read sequences were aligned to human reference genome assembly GRCh37/hg19. All classes of somatic mutations were called, subtracting variation of the primary hiPSC parental clone (see methods section in Example 2; Table 2, Table 3, FIG. 5, pilot results on FIG. 21). Rearrangements were too infrequent to decipher specific patterns.

TABLE 2
List of gene knockout subclones genotyped.

| Sample | Gene KO | Clonality | Sub N | Indel N | Seq. X | Phys. X |
|---|---|---|---|---|---|---|
| MSK0.148_s1 | ATM | Clonal | 277 | 14 | 18.4742623 | 41.1540865 |
| MSK0.148_s2 | ATM | Clonal | 255 | 9 | 18.0509641 | 40.2944197 |
| MSK0.16_s1 | ATM | Polyclonal | 359 | 6 | 35.2519449 | 91.0439039 |
| MSK0.16_s2 | ATM | Clonal | 271 | 19 | 32.0221826 | 81.290546 |
| MSK0.2_s3 | ATP2B4 | Clonal | 161 | 7 | 32.3902859 | 70.7681399 |
| MSK0.2_s4 | ATP2B4 | Polyclonal | 359 | 16 | 31.8947464 | 68.1950639 |
| MSK0.2_s5 | ATP2B4 | Clonal | 263 | 15 | 32.361935 | 69.1393428 |
| MSK0.2_s7 | ATP2B4 | Polyclonal | 359 | 14 | 32.927265 | 70.8972294 |
| MSK0.5_s4 | ATP2B4 | Clonal | 146 | 9 | 34.0452389 | 72.9085348 |
| MSK0.5_s5 | ATP2B4 | Clonal | 238 | 8 | 28.7282119 | 61.0560421 |
| MSK0.5_s6 | ATP2B4 | Clonal | 256 | 11 | 33.4176321 | 71.9536009 |
| MSK0.5_s8 | ATP2B4 | Clonal | 306 | 14 | 34.513418 | 73.9519596 |
| MSK0.136_s1 | C1orf86 | Clonal | 181 | 7 | 19.2893685 | 49.4368816 |
| MSK0.136_s2 | C1orf86 | Clonal | 179 | 8 | 21.217554 | 53.4595478 |
| MSK0.139_s1 | C1orf86 | Clonal | 182 | 12 | 19.9599491 | 50.4366009 |
| MSK0.139_s2 | C1orf86 | Clonal | 203 | 8 | 20.0493146 | 50.4001632 |
| MSK0.113_s1 | C9orf142 | Clonal | 237 | 10 | 19.3814801 | 49.0952828 |
| MSK0.113_s2 | C9orf142 | Clonal | 205 | 9 | 19.322433 | 49.5333047 |
| MSK0.129_s1 | C9orf142 | Clonal | 198 | 8 | 19.8311875 | 51.30311 |
| MSK0.129_s2 | C9orf142 | Clonal | 231 | 8 | 19.3830828 | 50.0350227 |
| MSK0.41_s2 | DCLRE1A | Clonal | 159 | 5 | 19.2456812 | 47.3524146 |
| MSK0.41_s4 | DCLRE1A | Clonal | 161 | 7 | 18.3072932 | 45.180128 |
| MSK0.42_s2 | DCLRE1A | Clonal | 168 | 0 | 16.2556834 | 40.785775 |
| MSK0.42_s4 | DCLRE1A | Clonal | 139 | 4 | 16.2453805 | 40.5984537 |
| MSK0.71_s2 | EXO1 | Clonal | 1646 | 29 | 20.3401887 | 53.2778867 |
| MSK0.71_s3 | EXO1 | Clonal | 1095 | 29 | 24.0316567 | 61.7496537 |
| MSK0.71_s4 | EXO1 | Clonal | 1268 | 18 | 18.2445727 | 47.608795 |
| MSK0.122_s1 | FAN1 | Clonal | 204 | 9 | 19.3039008 | 48.6077438 |
| MSK0.122_s2 | FAN1 | Clonal | 194 | 6 | 17.5548029 | 45.6163538 |
| MSK0.19_s1 | FAN1 | Clonal | 250 | 13 | 35.3910408 | 92.1811586 |
| MSK0.19_s2 | FAN1 | Clonal | 248 | 13 | 34.189105 | 88.5422964 |
| MSK0.10_s1 | FANCM | Polyclonal | 247 | 5 | 34.3818135 | 93.4809744 |
| MSK0.10_s2 | FANCM | Clonal | 144 | 12 | 32.7018032 | 80.7996811 |
| MSK0.140_s1 | FANCM | Clonal | 198 | 4 | 18.1207495 | 42.270403 |
| MSK0.140_s2 | FANCM | Clonal | 197 | 9 | 17.5566377 | 41.7187082 |
| MSK0.126_s1 | MDC1 | Polyclonal | 161 | 4 | 18.5714849 | 48.0747965 |
| MSK0.126_s2 | MDC1 | Polyclonal | 177 | 2 | 19.0845575 | 48.9461776 |
| MSK0.126_s3 | MDC1 | Clonal | 191 | 8 | 18.3878781 | 46.2907698 |
| MSK0.126_s4 | MDC1 | Clonal | 168 | 7 | 17.3913737 | 45.0815329 |
| MSK0.172_s1 | MLH1 | Clonal | 2051 | 1530 | 16.7189266 | 46.6511186 |
| MSK0.172_s2 | MLH1 | Clonal | 1937 | 1935 | 18.3036111 | 46.9974422 |
| MSK0.173_s1 | MLH1 | Clonal | 1803 | 1912 | 20.5769445 | 53.8622911 |
| MSK0.173_s2 | MLH1 | Clonal | 1751 | 1648 | 18.6616104 | 49.8697745 |
| MSK0.120_s1 | MSH2 | Clonal | 2316 | 2122 | 19.6935244 | 50.3902229 |
| MSK0.120_s2 | MSH2 | Clonal | 2360 | 2106 | 19.8936718 | 51.1631821 |
| MSK0.120_s3 | MSH2 | Polyclonal | 2038 | 877 | 15.970413 | 37.091292 |
| MSK0.3_s4 | MSH6 | Clonal | 1790 | 637 | 34.1573755 | 73.0051387 |
| MSK0.3_s5 | MSH6 | Clonal | 2443 | 813 | 34.2049679 | 74.1556112 |
| MSK0.3_s6 | MSH6 | Clonal | 2701 | 947 | 31.3718377 | 66.8285129 |
| MSK0.3_s8 | MSH6 | Clonal | 2688 | 978 | 30.2215355 | 65.7252296 |
| MSK0.4_s2 | MSH6 | Clonal | 1503 | 561 | 33.6732993 | 72.4772583 |
| MSK0.4_s3 | MSH6 | Clonal | 2198 | 713 | 32.1295094 | 68.5620044 |
| MSK0.4_s4 | MSH6 | Clonal | 3001 | 1328 | 68.9391468 | 148.36072 |
| MSK0.4_s7 | MSH6 | Clonal | 2503 | 909 | 32.1830369 | 68.1713744 |
| MSK0.62_s3 | NBN | Clonal | 135 | 6 | 25.2486241 | 64.713135 |
| MSK0.62_s4 | NBN | Clonal | 178 | 9 | 21.5127007 | 55.7483456 |
| MSK0.65_s1 | NHEJ1 | Clonal | 215 | 14 | 33.5904638 | 84.9064318 |
| MSK0.65_s2 | NHEJ1 | Clonal | 258 | 11 | 33.998658 | 84.2667283 |
| MSK0.9_s1 | NHEJ1 | Clonal | 63 | 6 | 36.9160131 | 92.0649888 |
| MSK0.9_s2 | NHEJ1 | Clonal | 85 | 4 | 39.6303605 | 99.24829 |
| MSK0.106_s1 | OGG1 | Clonal | 451 | 7 | 16.5574924 | 42.9735372 |
| MSK0.106_s2 | OGG1 | Clonal | 434 | 5 | 18.8466201 | 48.1342677 |
| MSK0.25_s1 | OGG1 | Clonal | 717 | 22 | 34.1870852 | 88.7693025 |
| MSK0.25_s2 | OGG1 | Polyclonal | 865 | 7 | 31.2312615 | 80.4075251 |
| MSK0.128_s1 | PARP1 | Clonal | 331 | 13 | 19.4324189 | 49.7526538 |
| MSK0.128_s2 | PARP1 | Clonal | 212 | 18 | 19.817329 | 50.6387964 |
| MSK0.18_s2 | PARP1 | Clonal | 487 | 46 | 34.0149996 | 88.2883584 |
| MSK0.137_s1 | PARP2 | Clonal | 185 | 12 | 16.6288826 | 43.950209 |
| MSK0.137_s2 | PARP2 | Clonal | 202 | 10 | 21.0142698 | 53.488644 |
| MSK0.96_s1 | PARP2 | Clonal | 172 | 9 | 16.9712722 | 44.2247154 |
| MSK0.96_s2 | PARP2 | Polyclonal | 217 | 7 | 19.5784092 | 50.6959361 |
| MSK0.13_s1 | PIAS1 | Clonal | 126 | 11 | 31.5920373 | 81.4693412 |
| MSK0.13_s2 | PIAS1 | Clonal | 130 | 11 | 31.8294818 | 79.6611806 |
| MSK0.142_s1 | PIAS1 | Clonal | 163 | 6 | 17.0283551 | 39.8480025 |
| MSK0.142_s2 | PIAS1 | Clonal | 163 | 5 | 16.3388353 | 38.4095049 |
| MSK0.134_s1 | PIAS4 | Clonal | 151 | 5 | 18.9442183 | 48.5961497 |
| MSK0.134_s2 | PIAS4 | Clonal | 167 | 8 | 20.0451452 | 51.4606374 |
| MSK0.23_s1 | PIAS4 | Clonal | 243 | 13 | 34.2785901 | 89.0367719 |
| MSK0.23_s2 | PIAS4 | Clonal | 230 | 13 | 34.4600868 | 89.0767805 |
| MSK0.45_s2 | PIF1 | Clonal | 164 | 11 | 36.8055011 | 90.3663215 |
| MSK0.45_s4 | PIF1 | Clonal | 183 | 19 | 34.7337769 | 86.0347163 |
| MSK0.46_s2 | PIF1 | Clonal | 181 | 12 | 32.6020993 | 81.0980298 |
| MSK0.46_s4 | PIF1 | Clonal | 183 | 9 | 36.9017387 | 91.5991593 |
| MSK0.123_s1 | PMS1 | Clonal | 193 | 21 | 19.7509873 | 49.9601423 |
| MSK0.123_s2 | PMS1 | Clonal | 279 | 27 | 20.1427294 | 51.5853828 |
| MSK0.130_s1 | PMS1 | Clonal | 301 | 17 | 18.8476175 | 47.4985027 |
| MSK0.130_s2 | PMS1 | Clonal | 362 | 22 | 18.6441337 | 47.577422 |
| MSK0.170_s1 | PMS2 | Clonal | 1449 | 1167 | 18.5164868 | 49.4324026 |
| MSK0.170_s2 | PMS2 | Polyclonal | 1618 | 1048 | 21.3719327 | 55.4569325 |
| MSK0.171_s1 | PMS2 | Clonal | 1421 | 1261 | 19.7043677 | 51.7815503 |
| MSK0.171_s2 | PMS2 | Polyclonal | 1665 | 758 | 19.6876333 | 52.9904707 |
| MSK0.161_s1 | POLB | Clonal | 250 | 12 | 18.5410581 | 44.6492944 |
| MSK0.161_s2 | POLB | Clonal | 268 | 18 | 18.0053205 | 43.9886354 |
| MSK0.162_s1 | POLB | Clonal | 315 | 11 | 18.6288985 | 44.9852649 |
| MSK0.162_s2 | POLB | Clonal | 216 | 14 | 17.4074803 | 42.6242704 |
| MSK0.47_s2 | POLE3 | Clonal | 136 | 10 | 34.3329272 | 86.1968522 |
| MSK0.47_s4 | POLE3 | Clonal | 162 | 12 | 35.2914057 | 87.0255646 |
| MSK0.48_s2 | POLE3 | Clonal | 128 | 8 | 32.4253708 | 80.5388248 |
| MSK0.48_s4 | POLE3 | Clonal | 140 | 9 | 33.9383619 | 85.0069811 |
| MSK0.138_s1 | POLE4 | Clonal | 192 | 9 | 21.1392326 | 53.9141988 |
| MSK0.138_s2 | POLE4 | Clonal | 158 | 7 | 18.6676119 | 46.6632182 |
| MSK0.67_s1 | POLE4 | Polyclonal | 218 | 7 | 16.513049 | 42.5102317 |
| MSK0.67_s2 | POLE4 | Clonal | 192 | 8 | 16.477634 | 41.1442152 |
| MSK0.101_s1 | POLI | Clonal | 248 | 19 | 18.3330101 | 46.7226615 |
| MSK0.101_s2 | POLI | Polyclonal | 155 | 4 | 16.7471369 | 45.0355945 |
| MSK0.104_s1 | POLI | Clonal | 264 | 8 | 19.9510767 | 50.8847847 |
| MSK0.104_s2 | POLI | Clonal | 241 | 9 | 17.3800366 | 44.0064179 |
| MSK0.49_s2 | POLM | Polyclonal | 231 | 12 | 34.0457936 | 87.0879327 |
| MSK0.49_s4 | POLM | Polyclonal | 267 | 18 | 36.1216062 | 88.8799497 |
| MSK0.50_s2 | POLM | Clonal | 167 | 11 | 37.2897494 | 93.3278477 |
| MSK0.50_s4 | POLM | Polyclonal | 149 | 5 | 38.9088306 | 97.302964 |
| MSK0.107_s1 | POLN | Clonal | 168 | 11 | 17.1120397 | 43.9644689 |
| MSK0.107_s2 | POLN | Clonal | 198 | 14 | 18.1401563 | 46.757695 |
| MSK0.28_s1 | POLN | Clonal | 258 | 12 | 34.0344399 | 88.3131434 |
| MSK0.28_s2 | POLN | Clonal | 254 | 12 | 33.6285748 | 88.3607797 |
| MSK0.51_s2 | POLQ | Clonal | 195 | 17 | 39.7044473 | 98.9200154 |
| MSK0.51_s4 | POLQ | Clonal | 179 | 9 | 35.8197258 | 89.2162259 |
| MSK0.82_s1 | POLQ | Clonal | 143 | 5 | 17.7227322 | 46.1989928 |
| MSK0.82_s2 | POLQ | Clonal | 137 | 8 | 19.498886 | 50.3513414 |
| MSK0.133_s1 | PRIMPOL | Clonal | 149 | 11 | 17.910841 | 46.3683948 |
| MSK0.133_s2 | PRIMPOL | Polyclonal | 108 | 1 | 17.58242 | 44.919969 |
| MSK0.143_s1 | PRIMPOL | Clonal | 220 | 10 | 16.6749971 | 38.1303111 |
| MSK0.143_s2 | PRIMPOL | Clonal | 263 | 10 | 18.6438661 | 43.8211214 |
| MSK0.26_s2 | PRKDC | Clonal | 139 | 9 | 19.9521926 | 52.4444325 |
| MSK0.26_s3 | PRKDC | Clonal | 180 | 5 | 17.5271161 | 46.5382379 |
| MSK0.26_s4 | PRKDC | Clonal | 160 | 4 | 20.6343685 | 48.1916622 |
| MSK0.83_s1 | RAD18 | Polyclonal | 207 | 2 | 18.1192554 | 47.2945389 |
| MSK0.83_s2 | RAD18 | Clonal | 189 | 7 | 18.7044733 | 48.7210041 |
| MSK0.95_s1 | RAD18 | Clonal | 190 | 10 | 19.1640868 | 48.8744799 |
| MSK0.95_s2 | RAD18 | Clonal | 188 | 8 | 19.033052 | 48.6452964 |
| MSK0.154_s1 | RECQL5 | Clonal | 162 | 8 | 18.3767856 | 42.3380885 |
| MSK0.154_s2 | RECQL5 | Clonal | 153 | 4 | 17.2777745 | 39.7987259 |
| MSK0.21_s2 | RECQL5 | Clonal | 191 | 12 | 32.6278629 | 83.5054636 |
| MSK0.21_s3 | RECQL5 | Clonal | 220 | 12 | 33.1694361 | 85.0588022 |
| MSK0.52_s1 | REV1 | Polyclonal | 68 | 1 | 17.8668558 | 41.3493478 |
| MSK0.52_s2 | REV1 | Clonal | 186 | 14 | 32.271502 | 82.2016495 |
| MSK0.52_s3 | REV1 | Polyclonal | 122 | 4 | 16.0630707 | 37.9742354 |
| MSK0.52_s4 | REV1 | Clonal | 176 | 10 | 34.5542697 | 86.4687518 |
| MSK0.116_s1 | RNF168 | Clonal | 739 | 12 | 17.227062 | 43.55759 |
| MSK0.116_s2 | RNF168 | Clonal | 775 | 17 | 21.9106507 | 53.0234817 |
| MSK0.14_s1 | RNF168 | Clonal | 272 | 10 | 33.5871917 | 91.2184022 |
| MSK0.14_s2 | RNF168 | Clonal | 271 | 8 | 35.379395 | 91.7825097 |
| MSK0.108_s1 | RNF8 | Polyclonal | 251 | 5 | 19.4782038 | 50.3556564 |
| MSK0.108_s2 | RNF8 | Clonal | 231 | 5 | 17.7241732 | 45.5705954 |
| MSK0.12_s1 | RNF8 | Clonal | 145 | 7 | 34.7680219 | 91.6383874 |
| MSK0.12_s2 | RNF8 | Clonal | 111 | 3 | 31.7321506 | 78.671792 |
| MSK0.145_s1 | SETX | Polyclonal | 197 | 10 | 20.6953707 | 45.7682939 |
| MSK0.145_s2 | SETX | Clonal | 184 | 10 | 22.2117972 | 49.1513728 |
| MSK0.165_s1 | SETX | Clonal | 171 | 6 | 17.8573038 | 44.5037228 |
| MSK0.165_s2 | SETX | Clonal | 158 | 8 | 18.8405231 | 47.5899153 |
| MSK0.135_s1 | TDG | Polyclonal | 365 | 5 | 19.7745449 | 50.4911497 |
| MSK0.135_s2 | TDG | Clonal | 275 | 5 | 19.6142943 | 49.948971 |
| MSK0.135_s3 | TDG | Clonal | 249 | 8 | 16.9103534 | 44.0561604 |
| MSK0.135_s4 | TDG | Clonal | 200 | 11 | 18.9386833 | 47.3773368 |
| MSK0.69_s1 | TP53 | Polyclonal | 278 | 11 | 36.6415783 | 90.0883166 |
| MSK0.69_s2 | TP53 | Polyclonal | 219 | 8 | 33.4752946 | 83.6337446 |
| MSK0.70_s2 | TP53 | Polyclonal | 365 | 18 | 38.3196439 | 94.6106111 |
| MSK0.24_s1 | TP53BP1 | Clonal | 262 | 9 | 34.7086888 | 90.5893625 |
| MSK0.24_s2 | TP53BP1 | Clonal | 310 | 8 | 34.1336173 | 88.4388704 |
| MSK0.94_s1 | TP53BP1 | Clonal | 163 | 3 | 18.8865399 | 47.4554116 |
| MSK0.94_s2 | TP53BP1 | Clonal | 169 | 6 | 17.5195603 | 45.0063997 |
| MSK0.6_s3 | UNG | Clonal | 263 | 9 | 34.5315961 | 75.1130793 |
| MSK0.6_s4 | UNG | Clonal | 282 | 7 | 35.4069301 | 75.5116155 |
| MSK0.6_s5 | UNG | Clonal | 361 | 9 | 32.2560337 | 69.1822516 |
| MSK0.6_s6 | UNG | Clonal | 389 | 17 | 34.8995042 | 73.910717 |
| MSK0.55_s2 | WRN | Clonal | 147 | 9 | 31.2257859 | 78.7490367 |
| MSK0.55_s4 | WRN | Clonal | 124 | 12 | 38.3117226 | 96.7400578 |
| MSK0.56_s2 | WRN | Clonal | 199 | 10 | 36.485968 | 90.289262 |
| MSK0.56_s4 | WRN | Clonal | 193 | 18 | 34.2962403 | 85.3939196 |
| MSK0.77_s1 | XRCC4 | Clonal | 238 | 11 | 36.69053 | 91.1107417 |
| MSK0.77_s2 | XRCC4 | Clonal | 217 | 15 | 36.3171881 | 90.4527551 |
| MSK0.78_s1 | XRCC4 | Clonal | 292 | 17 | 36.1428736 | 91.2797066 |
| MSK0.78_s2 | XRCC4 | Clonal | 262 | 9 | 36.499629 | 90.701515 |

Sub N = number of substitutions, Indel N = number of indels, Seq. X = sequencing fold coverage, Phys. X = physical sequence coverage.

TABLE 3
Classes of somatic mutations called.

| Mutation | | | | | | | | |
|---|---|---|---|---|---|---|---|---|
| Type | UNG | OGG1 | POLB | TDG | PARP1 | PARP2 | MDC1 | RNF168 |
| A[C > A]A | 6, 11, 12, 15 | 52, 57, 98, 116 | 14, 17, 16, 5 | 19, 20, 14, 11 | 15, 7, 29 | 11, 12, 11, 17 | 8, 15, 11, 7 | 26, 21, 7, 12 |
| A[C > A]C | 1, 0, 2, 1 | 2, 4, 5, 7 | 2, 0, 1, 4 | 1, 5, 2, 2 | 5, 1, 1 | 2, 1, 0, 1 | 0, 1, 1, 1 | 14, 8, 1, 1 |
| A[C > A]G | 0, 1, 0, 0 | 4, 2, 1, 2 | 0, 0, 0, 0 | 3, 2, 0, 1 | 2, 1, 0 | 1, 1, 1, 3 | 1, 0, 1, 0 | 5, 4, 1, 3 |
| A[C > A]T | 1, 2, 8, 4 | 10, 12, 13, 22 | 3, 8, 5, 8 | 12, 5, 5, 6 | 10, 3, 15 | 4, 8, 4, 8 | 6, 2, 8, 0 | 10, 10, 1, 4 |
| A[C > G]A | 0, 1, 1, 1 | 0, 1, 1, 3 | 2, 2, 0, 4 | 0, 4, 1, 1 | 5, 0, 1 | 2, 0, 1, 0 | 1, 1, 0, 1 | 10, 7, 1, 3 |
| A[C > G]C | 0, 0, 1, 1 | 0, 0, 0, 2 | 3, 0, 2, 1 | 1, 1, 2, 0 | 3, 0, 2 | 0, 1, 1, 1 | 0, 1, 0, 0 | 5, 1, 1, 1 |
| A[C > G]G | 1, 2, 0, 0 | 0, 1, 0, 0 | 1, 1, 1, 1 | 0, 0, 1, 0 | 1, 0, 1 | 0, 1, 2, 0 | 1, 2, 0, 0 | 9, 3, 5, 5 |
| A[C > G]T | 0, 0, 1, 0 | 0, 1, 0, 3 | 0, 1, 0, 1 | 0, 0, 0, 1 | 0, 0, 2 | 0, 0, 0, 0 | 3, 0, 2, 0 | 7, 14, 5, 4 |
| A[C > T]A | 18, 17, 23, 27 | 3, 6, 7, 7 | 5, 3, 0, 4 | 9, 12, 7, 7 | 8, 5, 8 | 5, 3, 3, 4 | 2, 5, 3, 2 | 18, 22, 7, 7 |
| A[C > T]C | 5, 12, 12, 9 | 3, 3, 1, 2 | 0, 0, 4, 3 | 1, 3, 3, 2 | 5, 1, 6 | 1, 6, 2, 2 | 0, 5, 1, 0 | 4, 7, 4, 1 |
| A[C > T]G | 3, 2, 6, 6 | 1, 7, 2, 3 | 1, 4, 4, 1 | 3, 4, 1, 3 | 1, 2, 8 | 5, 0, 4, 0 | 2, 2, 3, 1 | 6, 8, 7, 1 |
| A[C > T]T | 9, 3, 7, 3 | 2, 0, 3, 6 | 0, 3, 4, 1 | 4, 2, 5, 1 | 0, 3, 7 | 6, 4, 0, 1 | 1, 0, 1, 3 | 7, 8, 3, 5 |
| A[T > A]A | 1, 1, 0, 0 | 2, 1, 1, 0 | 3, 1, 0, 0 | 1, 1, 1, 0 | 0, 3, 3 | 1, 0, 2, 3 | 2, 2, 0, 1 | 5, 6, 1, 0 |
| A[T > A]C | 0, 0, 0, 1 | 0, 0, 0, 0 | 0, 1, 1, 1 | 0, 0, 0, 0 | 0, 0, 3 | 0, 0, 0, 1 | 1, 1, 0, 0 | 2, 1, 0, 1 |
| A[T > A]G | 1, 1, 0, 2 | 0, 2, 0, 1 | 1, 1, 0, 0 | 2, 1, 0, 1 | 1, 1, 0 | 0, 1, 0, 0 | 0, 0, 0, 0 | 4, 3, 0, 1 |
| A[T > A]T | 1, 1, 1, 1 | 1, 0, 0, 3 | 3, 1, 1, 1 | 1, 2, 1, 2 | 0, 2, 3 | 2, 1, 1, 1 | 3, 0, 0, 1 | 6, 9, 4, 3 |
| A[T > C]A | 3, 2, 3, 2 | 1, 3, 4, 5 | 6, 8, 0, 3 | 3, 3, 3, 3 | 8, 3, 5 | 4, 6, 2, 3 | 4, 3, 0, 3 | 27, 31, 16, 13 |
| A[T > C]C | 0, 0, 0, 0 | 0, 0, 1, 2 | 0, 0, 2, 0 | 1, 4, 0, 1 | 1, 2, 2 | 0, 0, 0, 0 | 0, 0, 0, 0 | 5, 3, 2, 2 |
| A[T > C]G | 1, 1, 2, 1 | 0, 1, 1, 3 | 2, 2, 1, 2 | 4, 0, 0, 0 | 2, 1, 4 | 1, 1, 1, 1 | 3, 1, 2, 1 | 10, 19, 4, 5 |
| A[T > C]T | 3, 5, 1, 4 | 1, 0, 3, 5 | 1, 3, 3, 4 | 1, 1, 1, 1 | 2, 1, 6 | 1, 2, 2, 3 | 2, 1, 1, 0 | 16, 13, 5, 5 |
| A[T > G]A | 0, 0, 0, 0 | 0, 0, 0, 0 | 0, 0, 0, 1 | 1, 1, 0, 0 | 0, 0, 1 | 0, 0, 0, 0 | 0, 0, 0, 1 | 4, 1, 1, 0 |
| A[T > G]C | 0, 0, 0, 0 | 0, 0, 1, 0 | 0, 0, 1, 0 | 0, 1, 0, 0 | 0, 0, 1 | 0, 0, 0, 0 | 0, 0, 0, 0 | 1, 2, 0, 1 |
| A[T > G]G | 1, 0, 0, 0 | 0, 0, 1, 0 | 1, 1, 1, 1 | 0, 1, 0, 1 | 0, 1, 2 | 0, 1, 0, 1 | 0, 0, 0, 0 | 2, 3, 3, 0 |
| A[T > G]T | 1, 1, 0, 2 | 3, 0, 2, 1 | 1, 1, 0, 0 | 0, 1, 0, 0 | 1, 2, 1 | 0, 0, 0, 2 | 0, 2, 1, 1 | 5, 3, 2, 2 |
| C[C > A]A | 7, 7, 4, 7 | 17, 14, 22, 31 | 8, 13, 20, 9 | 11, 12, 11, 7 | 16, 8, 14 | 8, 10, 8, 8 | 5, 6, 5, 9 | 12, 17, 6, 5 |
| C[C > A]C | 0, 0, 3, 1 | 1, 1, 1, 2 | 2, 3, 3, 1 | 2, 3, 3, 1 | 1, 1, 4 | 2, 1, 2, 2 | 1, 0, 3, 0 | 7, 10, 2, 3 |
| C[C > A]G | 0, 2, 1, 2 | 2, 0, 8, 5 | 1, 1, 3, 0 | 1, 1, 1, 2 | 1, 2, 1 | 1, 3, 2, 2 | 0, 1, 1, 0 | 8, 4, 1, 0 |
| C[C > A]T | 1, 5, 2, 5 | 9, 9, 7, 8 | 4, 5, 5, 10 | 6, 8, 5, 6 | 4, 5, 7 | 5, 2, 3, 3 | 2, 3, 4, 6 | 11, 9, 6, 5 |
| C[C > G]A | 0, 0, 1, 1 | 0, 0, 2, 1 | 0, 0, 0, 0 | 4, 0, 0, 1 | 0, 3, 0 | 0, 1, 0, 1 | 0, 0, 0, 1 | 4, 9, 1, 3 |
| C[C > G]C | 0, 1, 1, 2 | 0, 0, 0, 0 | 0, 1, 1, 1 | 2, 3, 0, 0 | 2, 0, 1 | 0, 0, 0, 1 | 0, 0, 0, 0 | 5, 3, 0, 1 |
| C[C > G]G | 0, 0, 0, 0 | 0, 0, 1, 1 | 0, 0, 1, 0 | 0, 0, 0, 0 | 0, 0, 0 | 0, 0, 0, 0 | 0, 0, 0, 0 | 2, 2, 1, 2 |
| C[C > G]T | 0, 0, 0, 1 | 0, 0, 1, 2 | 1, 0, 0, 0 | 1, 0, 1, 1 | 4, 0, 2 | 0, 0, 0, 1 | 0, 1, 2, 2 | 11, 6, 4, 0 |
| C[C > T]A | 18, 17, 16, 11 | 7, 5, 4, 6 | 6, 14, 4, 3 | 4, 12, 7, 4 | 4, 6, 9 | 4, 4, 2, 5 | 4, 3, 2, 5 | 19, 11, 2, 6 |
| C[C > T]C | 9, 6, 12, 13 | 1, 1, 5, 2 | 2, 4, 4, 2 | 5, 5, 7, 2 | 4, 2, 4 | 1, 2, 2, 3 | 3, 3, 2, 2 | 8, 10, 2, 1 |
| C[C > T]G | 2, 2, 2, 3 | 0, 0, 5, 4 | 3, 2, 4, 3 | 4, 3, 5, 7 | 2, 3, 7 | 2, 2, 2, 2 | 1, 1, 2, 2 | 10, 5, 1, 4 |
| C[C > T]T | 5, 7, 9, 11 | 4, 0, 4, 5 | 5, 4, 4, 3 | 4, 3, 3, 0 | 6, 2, 7 | 2, 5, 1, 0 | 0, 1, 2, 2 | 11, 13, 4, 2 |
| C[T > A]A | 1, 0, 1, 1 | 1, 0, 0, 1 | 0, 1, 2, 0 | 1, 0, 0, 1 | 1, 0, 2 | 1, 0, 0, 1 | 2, 0, 1, 0 | 11, 4, 3, 1 |
| C[T > A]C | 0, 0, 0, 2 | 0, 0, 0, 1 | 2, 0, 0, 1 | 1, 0, 0, 0 | 0, 1, 2 | 0, 0, 0, 0 | 0, 0, 0, 0 | 1, 4, 1, 1 |
| C[T > A]G | 0, 0, 0, 0 | 0, 0, 1, 1 | 2, 0, 0, 0 | 1, 0, 1, 0 | 0, 1, 3 | 0, 0, 0, 0 | 0, 1, 0, 0 | 6, 5, 3, 1 |
| C[T > A]T | 0, 0, 2, 1 | 0, 0, 0, 1 | 4, 1, 1, 0 | 0, 1, 0, 0 | 1, 0, 1 | 1, 0, 0, 1 | 0, 2, 1, 0 | 4, 9, 2, 0 |
| C[T > C]A | 1, 3, 1, 0 | 1, 2, 0, 0 | 1, 1, 1, 3 | 1, 1, 0, 1 | 4, 0, 2 | 1, 0, 1, 1 | 1, 1, 2, 1 | 8, 6, 3, 4 |
| C[T > C]C | 0, 0, 0, 1 | 0, 0, 0, 1 | 0, 0, 3, 0 | 0, 1, 0, 1 | 0, 0, 2 | 0, 1, 0, 1 | 0, 0, 0, 0 | 3, 2, 2, 1 |
| C[T > C]G | 1, 1, 0, 1 | 0, 2, 2, 4 | 2, 0, 0, 0 | 2, 2, 0, 0 | 2, 1, 4 | 1, 2, 0, 0 | 0, 0, 0, 1 | 5, 4, 0, 1 |
| C[T > C]T | 1, 0, 0, 1 | 0, 0, 1, 2 | 1, 0, 0, 4 | 0, 0, 1, 0 | 3, 0, 1 | 0, 2, 1, 2 | 1, 0, 1, 2 | 3, 9, 5, 4 |
| C[T > G]A | 0, 0, 0, 1 | 0, 0, 0, 0 | 1, 0, 0, 0 | 0, 1, 0, 0 | 0, 0, 1 | 0, 0, 0, 0 | 0, 0, 0, 1 | 1, 1, 0, 1 |
| C[T > G]C | 2, 0, 1, 1 | 0, 0, 1, 0 | 0, 0, 0, 0 | 0, 0, 0, 0 | 0, 0, 0 | 0, 0, 0, 0 | 0, 0, 0, 0 | 3, 0, 1, 1 |
| C[T > G]G | 0, 0, 2, 0 | 0, 0, 0, 0 | 0, 0, 0, 0 | 0, 0, 0, 0 | 0, 0, 1 | 0, 0, 0, 0 | 0, 0, 2, 0 | 5, 5, 0, 3 |
| C[T > G]T | 0, 0, 1, 2 | 0, 0, 0, 0 | 1, 1, 1, 0 | 2, 1, 1, 0 | 0, 0, 1 | 0, 0, 0, 0 | 2, 0, 0, 0 | 2, 3, 0, 1 |
| G[C > A]A | 16, 18, 29, 21 | 145, 153, 242, 297 | 48, 41, 71, 28 | 49, 38, 43, 22 | 43, 42, 81 | 28, 26, 29, 33 | 17, 31, 41, 30 | 35, 36, 11, 12 |
| G[C > A]C | 0, 0, 2, 3 | 5, 9, 15, 11 | 3, 1, 3, 3 | 7, 3, 3, 2 | 6, 3, 5 | 1, 2, 0, 1 | 2, 0, 1, 4 | 2, 9, 0, 2 |
| G[C > A]G | 1, 2, 0, 1 | 6, 5, 8, 6 | 1, 1, 1, 1 | 4, 4, 1, 1 | 3, 1, 2 | 3, 1, 0, 2 | 1, 3, 1, 2 | 4, 0, 1, 3 |
| G[C > A]T | 7, 8, 15, 11 | 48, 37, 62, 75 | 16, 17, 27, 18 | 22, 10, 16, 9 | 18, 7, 34 | 11, 17, 17, 14 | 6, 14, 15, 9 | 12, 12, 6, 7 |
| G[C > G]A | 0, 1, 2, 1 | 1, 2, 0, 2 | 0, 0, 1, 1 | 2, 1, 1, 0 | 0, 1, 2 | 0, 0, 0, 0 | 0, 1, 0, 1 | 6, 10, 0, 3 |
| G[C > G]C | 1, 1, 0, 0 | 1, 0, 0, 1 | 0, 1, 0, 1 | 0, 0, 0, 1 | 1, 0, 1 | 0, 0, 0, 0 | 0, 0, 0, 0 | 4, 3, 1, 1 |
| G[C > G]G | 0, 0, 0, 0 | 0, 0, 1, 0 | 0, 0, 0, 0 | 0, 0, 0, 0 | 0, 1, 0 | 0, 0, 0, 0 | 0, 1, 0, 0 | 1, 3, 0, 2 |
| G[C > G]T | 0, 0, 3, 1 | 0, 0, 0, 0 | 2, 0, 1, 1 | 2, 1, 1, 0 | 2, 0, 2 | 0, 1, 0, 1 | 0, 0, 0, 0 | 5, 3, 4, 4 |
| G[C > T]A | 14, 3, 18, 17 | 1, 2, 7, 4 | 1, 3, 2, 2 | 6, 5, 5, 3 | 6, 9, 5 | 2, 2, 3, 3 | 4, 3, 0, 4 | 10, 23, 5, 3 |
| G[C > T]C | 3, 8, 4, 9 | 1, 1, 1, 5 | 4, 2, 3, 3 | 4, 5, 1, 2 | 9, 1, 3 | 3, 1, 0, 4 | 3, 3, 1, 0 | 4, 9, 2, 1 |
| G[C > T]G | 3, 1, 3, 3 | 5, 4, 3, 3 | 3, 5, 4, 1 | 6, 1, 0, 1 | 4, 3, 2 | 0, 0, 1, 2 | 0, 3, 1, 0 | 2, 2, 5, 1 |
| G[C > T]T | 5, 1, 11, 10 | 2, 0, 5, 1 | 2, 6, 0, 1 | 3, 0, 4, 1 | 4, 3, 5 | 1, 2, 2, 2 | 1, 3, 0, 0 | 7, 13, 4, 5 |
| G[T > A]A | 0, 0, 0, 0 | 0, 0, 0, 0 | 0, 1, 1, 0 | 2, 0, 1, 1 | 2, 1, 1 | 0, 0, 0, 0 | 1, 0, 1, 0 | 6, 5, 1, 0 |
| G[T > A]C | 0, 0, 0, 1 | 1, 0, 0, 0 | 0, 0, 1, 0 | 1, 2, 0, 0 | 2, 0, 0 | 0, 0, 1, 0 | 0, 2, 1, 0 | 2, 5, 0, 0 |
| G[T > A]G | 0, 1, 1, 0 | 0, 0, 0, 1 | 1, 0, 0, 0 | 0, 1, 0, 0 | 4, 0, 2 | 0, 0, 0, 0 | 0, 0, 0, 1 | 2, 4, 2, 0 |
| G[T > A]T | 3, 1, 0, 0 | 0, 0, 0, 0 | 0, 0, 0, 0 | 0, 1, 2, 1 | 0, 0, 0 | 1, 1, 1, 0 | 0, 1, 0, 0 | 4, 4, 3, 1 |
| G[T > C]A | 0, 1, 2, 0 | 0, 0, 3, 0 | 4, 2, 1, 0 | 1, 1, 0, 2 | 2, 0, 2 | 0, 2, 1, 0 | 0, 0, 1, 0 | 7, 11, 1, 1 |
| G[T > C]C | 0, 0, 0, 1 | 1, 0, 1, 2 | 0, 0, 1, 1 | 0, 1, 1, 0 | 0, 0, 1 | 0, 0, 0, 0 | 0, 0, 0, 0 | 4, 3, 1, 0 |
| G[T > C]G | 0, 0, 1, 1 | 0, 1, 1, 0 | 0, 0, 0, 0 | 1, 0, 0, 1 | 1, 1, 1 | 1, 1, 1, 0 | 1, 0, 0, 0 | 4, 3, 2, 2 |
| G[T > C]T | 2, 0, 0, 1 | 1, 0, 0, 0 | 2, 2, 2, 0 | 0, 1, 1, 2 | 0, 0, 6 | 1, 3, 0, 1 | 0, 1, 0, 1 | 4, 5, 3, 2 |
| G[T > G]A | 1, 0, 0, 0 | 1, 0, 0, 0 | 0, 0, 0, 0 | 2, 0, 0, 0 | 1, 1, 0 | 0, 0, 0, 1 | 0, 0, 0, 0 | 2, 0, 0, 0 |
| G[T > G]C | 0, 1, 0, 1 | 0, 0, 0, 0 | 0, 1, 0, 1 | 1, 0, 0, 0 | 0, 0, 0 | 0, 0, 0, 0 | 0, 0, 0, 0 | 2, 2, 0, 0 |
| G[T > G]G | 0, 0, 1, 0 | 0, 0, 0, 0 | 0, 0, 0, 0 | 0, 0, 0, 0 | 1, 0, 0 | 0, 0, 1, 1 | 1, 0, 1, 0 | 3, 3, 2, 2 |
| G[T > G]T | 1, 1, 0, 1 | 0, 0, 0, 0 | 0, 0, 0, 0 | 0, 0, 0, 0 | 0, 1, 0 | 0, 0, 0, 0 | 1, 1, 0, 0 | 1, 3, 1, 1 |
| T[C > A]A | 7, 11, 13, 13 | 26, 20, 35, 40 | 14, 15, 13, 15 | 25, 4, 17, 13 | 23, 11, 25 | 10, 12, 13, 13 | 10, 13, 14, 8 | 29, 14, 8, 11 |
| T[C > A]C | 1, 3, 4, 4 | 8, 4, 13, 13 | 2, 3, 4, 1 | 9, 2, 4, 1 | 2, 4, 7 | 2, 4, 0, 5 | 4, 3, 4, 2 | 10, 19, 7, 6 |
| T[C > A]G | 1, 0, 1, 0 | 2, 4, 6, 5 | 0, 0, 1, 0 | 5, 3, 1, 1 | 3, 3, 3 | 1, 1, 2, 4 | 0, 1, 2, 1 | 5, 3, 2, 1 |
| T[C > A]T | 13, 20, 27, 28 | 47, 39, 71, 76 | 19, 27, 37, 27 | 42, 21, 25, 31 | 25, 21, 51 | 27, 23, 24, 18 | 24, 13, 23, 28 | 33, 35, 10, 12 |
| T[C > G]A | 1, 1, 2, 3 | 0, 0, 1, 0 | 0, 3, 0, 0 | 3, 0, 2, 2 | 1, 0, 3 | 2, 2, 0, 0 | 1, 0, 0, 0 | 6, 8, 4, 4 |
| T[C > G]C | 0, 0, 1, 2 | 1, 0, 0, 0 | 0, 0, 0, 1 | 1, 1, 1, 2 | 1, 0, 4 | 0, 0, 0, 0 | 1, 1, 0, 1 | 5, 8, 0, 2 |
| T[C > G]G | 2, 0, 0, 1 | 0, 0, 1, 0 | 0, 0, 1, 1 | 1, 0, 0, 0 | 1, 0, 0 | 1, 0, 1, 0 | 1, 0, 1, 0 | 5, 2, 0, 2 |
| T[C > G]T | 0, 0, 2, 4 | 1, 2, 1, 1 | 3, 2, 1, 1 | 2, 4, 0, 0 | 2, 2, 4 | 0, 1, 1, 0 | 0, 1, 1, 2 | 14, 16, 4, 5 |
| T[C > T]A | 13, 12, 16, 21 | 1, 4, 4, 11 | 5, 3, 9, 1 | 8, 2, 1, 2 | 2, 5, 4 | 3, 6, 2, 3 | 6, 0, 2, 4 | 19, 17, 4, 3 |
| T[C > T]C | 8, 5, 12, 11 | 3, 2, 3, 7 | 6, 3, 4, 4 | 5, 3, 4, 1 | 5, 0, 7 | 1, 1, 0, 3 | 1, 2, 4, 0 | 10, 11, 0, 7 |
| T[C > T]G | 3, 2, 1, 2 | 1, 2, 2, 3 | 0, 2, 3, 3 | 3, 5, 3, 2 | 3, 5, 3 | 1, 0, 2, 5 | 1, 1, 1, 3 | 3, 4, 6, 2 |
| T[C > T]T | 6, 12, 10, 11 | 3, 1, 3, 5 | 2, 4, 5, 2 | 2, 4, 3, 2 | 4, 0, 10 | 1, 2, 1, 4 | 0, 2, 1, 0 | 11, 8, 5, 6 |
| T[T > A]A | 1, 1, 0, 2 | 5, 1, 1, 0 | 1, 3, 4, 1 | 1, 2, 2, 5 | 3, 4, 1 | 1, 1, 1, 1 | 0, 0, 0, 2 | 8, 8, 4, 5 |
| T[T > A]C | 0, 1, 0, 1 | 0, 1, 3, 1 | 1, 0, 1, 0 | 1, 2, 0, 0 | 1, 0, 1 | 1, 0, 0, 0 | 0, 1, 0, 1 | 9, 10, 2, 3 |
| T[T > A]G | 0, 1, 2, 0 | 2, 1, 1, 1 | 3, 0, 1, 1 | 1, 1, 0, 0 | 0, 1, 1 | 0, 2, 0, 1 | 1, 0, 0, 0 | 5, 7, 1, 1 |
| T[T > A]T | 1, 1, 1, 4 | 2, 0, 2, 1 | 0, 1, 3, 2 | 1, 1, 3, 3 | 3, 0, 2 | 0, 1, 1, 0 | 2, 2, 1, 1 | 8, 12, 9, 4 |
| T[T > C]A | 3, 4, 3, 2 | 1, 1, 2, 5 | 6, 5, 3, 3 | 10, 3, 3, 1 | 8, 2, 7 | 4, 1, 4, 1 | 2, 2, 3, 0 | 15, 19, 11, 6 |
| T[T > C]C | 2, 2, 0, 0 | 0, 0, 2, 3 | 0, 1, 0, 1 | 2, 0, 1, 0 | 0, 0, 2 | 0, 1, 0, 3 | 1, 0, 0, 1 | 3, 2, 2, 1 |
| T[T > C]G | 4, 0, 0, 2 | 0, 2, 2, 2 | 2, 2, 0, 1 | 4, 1, 3, 2 | 2, 1, 0 | 1, 1, 0, 1 | 1, 0, 2, 1 | 2, 7, 0, 2 |
| T[T > C]T | 0, 5, 2, 3 | 0, 0, 2, 5 | 1, 1, 0, 5 | 1, 4, 2, 4 | 3, 3, 5 | 0, 1, 1, 1 | 0, 0, 1, 3 | 15, 16, 2, 2 |
| T[T > G]A | 1, 1, 1, 0 | 0, 0, 2, 2 | 1, 0, 1, 0 | 1, 0, 0, 0 | 1, 0, 3 | 1, 1, 0, 0 | 0, 0, 0, 0 | 2, 3, 1, 0 |
| T[T > G]C | 0, 0, 0, 1 | 0, 0, 0, 0 | 0, 0, 0, 0 | 0, 2, 0, 0 | 1, 0, 0 | 0, 0, 0, 0 | 0, 0, 1, 0 | 4, 4, 0, 0 |
| T[T > G]G | 0, 1, 0, 1 | 0, 0, 1, 2 | 0, 2, 2, 0 | 3, 0, 0, 0 | 2, 1, 3 | 1, 0, 0, 1 | 2, 0, 1, 1 | 6, 3, 0, 1 |
| T[T > G]T | 1, 2, 2, 2 | 1, 1, 1, 4 | 4, 0, 2, 1 | 2, 5, 1, 1 | 1, 0, 1 | 2, 0, 1, 2 | 3, 2, 0, 0 | 11, 10, 4, 1 |

| Mutation | | | | | | |
|---|---|---|---|---|---|---|
| Type | RNF8 | TP53 | ATM | NBN | TP53BP1 | POLE3 |
| A[C > A]A | 12, 19, 8, 3 | 16, 16, 16, 18, 26, 7, 11 | 15, 14, 36, 17 | 10, 7 | 18, 26, 7, 11 | 9, 10, 6, 10 |
| A[C > A]C | 1, 2, 1, 0 | 1, 3, 0, 2, 2, 0, 1 | 1, 0, 1, 2 | 0, 0 | 2, 2, 0, 1 | 3, 2, 0, 0 |
| A[C > A]G | 1, 2, 1, 0 | 0, 3, 3, 1, 1, 0, 0 | 1, 2, 2, 0 | 0, 1 | 1, 1, 0, 0 | 0, 0, 0, 0 |
| A[C > A]T | 7, 4, 4, 2 | 5, 6, 5, 12, 9, 4, 2 | 4, 10, 10, 8 | 3, 6 | 12, 9, 4, 2 | 1, 2, 1, 2 |
| A[C > G]A | 4, 0, 1, 0 | 0, 1, 2, 1, 1, 0, 2 | 2, 1, 1, 2 | 3, 1 | 1, 1, 0, 2 | 1, 0, 1, 2 |
| A[C > G]C | 0, 2, 0, 1 | 1, 0, 0, 1, 0, 0, 1 | 0, 0, 0, 0 | 0, 1 | 1, 0, 0, 1 | 0, 1, 0, 1 |
| A[C > G]G | 0, 1, 0, 1 | 0, 0, 1, 1, 0, 0, 0 | 0, 0, 0, 1 | 0, 2 | 1, 0, 0, 0 | 1, 0, 0, 0 |
| A[C > G]T | 2, 0, 0, 0 | 3, 3, 1, 2, 0, 1, 0 | 1, 0, 3, 1 | 0, 2 | 2, 0, 1, 0 | 0, 0, 1, 0 |
| A[C > T]A | 8, 4, 5, 3 | 8, 4, 7, 3, 5, 3, 6 | 5, 8, 6, 4 | 5, 3 | 3, 5, 3, 6 | 3, 2, 3, 4 |
| A[C > T]C | 2, 1, 2, 1 | 5, 1, 2, 2, 1, 1, 0 | 2, 1, 0, 3 | 1, 1 | 2, 1, 1, 0 | 1, 4, 2, 2 |
| A[C > T]G | 4, 1, 1, 1 | 3, 5, 5, 2, 4, 2, 2 | 4, 0, 2, 2 | 2, 4 | 2, 4, 2, 2 | 0, 0, 0, 2 |
| A[C > T]T | 4, 3, 1, 2 | 3, 1, 3, 4, 1, 1, 2 | 3, 3, 4, 1 | 3, 2 | 4, 1, 1, 2 | 1, 1, 3, 2 |
| A[T > A]A | 0, 0, 0, 1 | 1, 0, 1, 0, 0, 0, 0 | 0, 2, 1, 1 | 0, 0 | 0, 0, 0, 0 | 0, 0, 1, 1 |
| A[T > A]C | 1, 1, 0, 0 | 0, 0, 0, 1, 0, 0, 0 | 2, 1, 0, 0 | 1, 0 | 1, 0, 0, 0 | 0, 1, 0, 1 |
| A[T > A]G | 1, 1, 1, 1 | 1, 1, 1, 0, 1, 0, 1 | 0, 1, 1, 1 | 0, 1 | 0, 1, 0, 1 | 0, 0, 0, 0 |
| A[T > A]T | 1, 1, 0, 0 | 4, 2, 5, 1, 1, 1, 1 | 5, 2, 1, 5 | 3, 1 | 1, 1, 1, 1 | 2, 1, 0, 0 |
| A[T > C]A | 4, 2, 1, 2 | 3, 5, 2, 0, 2, 0, 1 | 1, 3, 3, 0 | 3, 9 | 0, 2, 0, 1 | 1, 1, 1, 1 |
| A[T > C]C | 0, 0, 1, 0 | 0, 0, 0, 1, 0, 0, 0 | 0, 1, 1, 1 | 1, 0 | 1, 0, 0, 0 | 0, 0, 1, 1 |
| A[T > C]G | 2, 2, 0, 0 | 1, 0, 2, 1, 1, 0, 0 | 3, 1, 1, 3 | 1, 1 | 1, 1, 0, 0 | 0, 1, 1, 1 |
| A[T > C]T | 1, 2, 1, 0 | 1, 1, 1, 4, 0, 1, 0 | 1, 6, 2, 0 | 1, 2 | 4, 0, 1, 0 | 3, 1, 0, 1 |
| A[T > G]A | 0, 1, 0, 0 | 0, 1, 0, 1, 0, 0, 0 | 0, 0, 0, 0 | 0, 0 | 1, 0, 0, 0 | 0, 0, 0, 0 |
| A[T > G]C | 0, 0, 0, 0 | 0, 1, 0, 0, 0, 0, 0 | 2, 0, 1, 0 | 0, 0 | 0, 0, 0, 0 | 0, 0, 0, 0 |
| A[T > G]G | 0, 0, 0, 0 | 0, 0, 0, 0, 0, 0, 0 | 0, 0, 0, 0 | 0, 1 | 0, 0, 0, 0 | 0, 1, 0, 0 |
| A[T > G]T | 1, 2, 0, 1 | 0, 3, 2, 0, 0, 0, 0 | 1, 0, 2, 0 | 0, 0 | 0, 0, 0, 0 | 0, 2, 0, 0 |
| C[C > A]A | 8, 14, 6, 1 | 8, 10, 16, 11, 9, 9, 6 | 12, 10, 10, 6 | 2, 10 | 11, 9, 9, 6 | 5, 3, 5, 8 |
| C[C > A]C | 2, 2, 0, 1 | 0, 1, 1, 0, 3, 1, 0 | 0, 0, 4, 2 | 2, 1 | 0, 3, 1, 0 | 2, 0, 2, 1 |
| C[C > A]G | 0, 0, 0, 0 | 1, 1, 2, 0, 1, 0, 0 | 1, 0, 3, 1 | 1, 0 | 0, 1, 0, 0 | 1, 0, 1, 0 |
| C[C > A]T | 5, 7, 2, 3 | 5, 3, 8, 7, 11, 2, 1 | 3, 2, 11, 2 | 3, 3 | 7, 11, 2, 1 | 6, 3, 3, 1 |
| C[C > G]A | 1, 2, 0, 0 | 3, 1, 0, 0, 0, 0, 1 | 0, 2, 0, 0 | 1, 0 | 0, 0, 0, 1 | 0, 1, 0, 0 |
| C[C > G]C | 1, 1, 0, 0 | 1, 3, 0, 1, 0, 0, 0 | 1, 1, 2, 0 | 0, 3 | 1, 0, 0, 0 | 0, 0, 0, 0 |
| C[C > G]G | 1, 0, 0, 0 | 0, 0, 1, 0, 0, 0, 1 | 0, 0, 0, 1 | 0, 0 | 0, 0, 0, 1 | 0, 0, 0, 0 |
| C[C > G]T | 0, 1, 2, 0 | 1, 2, 0, 4, 0, 1, 0 | 0, 3, 1, 1 | 0, 0 | 4, 0, 1, 0 | 0, 0, 0, 1 |
| C[C > T]A | 5, 5, 2, 1 | 6, 3, 9, 4, 8, 2, 5 | 4, 2, 3, 6 | 3, 2 | 4, 8, 2, 5 | 3, 7, 6, 2 |
| C[C > T]C | 0, 7, 3, 2 | 3, 0, 5, 3, 5, 2, 3 | 3, 8, 7, 4 | 0, 6 | 3, 5, 2, 3 | 3, 4, 2, 3 |
| C[C > T]G | 1, 3, 0, 1 | 2, 3, 7, 1, 4, 2, 1 | 4, 3, 0, 2 | 0, 0 | 1, 4, 2, 1 | 3, 4, 2, 3 |
| C[C > T]T | 4, 2, 1, 5 | 6, 3, 2, 4, 6, 3, 3 | 2, 0, 2, 2 | 2, 3 | 4, 6, 3, 3 | 2, 1, 1, 1 |
| C[T > A]A | 2, 0, 0, 0 | 1, 0, 3, 0, 1, 0, 0 | 1, 0, 1, 0 | 0, 2 | 0, 1, 0, 0 | 0, 0, 0, 0 |
| C[T > A]C | 0, 1, 0, 1 | 2, 1, 0, 1, 0, 1, 1 | 2, 0, 0, 0 | 1, 0 | 1, 0, 1, 1 | 0, 1, 0, 2 |
| C[T > A]G | 0, 0, 0, 0 | 0, 1, 2, 2, 0, 1, 0 | 0, 1, 1, 0 | 1, 0 | 2, 0, 1, 0 | 0, 0, 1, 1 |
| C[T > A]T | 2, 0, 1, 0 | 3, 0, 2, 1, 0, 1, 1 | 1, 0, 1, 0 | 0, 0 | 1, 0, 1, 1 | 0, 1, 1, 0 |
| C[T > C]A | 0, 1, 0, 0 | 1, 0, 2, 2, 0, 0, 1 | 1, 1, 1, 0 | 1, 1 | 2, 0, 0, 1 | 0, 1, 1, 1 |
| C[T > C]C | 1, 1, 0, 0 | 1, 0, 2, 1, 0, 0, 0 | 0, 0, 1, 0 | 0, 0 | 1, 0, 0, 0 | 1, 1, 0, 0 |
| C[T > C]G | 2, 1, 2, 0 | 0, 0, 3, 1, 2, 3, 0 | 1, 0, 1, 0 | 1, 0 | 1, 2, 3, 0 | 0, 1, 0, 0 |
| C[T > C]T | 1, 0, 0, 1 | 2, 0, 2, 0, 3, 0, 1 | 3, 1, 1, 0 | 1, 0 | 0, 3, 0, 1 | 1, 0, 1, 1 |
| C[T > G]A | 0, 1, 0, 0 | 0, 0, 0, 0, 1, 0, 0 | 0, 1, 0, 0 | 0, 0 | 0, 1, 0, 0 | 0, 0, 0, 0 |
| C[T > G]C | 0, 0, 0, 0 | 0, 0, 1, 0, 0, 0, 0 | 2, 0, 0, 0 | 0, 0 | 0, 0, 0, 0 | 0, 1, 0, 1 |
| C[T > G]G | 0, 1, 0, 0 | 0, 0, 0, 1, 0, 0, 0 | 0, 0, 1, 0 | 1, 1 | 1, 0, 0, 0 | 0, 0, 0, 0 |
| C[T > G]T | 0, 0, 0, 0 | 0, 0, 1, 0, 3, 1, 1 | 2, 0, 0, 0 | 1, 0 | 0, 3, 1, 1 | 0, 1, 0, 0 |
| G[C > A]A | 34, 26, 28, 21 | 47, 26, 63, 35, 47, 32, 24 | 48, 37, 56, 53 | 9, 19 | 35, 47, 32, 24 | 16, 18, 19, 25 |
| G[C > A]C | 2, 3, 0, 2 | 3, 2, 4, 2, 1, 1, 1 | 2, 1, 6, 5 | 1, 3 | 2, 1, 1, 1 | 1, 2, 1, 0 |
| G[C > A]G | 2, 0, 0, 1 | 0, 4, 1, 4, 2, 2, 1 | 2, 1, 1, 3 | 1, 4 | 4, 2, 2, 1 | 0, 1, 2, 1 |
| G[C > A]T | 13, 14, 10, 7 | 16, 17, 23, 7, 19, 12, 13 | 15, 10, 16, 16 | 6, 9 | 7, 19, 12, 13 | 6, 8, 8, 5 |
| G[C > G]A | 0, 0, 0, 0 | 0, 0, 0, 0, 0, 0, 0 | 2, 1, 1, 2 | 0, 1 | 0, 0, 0, 0 | 0, 1, 0, 0 |
| G[C > G]C | 2, 0, 0, 0 | 0, 1, 0, 0, 0, 0, 0 | 2, 1, 2, 1 | 0, 0 | 0, 0, 0, 0 | 0, 0, 0, 0 |
| G[C > G]G | 0, 0, 0, 0 | 0, 0, 0, 0, 1, 0, 0 | 1, 0, 0, 0 | 0, 0 | 0, 1, 0, 0 | 0, 1, 1, 0 |
| G[C > G]T | 2, 0, 0, 0 | 1, 1, 2, 0, 2, 2, 0 | 1, 0, 0, 1 | 0, 0 | 0, 2, 2, 0 | 0, 0, 0, 1 |
| G[C > T]A | 9, 3, 2, 1 | 5, 0, 5, 5, 3, 1, 0 | 4, 5, 3, 4 | 5, 1 | 5, 3, 1, 0 | 3, 2, 1, 2 |
| G[C > T]C | 4, 2, 3, 0 | 1, 3, 6, 0, 1, 1, 3 | 0, 2, 2, 2 | 1, 3 | 0, 1, 1, 3 | 1, 1, 2, 0 |
| G[C > T]G | 1, 0, 2, 2 | 4, 6, 2, 2, 1, 2, 5 | 2, 5, 4, 2 | 0, 1 | 2, 1, 2, 5 | 0, 0, 2, 0 |
| G[C > T]T | 3, 3, 1, 3 | 4, 0, 4, 4, 5, 2, 3 | 3, 1, 4, 1 | 1, 0 | 4, 5, 2, 3 | 2, 1, 3, 1 |
| G[T > A]A | 1, 1, 0, 0 | 1, 1, 1, 0, 0, 0, 0 | 0, 0, 0, 0 | 0, 0 | 0, 0, 0, 0 | 0, 0, 0, 0 |
| G[T > A]C | 1, 0, 0, 0 | 0, 0, 1, 0, 0, 0, 0 | 1, 1, 0, 1 | 1, 0 | 0, 0, 0, 0 | 0, 0, 0, 0 |
| G[T > A]G | 1, 0, 0, 0 | 0, 2, 2, 1, 0, 0, 0 | 1, 1, 1, 1 | 0, 0 | 1, 0, 0, 0 | 0, 0, 0, 0 |
| G[T > A]T | 0, 1, 0, 0 | 0, 0, 0, 0, 0, 0, 0 | 0, 0, 0, 1 | 0, 0 | 0, 0, 0, 0 | 1, 0, 0, 1 |
| G[T > C]A | 2, 1, 0, 1 | 0, 2, 2, 2, 2, 0, 1 | 0, 2, 2, 1 | 1, 0 | 2, 2, 0, 1 | 1, 2, 0, 0 |
| G[T > C]C | 1, 0, 0, 0 | 1, 2, 0, 0, 0, 0, 0 | 0, 0, 0, 1 | 1, 0 | 0, 0, 0, 0 | 1, 0, 0, 0 |
| G[T > C]G | 1, 0, 0, 0 | 0, 1, 1, 2, 1, 0, 1 | 0, 0, 1, 0 | 0, 0 | 2, 1, 0, 1 | 1, 0, 0, 0 |
| G[T > C]T | 2, 3, 2, 0 | 0, 1, 1, 2, 1, 0, 0 | 1, 3, 1, 1 | 1, 2 | 2, 1, 0, 0 | 1, 0, 0, 0 |
| G[T > G]A | 0, 0, 0, 0 | 0, 0, 0, 0, 0, 0, 0 | 0, 0, 0, 1 | 1, 0 | 0, 0, 0, 0 | 0, 0, 0, 0 |
| G[T > G]C | 0, 0, 0, 1 | 0, 0, 0, 0, 0, 0, 0 | 0, 0, 1, 0 | 0, 0 | 0, 0, 0, 0 | 0, 0, 0, 0 |
| G[T > G]G | 0, 0, 0, 0 | 0, 0, 0, 0, 0, 0, 0 | 1, 0, 0, 0 | 0, 0 | 0, 0, 0, 0 | 0, 1, 0, 0 |
| G[T > G]T | 0, 0, 0, 0 | 0, 0, 1, 0, 1, 0, 0 | 0, 0, 2, 1 | 0, 0 | 0, 1, 0, 0 | 0, 0, 0, 0 |
| T[C > A]A | 20, 16, 11, 6 | 11, 10, 14, 20, 29, 11, 12 | 19, 20, 22, 16 | 7, 11 | 20, 29, 11, 12 | 9, 13, 10, 11 |
| T[C > A]C | 3, 4, 3, 0 | 3, 7, 9, 7, 7, 4, 1 | 4, 7, 10, 8 | 1, 3 | 7, 7, 4, 1 | 5, 5, 2, 0 |
| T[C > A]G | 0, 0, 0, 0 | 2, 0, 0, 6, 1, 3, 3 | 0, 2, 3, 1 | 0, 0 | 6, 1, 3, 3 | 0, 0, 0, 3 |
| T[C > A]T | 28, 26, 14, 19 | 48, 20, 46, 39, 37, 25, 20 | 35, 32, 59, 34 | 18, 15 | 39, 37, 25, 20 | 15, 25, 20, 16 |
| T[C > G]A | 1, 0, 1, 0 | 1, 0, 2, 0, 2, 0, 2 | 0, 2, 2, 0 | 0, 1 | 0, 2, 0, 2 | 0, 0, 0, 0 |
| T[C > G]C | 2, 5, 0, 0 | 2, 1, 0, 1, 2, 0, 0 | 2, 0, 1, 0 | 0, 0 | 1, 2, 0, 0 | 0, 0, 0, 1 |
| T[C > G]G | 0, 0, 0, 0 | 0, 0, 1, 0, 1, 0, 0 | 1, 1, 0, 1 | 0, 0 | 0, 1, 0, 0 | 0, 0, 0, 0 |
| T[C > G]T | 1, 2, 1, 0 | 2, 2, 0, 1, 1, 0, 0 | 5, 1, 1, 2 | 0, 2 | 1, 1, 0, 0 | 2, 1, 0, 1 |
| T[C > T]A | 3, 5, 4, 4 | 5, 1, 6, 3, 4, 4, 3 | 7, 4, 4, 8 | 3, 2 | 3, 4, 4, 3 | 3, 1, 2, 5 |
| T[C > T]C | 6, 1, 2, 0 | 2, 4, 4, 1, 2, 0, 4 | 3, 6, 1, 4 | 2, 4 | 1, 2, 0, 4 | 2, 4, 3, 0 |
| T[C > T]G | 1, 1, 1, 0 | 2, 2, 6, 3, 2, 1, 1 | 2, 2, 3, 3 | 1, 1 | 3, 2, 1, 1 | 6, 3, 0, 1 |
| T[C > T]T | 2, 6, 1, 0 | 0, 0, 8, 0, 2, 1, 2 | 4, 2, 2, 4 | 1, 1 | 0, 2, 1, 2 | 2, 4, 2, 0 |
| T[T > A]A | 3, 1, 0, 1 | 2, 3, 1, 2, 1, 0, 2 | 1, 1, 4, 0 | 2, 3 | 2, 1, 0, 2 | 1, 0, 0, 3 |
| T[T > A]C | 0, 2, 0, 0 | 2, 0, 1, 0, 1, 0, 0 | 0, 0, 0, 0 | 1, 0 | 0, 1, 0, 0 | 0, 1, 0, 0 |
| T[T > A]G | 0, 0, 0, 0 | 0, 2, 0, 0, 1, 0, 1 | 2, 0, 2, 2 | 1, 0 | 0, 1, 0, 1 | 0, 1, 0, 0 |
| T[T > A]T | 3, 1, 0, 2 | 4, 1, 2, 4, 3, 0, 4 | 2, 1, 1, 1 | 1, 1 | 4, 3, 0, 4 | 0, 1, 1, 0 |
| T[T > C]A | 0, 1, 4, 1 | 4, 3, 7, 4, 3, 4, 2 | 1, 3, 7, 2 | 2, 3 | 4, 3, 4, 2 | 1, 0, 0, 1 |
| T[T > C]C | 1, 0, 0, 1 | 0, 2, 0, 0, 2, 0, 2 | 2, 0, 1, 0 | 2, 2 | 0, 2, 0, 2 | 0, 1, 0, 0 |
| T[T > C]G | 1, 0, 1, 0 | 2, 0, 1, 1, 0, 2, 1 | 1, 1, 1, 1 | 0, 2 | 1, 0, 2, 1 | 2, 0, 0, 1 |
| T[T > C]T | 3, 1, 4, 1 | 0, 2, 5, 1, 4, 0, 1 | 2, 4, 3, 4 | 3, 4 | 1, 4, 0, 1 | 0, 1, 1, 2 |
| T[T > G]A | 0, 0, 1, 0 | 0, 0, 0, 0, 2, 0, 0 | 0, 1, 0, 0 | 2, 0 | 0, 2, 0, 0 | 0, 0, 0, 0 |
| T[T > G]C | 0, 1, 1, 2 | 0, 0, 1, 0, 0, 1, 0 | 0, 0, 0, 1 | 0, 0 | 0, 0, 1, 0 | 0, 0, 0, 1 |
| T[T > G]G | 0, 0, 0, 0 | 0, 0, 1, 0, 0, 1, 0 | 1, 1, 0, 0 | 1, 2 | 0, 0, 1, 0 | 0, 2, 1, 0 |
| T[T > G]T | 0, 0, 2, 0 | 1, 1, 2, 1, 4, 1, 0 | 1, 2, 1, 2 | 0, 1 | 1, 4, 1, 0 | 1, 1, 0, 1 |

| Mutation | | | | | |
|---|---|---|---|---|---|
| Type | ATP2B4 | POLE4 | PIAS1 | PIAS4 | C1orf86 |
| A[C > A]A | 11, 11, 4, 23, 10, 18, 13, 26 | 10, 13, 14, 16 | 8, 5, 5, 10 | 6, 14, 22, 16 | 11, 7, 11, 13 |
| A[C > A]C | 2, 1, 0, 5, 0, 1, 1, 2 | 1, 1, 2, 2 | 0, 0, 1, 1 | 0, 2, 0, 2 | 1, 0, 3, 1 |
| A[C > A]G | 0, 0, 1, 3, 1, 0, 1, 0 | 1, 0, 1, 1 | 0, 0, 0, 0 | 1, 0, 0, 0 | 0, 0, 0, 1 |
| A[C > A]T | 1, 3, 1, 7, 7, 6, 5, 3 | 3, 3, 4, 2 | 1, 2, 3, 3 | 2, 3, 7, 7 | 7, 2, 3, 8 |
| A[C > G]A | 0, 0, 0, 3, 0, 0, 0, 2 | 0, 2, 1, 1 | 0, 0, 1, 1 | 0, 1, 1, 3 | 0, 0, 0, 0 |
| A[C > G]C | 1, 0, 0, 0, 0, 2, 1, 1 | 0, 0, 1, 0 | 0, 1, 1, 1 | 1, 0, 0, 0 | 0, 0, 0, 1 |
| A[C > G]G | 0, 0, 0, 2, 0, 0, 1, 0 | 1, 0, 1, 0 | 1, 1, 0, 1 | 0, 1, 1, 0 | 0, 0, 0, 0 |
| A[C > G]T | 0, 0, 0, 0, 1, 1, 2, 1 | 0, 0, 1, 3 | 0, 1, 0, 0 | 1, 1, 2, 2 | 1, 0, 1, 1 |
| A[C > T]A | 6, 8, 2, 8, 6, 7, 6, 7 | 5, 1, 6, 7 | 4, 4, 7, 2 | 3, 5, 7, 11 | 7, 3, 1, 2 |
| A[C > T]C | 2, 0, 3, 1, 1, 3, 3, 6 | 5, 2, 2, 1 | 0, 0, 3, 3 | 3, 3, 1, 2 | 4, 1, 2, 1 |
| A[C > T]G | 2, 5, 1, 3, 1, 1, 3, 1 | 2, 4, 5, 0 | 0, 0, 3, 1 | 4, 3, 4, 1 | 4, 1, 3, 0 |
| A[C > T]T | 3, 1, 2, 2, 0, 3, 1, 4 | 4, 2, 2, 3 | 1, 1, 2, 0 | 2, 0, 2, 0 | 0, 3, 3, 4 |
| A[T > A]A | 0, 1, 0, 1, 0, 1, 2, 3 | 0, 1, 1, 1 | 0, 1, 0, 1 | 0, 0, 1, 1 | 0, 0, 1, 1 |
| A[T > A]C | 0, 0, 0, 0, 0, 1, 1, 2 | 0, 0, 1, 0 | 0, 0, 0, 1 | 0, 0, 0, 0 | 0, 0, 0, 0 |
| A[T > A]G | 0, 0, 0, 1, 0, 0, 2, 0 | 2, 0, 2, 1 | 1, 0, 0, 0 | 0, 0, 0, 0 | 1, 0, 1, 2 |
| A[T > A]T | 1, 2, 0, 1, 1, 0, 1, 3 | 2, 1, 6, 1 | 0, 0, 0, 2 | 0, 2, 1, 0 | 4, 0, 1, 3 |
| A[T > C]A | 1, 2, 0, 1, 0, 4, 5, 4 | 2, 2, 1, 4 | 1, 3, 2, 1 | 0, 1, 6, 4 | 0, 2, 1, 8 |
| A[T > C]C | 0, 1, 0, 0, 0, 1, 1, 0 | 0, 1, 1, 2 | 0, 0, 1, 0 | 0, 0, 1, 1 | 1, 0, 1, 2 |
| A[T > C]G | 0, 3, 1, 1, 0, 4, 1, 2 | 1, 2, 2, 1 | 1, 2, 1, 1 | 1, 0, 2, 1 | 1, 0, 0, 0 |
| A[T > C]T | 2, 1, 2, 1, 0, 2, 2, 0 | 2, 0, 1, 0 | 3, 1, 1, 0 | 1, 2, 2, 1 | 0, 3, 1, 3 |
| A[T > G]A | 0, 0, 1, 2, 1, 0, 0, 3 | 0, 0, 0, 1 | 0, 0, 0, 1 | 1, 0, 0, 0 | 0, 1, 1, 0 |
| A[T > G]C | 0, 0, 1, 0, 0, 0, 0, 0 | 0, 0, 0, 0 | 0, 0, 0, 0 | 0, 0, 0, 0 | 0, 0, 0, 0 |
| A[T > G]G | 0, 0, 0, 0, 0, 0, 0, 0 | 0, 0, 0, 0 | 0, 0, 1, 0 | 0, 0, 0, 0 | 1, 2, 1, 0 |
| A[T > G]T | 0, 0, 0, 1, 0, 1, 1, 1 | 0, 1, 0, 1 | 0, 0, 0, 1 | 0, 0, 0, 0 | 3, 0, 1, 1 |
| C[C > A]A | 3, 12, 8, 15, 5, 9, 9, 9 | 9, 5, 6, 7 | 2, 3, 12, 8 | 1, 1, 9, 6 | 5, 9, 10, 10 |
| C[C > A]C | 3, 0, 2, 1, 0, 2, 4, 1 | 1, 2, 2, 1 | 3, 0, 2, 1 | 0, 1, 0, 1 | 1, 3, 0, 0 |
| C[C > A]G | 2, 1, 1, 2, 0, 2, 1, 0 | 0, 0, 1, 0 | 1, 0, 1, 2 | 0, 1, 1, 0 | 1, 1, 0, 1 |
| C[C > A]T | 3, 6, 1, 10, 1, 4, 3, 3 | 1, 1, 1, 5 | 3, 5, 4, 3 | 5, 5, 2, 10 | 7, 8, 4, 4 |
| C[C > G]A | 0, 1, 0, 1, 0, 1, 1, 0 | 2, 0, 1, 4 | 0, 0, 0, 0 | 0, 0, 0, 0 | 1, 0, 0, 1 |
| C[C > G]C | 0, 0, 0, 1, 1, 0, 0, 1 | 0, 0, 0, 0 | 1, 1, 1, 0 | 1, 1, 1, 0 | 2, 0, 0, 0 |
| C[C > G]G | 1, 1, 0, 1, 0, 0, 1, 1 | 0, 0, 0, 1 | 0, 1, 0, 0 | 0, 0, 0, 0 | 0, 0, 1, 0 |
| C[C > G]T | 1, 0, 0, 1, 0, 0, 1, 1 | 2, 0, 4, 0 | 0, 0, 0, 0 | 0, 2, 0, 2 | 0, 0, 0, 2 |
| C[C > T]A | 5, 6, 4, 11, 3, 4, 5, 16 | 6, 1, 4, 4 | 1, 3, 7, 3 | 4, 5, 4, 2 | 1, 16, 4, 5 |
| C[C > T]C | 2, 1, 4, 4, 2, 2, 4, 2 | 4, 3, 0, 3 | 1, 2, 1, 4 | 3, 3, 1, 4 | 1, 1, 4, 2 |
| C[C > T]G | 0, 1, 2, 2, 1, 0, 4, 6 | 5, 4, 1, 2 | 2, 3, 1, 1 | 2, 0, 6, 2 | 2, 1, 1, 2 |
| C[C > T]T | 3, 2, 2, 3, 1, 2, 1, 1 | 3, 2, 3, 3 | 4, 5, 2, 0 | 1, 4, 3, 0 | 1, 3, 2, 4 |
| C[T > A]A | 0, 1, 0, 1, 0, 0, 1, 0 | 0, 0, 3, 1 | 0, 0, 1, 0 | 0, 0, 1, 1 | 0, 1, 0, 0 |
| C[T > A]C | 0, 0, 1, 1, 0, 0, 0, 0 | 1, 1, 1, 0 | 0, 0, 1, 1 | 0, 0, 0, 0 | 0, 0, 0, 2 |
| C[T > A]G | 3, 1, 0, 0, 0, 0, 1, 1 | 2, 1, 1, 0 | 0, 0, 0, 0 | 1, 0, 2, 0 | 1, 0, 1, 0 |
| C[T > A]T | 0, 1, 0, 2, 0, 2, 1, 0 | 0, 1, 0, 1 | 1, 1, 2, 0 | 0, 0, 1, 2 | 1, 2, 0, 0 |
| C[T > C]A | 1, 2, 0, 1, 1, 3, 3, 2 | 2, 1, 3, 2 | 0, 1, 1, 4 | 3, 1, 1, 2 | 0, 0, 1, 1 |
| C[T > C]C | 0, 0, 0, 1, 1, 0, 0, 1 | 0, 0, 1, 3 | 0, 2, 0, 1 | 0, 0, 2, 2 | 1, 0, 1, 0 |
| C[T > C]G | 0, 1, 0, 1, 3, 2, 1, 1 | 1, 0, 0, 1 | 0, 0, 0, 1 | 0, 0, 2, 3 | 0, 1, 0, 0 |
| C[T > C]T | 1, 1, 1, 1, 1, 1, 1, 1 | 0, 1, 0, 0 | 1, 1, 0, 0 | 1, 0, 1, 0 | 3, 1, 1, 1 |
| C[T > G]A | 0, 1, 1, 1, 0, 0, 0, 0 | 1, 0, 0, 0 | 0, 0, 0, 0 | 0, 0, 0, 1 | 0, 0, 0, 1 |
| C[T > G]C | 0, 0, 0, 0, 0, 0, 0, 1 | 0, 0, 0, 0 | 0, 0, 0, 0 | 0, 0, 1, 0 | 0, 0, 0, 0 |
| C[T > G]G | 0, 0, 0, 2, 1, 0, 0, 0 | 0, 0, 0, 0 | 0, 0, 0, 0 | 0, 0, 1, 0 | 1, 1, 0, 0 |
| C[T > G]T | 1, 0, 0, 1, 1, 2, 0, 0 | 0, 0, 1, 0 | 1, 0, 1, 0 | 0, 0, 0, 0 | 0, 0, 0, 0 |
| G[C > A]A | 21, 45, 28, 58, 26, 35, 38, 46 | 21, 31, 22, 26 | 26, 18, 23, 28 | 24, 22, 39, 41 | 28, 20, 21, 29 |
| G[C > A]C | 1, 4, 2, 4, 3, 0, 3, 0 | 1, 2, 3, 3 | 1, 1, 3, 1 | 1, 2, 2, 0 | 2, 1, 0, 1 |
| G[C > A]G | 2, 2, 0, 1, 2, 1, 1, 1 | 0, 0, 2, 0 | 0, 1, 2, 1 | 1, 2, 0, 1 | 3, 2, 2, 0 |
| G[C > A]T | 11, 11, 8, 24, 10, 10, 14, 23 | 12, 8, 14, 7 | 8, 10, 7, 9 | 9, 14, 10, 13 | 9, 12, 7, 8 |
| G[C > G]A | 0, 0, 0, 1, 1, 2, 0, 0 | 0, 0, 1, 0 | 0, 0, 1, 0 | 0, 1, 2, 1 | 0, 1, 1, 0 |
| G[C > G]C | 0, 0, 0, 1, 1, 0, 0, 0 | 0, 0, 0, 1 | 0, 0, 0, 0 | 0, 0, 1, 0 | 0, 1, 0, 0 |
| G[C > G]G | 0, 1, 0, 0, 0, 0, 0, 0 | 0, 1, 0, 0 | 0, 0, 1, 2 | 0, 0, 2, 0 | 0, 0, 0, 0 |
| G[C > G]T | 0, 0, 2, 0, 0, 0, 1, 1 | 0, 0, 0, 2 | 0, 0, 0, 0 | 0, 0, 1, 1 | 0, 2, 1, 1 |
| G[C > T]A | 2, 1, 3, 2, 1, 4, 5, 4 | 2, 2, 3, 3 | 0, 0, 2, 4 | 5, 1, 4, 4 | 2, 3, 2, 1 |
| G[C > T]C | 6, 1, 1, 2, 1, 2, 1, 2 | 1, 2, 3, 2 | 4, 1, 0, 1 | 0, 1, 3, 1 | 3, 2, 6, 8 |
| G[C > T]G | 1, 6, 3, 4, 1, 1, 4, 3 | 0, 0, 2, 0 | 1, 2, 4, 0 | 3, 4, 1, 4 | 3, 0, 1, 1 |
| G[C > T]T | 2, 1, 1, 0, 1, 0, 3, 0 | 3, 0, 3, 6 | 1, 2, 0, 1 | 2, 3, 2, 1 | 0, 2, 0, 1 |
| G[T > A]A | 1, 0, 0, 0, 0, 0, 0, 0 | 1, 0, 0, 2 | 0, 0, 0, 0 | 0, 0, 0, 0 | 0, 0, 0, 0 |
| G[T > A]C | 1, 0, 0, 0, 1, 0, 0, 0 | 1, 0, 0, 1 | 0, 0, 0, 0 | 0, 1, 0, 0 | 0, 0, 1, 0 |
| G[T > A]G | 0, 0, 0, 0, 0, 0, 1, 0 | 1, 0, 0, 0 | 0, 0, 0, 0 | 0, 0, 2, 0 | 0, 0, 0, 0 |
| G[T > A]T | 0, 0, 1, 2, 0, 0, 0, 0 | 0, 1, 1, 0 | 0, 2, 0, 0 | 0, 1, 0, 0 | 1, 0, 3, 0 |
| G[T > C]A | 0, 0, 0, 1, 0, 0, 0, 0 | 3, 0, 0, 3 | 2, 0, 0, 0 | 0, 0, 1, 2 | 1, 1, 0, 0 |
| G[T > C]C | 0, 0, 0, 2, 0, 0, 0, 0 | 1, 1, 1, 2 | 2, 0, 0, 1 | 0, 0, 0, 0 | 0, 0, 0, 1 |
| G[T > C]G | 0, 0, 0, 1, 1, 0, 1, 1 | 0, 0, 0, 0 | 1, 3, 0, 1 | 1, 1, 1, 1 | 0, 1, 1, 1 |
| G[T > C]T | 0, 1, 0, 1, 0, 1, 0, 0 | 1, 0, 0, 2 | 0, 0, 0, 1 | 1, 2, 0, 3 | 1, 1, 0, 0 |
| G[T > G]A | 0, 1, 0, 1, 0, 0, 0, 0 | 0, 0, 0, 1 | 0, 0, 0, 0 | 0, 0, 0, 1 | 0, 0, 0, 0 |
| G[T > G]C | 0, 1, 0, 0, 2, 0, 0, 0 | 0, 0, 0, 0 | 0, 0, 0, 0 | 0, 0, 1, 0 | 0, 0, 0, 0 |
| G[T > G]G | 0, 1, 0, 0, 0, 0, 0, 0 | 0, 0, 0, 0 | 0, 0, 1, 0 | 0, 0, 0, 1 | 0, 1, 0, 0 |
| G[T > G]T | 0, 0, 0, 1, 0, 0, 0, 1 | 1, 0, 0, 0 | 0, 2, 0, 1 | 0, 1, 1, 1 | 1, 0, 0, 1 |
| T[C > A]A | 6, 17, 5, 24, 8, 16, 12, 21 | 8, 2, 11, 7 | 8, 5, 10, 16 | 15, 8, 10, 9 | 9, 14, 14, 14 |
| T[C > A]C | 3, 4, 3, 15, 3, 3, 10, 8 | 1, 3, 3, 1 | 3, 0, 10, 1 | 2, 3, 4, 1 | 5, 4, 5, 7 |
| T[C > A]G | 1, 0, 0, 2, 1, 3, 2, 2 | 0, 1, 1, 0 | 1, 1, 0, 0 | 3, 1, 0, 1 | 1, 0, 1, 0 |
| T[C > A]T | 25, 31, 13, 47, 17, 29, 25, 34 | 35, 21, 24, 17 | 12, 12, 11, 12 | 20, 20, 23, 23 | 14, 19, 22, 19 |
| T[C > G]A | 0, 0, 0, 1, 0, 1, 1, 1 | 1, 1, 3, 0 | 1, 2, 0, 0 | 1, 0, 2, 0 | 0, 1, 2, 1 |
| T[C > G]C | 0, 0, 1, 2, 0, 0, 0, 2 | 0, 0, 0, 0 | 1, 1, 0, 0 | 0, 0, 0, 1 | 0, 0, 0, 1 |
| T[C > G]G | 0, 0, 0, 2, 0, 1, 0, 0 | 0, 1, 0, 0 | 0, 0, 0, 0 | 0, 0, 0, 0 | 0, 0, 0, 0 |
| T[C > G]T | 1, 1, 3, 1, 0, 3, 3, 2 | 0, 1, 3, 1 | 1, 1, 1, 0 | 0, 0, 5, 1 | 2, 0, 1, 2 |
| T[C > T]A | 3, 2, 4, 2, 2, 7, 6, 6 | 3, 2, 5, 4 | 3, 4, 2, 3 | 3, 3, 4, 3 | 3, 3, 3, 1 |
| T[C > T]C | 3, 1, 2, 3, 1, 4, 3, 4 | 3, 1, 2, 3 | 4, 0, 4, 4 | 0, 4, 2, 2 | 1, 2, 2, 6 |
| T[C > T]G | 2, 1, 2, 4, 1, 2, 1, 4 | 0, 1, 1, 3 | 1, 1, 1, 3 | 2, 3, 3, 3 | 1, 2, 1, 2 |
| T[C > T]T | 0, 2, 0, 2, 0, 3, 5, 1 | 1, 3, 2, 1 | 1, 1, 0, 0 | 3, 1, 2, 8 | 1, 2, 2, 0 |
| T[T > A]A | 0, 2, 0, 0, 1, 1, 2, 3 | 2, 2, 1, 0 | 0, 0, 1, 1 | 1, 0, 3, 2 | 1, 1, 1, 1 |
| T[T > A]C | 0, 0, 0, 1, 0, 0, 0, 0 | 0, 0, 1, 0 | 0, 0, 0, 0 | 1, 0, 0, 0 | 1, 0, 1, 1 |
| T[T > A]G | 2, 0, 0, 1, 0, 1, 0, 0 | 2, 0, 3, 1 | 0, 1, 0, 0 | 0, 1, 1, 1 | 2, 0, 1, 0 |
| T[T > A]T | 0, 2, 1, 1, 2, 3, 3, 1 | 1, 0, 1, 0 | 0, 3, 0, 0 | 2, 1, 2, 2 | 2, 2, 5, 0 |
| T[T > C]A | 1, 2, 1, 6, 5, 1, 2, 2 | 4, 4, 5, 2 | 1, 1, 3, 2 | 2, 1, 5, 2 | 3, 1, 3, 0 |
| T[T > C]C | 0, 0, 0, 0, 0, 0, 1, 1 | 0, 1, 0, 1 | 0, 2, 0, 1 | 0, 0, 3, 0 | 0, 0, 0, 2 |
| T[T > C]G | 1, 2, 0, 3, 1, 1, 1, 1 | 0, 2, 2, 0 | 1, 0, 1, 6 | 0, 0, 1, 1 | 1, 0, 1, 0 |
| T[T > C]T | 1, 2, 0, 1, 0, 5, 5, 1 | 0, 2, 2, 3 | 1, 2, 3, 1 | 0, 2, 0, 2 | 2, 4, 2, 2 |
| T[T > G]A | 0, 2, 1, 1, 0, 0, 0, 0 | 0, 0, 2, 0 | 0, 0, 2, 1 | 0, 0, 0, 1 | 2, 0, 0, 0 |
| T[T > G]C | 0, 0, 0, 1, 1, 1, 0, 0 | 0, 2, 2, 1 | 0, 0, 1, 1 | 1, 0, 0, 0 | 1, 0, 0, 0 |
| T[T > G]G | 0, 0, 0, 1, 0, 1, 0, 0 | 0, 0, 0, 0 | 0, 0, 0, 0 | 0, 1, 0, 0 | 1, 0, 2, 0 |
| T[T > G]T | 0, 1, 2, 1, 1, 1, 2, 1 | 2, 1, 1, 0 | 0, 1, 0, 0 | 0, 0, 2, 2 | 0, 1, 1, 3 |

| Mutation | | | | | | |
|---|---|---|---|---|---|---|
| Type | DCLRE1A | FAN1 | FANCM | PIF1 | SETX | RECQL5 |
| A[C > A]A | 10, 7, 10, 14 | 17, 13, 22, 13 | 14, 7, 12, 11 | 8, 10, 11, 4 | 9, 9, 13, 4 | 14, 12, 9, 20 |
| A[C > A]C | 0, 0, 0, 0 | 2, 1, 0, 2 | 4, 0, 1, 1 | 2, 0, 1, 2 | 1, 1, 1, 0 | 2, 2, 0, 2 |
| A[C > A]G | 0, 0, 0, 0 | 0, 1, 1, 0 | 1, 1, 1, 0 | 1, 1, 1, 0 | 0, 0, 1, 0 | 0, 1, 0, 1 |
| A[C > A]T | 3, 1, 2, 1 | 2, 3, 7, 5 | 2, 6, 2, 7 | 6, 3, 2, 3 | 3, 2, 5, 1 | 2, 3, 6, 4 |
| A[C > G]A | 1, 1, 0, 1 | 0, 2, 0, 1 | 0, 0, 1, 0 | 0, 2, 0, 2 | 1, 2, 0, 1 | 0, 1, 1, 2 |
| A[C > G]C | 1, 1, 0, 0 | 0, 0, 0, 1 | 0, 0, 1, 0 | 1, 0, 0, 0 | 0, 1, 2, 0 | 0, 0, 2, 0 |
| A[C > G]G | 1, 0, 0, 0 | 0, 2, 0, 0 | 2, 1, 2, 0 | 0, 1, 0, 0 | 0, 1, 0, 0 | 0, 0, 0, 1 |
| A[C > G]T | 0, 1, 0, 0 | 0, 2, 0, 1 | 2, 0, 1, 0 | 0, 0, 1, 1 | 1, 0, 1, 2 | 1, 1, 0, 0 |
| A[C > T]A | 3, 4, 5, 4 | 6, 5, 7, 5 | 6, 4, 8, 6 | 4, 2, 12, 7 | 3, 5, 4, 5 | 3, 3, 7, 6 |
| A[C > T]C | 2, 4, 4, 1 | 1, 1, 3, 1 | 0, 0, 3, 3 | 1, 2, 1, 6 | 4, 3, 4, 2 | 3, 0, 2, 1 |
| A[C > T]G | 5, 0, 4, 3 | 5, 3, 4, 8 | 3, 0, 2, 3 | 2, 2, 6, 3 | 2, 1, 1, 1 | 1, 2, 0, 4 |
| A[C > T]T | 0, 2, 3, 1 | 2, 1, 4, 3 | 6, 2, 3, 1 | 1, 1, 1, 1 | 1, 1, 0, 2 | 0, 1, 5, 3 |
| A[T > A]A | 0, 1, 0, 0 | 0, 1, 1, 2 | 1, 0, 0, 0 | 0, 0, 0, 1 | 1, 0, 1, 2 | 1, 1, 2, 0 |
| A[T > A]C | 0, 0, 0, 0 | 0, 0, 0, 0 | 0, 0, 0, 1 | 0, 0, 0, 0 | 0, 1, 1, 0 | 0, 0, 0, 0 |
| A[T > A]G | 0, 0, 1, 0 | 0, 1, 0, 0 | 1, 0, 2, 0 | 1, 1, 2, 2 | 0, 0, 0, 0 | 0, 0, 0, 2 |
| A[T > A]T | 1, 0, 0, 0 | 1, 1, 4, 0 | 1, 2, 0, 1 | 2, 0, 1, 2 | 2, 2, 2, 1 | 2, 0, 3, 1 |
| A[T > C]A | 1, 1, 1, 1 | 4, 0, 4, 5 | 4, 3, 1, 2 | 1, 3, 3, 2 | 3, 2, 3, 1 | 1, 2, 3, 1 |
| A[T > C]C | 1, 1, 1, 0 | 1, 1, 0, 1 | 1, 0, 0, 0 | 1, 1, 2, 0 | 0, 2, 0, 0 | 0, 0, 0, 0 |
| A[T > C]G | 3, 1, 2, 1 | 0, 3, 1, 3 | 1, 0, 2, 1 | 0, 0, 0, 0 | 2, 0, 2, 0 | 2, 1, 0, 0 |
| A[T > C]T | 3, 0, 0, 1 | 2, 3, 0, 3 | 5, 0, 4, 2 | 1, 1, 1, 1 | 1, 1, 1, 1 | 2, 1, 3, 0 |
| A[T > G]A | 0, 0, 1, 0 | 0, 1, 0, 1 | 0, 0, 0, 0 | 1, 0, 0, 0 | 0, 2, 0, 0 | 0, 0, 2, 1 |
| A[T > G]C | 0, 1, 0, 0 | 0, 0, 0, 0 | 0, 1, 0, 0 | 1, 0, 0, 1 | 1, 0, 0, 0 | 0, 0, 0, 0 |
| A[T > G]G | 0, 0, 0, 0 | 0, 0, 0, 1 | 1, 0, 1, 0 | 0, 1, 1, 1 | 0, 1, 0, 0 | 0, 0, 0, 0 |
| A[T > G]T | 1, 0, 0, 1 | 1, 1, 4, 1 | 2, 0, 0, 1 | 1, 0, 0, 1 | 2, 0, 2, 2 | 0, 0, 0, 0 |
| C[C > A]A | 7, 2, 1, 8 | 5, 8, 10, 3 | 5, 1, 6, 7 | 11, 6, 4, 3 | 10, 4, 5, 5 | 8, 3, 9, 6 |
| C[C > A]C | 2, 2, 3, 0 | 1, 0, 0, 1 | 3, 1, 0, 2 | 1, 0, 1, 2 | 1, 0, 0, 1 | 0, 0, 3, 2 |
| C[C > A]G | 1, 2, 1, 2 | 2, 0, 2, 1 | 1, 0, 0, 1 | 0, 1, 1, 1 | 0, 0, 4, 2 | 1, 1, 0, 0 |
| C[C > A]T | 2, 1, 0, 2 | 5, 5, 4, 4 | 9, 2, 6, 3 | 7, 3, 1, 7 | 4, 2, 6, 6 | 3, 4, 5, 8 |
| C[C > G]A | 0, 1, 0, 0 | 0, 0, 0, 0 | 1, 1, 1, 1 | 0, 0, 0, 0 | 0, 0, 0, 1 | 1, 0, 0, 0 |
| C[C > G]C | 0, 0, 0, 0 | 0, 1, 0, 0 | 3, 0, 1, 0 | 1, 0, 0, 0 | 1, 1, 0, 0 | 0, 1, 2, 0 |
| C[C > G]G | 0, 1, 0, 1 | 0, 0, 0, 0 | 1, 0, 2, 0 | 0, 0, 1, 0 | 0, 0, 0, 0 | 0, 0, 0, 1 |
| C[C > G]T | 1, 1, 1, 1 | 2, 3, 0, 1 | 3, 0, 0, 2 | 1, 0, 0, 3 | 0, 0, 0, 0 | 1, 0, 1, 1 |
| C[C > T]A | 3, 1, 4, 4 | 3, 1, 4, 1 | 6, 3, 4, 5 | 3, 1, 7, 11 | 4, 6, 3, 3 | 3, 3, 4, 3 |
| C[C > T]C | 5, 1, 3, 0 | 3, 1, 2, 1 | 3, 1, 1, 2 | 1, 8, 2, 3 | 0, 2, 2, 0 | 4, 3, 0, 5 |
| C[C > T]G | 0, 2, 4, 8 | 4, 4, 3, 3 | 4, 4, 1, 3 | 1, 1, 2, 0 | 2, 3, 1, 2 | 1, 2, 4, 6 |
| C[C > T]T | 1, 8, 0, 0 | 7, 0, 4, 6 | 2, 4, 5, 6 | 1, 3, 1, 2 | 1, 3, 0, 3 | 0, 0, 3, 2 |
| C[T > A]A | 0, 0, 0, 0 | 1, 1, 1, 1 | 0, 0, 1, 0 | 1, 0, 0, 0 | 2, 1, 0, 1 | 3, 2, 0, 0 |
| C[T > A]C | 0, 1, 0, 0 | 0, 0, 1, 2 | 0, 0, 0, 0 | 0, 0, 1, 0 | 0, 0, 1, 1 | 1, 0, 0, 1 |
| C[T > A]G | 1, 0, 0, 0 | 0, 0, 0, 2 | 1, 0, 2, 2 | 0, 1, 0, 0 | 0, 1, 0, 0 | 0, 0, 0, 0 |
| C[T > A]T | 0, 0, 1, 0 | 0, 0, 0, 0 | 3, 1, 0, 1 | 1, 0, 1, 0 | 0, 2, 1, 0 | 1, 0, 0, 0 |
| C[T > C]A | 1, 3, 2, 0 | 1, 2, 2, 0 | 2, 1, 2, 0 | 0, 1, 1, 1 | 2, 1, 0, 0 | 0, 0, 2, 0 |
| C[T > C]C | 1, 1, 0, 0 | 0, 1, 0, 0 | 1, 1, 0, 0 | 0, 1, 1, 0 | 1, 1, 2, 0 | 1, 0, 1, 1 |
| C[T > C]G | 0, 0, 3, 0 | 1, 2, 2, 1 | 2, 0, 1, 1 | 0, 2, 0, 0 | 1, 2, 1, 0 | 2, 0, 0, 1 |
| C[T > C]T | 0, 0, 0, 1 | 2, 0, 0, 2 | 2, 1, 1, 2 | 2, 0, 2, 3 | 1, 0, 0, 0 | 2, 0, 0, 0 |
| C[T > G]A | 0, 0, 0, 0 | 0, 0, 0, 0 | 0, 0, 0, 0 | 0, 0, 0, 0 | 0, 0, 0, 0 | 0, 1, 0, 0 |
| C[T > G]C | 0, 1, 3, 0 | 0, 0, 0, 1 | 0, 0, 0, 0 | 0, 0, 0, 1 | 0, 1, 0, 0 | 0, 0, 0, 0 |
| C[T > G]G | 0, 0, 1, 1 | 1, 0, 0, 1 | 1, 0, 1, 1 | 0, 0, 0, 0 | 1, 1, 0, 0 | 0, 1, 0, 1 |
| C[T > G]T | 0, 1, 0, 2 | 1, 1, 0, 1 | 1, 0, 0, 0 | 0, 1, 0, 1 | 3, 0, 1, 0 | 0, 1, 0, 0 |
| G[C > A]A | 21, 24, 26, 17 | 28, 28, 34, 38 | 23, 14, 21, 25 | 25, 24, 23, 23 | 28, 23, 24, 31 | 23, 30, 23, 29 |
| G[C > A]C | 3, 1, 1, 2 | 2, 2, 2, 2 | 2, 3, 5, 3 | 0, 1, 1, 4 | 2, 1, 2, 1 | 2, 1, 2, 2 |
| G[C > A]G | 2, 1, 0, 0 | 0, 0, 1, 1 | 1, 3, 1, 1 | 2, 2, 2, 1 | 0, 3, 0, 1 | 3, 0, 1, 1 |
| G[C > A]T | 6, 10, 11, 4 | 7, 8, 14, 15 | 14, 4, 12, 12 | 7, 15, 9, 7 | 11, 17, 9, 7 | 7, 5, 14, 17 |
| G[C > G]A | 0, 0, 0, 3 | 0, 0, 0, 1 | 0, 2, 0, 2 | 1, 0, 1, 1 | 0, 1, 0, 1 | 0, 0, 1, 0 |
| G[C > G]C | 1, 0, 0, 0 | 1, 0, 1, 0 | 0, 0, 1, 0 | 0, 0, 0, 0 | 2, 0, 0, 0 | 0, 0, 0, 0 |
| G[C > G]G | 0, 0, 0, 1 | 0, 0, 0, 0 | 0, 1, 0, 1 | 0, 0, 0, 0 | 0, 0, 1, 0 | 0, 0, 0, 0 |
| G[C > G]T | 0, 0, 1, 0 | 0, 0, 0, 0 | 2, 0, 0, 0 | 0, 0, 0, 0 | 1, 0, 0, 0 | 0, 0, 0, 2 |
| G[C > T]A | 3, 1, 0, 1 | 2, 2, 1, 2 | 3, 2, 3, 3 | 0, 4, 1, 3 | 3, 4, 2, 2 | 3, 3, 3, 3 |
| G[C > T]C | 0, 0, 4, 0 | 0, 2, 2, 4 | 0, 3, 3, 4 | 3, 2, 1, 5 | 2, 2, 0, 2 | 0, 2, 2, 1 |
| G[C > T]G | 1, 3, 3, 2 | 2, 3, 0, 1 | 2, 2, 2, 0 | 1, 3, 2, 2 | 2, 1, 1, 4 | 1, 0, 1, 2 |
| G[C > T]T | 2, 1, 2, 2 | 1, 1, 2, 3 | 5, 1, 3, 3 | 2, 3, 2, 0 | 1, 1, 2, 0 | 3, 2, 0, 4 |
| G[T > A]A | 0, 0, 0, 0 | 0, 1, 1, 0 | 1, 1, 0, 0 | 0, 0, 0, 0 | 1, 0, 1, 2 | 1, 1, 1, 0 |
| G[T > A]C | 0, 0, 0, 0 | 0, 0, 1, 2 | 1, 1, 0, 1 | 0, 0, 0, 1 | 0, 0, 0, 0 | 0, 0, 0, 1 |
| G[T > A]G | 2, 0, 3, 0 | 0, 1, 0, 0 | 0, 0, 0, 0 | 0, 0, 1, 0 | 1, 0, 0, 0 | 0, 0, 0, 0 |
| G[T > A]T | 2, 0, 0, 0 | 0, 0, 0, 0 | 2, 1, 1, 2 | 1, 0, 0, 0 | 0, 0, 0, 0 | 0, 1, 0, 1 |
| G[T > C]A | 1, 0, 1, 2 | 0, 0, 3, 2 | 0, 1, 1, 0 | 1, 0, 0, 0 | 2, 1, 0, 0 | 0, 2, 2, 0 |
| G[T > C]C | 1, 1, 0, 0 | 2, 0, 0, 0 | 0, 0, 0, 0 | 0, 0, 0, 0 | 0, 0, 1, 0 | 0, 1, 1, 1 |
| G[T > C]G | 2, 2, 0, 1 | 0, 1, 0, 1 | 2, 0, 1, 1 | 0, 0, 3, 0 | 0, 0, 0, 0 | 0, 1, 0, 0 |
| G[T > C]T | 1, 0, 1, 1 | 0, 0, 2, 1 | 1, 0, 1, 0 | 0, 1, 2, 2 | 1, 2, 0, 1 | 1, 0, 0, 1 |
| G[T > G]A | 0, 0, 0, 0 | 0, 1, 0, 0 | 0, 0, 0, 0 | 0, 0, 0, 0 | 0, 0, 0, 0 | 0, 0, 0, 0 |
| G[T > G]C | 0, 1, 0, 0 | 0, 0, 0, 0 | 0, 0, 0, 0 | 0, 0, 0, 0 | 0, 1, 0, 0 | 0, 0, 0, 0 |
| G[T > G]G | 0, 0, 0, 0 | 0, 0, 0, 0 | 2, 0, 0, 0 | 1, 0, 0, 1 | 0, 0, 0, 0 | 0, 0, 0, 0 |
| G[T > G]T | 1, 0, 1, 0 | 0, 0, 0, 1 | 0, 0, 0, 0 | 0, 0, 1, 0 | 0, 0, 0, 0 | 0, 0, 0, 0 |
| T[C > A]A | 6, 14, 11, 9 | 15, 10, 20, 14 | 7, 7, 14, 11 | 14, 13, 9, 8 | 14, 10, 6, 8 | 8, 8, 11, 12 |
| T[C > A]C | 1, 2, 3, 0 | 5, 5, 3, 4 | 0, 2, 6, 3 | 2, 3, 1, 5 | 2, 5, 2, 3 | 1, 4, 1, 6 |
| T[C > A]G | 1, 1, 0, 2 | 0, 2, 2, 2 | 1, 0, 2, 0 | 1, 4, 2, 1 | 0, 0, 2, 0 | 0, 1, 1, 1 |
| T[C > A]T | 23, 17, 18, 18 | 24, 31, 39, 36 | 23, 16, 17, 19 | 20, 25, 21, 20 | 24, 21, 24, 29 | 22, 16, 25, 31 |
| T[C > G]A | 0, 0, 1, 0 | 0, 1, 0, 0 | 2, 3, 0, 0 | 1, 0, 0, 1 | 1, 1, 0, 0 | 2, 0, 0, 1 |
| T[C > G]C | 2, 1, 0, 0 | 0, 0, 0, 0 | 0, 1, 0, 0 | 0, 1, 1, 1 | 1, 0, 2, 1 | 3, 1, 1, 0 |
| T[C > G]G | 0, 1, 2, 0 | 0, 1, 0, 0 | 2, 0, 0, 1 | 0, 1, 0, 0 | 2, 0, 0, 0 | 0, 0, 0, 1 |
| T[C > G]T | 1, 0, 0, 0 | 2, 1, 0, 1 | 1, 0, 2, 2 | 1, 0, 1, 0 | 3, 1, 2, 0 | 1, 0, 1, 0 |
| T[C > T]A | 1, 2, 2, 1 | 6, 1, 3, 4 | 6, 3, 5, 2 | 1, 7, 6, 3 | 6, 1, 2, 2 | 2, 3, 6, 3 |
| T[C > T]C | 0, 3, 1, 1 | 3, 3, 2, 1 | 2, 4, 1, 4 | 4, 1, 2, 2 | 3, 4, 0, 2 | 0, 3, 1, 3 |
| T[C > T]G | 1, 0, 1, 1 | 1, 1, 2, 2 | 1, 1, 2, 0 | 0, 1, 3, 1 | 0, 2, 4, 1 | 2, 1, 2, 1 |
| T[C > T]T | 1, 1, 1, 1 | 3, 4, 4, 3 | 4, 3, 1, 2 | 2, 5, 3, 1 | 0, 0, 4, 1 | 1, 1, 3, 2 |
| T[T > A]A | 1, 2, 1, 0 | 4, 3, 0, 2 | 0, 2, 0, 2 | 0, 0, 0, 2 | 2, 0, 1, 0 | 4, 3, 2, 1 |
| T[T > A]C | 0, 1, 0, 2 | 1, 0, 1, 1 | 5, 0, 1, 0 | 1, 0, 0, 1 | 1, 0, 0, 0 | 0, 0, 0, 1 |
| T[T > A]G | 1, 0, 0, 1 | 1, 0, 0, 2 | 1, 0, 0, 0 | 0, 0, 0, 2 | 1, 0, 2, 1 | 0, 3, 1, 2 |
| T[T > A]T | 0, 3, 1, 1 | 0, 0, 0, 3 | 2, 2, 1, 2 | 1, 0, 1, 0 | 0, 1, 2, 1 | 0, 1, 0, 0 |
| T[T > C]A | 2, 2, 5, 2 | 5, 3, 5, 7 | 12, 5, 3, 5 | 0, 4, 4, 1 | 5, 5, 1, 2 | 1, 1, 3, 1 |
| T[T > C]C | 1, 1, 1, 0 | 1, 0, 1, 1 | 0, 2, 0, 0 | 1, 1, 1, 1 | 0, 1, 1, 1 | 0, 0, 1, 0 |
| T[T > C]G | 3, 2, 2, 2 | 2, 1, 1, 1 | 3, 1, 1, 2 | 1, 1, 1, 3 | 1, 1, 0, 3 | 2, 1, 1, 0 |
| T[T > C]T | 2, 3, 0, 1 | 2, 0, 2, 0 | 2, 5, 4, 2 | 3, 1, 2, 1 | 1, 3, 1, 0 | 0, 1, 0, 1 |
| T[T > G]A | 1, 2, 1, 0 | 1, 0, 1, 1 | 0, 0, 0, 0 | 0, 0, 0, 1 | 0, 1, 0, 1 | 1, 0, 0, 0 |
| T[T > G]C | 0, 1, 0, 1 | 0, 0, 1, 0 | 0, 0, 2, 2 | 0, 0, 0, 0 | 0, 1, 0, 0 | 0, 1, 0, 0 |
| T[T > G]G | 0, 0, 0, 0 | 0, 0, 0, 0 | 1, 0, 0, 0 | 0, 0, 1, 0 | 0, 0, 0, 0 | 2, 1, 0, 1 |
| T[T > G]T | 1, 2, 2, 1 | 1, 1, 3, 0 | 1, 0, 0, 1 | 1, 0, 1, 0 | 2, 1, 1, 0 | 1, 1, 1, 1 |

| Mutation | | | | | | |
|---|---|---|---|---|---|---|
| Type | WRN | EXO1 | POLN | C9orf142 | MLH1 | NHEJ1 |
| A[C > A]A | 10, 9, 15, 9 | 47, 26, 21 | 14, 15, 13, 16 | 18, 16, 12, 12 | 19, 8, 10, 9 | 19, 15, 7, 7 |
| A[C > A]C | 1, 0, 0, 1 | 22, 19, 19 | 0, 0, 5, 3 | 0, 1, 4, 2 | 8, 4, 7, 7 | 0, 1, 0, 1 |
| A[C > A]G | 0, 0, 1, 3 | 11, 9, 6 | 0, 0, 0, 1 | 1, 2, 0, 1 | 2, 2, 0, 3 | 0, 1, 0, 0 |
| A[C > A]T | 3, 3, 6, 3 | 23, 21, 18 | 4, 7, 5, 8 | 9, 3, 2, 9 | 17, 16, 14, 16 | 4, 3, 1, 0 |
| A[C > G]A | 0, 0, 0, 2 | 16, 10, 13 | 0, 1, 0, 2 | 2, 0, 1, 1 | 8, 4, 9, 6 | 0, 3, 0, 0 |
| A[C > G]C | 0, 1, 0, 1 | 14, 15, 7 | 0, 0, 3, 0 | 1, 1, 1, 0 | 0, 1, 6, 4 | 0, 0, 0, 0 |
| A[C > G]G | 0, 2, 1, 0 | 15, 7, 9 | 0, 1, 1, 1 | 1, 1, 0, 1 | 1, 0, 4, 0 | 0, 0, 0, 0 |
| A[C > G]T | 1, 1, 0, 0 | 10, 14, 8 | 0, 1, 0, 0 | 1, 0, 1, 1 | 5, 6, 11, 6 | 1, 0, 0, 0 |
| A[C > T]A | 5, 1, 6, 4 | 20, 19, 25 | 6, 4, 7, 13 | 6, 1, 4, 4 | 128, 116, 97, 119 | 8, 9, 0, 1 |
| A[C > T]C | 1, 2, 4, 1 | 19, 7, 13 | 3, 2, 2, 4 | 5, 4, 1, 2 | 30, 43, 25, 29 | 3, 2, 0, 0 |
| A[C > T]G | 4, 1, 5, 3 | 14, 14, 12 | 6, 5, 2, 3 | 2, 1, 2, 1 | 68, 60, 56, 50 | 1, 1, 1, 2 |
| A[C > T]T | 3, 1, 2, 3 | 29, 20, 22 | 4, 2, 4, 1 | 7, 1, 3, 3 | 75, 63, 47, 48 | 1, 1, 1, 2 |
| A[T > A]A | 0, 0, 1, 0 | 22, 9, 15 | 0, 0, 0, 2 | 0, 0, 0, 1 | 3, 3, 1, 5 | 3, 0, 0, 0 |
| A[T > A]C | 0, 0, 0, 0 | 11, 8, 7 | 0, 0, 0, 0 | 0, 1, 0, 0 | 0, 2, 6, 1 | 0, 0, 0, 0 |
| A[T > A]G | 1, 0, 1, 0 | 15, 7, 7 | 0, 0, 1, 0 | 0, 1, 0, 0 | 2, 2, 2, 3 | 1, 0, 0, 0 |
| A[T > A]T | 1, 1, 0, 0 | 15, 8, 9 | 0, 0, 0, 1 | 0, 0, 1, 0 | 35, 36, 38, 30 | 1, 2, 0, 2 |
| A[T > C]A | 4, 0, 0, 5 | 53, 41, 53 | 2, 4, 4, 1 | 1, 3, 4, 4 | 58, 39, 59, 32 | 1, 0, 1, 1 |
| A[T > C]C | 0, 0, 1, 1 | 12, 5, 11 | 0, 0, 0, 1 | 0, 0, 0, 0 | 15, 13, 22, 15 | 2, 0, 0, 0 |
| A[T > C]G | 0, 1, 0, 1 | 17, 15, 19 | 3, 0, 0, 0 | 1, 2, 0, 4 | 60, 61, 56, 56 | 0, 4, 1, 0 |
| A[T > C]T | 1, 0, 0, 1 | 31, 17, 20 | 4, 2, 4, 3 | 2, 2, 1, 2 | 17, 18, 6, 13 | 3, 3, 0, 0 |
| A[T > G]A | 0, 0, 0, 0 | 8, 3, 4 | 0, 0, 0, 0 | 0, 1, 0, 0 | 0, 1, 1, 0 | 0, 0, 0, 0 |
| A[T > G]C | 0, 0, 1, 0 | 0, 1, 3 | 0, 0, 0, 0 | 0, 1, 1, 0 | 2, 0, 1, 1 | 0, 1, 0, 0 |
| A[T > G]G | 0, 0, 0, 1 | 4, 7, 6 | 0, 1, 2, 1 | 0, 0, 0, 0 | 0, 3, 1, 0 | 0, 0, 0, 0 |
| A[T > G]T | 0, 0, 0, 2 | 8, 6, 5 | 0, 0, 3, 0 | 0, 0, 1, 0 | 2, 4, 3, 3 | 0, 1, 0, 0 |
| C[C > A]A | 5, 6, 9, 4 | 30, 12, 18 | 6, 7, 9, 10 | 10, 8, 13, 6 | 35, 34, 42, 34 | 10, 7, 7, 3 |
| C[C > A]C | 2, 0, 1, 4 | 24, 11, 16 | 0, 1, 2, 2 | 0, 0, 0, 1 | 59, 63, 50, 53 | 0, 1, 0, 0 |
| C[C > A]G | 0, 1, 0, 1 | 14, 12, 11 | 1, 2, 2, 0 | 1, 2, 2, 1 | 20, 6, 10, 7 | 1, 1, 0, 0 |
| C[C > A]T | 3, 4, 2, 4 | 18, 16, 13 | 3, 6, 8, 7 | 5, 5, 3, 6 | 142, 165, 146, 139 | 2, 4, 0, 2 |
| C[C > G]A | 0, 1, 0, 0 | 13, 9, 17 | 0, 1, 0, 0 | 0, 1, 0, 2 | 2, 1, 0, 1 | 0, 1, 0, 1 |
| C[C > G]C | 1, 0, 0, 1 | 13, 4, 3 | 0, 1, 0, 1 | 4, 0, 0, 0 | 0, 1, 0, 0 | 0, 0, 0, 0 |
| C[C > G]G | 0, 0, 1, 0 | 3, 3, 5 | 0, 0, 0, 0 | 1, 0, 0, 0 | 2, 1, 0, 0 | 0, 0, 0, 0 |
| C[C > G]T | 0, 0, 0, 1 | 16, 10, 13 | 0, 0, 0, 0 | 0, 0, 0, 0 | 2, 1, 0, 2 | 1, 1, 0, 1 |
| C[C > T]A | 2, 2, 4, 4 | 30, 21, 26 | 5, 2, 9, 3 | 3, 4, 1, 4 | 32, 27, 24, 19 | 5, 2, 0, 0 |
| C[C > T]C | 4, 3, 1, 2 | 23, 8, 12 | 2, 1, 1, 3 | 2, 4, 6, 4 | 23, 27, 24, 31 | 4, 4, 1, 0 |
| C[C > T]G | 2, 0, 3, 1 | 14, 11, 10 | 1, 4, 5, 6 | 5, 3, 1, 4 | 45, 45, 33, 43 | 3, 0, 1, 1 |
| C[C > T]T | 2, 0, 1, 0 | 22, 18, 26 | 2, 3, 2, 2 | 2, 1, 3, 2 | 37, 26, 33, 22 | 0, 3, 1, 3 |
| C[T > A]A | 1, 0, 1, 0 | 18, 12, 12 | 0, 1, 0, 3 | 0, 0, 1, 0 | 3, 0, 2, 2 | 0, 0, 0, 1 |
| C[T > A]C | 0, 0, 2, 0 | 15, 11, 8 | 0, 0, 0, 1 | 0, 0, 0, 0 | 3, 5, 4, 3 | 1, 1, 0, 0 |
| C[T > A]G | 1, 0, 2, 2 | 11, 12, 9 | 0, 2, 0, 0 | 1, 1, 2, 1 | 3, 3, 2, 3 | 2, 0, 0, 0 |
| C[T > A]T | 0, 1, 0, 1 | 18, 7, 7 | 1, 1, 0, 0 | 3, 0, 0, 1 | 3, 0, 3, 1 | 0, 1, 0, 0 |
| C[T > C]A | 1, 0, 1, 0 | 35, 13, 28 | 0, 2, 3, 2 | 1, 2, 1, 1 | 39, 32, 29, 31 | 2, 0, 0, 0 |
| C[T > C]C | 0, 0, 1, 0 | 15, 11, 10 | 1, 0, 2, 0 | 0, 2, 0, 0 | 23, 17, 14, 19 | 1, 0, 0, 0 |
| C[T > C]G | 0, 0, 0, 1 | 14, 9, 8 | 0, 0, 2, 1 | 2, 2, 1, 0 | 64, 60, 46, 54 | 3, 1, 0, 0 |
| C[T > C]T | 2, 1, 0, 0 | 8, 14, 14 | 0, 3, 3, 1 | 0, 0, 0, 0 | 20, 24, 17, 26 | 0, 4, 0, 0 |
| C[T > G]A | 1, 0, 0, 0 | 4, 2, 1 | 0, 1, 1, 1 | 0, 1, 0, 1 | 1, 2, 0, 2 | 0, 1, 0, 0 |
| C[T > G]C | 0, 0, 0, 0 | 5, 0, 7 | 0, 0, 0, 2 | 1, 0, 0, 0 | 6, 6, 11, 5 | 0, 0, 0, 0 |
| C[T > G]G | 0, 0, 0, 0 | 7, 10, 7 | 0, 0, 1, 2 | 0, 0, 2, 0 | 11, 8, 8, 5 | 1, 0, 0, 0 |
| C[T > G]T | 0, 0, 1, 0 | 5, 4, 7 | 0, 0, 0, 0 | 0, 1, 0, 0 | 8, 6, 8, 18 | 0, 1, 0, 0 |
| G[C > A]A | 14, 25, 37, 31 | 61, 31, 58 | 23, 26, 29, 43 | 36, 26, 32, 38 | 26, 29, 22, 20 | 27, 30, 12, 12 |
| G[C > A]C | 1, 0, 2, 6 | 18, 17, 16 | 1, 0, 5, 2 | 0, 0, 0, 2 | 7, 9, 10, 5 | 6, 3, 0, 0 |
| G[C > A]G | 2, 2, 1, 0 | 11, 5, 3 | 1, 3, 4, 1 | 3, 1, 0, 3 | 2, 1, 2, 2 | 3, 2, 0, 0 |
| G[C > A]T | 7, 4, 17, 12 | 44, 26, 32 | 8, 14, 13, 15 | 7, 8, 12, 10 | 22, 19, 45, 20 | 8, 16, 8, 7 |
| G[C > G]A | 1, 0, 0, 0 | 8, 6, 12 | 0, 0, 1, 2 | 0, 1, 0, 1 | 3, 1, 2, 3 | 0, 0, 0, 0 |
| G[C > G]C | 1, 0, 0, 2 | 10, 5, 7 | 0, 0, 1, 0 | 0, 0, 0, 0 | 4, 5, 4, 0 | 0, 0, 0, 1 |
| G[C > G]G | 1, 1, 0, 0 | 3, 0, 3 | 0, 0, 0, 0 | 1, 0, 0, 0 | 1, 0, 0, 0 | 0, 0, 0, 0 |
| G[C > G]T | 0, 0, 0, 0 | 16, 9, 11 | 0, 0, 1, 0 | 1, 0, 0, 0 | 7, 4, 3, 3 | 0, 0, 0, 0 |
| G[C > T]A | 0, 2, 2, 2 | 35, 26, 17 | 4, 4, 2, 0 | 1, 2, 3, 6 | 127, 152, 129, 127 | 2, 1, 1, 0 |
| G[C > T]C | 1, 2, 0, 1 | 24, 5, 12 | 2, 0, 3, 1 | 3, 6, 2, 1 | 92, 83, 79, 98 | 2, 0, 0, 0 |
| G[C > T]G | 1, 4, 1, 4 | 11, 5, 15 | 3, 3, 4, 4 | 3, 2, 0, 0 | 90, 103, 53, 68 | 3, 2, 1, 1 |
| G[C > T]T | 0, 1, 4, 1 | 24, 17, 24 | 2, 1, 4, 1 | 1, 3, 4, 1 | 111, 113, 87, 85 | 1, 3, 0, 0 |
| G[T > A]A | 0, 0, 0, 0 | 8, 8, 8 | 2, 0, 1, 1 | 1, 0, 0, 0 | 1, 0, 3, 0 | 0, 0, 0, 0 |
| G[T > A]C | 0, 0, 0, 1 | 12, 8, 6 | 0, 0, 0, 0 | 0, 0, 2, 0 | 3, 0, 2, 2 | 0, 0, 0, 0 |
| G[T > A]G | 0, 0, 0, 0 | 15, 6, 10 | 0, 0, 0, 1 | 0, 0, 0, 0 | 2, 1, 4, 0 | 0, 1, 0, 0 |
| G[T > A]T | 0, 1, 2, 0 | 13, 4, 7 | 0, 0, 1, 0 | 0, 1, 0, 0 | 5, 2, 6, 5 | 0, 0, 0, 1 |
| G[T > C]A | 0, 0, 0, 0 | 10, 8, 11 | 0, 0, 0, 0 | 1, 0, 0, 1 | 28, 21, 24, 17 | 2, 1, 0, 0 |
| G[T > C]C | 1, 1, 0, 0 | 5, 1, 6 | 0, 1, 1, 1 | 0, 0, 0, 0 | 13, 13, 9, 6 | 0, 1, 0, 1 |
| G[T > C]G | 0, 1, 0, 0 | 6, 4, 4 | 0, 1, 0, 2 | 0, 0, 0, 0 | 28, 22, 38, 38 | 2, 1, 0, 0 |
| G[T > C]T | 1, 0, 0, 1 | 12, 6, 6 | 1, 1, 0, 1 | 2, 2, 1, 0 | 12, 13, 11, 8 | 0, 2, 0, 2 |
| G[T > G]A | 0, 0, 0, 0 | 6, 4, 2 | 0, 0, 0, 0 | 0, 0, 0, 0 | 0, 0, 0, 0 | 1, 1, 0, 0 |
| G[T > G]C | 0, 0, 0, 0 | 1, 2, 1 | 0, 0, 1, 0 | 0, 0, 0, 0 | 2, 0, 0, 1 | 0, 0, 0, 0 |
| G[T > G]G | 0, 0, 0, 0 | 5, 5, 7 | 0, 0, 0, 0 | 0, 0, 2, 0 | 1, 0, 1, 0 | 1, 0, 0, 1 |
| G[T > G]T | 0, 0, 0, 0 | 6, 4, 4 | 0, 0, 1, 0 | 0, 1, 0, 0 | 2, 1, 3, 2 | 0, 0, 0, 1 |
| T[C > A]A | 5, 7, 9, 15 | 39, 31, 32 | 8, 10, 12, 11 | 13, 13, 17, 15 | 24, 9, 14, 17 | 20, 27, 2, 7 |
| T[C > A]C | 5, 3, 4, 2 | 28, 16, 25 | 4, 9, 5, 6 | 4, 5, 3, 3 | 14, 10, 9, 13 | 4, 7, 2, 2 |
| T[C > A]G | 1, 1, 0, 2 | 5, 5, 4 | 2, 1, 2, 1 | 3, 1, 0, 1 | 2, 3, 2, 1 | 1, 3, 2, 0 |
| T[C > A]T | 20, 12, 27, 25 | 51, 37, 45 | 18, 20, 24, 23 | 25, 24, 23, 38 | 60, 44, 46, 34 | 22, 42, 8, 9 |
| T[C > G]A | 1, 0, 0, 0 | 15, 16, 8 | 1, 1, 1, 1 | 2, 0, 0, 0 | 0, 1, 2, 0 | 1, 0, 0, 0 |
| T[C > G]C | 0, 1, 0, 1 | 8, 4, 7 | 0, 0, 2, 0 | 0, 0, 0, 0 | 2, 0, 1, 2 | 0, 0, 0, 1 |
| T[C > G]G | 0, 0, 0, 0 | 1, 4, 6 | 0, 0, 0, 0 | 2, 0, 0, 1 | 0, 0, 0, 0 | 0, 0, 0, 1 |
| T[C > G]T | 3, 2, 1, 0 | 16, 15, 14 | 1, 4, 0, 2 | 0, 0, 2, 1 | 4, 5, 2, 6 | 1, 0, 0, 1 |
| T[C > T]A | 3, 3, 2, 2 | 37, 25, 23 | 2, 3, 5, 7 | 4, 3, 3, 5 | 24, 18, 22, 23 | 4, 7, 2, 0 |
| T[C > T]C | 3, 1, 1, 2 | 19, 15, 13 | 1, 0, 3, 3 | 3, 5, 0, 1 | 23, 25, 21, 17 | 2, 6, 0, 1 |
| T[C > T]G | 1, 1, 0, 2 | 12, 7, 6 | 1, 1, 5, 2 | 3, 2, 0, 3 | 20, 24, 22, 13 | 2, 1, 0, 0 |
| T[C > T]T | 3, 1, 0, 1 | 22, 16, 18 | 1, 3, 0, 2 | 3, 5, 0, 2 | 24, 24, 30, 19 | 3, 4, 0, 0 |
| T[T > A]A | 2, 0, 2, 5 | 26, 18, 21 | 3, 1, 1, 3 | 1, 1, 2, 0 | 10, 6, 8, 3 | 4, 0, 0, 0 |
| T[T > A]C | 1, 0, 0, 0 | 22, 18, 15 | 0, 0, 0, 0 | 0, 2, 1, 3 | 3, 0, 1, 4 | 1, 1, 1, 1 |
| T[T > A]G | 1, 0, 0, 0 | 13, 12, 15 | 0, 1, 1, 0 | 0, 4, 3, 1 | 2, 1, 1, 2 | 0, 0, 0, 0 |
| T[T > A]T | 0, 0, 2, 2 | 24, 22, 19 | 0, 0, 3, 1 | 2, 0, 2, 3 | 7, 4, 5, 8 | 1, 4, 0, 0 |
| T[T > C]A | 3, 4, 3, 3 | 48, 22, 30 | 2, 2, 5, 1 | 6, 3, 3, 3 | 19, 23, 19, 18 | 2, 6, 0, 0 |
| T[T > C]C | 0, 0, 2, 1 | 6, 8, 6 | 1, 1, 2, 2 | 0, 2, 0, 1 | 20, 16, 13, 17 | 0, 1, 0, 1 |
| T[T > C]G | 0, 0, 2, 0 | 18, 9, 9 | 1, 0, 2, 2 | 2, 1, 2, 0 | 32, 36, 31, 29 | 0, 1, 1, 2 |
| T[T > C]T | 1, 0, 1, 2 | 18, 9, 26 | 2, 2, 3, 4 | 4, 2, 3, 3 | 25, 20, 22, 26 | 3, 1, 0, 1 |
| T[T > G]A | 0, 0, 1, 0 | 13, 6, 4 | 0, 1, 2, 0 | 0, 0, 0, 0 | 0, 0, 0, 2 | 0, 1, 0, 1 |
| T[T > G]C | 0, 0, 0, 1 | 5, 2, 5 | 0, 0, 0, 0 | 0, 0, 1, 0 | 4, 1, 0, 3 | 1, 0, 0, 0 |
| T[T > G]G | 1, 0, 1, 0 | 12, 4, 6 | 2, 1, 0, 0 | 0, 0, 0, 0 | 4, 6, 1, 2 | 0, 1, 0, 0 |
| T[T > G]T | 0, 2, 1, 0 | 13, 14, 9 | 3, 0, 2, 1 | 2, 1, 0, 2 | 2, 3, 6, 6 | 1, 1, 1, 2 |

| Mutation | | | | | |
|---|---|---|---|---|---|
| Type | MSH6 | MSH2 | PMS1 | PMS2 | POLM |
| A[C > A]A | 6, 8, 17, 18, 10, 15, 17, 18 | 10, 26, 14 | 10, 21, 15, 1 | 12, 21, 17, 26 | 26, 12, 8, 5 |
| A[C > A]C | 10, 6, 9, 8, 4, 4, 9, 7 | 5, 5, 3 | 2, 1, 2, 1 | 7, 5, 4, 5 | 1, 3, 1, 0 |
| A[C > A]G | 0, 2, 2, 4, 3, 1, 2, 3 | 1, 1, 2 | 1, 1, 0, 0 | 1, 1, 1, 3 | 0, 2, 0, 1 |
| A[C > A]T | 11, 29, 19, 23, 19, 16, 23, 22 | 18, 26, 16 | 6, 5, 7, 6 | 8, 17, 15, 16 | 7, 8, 5, 2 |
| A[C > G]A | 3, 4, 5, 2, 2, 9, 7, 7 | 7, 5, 4 | 0, 0, 0, 0 | 3, 5, 4, 4 | 0, 0, 2, 2 |
| A[C > G]C | 2, 3, 3, 3, 2, 2, 1, 2 | 2, 3, 2 | 1, 2, 1, 1 | 5, 5, 5, 2 | 0, 3, 0, 1 |
| A[C > G]G | 1, 0, 0, 1, 1, 1, 0, 1 | 0, 2, 0 | 0, 1, 1, 0 | 0, 2, 1, 2 | 0, 2, 0, 0 |
| A[C > G]T | 10, 10, 17, 11, 11, 8, 8, 7 | 11, 10, 7 | 1, 2, 0, 0 | 5, 9, 9, 11 | 0, 3, 3, 0 |
| A[C > T]A | 100, 146, 167, 160, 84, 127, | 133, 157, 143 | 15, 11, 23, 17 | 23, 18, 22, 16 | 2, 5, 6, 3 |
| | 183, 142 | | | | |
| A[C > T]C | 33, 48, 57, 48, 24, 47, 49, 39 | 40, 49, 36 | 4, 1, 3, 2 | 12, 17, 16, 21 | 1, 2, 2, 1 |
| A[C > T]G | 35, 68, 62, 74, 31, 55, 73, 74 | 74, 68, 65 | 11, 16, 12, 28 | 14, 20, 23, 32 | 2, 3, 1, 2 |
| A[C > T]T | 49, 72, 59, 85, 36, 46, 76, 58 | 75, 64, 62 | 3, 2, 3, 5 | 11, 13, 9, 17 | 2, 5, 2, 5 |
| A[T > A]A | 0, 3, 0, 4, 1, 4, 1, 4 | 3, 1, 2 | 2, 2, 2, 0 | 6, 5, 3, 6 | 0, 0, 2, 0 |
| A[T > A]C | 5, 4, 5, 4, 1, 1, 5, 5 | 2, 1, 2 | 1, 0, 1, 1 | 5, 1, 4, 3 | 0, 0, 1, 2 |
| A[T > A]G | 3, 0, 2, 0, 0, 2, 2, 2 | 2, 1, 1 | 0, 1, 0, 1 | 0, 3, 1, 1 | 1, 0, 0, 4 |
| A[T > A]T | 27, 28, 44, 27, 27, 27, 44, 32 | 33, 36, 30 | 0, 0, 3, 0 | 34, 28, 29, 37 | 0, 2, 1, 3 |
| A[T > C]A | 35, 59, 59, 52, 26, 49, 70, 56 | 46, 47, 53 | 1, 0, 1, 1 | 90, 73, 68, 98 | 4, 2, 3, 1 |
| A[T > C]C | 15, 24, 33, 21, 12, 24, 38, 27 | 21, 27, 19 | 0, 1, 0, 1 | 48, 32, 30, 37 | 1, 0, 1, 1 |
| A[T > C]G | 62, 79, 95, 82, 52, 63, 96, 87 | 71, 53, 49 | 0, 1, 0, 4 | 105, 91, 81, 115 | 1, 0, 0, 0 |
| A[T > C]T | 17, 16, 27, 22, 15, 16, 21, 24 | 18, 23, 15 | 3, 1, 2, 2 | 25, 23, 18, 29 | 0, 3, 4, 1 |
| A[T > G]A | 2, 0, 0, 0, 1, 0, 0, 1 | 0, 1, 0 | 0, 0, 0, 0 | 0, 2, 1, 0 | 0, 1, 0, 0 |
| A[T > G]C | 1, 1, 2, 1, 2, 1, 3, 0 | 1, 2, 1 | 0, 1, 0, 0 | 1, 5, 3, 3 | 1, 0, 1, 0 |
| A[T > G]G | 5, 1, 0, 3, 2, 1, 2, 1 | 0, 2, 1 | 0, 0, 1, 1 | 1, 1, 1, 6 | 0, 0, 0, 0 |
| A[T > G]T | 6, 6, 4, 7, 6, 1, 7, 4 | 5, 7, 1 | 0, 0, 0, 0 | 8, 5, 10, 5 | 2, 1, 0, 1 |
| C[C > A]A | 38, 34, 48, 55, 24, 45, 68, 61 | 39, 36, 55 | 7, 11, 7, 7 | 23, 17, 13, 19 | 6, 6, 7, 6 |
| C[C > A]C | 44, 70, 75, 89, 20, 74, 100, 61 | 58, 65, 52 | 0, 4, 2, 1 | 24, 27, 18, 28 | 2, 4, 1, 0 |
| C[C > A]G | 11, 13, 19, 16, 5, 13, 18, 14 | 12, 12, 8 | 1, 0, 0, 2 | 11, 15, 10, 8 | 1, 0, 0, 0 |
| C[C > A]T | 175, 210, 253, 231, 125, 000, | 194, 224, 202 | 3, 5, 6, 6 | 44, 57, 41, 41 | 4, 5, 1, 4 |
| | 000, 000 | | | | |
| C[C > G]A | 0, 2, 1, 1, 0, 2, 0, 0 | 0, 1, 0 | 0, 0, 0, 1 | 1, 0, 0, 2 | 0, 0, 1, 0 |
| C[C > G]C | 0, 0, 0, 0, 1, 0, 1, 0 | 1, 2, 0 | 0, 0, 0, 0 | 0, 0, 0, 0 | 1, 0, 0, 0 |
| C[C > G]G | 0, 2, 0, 1, 2, 0, 2, 1 | 2, 0, 1 | 0, 0, 2, 1 | 3, 1, 0, 0 | 1, 1, 0, 1 |
| C[C > G]T | 2, 1, 1, 0, 2, 1, 2, 2 | 1, 0, 0 | 0, 1, 1, 0 | 0, 1, 0, 1 | 0, 0, 0, 0 |
| C[C > T]A | 18, 35, 35, 26, 11, 27, 26, 30 | 34, 32, 22 | 5, 8, 4, 8 | 13, 13, 11, 7 | 2, 4, 2, 6 |
| C[C > T]C | 21, 21, 32, 24, 16, 17, 28, 25 | 21, 30, 23 | 0, 2, 8, 5 | 14, 21, 18, 7 | 7, 7, 3, 3 |
| C[C > T]G | 20, 39, 42, 40, 19, 26, 53, 27 | 54, 47, 43 | 5, 10, 13, 21 | 11, 17, 18, 10 | 1, 3, 3, 3 |
| C[C > T]T | 11, 24, 23, 35, 11, 13, 30, 29 | 35, 36, 30 | 3, 4, 4, 4 | 12, 12, 18, 17 | 2, 1, 5, 2 |
| C[T > A]A | 1, 2, 1, 3, 0, 1, 1, 0 | 0, 0, 3 | 0, 0, 0, 2 | 1, 3, 1, 2 | 0, 1, 0, 2 |
| C[T > A]C | 3, 7, 5, 5, 2, 9, 12, 3 | 4, 5, 3 | 0, 0, 0, 0 | 7, 5, 3, 3 | 0, 0, 0, 0 |
| C[T > A]G | 1, 1, 0, 3, 5, 2, 1, 2 | 0, 2, 1 | 0, 0, 0, 1 | 3, 5, 1, 3 | 0, 0, 0, 0 |
| C[T > A]T | 0, 3, 5, 2, 3, 2, 2, 3 | 1, 2, 6 | 0, 2, 0, 0 | 3, 4, 2, 4 | 0, 1, 0, 1 |
| C[T > C]A | 29, 41, 58, 51, 25, 47, 71, 55 | 41, 26, 46 | 1, 1, 1, 0 | 52, 59, 53, 58 | 0, 2, 0, 0 |
| C[T > C]C | 24, 31, 45, 29, 18, 40, 54, 34 | 24, 31, 22 | 0, 0, 0, 1 | 44, 39, 39, 47 | 1, 1, 0, 0 |
| C[T > C]G | 78, 88, 116, 124, 58, 87, 102, 82 | 59, 83, 55 | 1, 1, 1, 0 | 98, 112, 87, 114 | 1, 1, 0, 0 |
| C[T > C]T | 23, 25, 34, 38, 22, 21, 41, 40 | 30, 29, 16 | 0, 1, 1, 3 | 40, 33, 37, 44 | 2, 1, 2, 0 |
| C[T > G]A | 1, 3, 1, 2, 2, 2, 1, 2 | 3, 2, 2 | 0, 0, 2, 0 | 1, 0, 0, 3 | 0, 0, 0, 0 |
| C[T > G]C | 9, 6, 9, 10, 5, 9, 10, 6 | 8, 7, 2 | 0, 0, 0, 0 | 9, 12, 6, 5 | 0, 0, 0, 0 |
| C[T > G]G | 7, 11, 14, 7, 7, 11, 13, 9 | 11, 6, 8 | 0, 0, 0, 1 | 5, 23, 7, 16 | 0, 0, 0, 0 |
| C[T > G]T | 5, 22, 8, 16, 10, 11, 20, 20 | 16, 12, 12 | 0, 2, 0, 0 | 10, 12, 16, 19 | 0, 0, 1, 1 |
| G[C > A]A | 40, 39, 48, 42, 25, 26, 35, 32 | 53, 35, 21 | 16, 26, 32, 42 | 13, 32, 33, 38 | 34, 39, 22, 19 |
| G[C > A]C | 12, 14, 7, 14, 5, 9, 13, 13 | 14, 12, 8 | 2, 1, 6, 3 | 7, 11, 5, 15 | 2, 8, 3, 0 |
| G[C > A]G | 4, 9, 4, 10, 1, 3, 3, 4 | 7, 6, 5 | 0, 1, 1, 1 | 2, 1, 1, 3 | 2, 1, 1, 0 |
| G[C > A]T | 28, 64, 50, 61, 23, 38, 49, 48 | 42, 41, 37 | 11, 17, 13, 25 | 15, 21, 34, 24 | 12, 11, 6, 9 |
| G[C > G]A | 0, 1, 3, 3, 1, 2, 3, 5 | 1, 1, 1 | 0, 0, 0, 0 | 1, 2, 2, 5 | 1, 1, 0, 1 |
| G[C > G]C | 3, 5, 2, 7, 3, 4, 2, 5 | 3, 3, 5 | 0, 0, 3, 0 | 8, 6, 3, 2 | 0, 1, 0, 1 |
| G[C > G]G | 0, 2, 2, 0, 0, 0, 0, 0 | 0, 0, 0 | 0, 0, 1, 0 | 1, 0, 2, 0 | 0, 1, 0, 0 |
| G[C > G]T | 6, 2, 3, 7, 2, 3, 5, 6 | 4, 3, 1 | 0, 1, 0, 0 | 6, 7, 3, 7 | 1, 0, 0, 0 |
| G[C > T]A | 121, 146, 185, 182, 80, 119, | 162, 158, 155 | 4, 11, 9, 6 | 14, 12, 8, 12 | 4, 2, 0, 5 |
| | 190, 156 | | | | |
| G[C > T]C | 83, 130, 128, 138, 86, 111, | 107, 112, 92 | 4, 1, 2, 2 | 21, 36, 28, 32 | 1, 3, 1, 0 |
| | 152, 146 | | | | |
| G[C > T]G | 52, 78, 83, 97, 50, 79, 89, 75 | 104, 77, 72 | 5, 11, 9, 13 | 43, 52, 47, 42 | 1, 3, 2, 2 |
| G[C > T]T | 80, 124, 123, 106, 60, 83, | 120, 132, 119 | 0, 3, 2, 2 | 18, 17, 21, 20 | 3, 5, 2, 1 |
| | 131, 104 | | | | |
| G[T > A]A | 0, 0, 1, 0, 0, 2, 0, 1 | 1, 0, 0 | 0, 0, 0, 0 | 1, 2, 0, 2 | 0, 2, 0, 1 |
| G[T > A]C | 2, 2, 2, 6, 1, 2, 2, 3 | 1, 3, 0 | 1, 0, 1, 0 | 5, 2, 3, 4 | 0, 0, 1, 0 |
| G[T > A]G | 0, 1, 1, 4, 2, 3, 4, 0 | 2, 0, 2 | 0, 0, 0, 0 | 1, 2, 3, 1 | 1, 1, 0, 0 |
| G[T > A]T | 4, 6, 3, 9, 4, 2, 2, 5 | 2, 6, 4 | 0, 0, 1, 0 | 3, 4, 5, 6 | 1, 4, 1, 0 |
| G[T > C]A | 29, 33, 41, 28, 23, 37, 53, 26 | 28, 21, 26 | 0, 0, 1, 1 | 46, 58, 42, 51 | 1, 0, 1, 0 |
| G[T > C]C | 18, 18, 17, 18, 4, 14, 23, 15 | 13, 30, 11 | 0, 0, 0, 0 | 27, 23, 14, 20 | 0, 0, 1, 0 |
| G[T > C]G | 30, 38, 51, 51, 25, 41, 55, 39 | 25, 30, 33 | 1, 1, 1, 3 | 33, 34, 41, 48 | 0, 0, 0, 0 |
| G[T > C]T | 17, 18, 19, 16, 14, 13, 21, 19 | 16, 15, 7 | 0, 0, 1, 2 | 35, 22, 23, 20 | 0, 0, 0, 0 |
| G[T > G]A | 0, 0, 0, 1, 0, 1, 0, 0 | 0, 1, 0 | 0, 0, 0, 0 | 0, 0, 1, 1 | 0, 1, 1, 0 |
| G[T > G]C | 0, 1, 1, 1, 1, 0, 1, 5 | 1, 0, 0 | 0, 0, 0, 0 | 0, 2, 1, 0 | 0, 0, 0, 0 |
| G[T > G]G | 0, 0, 0, 0, 0, 0, 2, 0 | 1, 0, 1 | 1, 1, 0, 0 | 0, 2, 2, 2 | 0, 0, 0, 0 |
| G[T > G]T | 2, 3, 1, 4, 3, 1, 3, 4 | 3, 0, 4 | 0, 0, 1, 1 | 5, 5, 2, 4 | 0, 0, 0, 2 |
| T[C > A]A | 17, 22, 19, 15, 7, 11, 21, 8 | 10, 13, 17 | 11, 12, 13, 18 | 12, 19, 11, 9 | 11, 17, 9, 7 |
| T[C > A]C | 13, 17, 13, 20, 6, 9, 29, 21 | 18, 15, 10 | 5, 9, 3, 9 | 7, 12, 11, 8 | 5, 5, 6, 2 |
| T[C > A]G | 5, 3, 4, 3, 3, 5, 5, 4 | 5, 6, 2 | 1, 4, 0, 3 | 1, 2, 5, 4 | 2, 3, 1, 0 |
| T[C > A]T | 42, 94, 71, 74, 52, 50, 70, 87 | 60, 95, 54 | 13, 34, 31, 40 | 38, 45, 43, 35 | 37, 38, 17, 16 |
| T[C > G]A | 0, 0, 0, 0, 1, 2, 1, 1 | 0, 0, 4 | 0, 0, 1, 0 | 1, 4, 1, 2 | 2, 1, 1, 1 |
| T[C > G]C | 1, 3, 0, 1, 1, 1, 0, 0 | 1, 3, 0 | 1, 0, 0, 0 | 3, 6, 1, 0 | 1, 0, 0, 0 |
| T[C > G]G | 0, 0, 0, 0, 0, 0, 1, 1 | 0, 1, 0 | 0, 1, 0, 1 | 1, 0, 0, 1 | 0, 0, 0, 0 |
| T[C > G]T | 3, 6, 4, 3, 1, 1, 4, 7 | 4, 3, 4 | 2, 0, 2, 1 | 3, 4, 0, 5 | 1, 1, 1, 2 |
| T[C > T]A | 20, 25, 23, 22, 9, 24, 35, 17 | 33, 26, 26 | 6, 4, 7, 13 | 7, 17, 9, 4 | 4, 2, 2, 1 |
| T[C > T]C | 13, 21, 25, 22, 13, 19, 26, 19 | 23, 19, 20 | 2, 0, 4, 5 | 8, 8, 11, 12 | 0, 0, 1, 3 |
| T[C > T]G | 12, 25, 15, 15, 15, 14, 24, 25 | 23, 24, 22 | 8, 11, 6, 3 | 2, 8, 6, 5 | 3, 0, 0, 0 |
| T[C > T]T | 17, 25, 19, 19, 10, 15, 24, 22 | 26, 21, 26 | 2, 1, 1, 4 | 11, 14, 12, 7 | 0, 0, 0, 1 |
| T[T > A]A | 7, 9, 14, 15, 5, 9, 17, 9 | 11, 3, 7 | 2, 0, 2, 2 | 9, 14, 14, 16 | 3, 1, 2, 1 |
| T[T > A]C | 2, 0, 4, 2, 1, 0, 3, 0 | 1, 0, 1 | 0, 0, 0, 1 | 2, 2, 1, 1 | 2, 0, 0, 1 |
| T[T > A]G | 0, 1, 0, 1, 1, 0, 1, 4 | 1, 0, 4 | 0, 1, 2, 0 | 0, 2, 0, 2 | 1, 3, 1, 1 |
| T[T > A]T | 7, 4, 14, 13, 4, 2, 12, 5 | 9, 10, 7 | 2, 0, 1, 3 | 10, 7, 12, 12 | 3, 3, 1, 2 |
| T[T > C]A | 32, 40, 51, 47, 27, 46, 51, 52 | 35, 42, 22 | 1, 1, 1, 0 | 48, 54, 48, 61 | 1, 5, 6, 1 |
| T[T > C]C | 22, 24, 31, 29, 15, 34, 40, 34 | 38, 20, 22 | 0, 1, 1, 1 | 44, 48, 37, 49 | 2, 0, 2, 1 |
| T[T > C]G | 33, 48, 46, 44, 28, 53, 73, 58 | 51, 25, 20 | 2, 1, 2, 0 | 34, 50, 57, 63 | 2, 2, 2, 1 |
| T[T > C]T | 22, 25, 36, 28, 24, 27, 39, 36 | 19, 29, 19 | 1, 2, 2, 5 | 40, 44, 36, 43 | 2, 4, 1, 2 |
| T[T > G]A | 0, 0, 0, 1, 0, 0, 2, 1 | 1, 0, 0 | 0, 0, 1, 1 | 0, 0, 3, 0 | 0, 0, 0, 1 |
| T[T > G]C | 1, 1, 0, 4, 1, 1, 2, 6 | 0, 1, 0 | 1, 1, 0, 1 | 3, 2, 0, 3 | 0, 0, 0, 0 |
| T[T > G]G | 0, 3, 1, 5, 1, 3, 2, 5 | 5, 2, 4 | 1, 0, 2, 0 | 1, 8, 4, 4 | 0, 2, 1, 0 |
| T[T > G]T | 2, 4, 6, 6, 4, 11, 5, 4 | 4, 9, 3 | 0, 0, 3, 1 | 6, 7, 2, 6 | 3, 3, 2, 0 |

| Mutation | | | | | | | |
|---|---|---|---|---|---|---|---|
| Type | POLQ | PRKDC | XRCC4 | POLI | PRIMPOL | RAD18 | REV1 |
| A[C > A]A | 11, 6, 10, 10 | 8, 14, 4 | 9, 20, 21, 22 | 13, 9, 19, 14 | 7, 7, 11, 16 | 10, 7, 12, 15 | 3, 8, 7, 10 |
| A[C > A]C | 1, 2, 4, 1 | 0, 1, 0 | 0, 1, 1, 1 | 1, 2, 2, 2 | 2, 0, 0, 0 | 0, 0, 0, 1 | 1, 1, 1, 1 |
| A[C > A]G | 0, 1, 0, 0 | 0, 1, 2 | 1, 0, 0, 1 | 0, 0, 0, 0 | 1, 1, 2, 0 | 2, 1, 0, 0 | 2, 0, 1, 0 |
| A[C > A]T | 5, 5, 3, 3 | 6, 4, 5 | 3, 4, 9, 5 | 4, 3, 6, 4 | 6, 1, 6, 4 | 3, 6, 5, 3 | 1, 3, 2, 2 |
| A[C > G]A | 0, 0, 1, 0 | 0, 1, 0 | 3, 0, 1, 1 | 0, 1, 1, 0 | 2, 2, 1, 8 | 2, 0, 0, 0 | 0, 0, 0, 3 |
| A[C > G]C | 1, 0, 1, 0 | 0, 0, 1 | 0, 1, 2, 0 | 0, 0, 0, 0 | 0, 0, 1, 1 | 0, 1, 1, 0 | 0, 0, 0, 2 |
| A[C > G]G | 1, 1, 0, 0 | 0, 1, 0 | 1, 0, 1, 0 | 1, 1, 1, 1 | 2, 1, 0, 0 | 0, 0, 0, 0 | 0, 2, 0, 2 |
| A[C > G]T | 0, 0, 1, 0 | 0, 0, 1 | 0, 1, 0, 3 | 1, 0, 0, 1 | 0, 0, 2, 1 | 1, 1, 0, 0 | 0, 1, 0, 2 |
| A[C > T]A | 4, 1, 1, 4 | 2, 2, 4 | 8, 5, 6, 6 | 7, 4, 5, 7 | 3, 4, 6, 6 | 4, 4, 6, 6 | 2, 3, 2, 6 |
| A[C > T]C | 3, 1, 1, 1 | 2, 1, 2 | 4, 1, 4, 7 | 4, 2, 2, 2 | 2, 1, 2, 4 | 1, 2, 0, 1 | 0, 1, 0, 3 |
| A[C > T]G | 2, 0, 2, 1 | 1, 1, 1 | 1, 5, 4, 6 | 3, 0, 2, 2 | 3, 3, 5, 4 | 2, 3, 2, 0 | 0, 3, 4, 2 |
| A[C > T]T | 3, 5, 2, 4 | 2, 0, 0 | 0, 1, 3, 0 | 5, 0, 6, 1 | 1, 3, 3, 2 | 4, 3, 3, 2 | 0, 1, 1, 4 |
| A[T > A]A | 1, 2, 2, 1 | 0, 0, 0 | 1, 2, 1, 5 | 1, 0, 0, 3 | 1, 0, 1, 2 | 2, 0, 2, 0 | 1, 0, 4, 1 |
| A[T > A]C | 0, 1, 0, 0 | 0, 1, 0 | 0, 2, 1, 2 | 0, 1, 2, 0 | 0, 0, 0, 0 | 0, 1, 0, 1 | 0, 0, 0, 0 |
| A[T > A]G | 1, 3, 0, 0 | 0, 0, 0 | 1, 0, 1, 0 | 2, 0, 0, 1 | 0, 1, 0, 1 | 0, 0, 0, 0 | 0, 1, 0, 1 |
| A[T > A]T | 2, 1, 2, 2 | 0, 0, 1 | 0, 0, 2, 2 | 0, 0, 4, 0 | 1, 2, 0, 4 | 1, 0, 0, 2 | 0, 0, 0, 4 |
| A[T > C]A | 5, 4, 3, 0 | 4, 0, 4 | 1, 2, 7, 1 | 5, 2, 3, 1 | 1, 1, 6, 3 | 3, 5, 2, 0 | 0, 1, 0, 2 |
| A[T > C]C | 0, 0, 0, 0 | 0, 2, 1 | 1, 0, 2, 0 | 1, 1, 1, 0 | 3, 1, 0, 1 | 0, 0, 0, 1 | 0, 1, 0, 0 |
| A[T > C]G | 1, 0, 0, 0 | 0, 3, 1 | 0, 0, 3, 1 | 0, 1, 1, 2 | 0, 0, 3, 2 | 2, 1, 2, 0 | 1, 1, 1, 0 |
| A[T > C]T | 1, 2, 0, 1 | 3, 1, 1 | 1, 2, 0, 2 | 6, 0, 4, 3 | 1, 1, 0, 4 | 2, 1, 0, 2 | 0, 1, 0, 1 |
| A[T > G]A | 0, 1, 0, 0 | 0, 0, 0 | 0, 1, 0, 0 | 0, 0, 0, 1 | 0, 1, 0, 0 | 0, 0, 0, 0 | 0, 0, 0, 0 |
| A[T > G]C | 0, 0, 0, 0 | 0, 0, 0 | 0, 0, 0, 0 | 0, 0, 0, 0 | 0, 0, 1, 1 | 0, 0, 0, 0 | 0, 0, 0, 0 |
| A[T > G]G | 1, 0, 0, 0 | 2, 0, 0 | 0, 0, 0, 1 | 0, 1, 0, 1 | 0, 0, 0, 1 | 0, 1, 0, 0 | 0, 0, 0, 0 |
| A[T > G]T | 1, 0, 1, 0 | 0, 0, 0 | 1, 0, 1, 0 | 1, 0, 1, 0 | 0, 1, 0, 4 | 0, 0, 1, 0 | 0, 0, 0, 0 |
| C[C > A]A | 7, 7, 5, 5 | 7, 10, 5 | 4, 7, 12, 12 | 12, 5, 13, 9 | 4, 1, 13, 14 | 5, 8, 6, 7 | 3, 3, 3, 3 |
| C[C > A]C | 0, 1, 0, 0 | 1, 1, 2 | 1, 1, 2, 3 | 3, 1, 4, 2 | 1, 0, 1, 1 | 1, 0, 1, 0 | 1, 3, 2, 3 |
| C[C > A]G | 2, 1, 1, 1 | 0, 1, 1 | 0, 1, 0, 1 | 3, 1, 0, 2 | 1, 0, 0, 1 | 2, 0, 0, 0 | 1, 1, 0, 0 |
| C[C > A]T | 2, 5, 4, 1 | 0, 4, 1 | 4, 6, 4, 9 | 6, 1, 5, 6 | 3, 2, 8, 7 | 3, 4, 7, 2 | 1, 3, 1, 4 |
| C[C > G]A | 1, 0, 0, 0 | 0, 0, 0 | 1, 2, 0, 1 | 0, 1, 1, 0 | 1, 0, 1, 0 | 1, 0, 1, 1 | 0, 1, 0, 0 |
| C[C > G]C | 1, 1, 1, 0 | 1, 0, 0 | 1, 0, 0, 0 | 1, 0, 0, 0 | 0, 0, 0, 2 | 0, 1, 0, 0 | 0, 0, 1, 0 |
| C[C > G]G | 0, 0, 0, 0 | 0, 0, 0 | 0, 0, 1, 0 | 1, 0, 0, 0 | 0, 0, 0, 0 | 0, 0, 0, 0 | 1, 1, 0, 2 |
| C[C > G]T | 2, 0, 1, 0 | 0, 0, 0 | 1, 0, 0, 1 | 1, 2, 2, 1 | 0, 0, 2, 2 | 0, 0, 0, 1 | 0, 0, 0, 0 |
| C[C > T]A | 1, 4, 2, 4 | 7, 3, 6 | 5, 6, 6, 3 | 3, 3, 9, 8 | 4, 5, 1, 3 | 6, 5, 3, 7 | 0, 6, 2, 6 |
| C[C > T]C | 5, 0, 1, 2 | 2, 5, 3 | 2, 1, 0, 5 | 6, 3, 2, 3 | 2, 2, 4, 2 | 3, 4, 2, 6 | 0, 4, 0, 3 |
| C[C > T]G | 2, 1, 2, 3 | 3, 2, 1 | 3, 3, 3, 4 | 2, 0, 4, 3 | 5, 3, 5, 2 | 3, 1, 3, 1 | 1, 2, 1, 3 |
| C[C > T]T | 4, 0, 3, 4 | 0, 2, 3 | 3, 2, 1, 0 | 4, 2, 5, 2 | 1, 3, 3, 4 | 1, 2, 1, 2 | 1, 1, 2, 0 |
| C[T > A]A | 1, 1, 0, 2 | 0, 0, 1 | 0, 1, 0, 1 | 3, 1, 1, 2 | 0, 0, 1, 1 | 0, 0, 1, 0 | 0, 0, 0, 2 |
| C[T > A]C | 0, 1, 0, 0 | 0, 0, 0 | 0, 1, 0, 2 | 0, 0, 0, 0 | 2, 1, 0, 0 | 0, 2, 0, 1 | 0, 0, 1, 0 |
| C[T > A]G | 1, 0, 0, 0 | 0, 0, 0 | 0, 0, 1, 0 | 0, 2, 0, 2 | 2, 0, 2, 3 | 0, 0, 0, 2 | 0, 0, 0, 0 |
| C[T > A]T | 0, 0, 0, 0 | 0, 0, 0 | 0, 1, 0, 0 | 0, 0, 1, 2 | 0, 0, 0, 0 | 0, 0, 0, 0 | 0, 1, 1, 4 |
| C[T > C]A | 1, 1, 2, 0 | 1, 0, 0 | 1, 2, 2, 0 | 0, 1, 1, 2 | 1, 0, 1, 1 | 1, 1, 2, 1 | 0, 1, 0, 0 |
| C[T > C]C | 0, 0, 0, 0 | 1, 0, 0 | 2, 2, 0, 0 | 0, 0, 1, 1 | 1, 0, 2, 0 | 1, 0, 0, 1 | 0, 1, 0, 0 |
| C[T > C]G | 1, 0, 2, 0 | 0, 1, 0 | 0, 0, 1, 1 | 2, 0, 3, 3 | 1, 0, 0, 2 | 0, 2, 0, 0 | 0, 1, 0, 0 |
| C[T > C]T | 0, 0, 0, 0 | 1, 0, 1 | 1, 1, 0, 1 | 1, 0, 0, 1 | 0, 0, 0, 3 | 0, 2, 2, 1 | 0, 1, 0, 0 |
| C[T > G]A | 0, 0, 0, 0 | 1, 0, 1 | 0, 0, 0, 0 | 0, 0, 0, 1 | 0, 0, 0, 0 | 0, 0, 0, 0 | 0, 0, 0, 0 |
| C[T > G]C | 0, 0, 0, 0 | 0, 0, 0 | 1, 0, 0, 0 | 0, 0, 0, 0 | 0, 0, 0, 0 | 1, 0, 1, 1 | 0, 0, 0, 0 |
| C[T > G]G | 1, 0, 1, 1 | 0, 2, 1 | 1, 0, 0, 0 | 1, 0, 0, 2 | 0, 0, 0, 1 | 0, 1, 0, 2 | 0, 0, 0, 0 |
| C[T > G]T | 0, 0, 0, 0 | 2, 0, 0 | 1, 1, 0, 0 | 2, 2, 0, 2 | 0, 1, 0, 0 | 0, 0, 1, 2 | 0, 0, 1, 0 |
| G[C > A]A | 30, 30, 17, 20 | 23, 20, 24 | 48, 37, 41, 39 | 31, 26, 31, 29 | 21, 14, 29, 29 | 32, 29, 28, 29 | 12, 33, 19, 17 |
| G[C > A]C | 3, 1, 1, 1 | 2, 4, 3 | 0, 2, 4, 1 | 1, 1, 1, 2 | 0, 1, 4, 1 | 2, 5, 4, 4 | 1, 0, 0, 4 |
| G[C > A]G | 0, 1, 0, 0 | 1, 0, 1 | 1, 1, 1, 0 | 1, 0, 0, 2 | 1, 0, 0, 2 | 1, 2, 2, 2 | 0, 0, 2, 3 |
| G[C > A]T | 13, 12, 10, 13 | 8, 11, 6 | 13, 13, 19, 15 | 14, 9, 17, 13 | 13, 2, 14, 16 | 14, 12, 13, 16 | 4, 10, 8, 4 |
| G[C > G]A | 0, 0, 0, 0 | 0, 0, 0 | 0, 2, 0, 0 | 1, 0, 1, 0 | 0, 1, 0, 0 | 0, 0, 0, 0 | 0, 0, 0, 0 |
| G[C > G]C | 0, 0, 0, 0 | 0, 0, 0 | 1, 0, 0, 0 | 0, 0, 0, 0 | 2, 0, 0, 0 | 1, 1, 0, 0 | 0, 1, 0, 0 |
| G[C > G]G | 1, 0, 0, 0 | 0, 0, 0 | 0, 0, 0, 0 | 0, 0, 1, 0 | 0, 0, 2, 0 | 0, 0, 0, 0 | 1, 1, 0, 1 |
| G[C > G]T | 0, 0, 0, 0 | 0, 1, 1 | 1, 0, 1, 1 | 1, 0, 0, 0 | 1, 0, 0, 0 | 1, 0, 0, 0 | 0, 0, 0, 1 |
| G[C > T]A | 4, 4, 2, 0 | 5, 1, 1 | 5, 6, 3, 3 | 3, 1, 1, 3 | 3, 0, 2, 5 | 2, 4, 0, 3 | 1, 4, 0, 5 |
| G[C > T]C | 0, 2, 2, 4 | 1, 0, 0 | 3, 1, 2, 1 | 2, 0, 0, 2 | 0, 1, 4, 0 | 1, 0, 1, 2 | 0, 1, 3, 3 |
| G[C > T]G | 2, 7, 3, 0 | 3, 3, 1 | 2, 3, 3, 1 | 1, 0, 0, 3 | 1, 0, 1, 5 | 1, 1, 3, 1 | 2, 3, 5, 3 |
| G[C > T]T | 1, 1, 2, 0 | 2, 2, 2 | 2, 0, 1, 3 | 3, 2, 4, 2 | 1, 3, 0, 1 | 3, 2, 4, 1 | 1, 2, 0, 3 |
| G[T > A]A | 1, 1, 0, 0 | 0, 0, 0 | 1, 0, 0, 0 | 2, 0, 1, 1 | 1, 0, 0, 1 | 0, 0, 0, 0 | 0, 0, 1, 1 |
| G[T > A]C | 0, 0, 0, 0 | 0, 0, 0 | 0, 0, 0, 0 | 0, 1, 1, 0 | 0, 0, 0, 0 | 2, 0, 0, 1 | 0, 0, 0, 1 |
| G[T > A]G | 0, 1, 0, 0 | 0, 0, 1 | 0, 1, 0, 0 | 0, 0, 1, 0 | 0, 1, 0, 1 | 1, 0, 0, 0 | 1, 0, 0, 1 |
| G[T > A]T | 1, 1, 0, 1 | 0, 0, 1 | 1, 0, 1, 0 | 1, 1, 1, 1 | 0, 0, 0, 0 | 0, 0, 0, 0 | 0, 0, 1, 0 |
| G[T > C]A | 0, 0, 0, 1 | 0, 0, 2 | 2, 1, 0, 0 | 1, 0, 0, 0 | 0, 0, 0, 1 | 2, 0, 0, 1 | 0, 0, 0, 0 |
| G[T > C]C | 1, 0, 0, 0 | 0, 0, 0 | 1, 0, 1, 0 | 0, 1, 0, 3 | 0, 0, 0, 1 | 0, 1, 1, 0 | 0, 0, 0, 2 |
| G[T > C]G | 0, 1, 0, 0 | 1, 0, 1 | 0, 0, 1, 1 | 2, 0, 1, 0 | 0, 0, 0, 2 | 0, 2, 1, 0 | 0, 0, 0, 0 |
| G[T > C]T | 0, 1, 0, 0 | 1, 0, 1 | 0, 0, 0, 0 | 0, 1, 0, 0 | 0, 0, 0, 1 | 0, 0, 0, 2 | 0, 0, 2, 1 |
| G[T > G]A | 0, 0, 0, 0 | 0, 0, 0 | 0, 0, 1, 0 | 0, 0, 0, 0 | 0, 0, 0, 0 | 0, 0, 0, 0 | 1, 0, 0, 0 |
| G[T > G]C | 0, 0, 0, 0 | 0, 0, 0 | 0, 0, 0, 0 | 0, 0, 1, 1 | 0, 0, 1, 0 | 0, 0, 0, 0 | 0, 0, 0, 0 |
| G[T > G]G | 0, 0, 0, 0 | 0, 1, 0 | 0, 0, 0, 1 | 0, 0, 0, 0 | 0, 0, 0, 0 | 0, 0, 0, 0 | 0, 0, 0, 0 |
| G[T > G]T | 0, 0, 0, 0 | 0, 2, 0 | 0, 0, 1, 0 | 0, 0, 0, 0 | 0, 0, 1, 0 | 0, 0, 1, 0 | 0, 1, 0, 1 |
| T[C > A]A | 12, 4, 5, 4 | 3, 18, 7 | 23, 9, 19, 21 | 15, 14, 15, 13 | 4, 6, 9, 16 | 12, 10, 7, 9 | 4, 7, 6, 9 |
| T[C > A]C | 3, 0, 4, 2 | 4, 3, 8 | 4, 4, 6, 5 | 2, 3, 6, 6 | 3, 2, 7, 6 | 9, 6, 8, 3 | 4, 4, 5, 2 |
| T[C > A]G | 0, 2, 1, 2 | 0, 0, 1 | 0, 2, 3, 1 | 0, 1, 2, 0 | 0, 2, 0, 1 | 1, 1, 1, 1 | 0, 2, 3, 0 |
| T[C > A]T | 23, 24, 20, 24 | 13, 33, 21 | 33, 24, 41, 30 | 29, 19, 30, 26 | 10, 13, 33, 26 | 33, 27, 31, 25 | 7, 31, 13, 20 |
| T[C > G]A | 1, 1, 0, 0 | 0, 0, 1 | 1, 0, 1, 3 | 1, 1, 0, 0 | 1, 0, 2, 1 | 1, 0, 1, 0 | 0, 0, 1, 0 |
| T[C > G]C | 0, 0, 0, 0 | 0, 0, 0 | 0, 0, 2, 0 | 1, 0, 1, 0 | 0, 0, 0, 0 | 1, 0, 0, 0 | 0, 0, 0, 0 |
| T[C > G]G | 0, 1, 1, 0 | 0, 0, 0 | 0, 0, 0, 2 | 0, 0, 0, 2 | 0, 0, 0, 0 | 0, 0, 0, 0 | 0, 0, 1, 3 |
| T[C > G]T | 2, 0, 1, 0 | 1, 0, 1 | 0, 0, 3, 2 | 0, 0, 0, 2 | 4, 0, 0, 2 | 0, 0, 3, 0 | 0, 2, 1, 1 |
| T[C > T]A | 2, 2, 2, 1 | 5, 2, 3 | 4, 3, 5, 4 | 3, 1, 2, 5 | 1, 4, 3, 8 | 3, 1, 2, 5 | 2, 7, 0, 3 |
| T[C > T]C | 3, 1, 1, 3 | 1, 2, 3 | 6, 2, 3, 5 | 4, 0, 4, 3 | 4, 1, 1, 2 | 3, 3, 2, 1 | 0, 2, 1, 1 |
| T[C > T]G | 1, 3, 1, 0 | 0, 2, 4 | 2, 2, 3, 3 | 0, 0, 3, 1 | 1, 1, 1, 3 | 1, 2, 2, 0 | 1, 3, 1, 3 |
| T[C > T]T | 2, 3, 1, 1 | 1, 3, 1 | 3, 3, 3, 2 | 5, 0, 5, 2 | 1, 1, 3, 1 | 3, 2, 2, 2 | 1, 6, 1, 0 |
| T[T > A]A | 2, 3, 3, 0 | 1, 0, 1 | 0, 2, 1, 1 | 0, 3, 2, 1 | 0, 3, 1, 3 | 3, 3, 2, 2 | 2, 3, 3, 2 |
| T[T > A]C | 0, 1, 0, 0 | 0, 0, 0 | 1, 0, 1, 0 | 1, 2, 1, 1 | 0, 0, 0, 0 | 0, 0, 0, 0 | 0, 1, 0, 1 |
| T[T > A]G | 0, 3, 0, 0 | 1, 1, 1 | 1, 0, 1, 0 | 3, 1, 1, 1 | 1, 0, 0, 0 | 0, 0, 0, 0 | 2, 0, 0, 0 |
| T[T > A]T | 0, 4, 0, 1 | 0, 1, 3 | 0, 2, 2, 1 | 1, 3, 5, 1 | 0, 0, 2, 0 | 1, 1, 0, 1 | 0, 0, 3, 1 |
| T[T > C]A | 1, 1, 0, 0 | 2, 1, 3 | 5, 4, 10, 0 | 5, 2, 3, 2 | 2, 2, 1, 5 | 3, 2, 1, 0 | 0, 2, 0, 1 |
| T[T > C]C | 4, 1, 1, 1 | 1, 1, 0 | 1, 3, 1, 0 | 0, 0, 2, 1 | 1, 0, 0, 0 | 0, 0, 0, 1 | 0, 0, 2, 0 |
| T[T > C]G | 0, 2, 1, 1 | 0, 0, 0 | 0, 1, 0, 1 | 4, 2, 2, 1 | 0, 0, 3, 1 | 1, 0, 0, 2 | 0, 1, 2, 1 |
| T[T > C]T | 0, 0, 2, 2 | 1, 3, 3 | 2, 2, 2, 4 | 3, 4, 2, 5 | 1, 0, 2, 1 | 1, 0, 0, 1 | 0, 3, 0, 0 |
| T[T > G]A | 0, 0, 0, 2 | 0, 0, 0 | 0, 0, 1, 1 | 0, 1, 2, 1 | 2, 0, 0, 3 | 0, 0, 0, 1 | 0, 0, 0, 1 |
| T[T > G]C | 0, 0, 1, 0 | 0, 0, 0 | 0, 0, 0, 0 | 0, 0, 1, 0 | 1, 0, 0, 0 | 1, 1, 0, 0 | 0, 0, 0, 0 |
| T[T > G]G | 1, 0, 1, 1 | 0, 0, 0 | 1, 1, 0, 2 | 1, 0, 0, 1 | 0, 0, 1, 0 | 0, 0, 2, 0 | 0, 1, 0, 0 |
| T[T > G]T | 5, 1, 1, 1 | 1, 2, 0 | 2, 0, 1, 0 | 0, 2, 1, 4 | 3, 1, 1, 0 | 0, 1, 1, 1 | 1, 0, 0, 0 |

In each column, commas separate values for different subclones.

We confirmed that mutational outcomes were neither due to off-target edits nor to the acquisition of new driver mutations (see Methods). We verified that knockouts were biallelic, confirmed this further by protein mass spectrometry, and ensured that subclones were derived from single cells in all comparative analyses (see Methods).

### Example 2—Mutational Consequences of Gene Knockouts

In this example, the inventors investigated whether knocking out the genes as described in Example 1 would produce a mutational signature.

Methods

See Example 1.

Proliferation assay. Cells were seeded at 5,500 per well on 96-w plates. Measurements were taken at 24 h intervals post-seeding over a period of 5 days according to manufacturer's instructions. Briefly, plates were removed from the incubator and allowed to equilibrate at room temperature for 30 minutes, and equal volume of CellTiter-Glo reagent (Promega) was added directly to the wells. Plates were incubated at room temperature for 2 minutes on a shaker and left to equilibrate for 10 minutes at 22° C. before luminescence was measured on PHERAstar FS microplate reader. Luminescence readings were normalized and presented as relative luminescence units (RLU) to time point 0 (to). Doubling time was calculated based on replicate-averaged readings on the linear portion of the proliferation curve (exponential phase) using formula:

$$\frac{24 hr \times log ( 2 )}{log ( Final Measurement ) - log ( Initial Measurement )}$$

Determination of gene knockout-associated mutational signatures. An intrinsic background mutagenesis exists in normal cells grown in culture. Knocking out a DNA repair gene that is involved in repairing endogenous DNA damage may result in increased unrepaired DNA damage and, thereby result in mutation accumulation with subsequent rounds of replication. Whole-genome sequencing of these knockouts can detect the mutations that occur as a result of being a specified knockout. If the mutation burden and the mutational profile of a knockout is significantly different from the control subclones which have only the background mutagenesis, it is most likely that there is gene knockout-associated mutagenesis. Based on this principle, our approach to identify gene knockout-associated mutational signature involved three steps: 1) we determined the background mutational signature; 2) we determined the difference between the mutational profile of knockout and background mutation profiles; 3) we removed the background mutation profile from mutation profile of the knockout subclone.

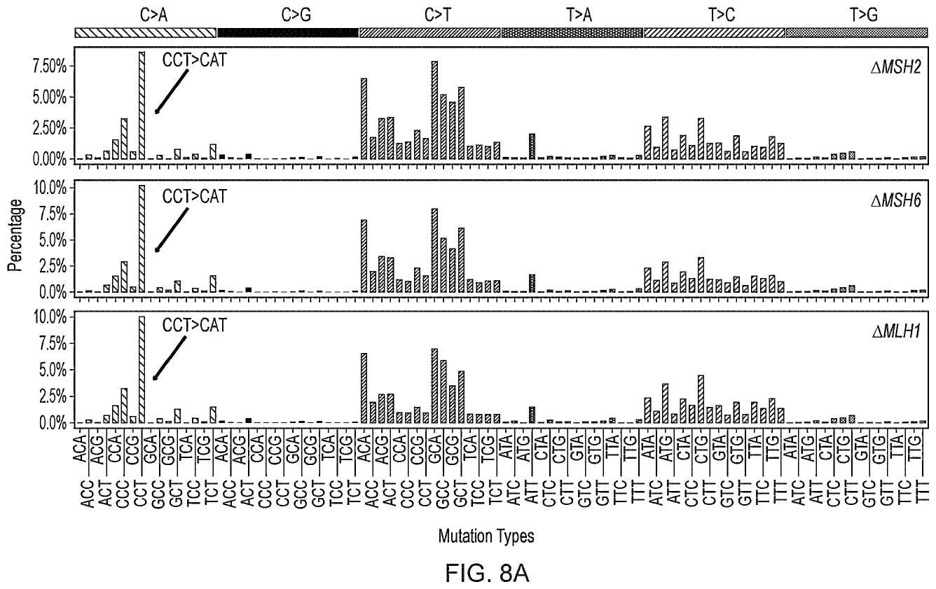

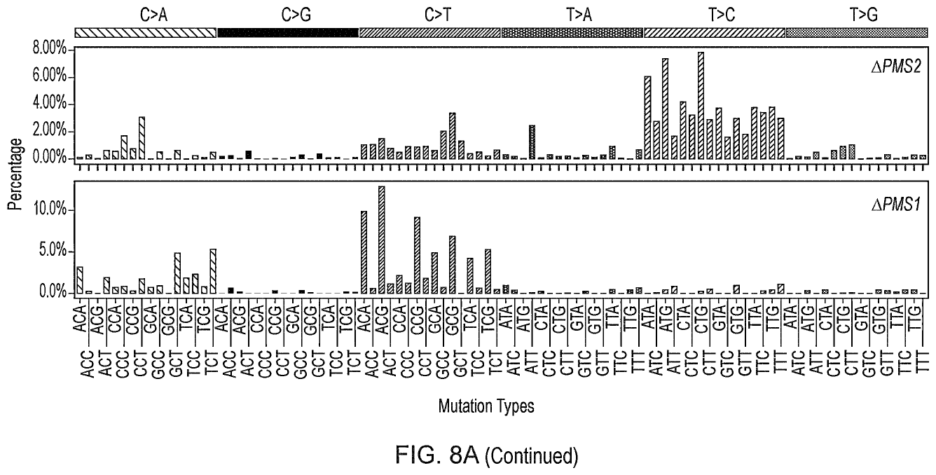

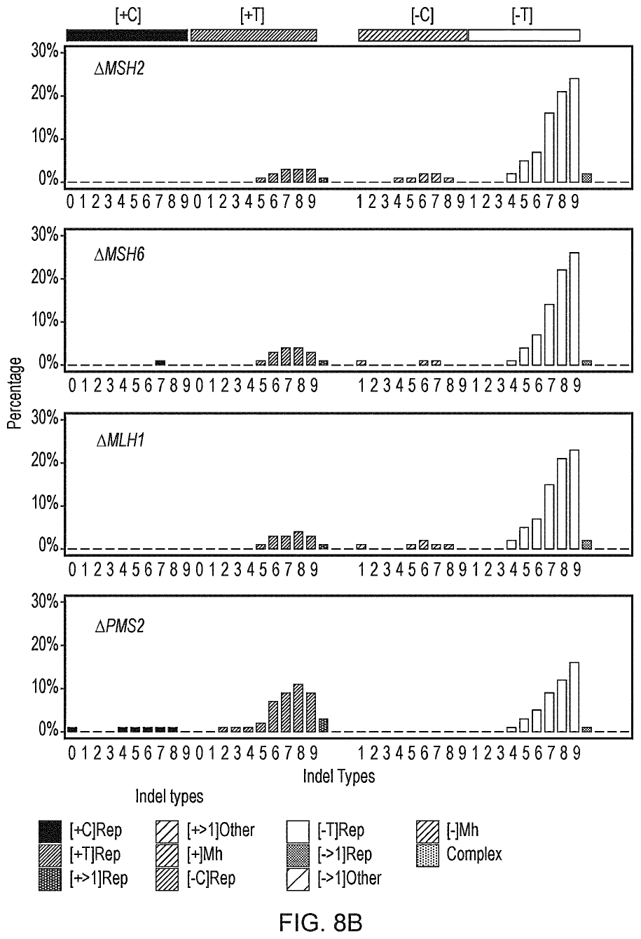

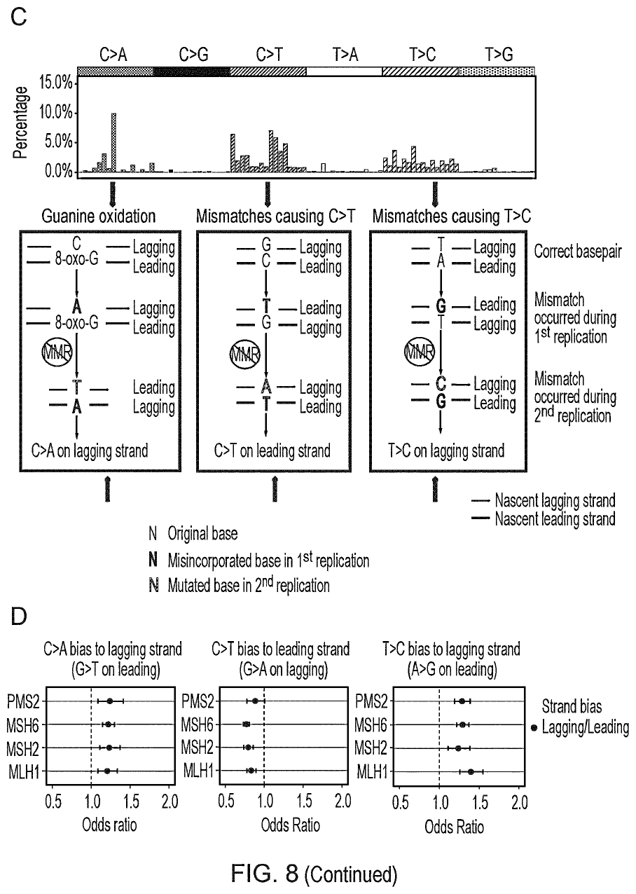

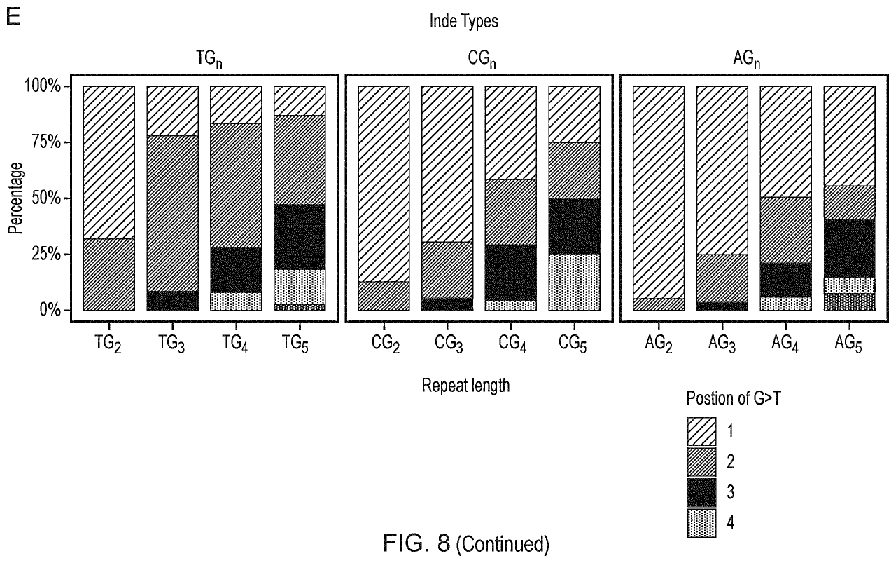

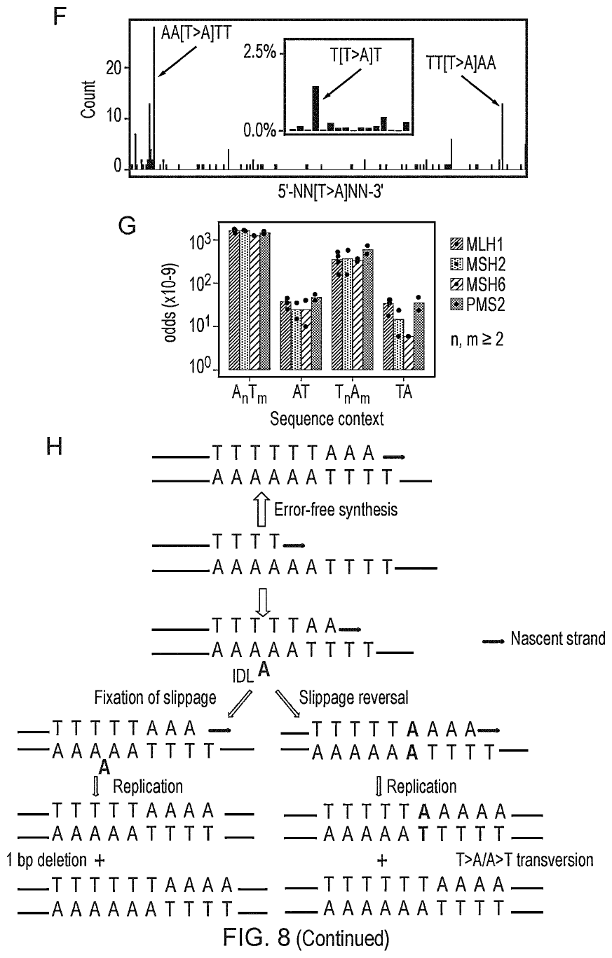

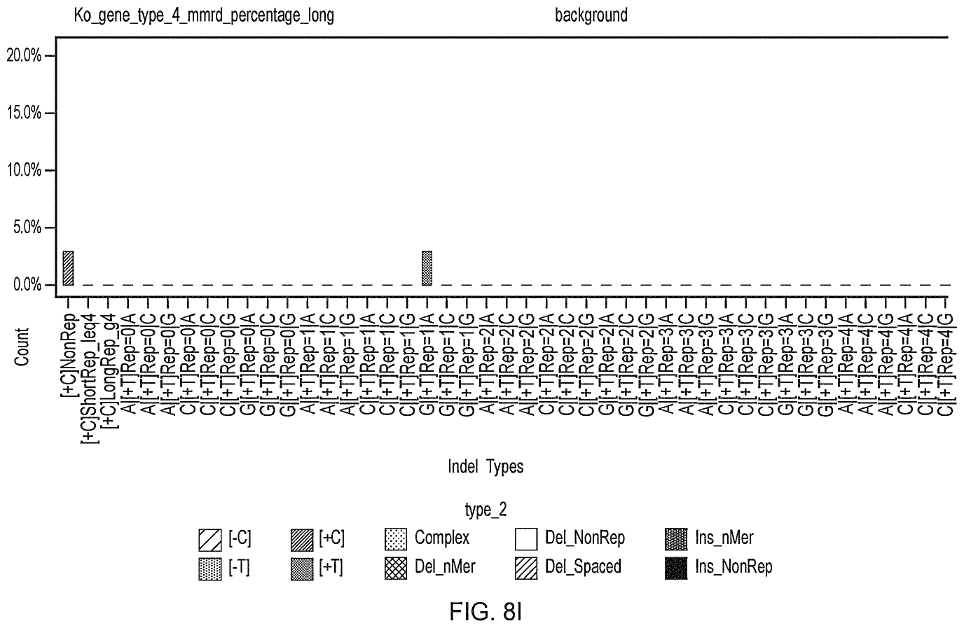

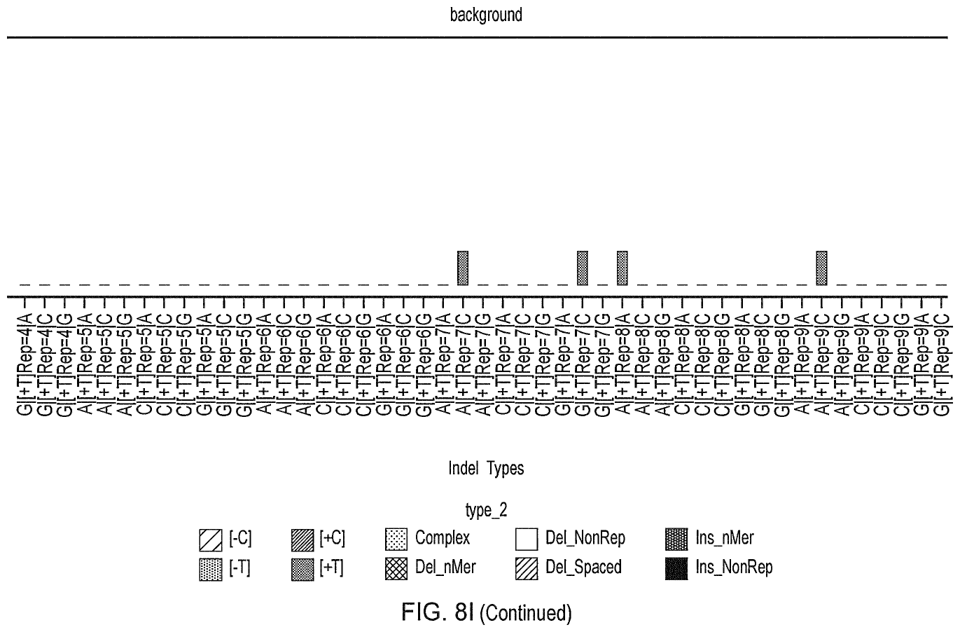

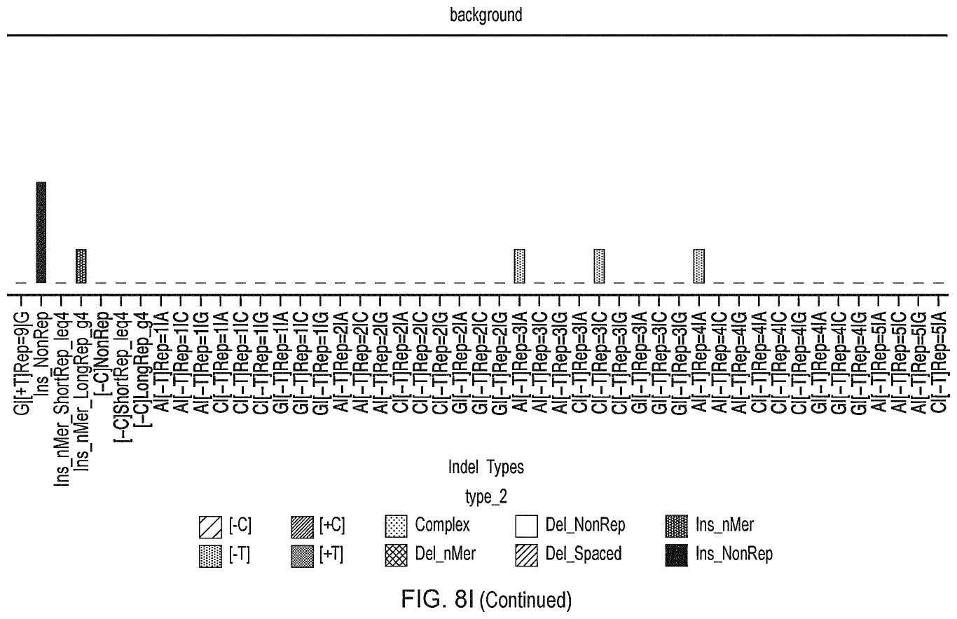

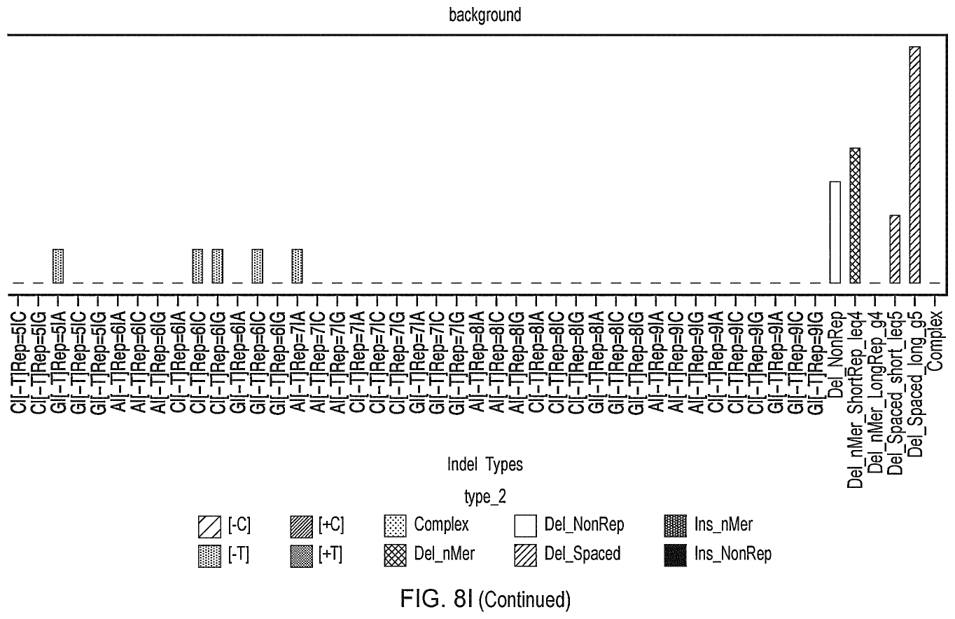

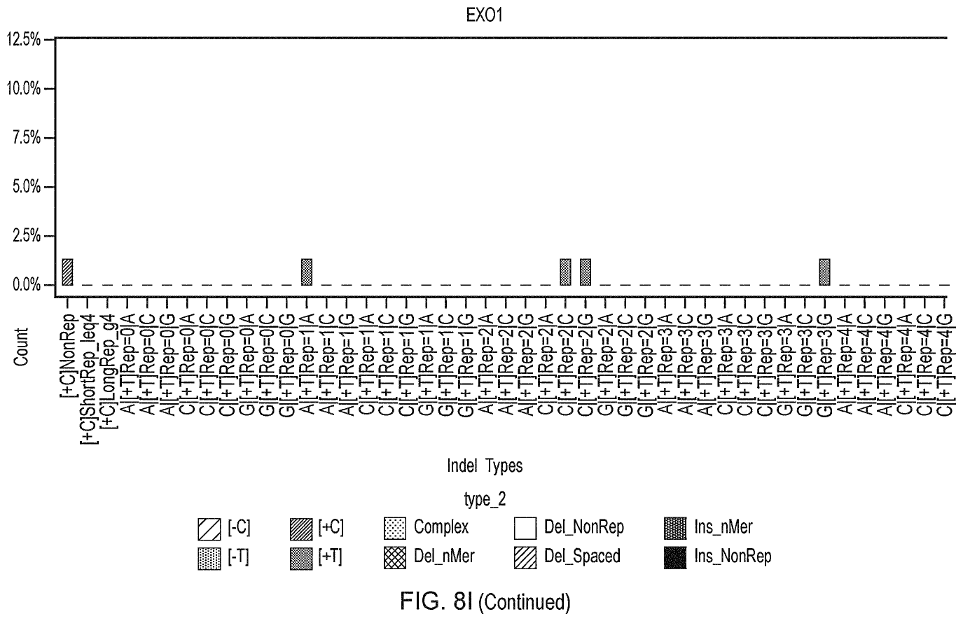

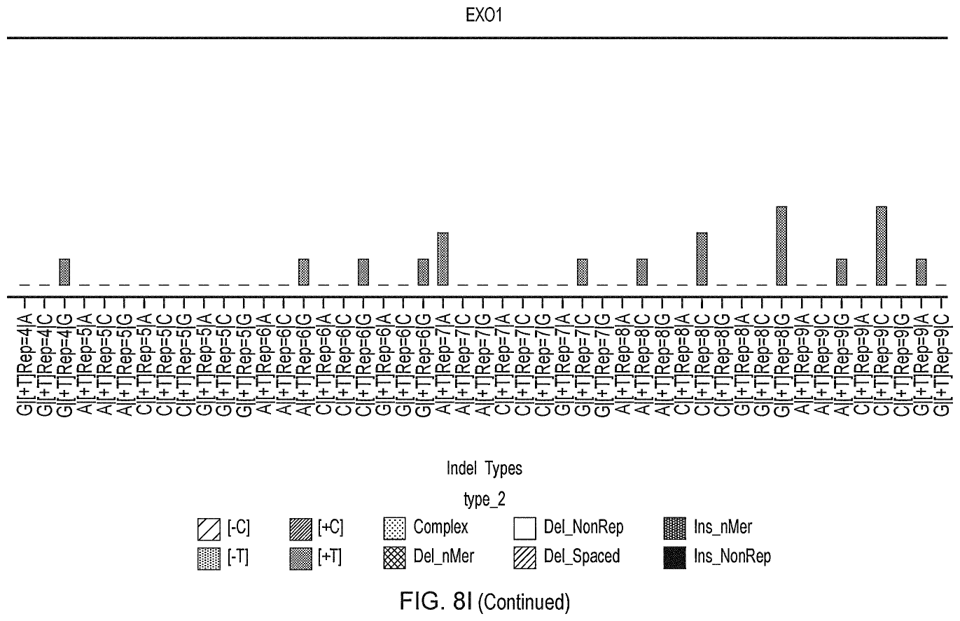

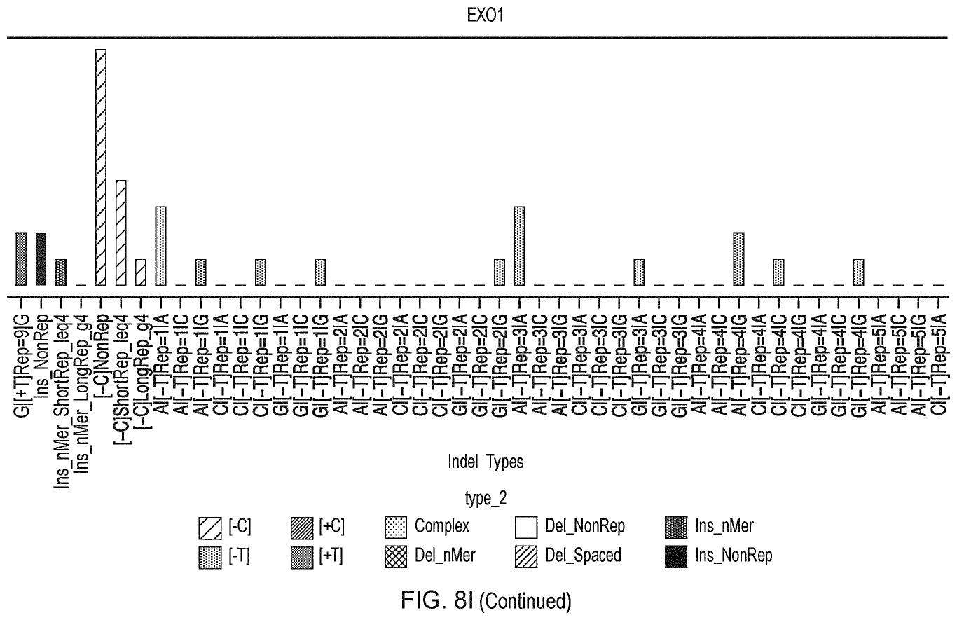

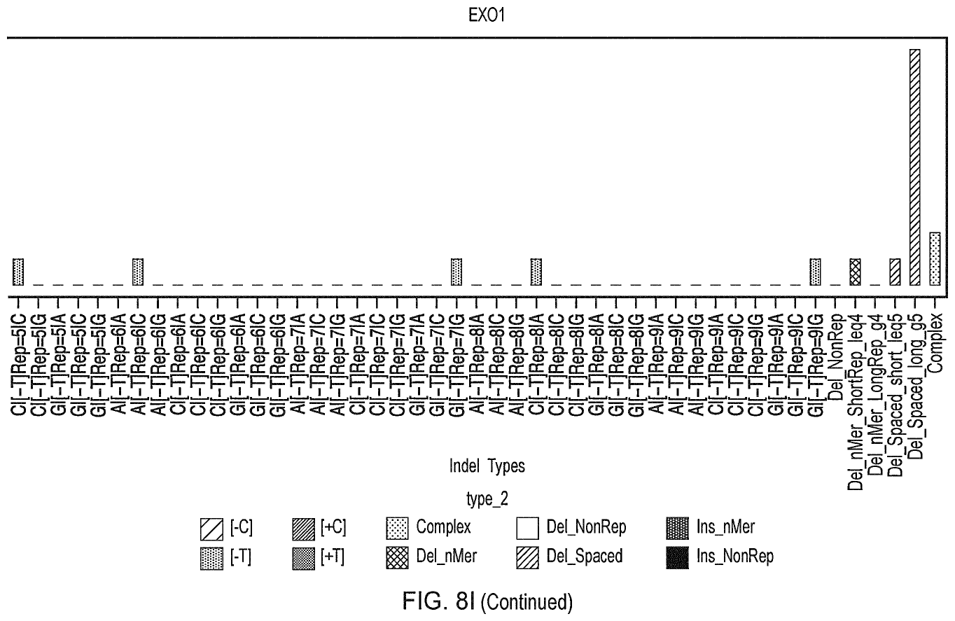

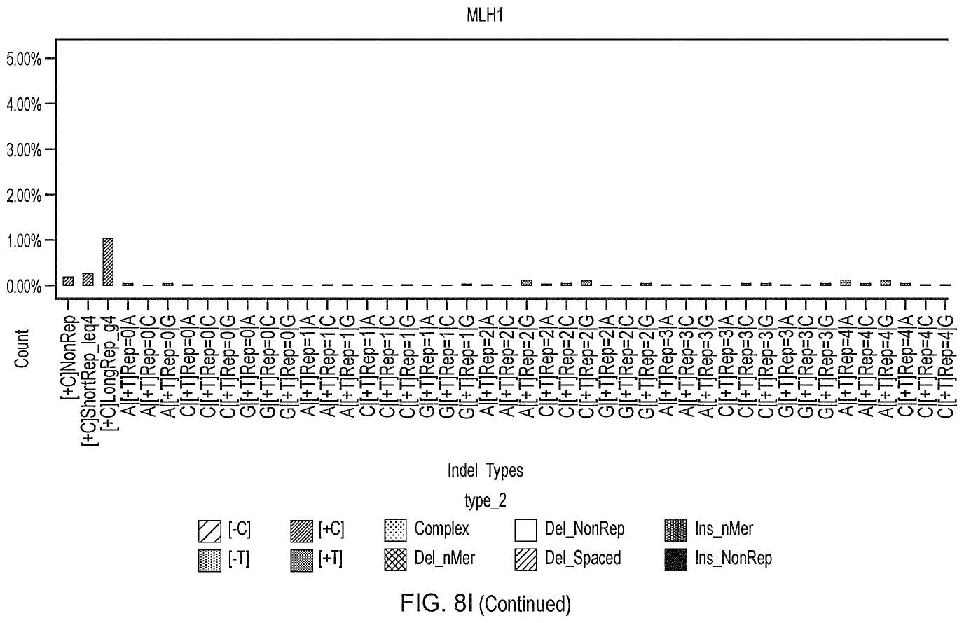

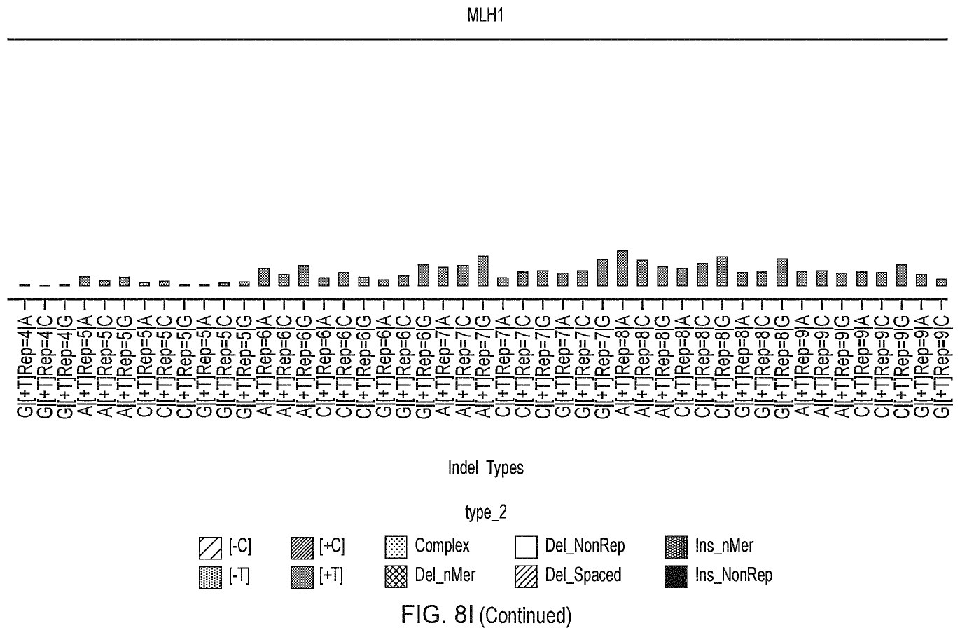

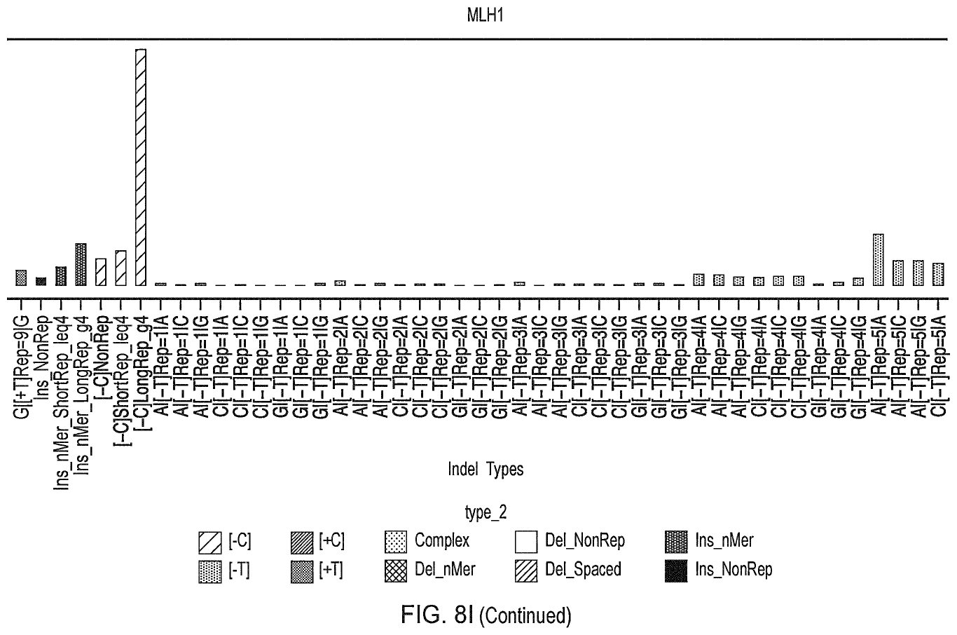

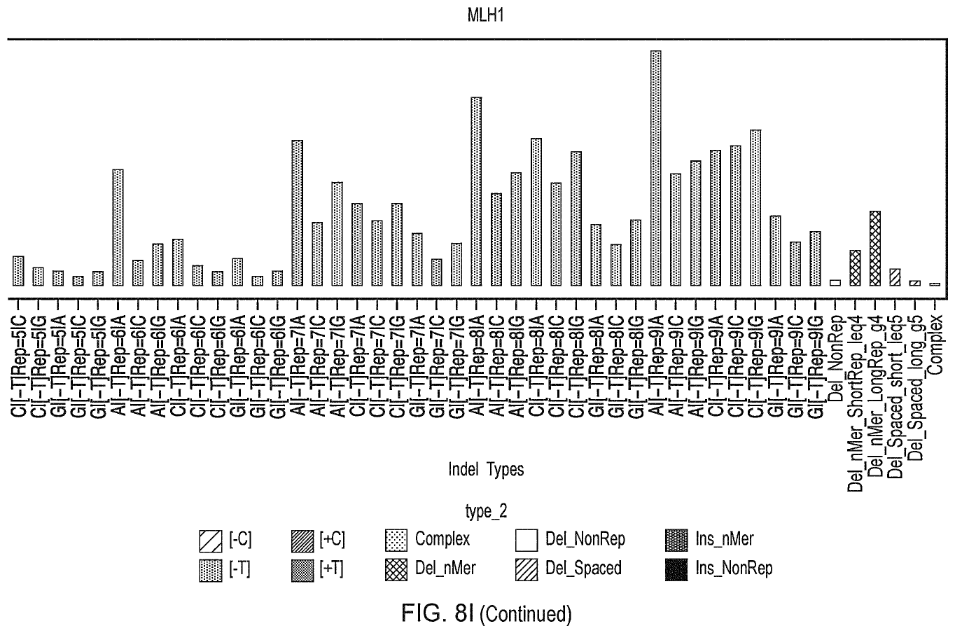

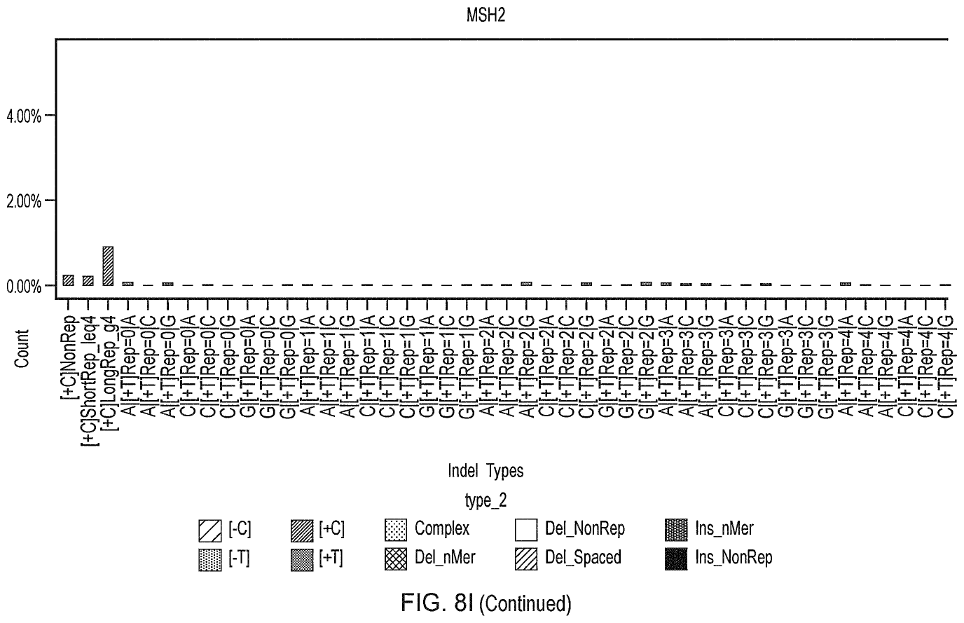

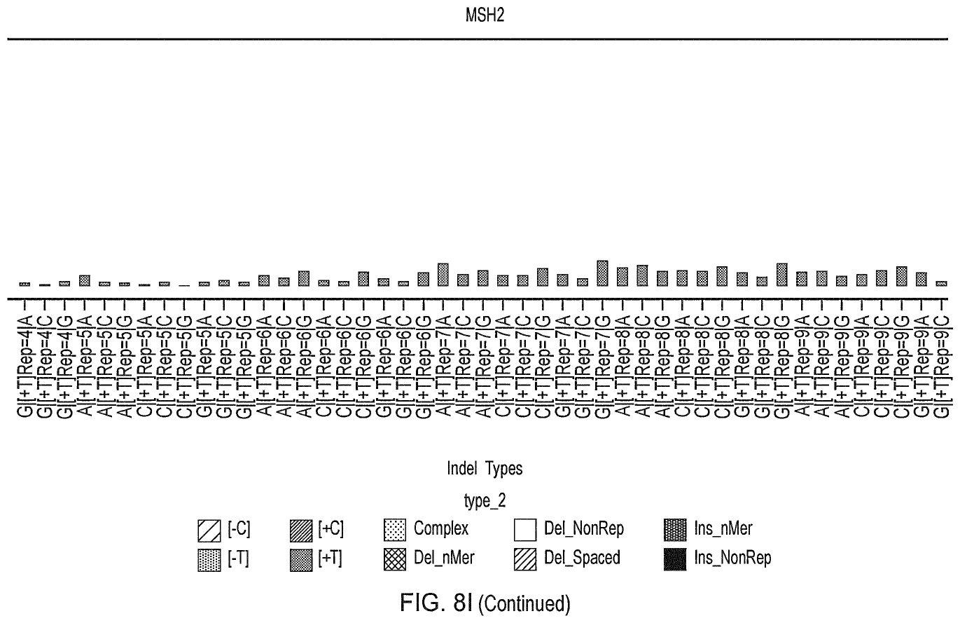

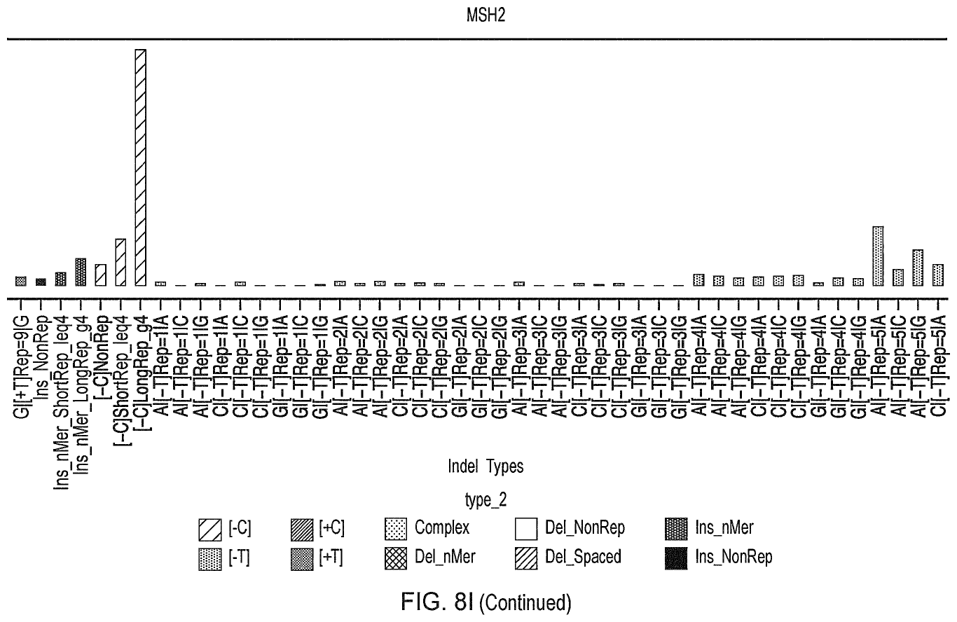

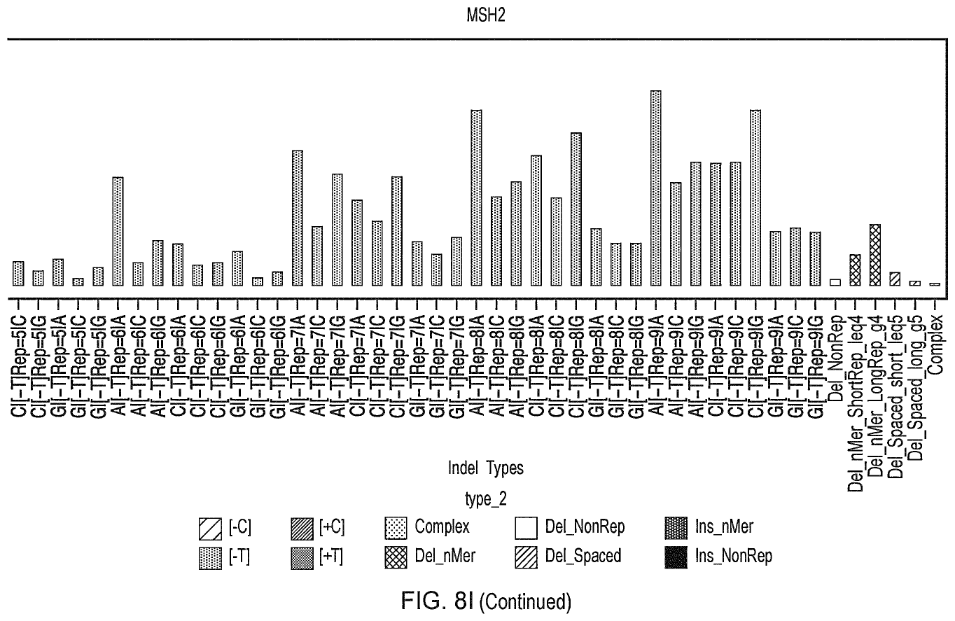

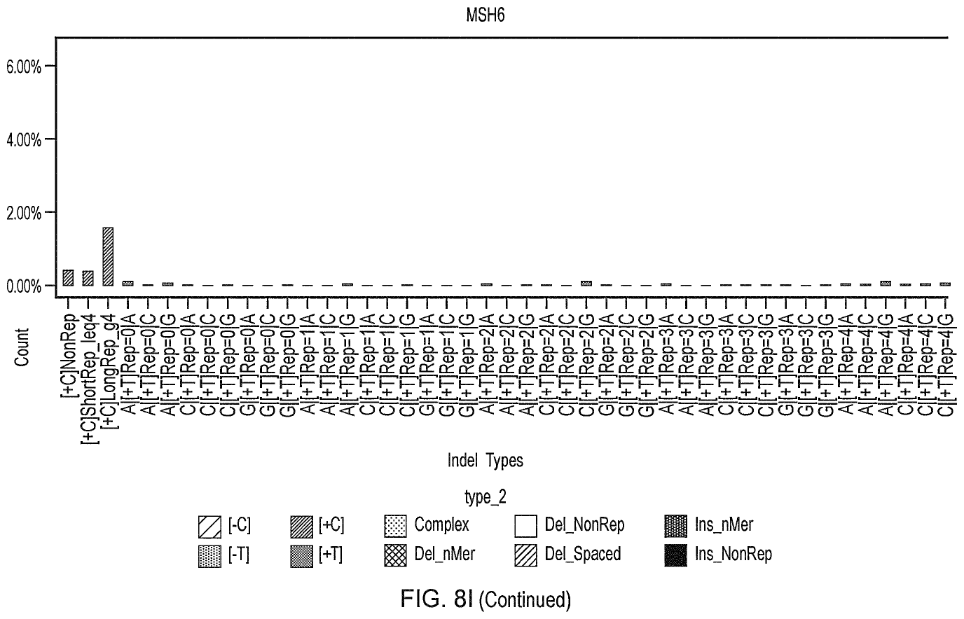

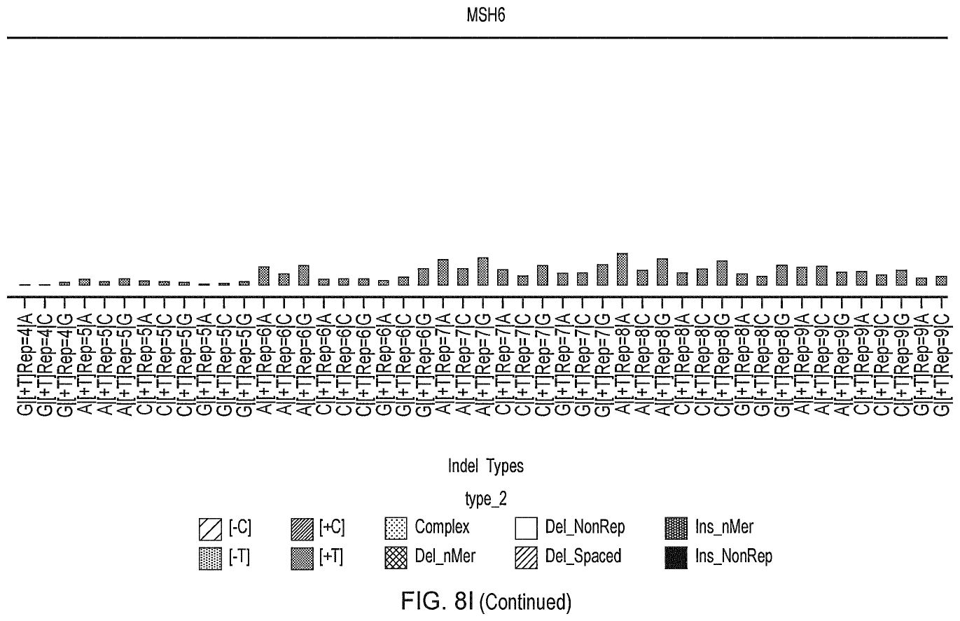

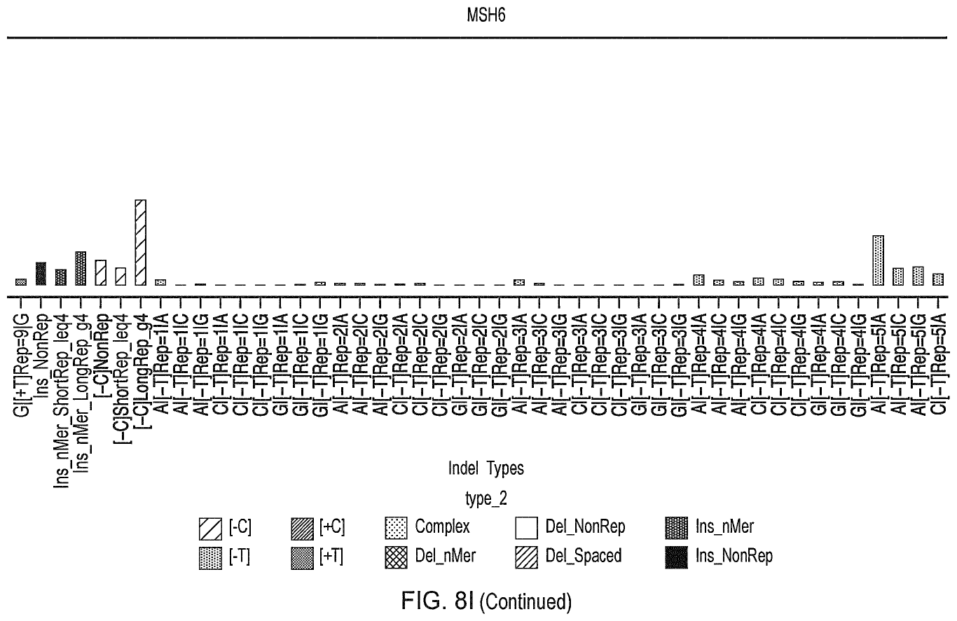

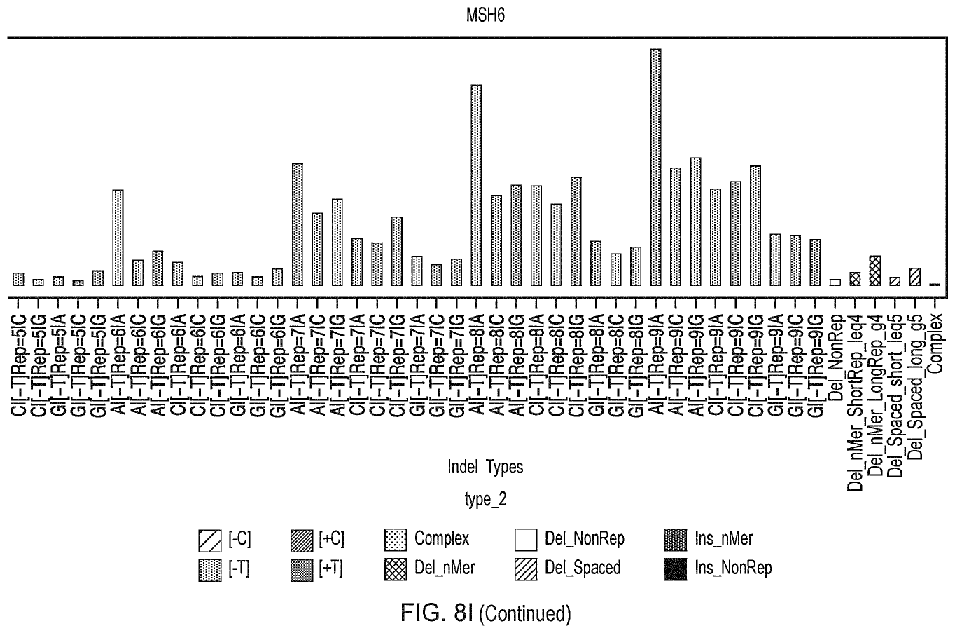

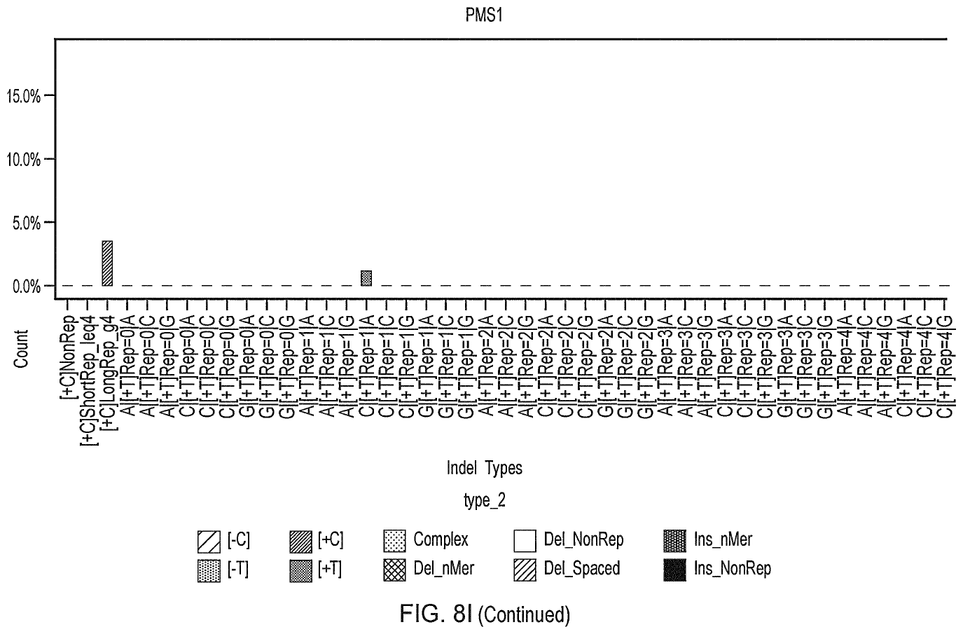

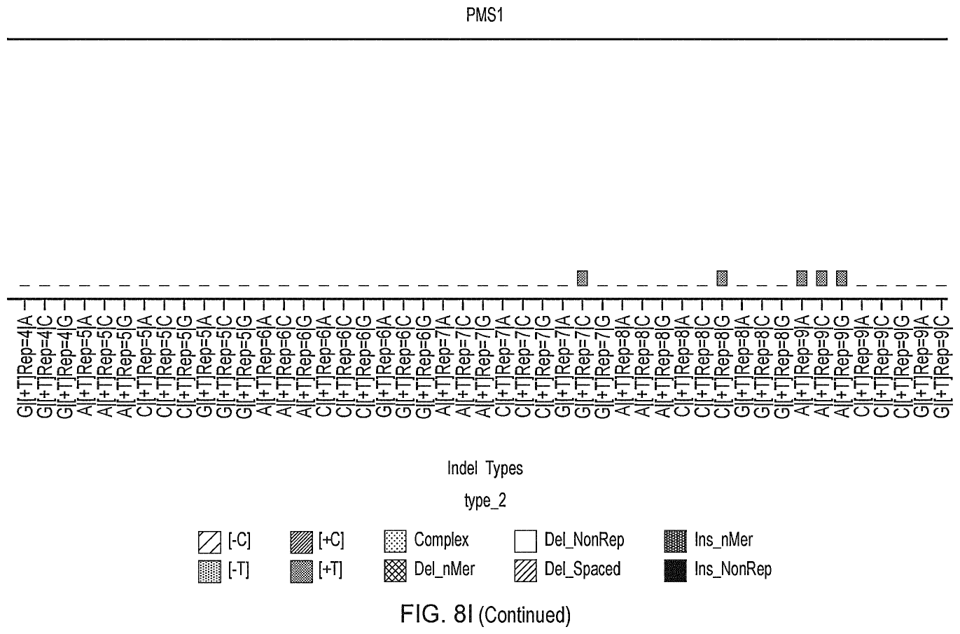

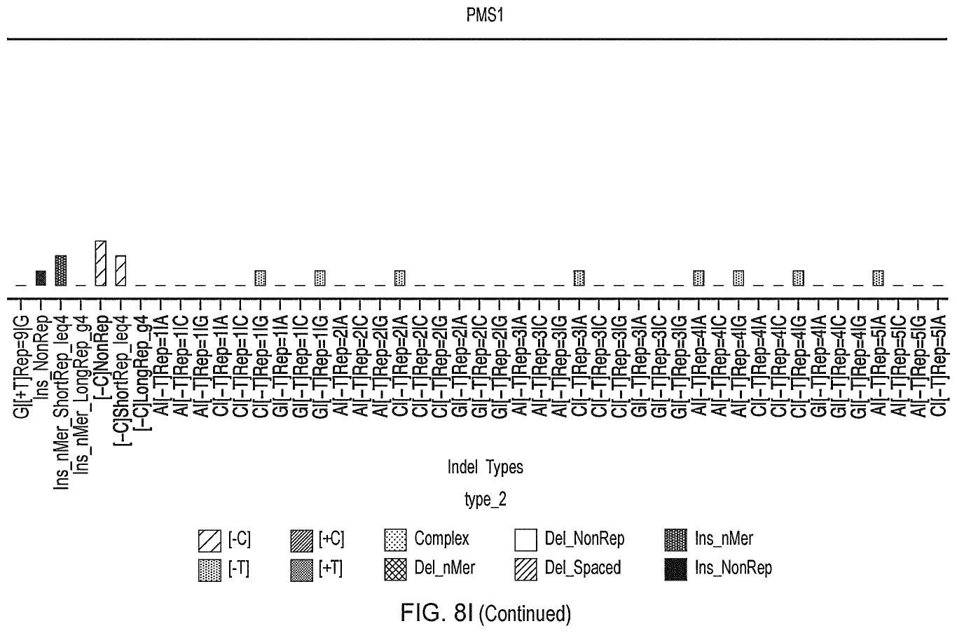

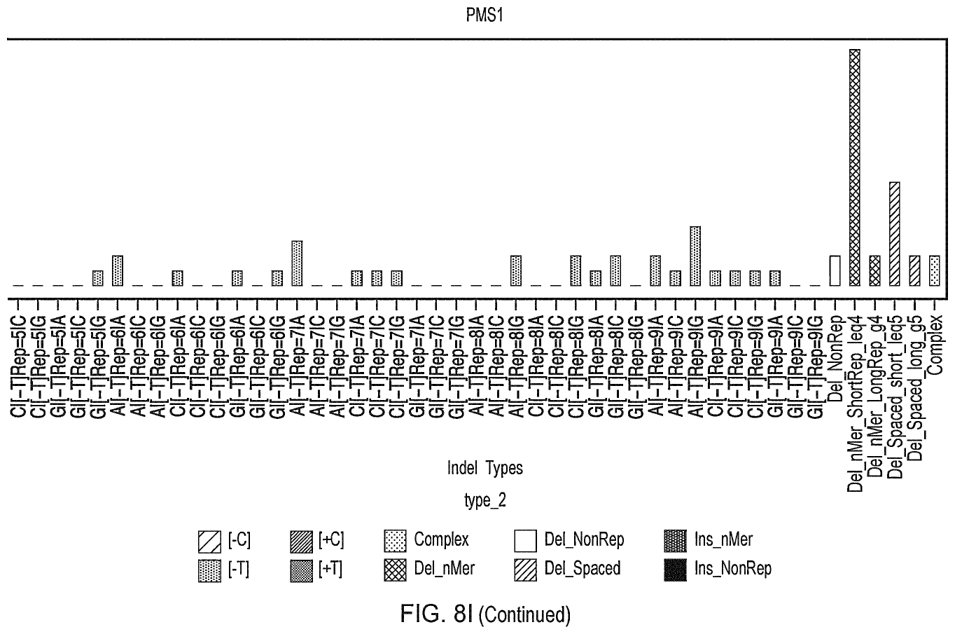

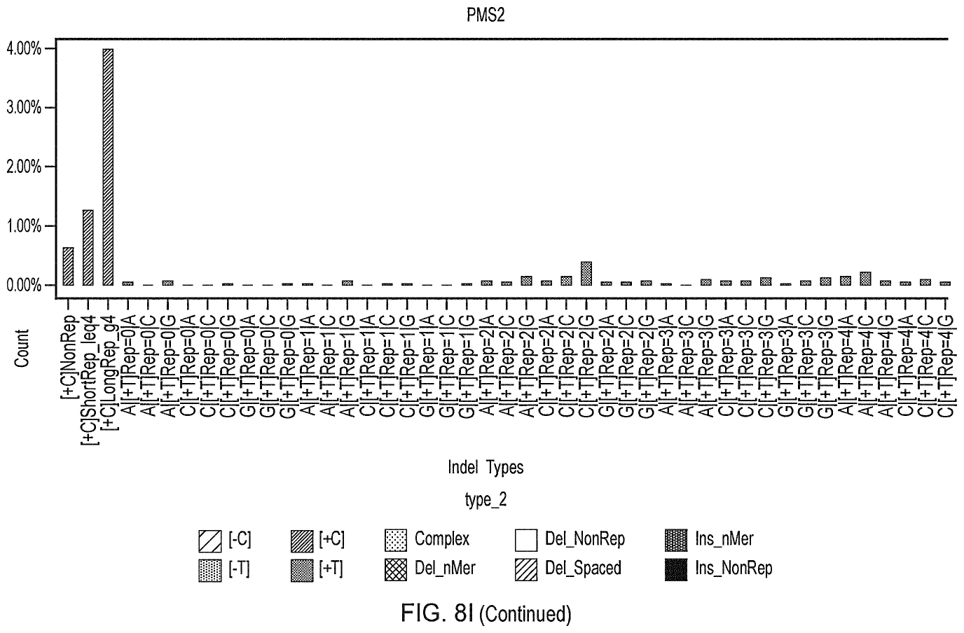

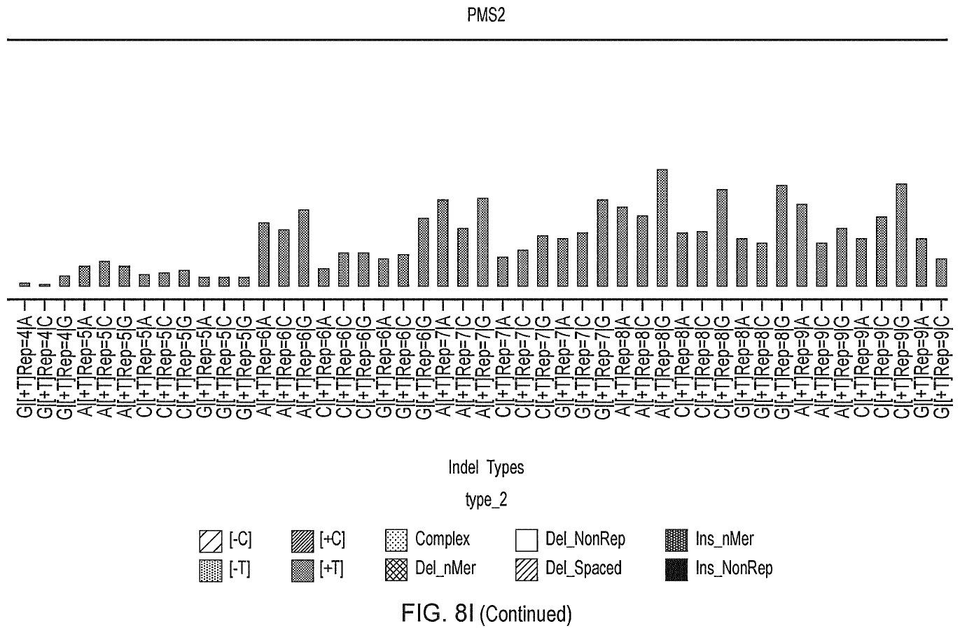

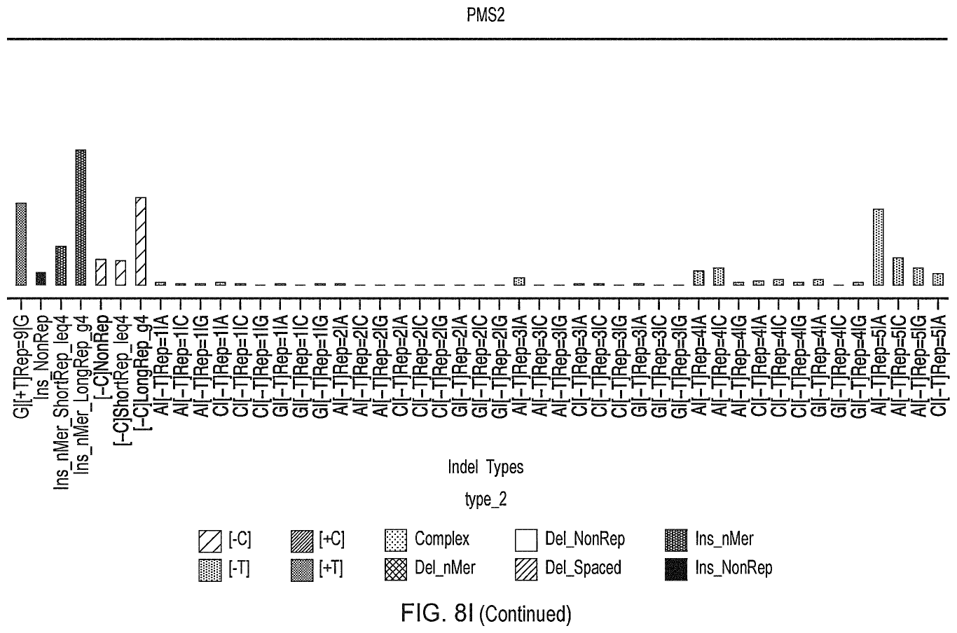

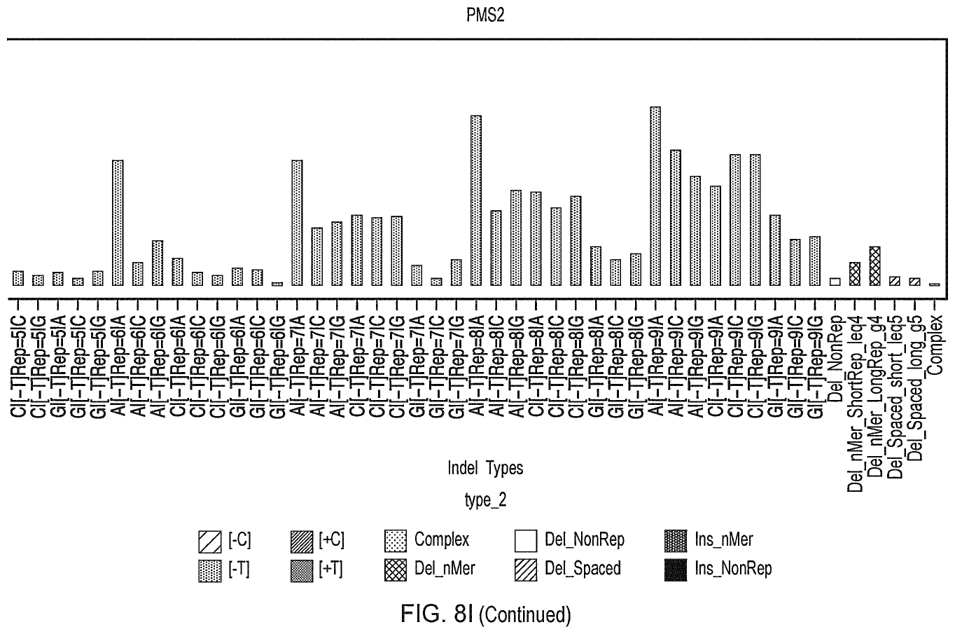

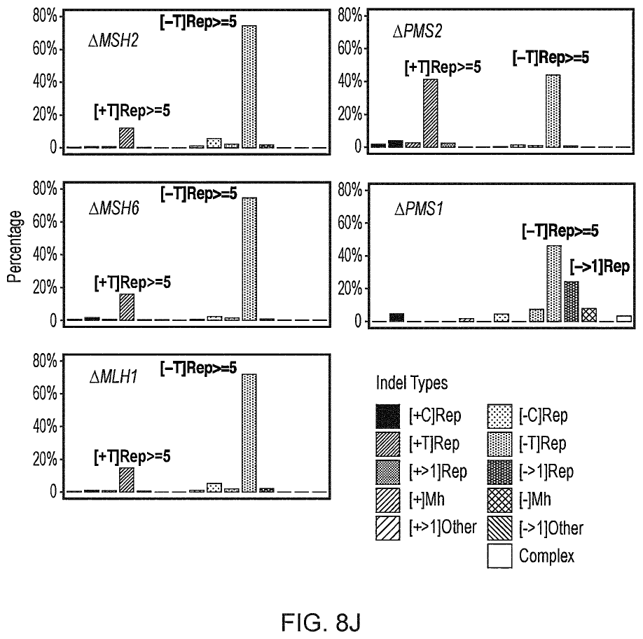

Substitution profiles were described according to the classical convention of 96 channels: the product of 6 types of substitution multiplied by 4 types of 5′ base (A,C,G,T) and 4 types of 3′ base (A,C,G,T). Indel profiles were described by type (insertion, deletion, complex), size (1-bp or longer) and flanking sequence (repeat-mediated, microhomology-mediated or other) of the indel. Here, we used two sets of indel channels. Set one contains 15 channels: 1 bp C/T insertion at short repetitive sequence (<5 bp), 1 bp C/T insertion at long repetitive sequence (>=5 bp), long insertions (>1 bp) at repetitive sequences, microhomology-mediated insertions, 1 bp C/T deletions at short repetitive sequence (<5 bp), 1 bp C/T deletions at long repetitive sequence (>=5 bp), long deletions (>1 bp) at repetitive sequences, microhomology-mediated deletions, other deletion and complex indels (see FIG. 8J). Set two contains 45 channels, in which the 1 bp C/T indels at repetitive sequences are further expanded according to the exact length of the repetitive sequences (FIG. 8B). Indel channel set one was applied to all knockout subclones, whilst channel set two was only applied to four MMR gene knockouts (ΔMLH1, ΔPMS2, ΔMSH2, ΔMSH6) to obtain a higher resolution of mutational signatures of MMR gene knockouts.

Note that for all mutational profiles obtained throughout these examples (whether from gene knockouts or from samples), the somatic mutational profiles (excluding germline mutations) are used.

Identifying background signatures. The mutational profile of control subclones were used to determine background mutagenesis. Aggregated substitution profiles of all control subclones (ΔATP2B4) were used as the background substitution mutational signature. Aggregated indel profiles of all subclones containing <=8 indels were used as the background indel mutational signature.

Distinguishing mutational profiles of control and gene-edited subclone profiles. Signal-to-noise ratio affects mutational signature detection. In this study, ‘noise’ is largely background mutagenesis. The averaged mutation burden caused by the background mutagenesis in control cells for substitution and indels are around 150 and 10, with standard deviation of 10 and 1.4, respectively. ‘Signal’ represents the elevated mutation burden caused by gene knockouts. The averaged mutation burden in knockouts range from 63 to 2360 for substitution, and 0 to 2122 for indels after 15 days in culture, as shown in Table 2.

The costs associated with whole genome sequencing is prohibitive, thus we have 2-4 subclones per knockout. The intrinsic fluctuation of detected mutation burden in each sample and the limited subclone numbers impose a greater uncertainty in mutational signature detection. Thus, to distinguish high-confidence mutational signatures from noise, we employed three different methods.

First, we evaluated the similarity of mutational profile between control and each gene knockout. According to the mutational profile of control subclones, p_{control}=[p_{control}¹,p_{control}², . . . , p_{control}^{K}]^{T}, for a given number of mutations N (0<N<10000), one could generate L bootstrapped samples:

$${M}_{N} = [ {m}_{1} , … , {m}_{l} , … , {m}_{L} ] = [ \begin{matrix}{m}_{1}^{1} & … & {m}_{L}^{1} \\ ⋮ & ⋱ & ⋮ \\ {m}_{1}^{K} & … & {m}_{L}^{K}\end{matrix} ] , \qquad ( 1 )$$

where Σₖ₌₁^{K}mₗ^{k}=N. One can calculate the cosine similarities (sₗ) between bootstrapped control samples (mₗ) and experimentally-obtained control profile (p_{control}) to obtain a distribution of cosine similarities P(S):

$${s}_{l} = \frac{{m}_{l} \cdot {p}_{control}}{‖ {m}_{l} ‖ ‖ {p}_{control} ‖} . \qquad ( 2 )$$

We can then calculate the cosine similarity (S_{knockout}) between control profile (p_{control}) and knockout profile (p_{knockout}). As shown in FIGS. 4C and 4D, when the mutation count is low, the bootstrapped samples are less similar to the actual control profile than the bootstrapped samples with higher mutation count. Comparing S_{knockout} and P(S) at a given mutation number, N_{knockout}, one could identify which gene knockouts having distinct mutational profiles from the control (p value of S_{knockout} is less than 0.01 in P(S)).

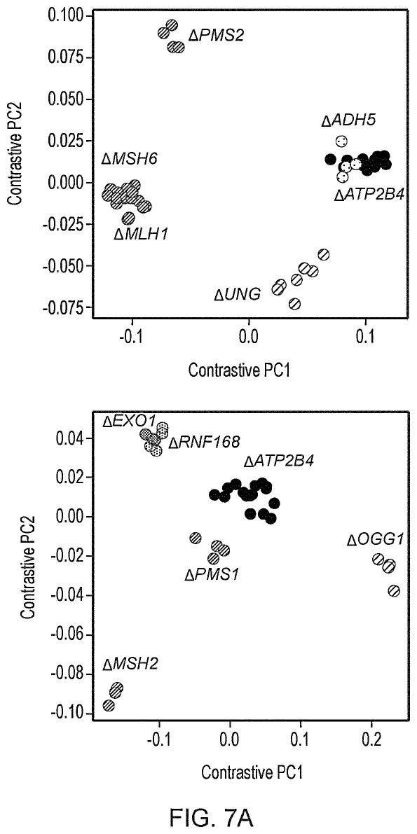

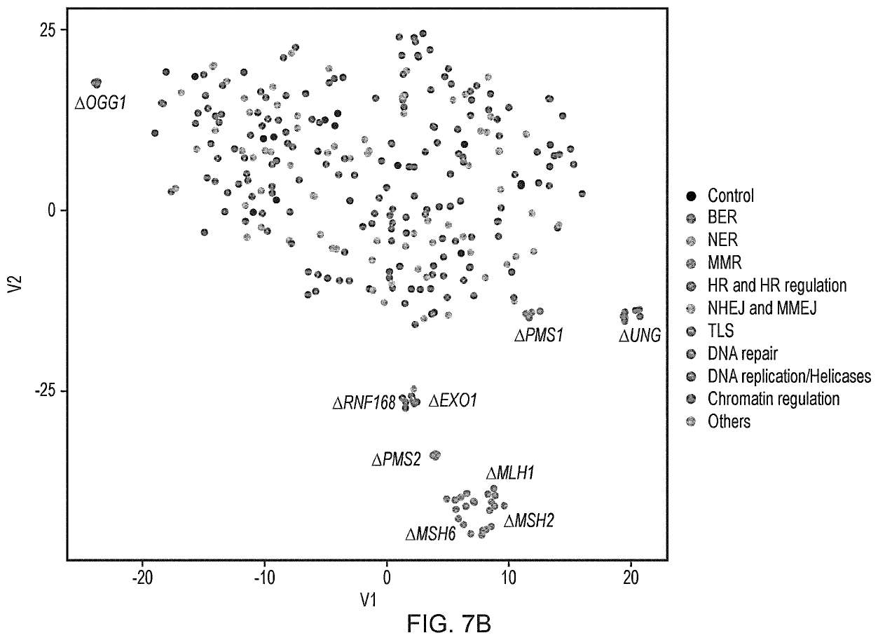

Second, we used contrastive principal component analysis (cPCA)(Abid, A. et al., 2018), which efficiently identified directions that were enriched in the knockouts relative to the background through eliminating confounding variations present in both (FIG. 7A), to recognize gene knockout-specific patterns from background signature.

Third, we used t-Distributed stochastic neighbor embedding (t-SNE)(van der Maaten, L. & Hinton, G. 2008), which is a visualization technique for viewing pairwise similarity data resulting from nonlinear dimensionality reduction based on probability distributions. In t-SNE implementation, mutational profiles that are similar to each other were plotted nearby each other, whereas profiles that are dissimilar are plotted distantly in a 2D space (FIG. 7B).

Subtraction of the background mutational signature from knockout mutation profile. The experiment-associated mutational signature can then be obtained by subtracting the background mutational signature from the mutational profile of treated subclones through quantile analysis. First, one can generate a set of bootstrap samples (e.g. 10,000 samples) of each treated subclone in order to determine the distribution of mutation number for each channel. This set of “hypothetical samples” aims to simulate the variability that may be present in a larger population of subclones, even though only 4 subclones could be generated for practical reasons. According to the distribution, the upper and lower boundaries (e.g., 99% CI) for each channel (e.g. each of the 96 channels for substitutions) can be identified for each treatment. The same process is applied to the control knockouts (ATP2B4) to estimate the expected background mutational signature variability. Based on the background mutational signature (average mutation signature in each of the channels, across the 4 control subclones) and averaged mutation burden (across the 4 control subclones; used as initial value), one can construct bootstrapped background profiles. The bootstrap background profiles can then be used to derive a centroid value across bootstrap background profiles, and this is subtracted from the centroid of bootstrap subclone samples. This process results in a mutational signature for each knockout, which is derived from all subclones for the knockout with variability estimated by bootstrapping, and adjusted to remove the estimated background contribution. Due to data noise, some channels may have negative values, in which case, the negative values are set to zero. Occasionally, the number of mutations in a few channels will fall outside the lower boundary after removing the background profile. To avoid negative values, the background mutation pattern is maintained but burden is scaled down through an automated iterative process.

Other software used. IntersectBed (Quinlan, A. R. & Hall, I. M., 2010) was used to identify mutations overlapping certain genomic features. All statistical analysis in these Examples were performed in R (Team, R. C. 2017). All plots were generated by ggplot2 (Wickham, H., 2009).

Results

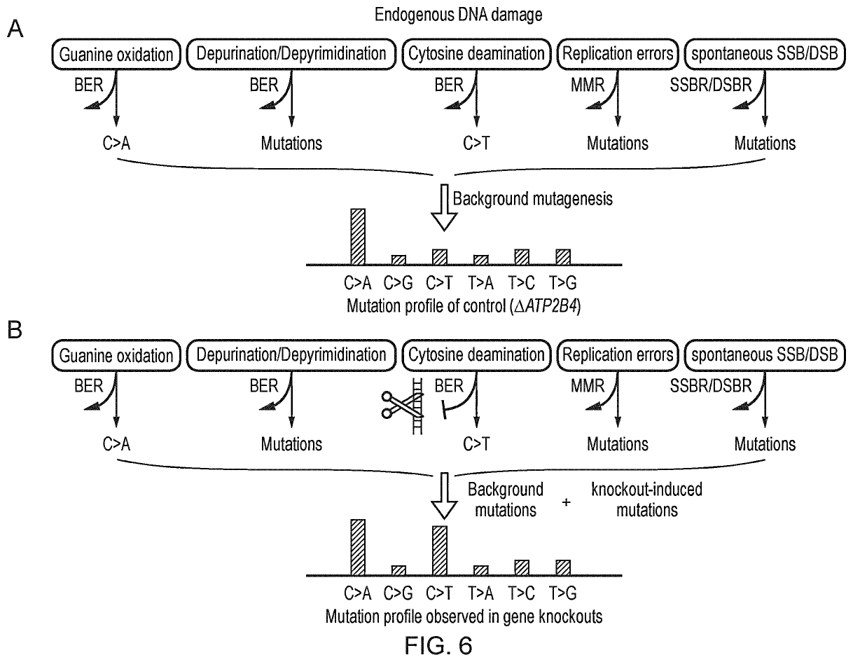

We reasoned that under the controlled experimental settings described in Example 1, if simply knocking-out a gene (in the absence of providing additional DNA damage) could produce a signature, then the gene is critical to maintaining genome stability from endogenous sources of DNA damage. It would manifest an increased mutation burden above background and/or an altered mutation profile (FIG. 6). We found background substitution and indel mutagenesis associated with growing cells in culture occurred at ˜150 substitutions and ˜10 indels per genome and was comparable across all subclones.

To address potential uncertainty associated with the relatively small number of subclones per knockout and variable mutation counts in each gene knockout (see Example 1 and Methods above), we generated bootstrapped control samples with variable mutation burdens (50-10,000). We calculated cosine similarities between each bootstrapped sample and the background control (ΔATP2B4) mutational signature (mean and standard deviations). A cosine similarity close to 1.0 indicates that the mutation profile of the bootstrapped sample is near-identical to the control signature. Cosine similarities could thus be considered across a range of mutation burdens (green line in FIG. 4C and light blue line in FIG. 4D). We next calculated cosine similarities between knockout profiles and controls (colored dots in FIGS. 4C and 4D). A knockout experiment that does not fall within the expected distribution of cosine similarities implies a mutation profile distinct from controls, i.e., the gene knockout is associated with a signature. For substitution signatures, two additional dimensionality reduction techniques, namely, contrastive principal component analysis (cPCA) and t-Distributed Stochastic Neighbor Embedding (t-SNE) were also applied to secure high confidence mutational signatures (FIG. 7, see Methods above). This stringent series of steps would likely dismiss weaker signals and thus be highly conservative at calling mutational signatures. These conservative methods were also applied to identify indel signatures (see Methods).

We identified nine single substitution, two double substitution and six indel signatures. Three gene knockouts, ΔOGG1, ΔUNG, and ΔRNF168, produced only substitution signatures. Six gene knockouts, ΔMSH2, ΔMSH6, ΔMLH1, ΔPMS2, ΔEXO1, and ΔPMS1, presented substitution and indel signatures. ΔEXO1 and ΔRNF168 also produced double substitution patterns. The average de novo mutation burden accumulated for these nine knockouts (FIG. 4E) ranged between 250-2,500 for substitutions and 5-2,100 for indels. Based on cell proliferation assays, mutation rates for each knockout were calculated and ranged between 6-129 substitutions and 0.39-126 indels per cell division (Table 4). In Examples 3 and 4, we dissect the experimentally-generated mutational signatures that are associated with genes involved in the mismatch repair (MMR) pathway. We compare them to one another and to previously published human cancer-derived mutational signatures, to gain insights into the sources of endogenous DNA damage and mutational mechanisms.

TABLE 4
Calculated mutation rates.

| | subs | subs | indels | Indels |
|---|---|---|---|---|
| Gene | rate mean | rate sd | rate mean | rate sd |
| EXO1 | 47.5265 | 9.706873119 | 0.387833333 | 0.11735559 |
| MLH1 | 115.2054653 | 7.958276188 | 114.7386042 | 13.1548119 |
| MSH2 | 128.9222667 | 0.392019999 | 126.4685333 | 1.54080139 |
| MSH6 | 95.70146528 | 15.67307091 | 35.58291667 | 2.32915081 |
| OGG1 | 15.96555556 | 1.275934903 | NA | NA |
| PMS1 | 5.647963194 | 2.284990391 | 0.541938194 | 0.23667469 |
| PMS2 | 75.27176667 | 7.330654568 | 69.03303056 | 7.30110929 |
| RNF168 | 31.79190417 | 1.659550153 | NA | NA |
| UNG | 6.330458333 | NA | NA | NA |

subs = substitution, sd = standard deviation.

### DISCUSSION

In standardized experiments performed in a diploid, non-transformed human stem cell model, biallelic gene knockouts that produce mutational signatures in the absence of administered DNA damage are indicative of genes that are important at maintaining the genome from intrinsic sources of DNA perturbations. We find signatures of substitutions and/or indels in nine genes: ΔOGG1, ΔUNG, ΔEXO1, ΔRNF168, ΔMLH1, ΔMSH2, ΔMSH6, ΔPMS2, and ΔPMS1, suggesting that proteins of these genes are critical guardians of the genome in non-transformed cells. Many gene knockouts did not show mutational signatures under these conditions. This does not mean that they are not important DNA repair proteins. There may be redundancy, or the gene may be crucial to the orchestration of DNA repair, even if itself is not imperative at directly preventing mutagenesis. It is also possible that some gene knockouts have very low rates of mutagenesis such that a statistically distinct mutational signature cannot be distinguished from background mutagenesis within our experimental time-frame. For genes involved in double-strand-break (DSB) repair, hiPSCs may not be permissive for surviving DSBs to report signatures. Other genes may require alternative forms of endogenous DNA damage that manifest in vivo but not in vitro, for example, aldehydes, tissue-specific products of cellular metabolism, and pathophysiological processes such as replication stress. Likewise, for genes in the nucleotide excision repair pathway, bulky DNA adducts, whether exogenous (e.g., ultraviolet damage) or endogenous (e.g., cyclopurines and by-products of lipid peroxidation) may be a pre-requisite before these compromised genes reveal associated signatures. While experimental modifications such as the addition of DNA damaging agents to increase mutation burden or using alternative cellular models, for example, cancer lines or cellular models of specific tissue-types, could amplify signal, they could also modify mutational outcomes, and that must be taken into consideration when interpreting data. Also, not all genes have been successfully knocked out in this endeavour and could have similarly important roles in directly preventing mutagenesis.

### Example 3—Multiple Endogenous Sources of DNA Damage Managed by Mismatch Repair

In this example, the inventors investigated in-depth the mutational signatures identified in Example 2 associated with genes involved in the MMR pathway.

Methods

See Examples 1 and 2.

Topography analysis of signatures. Strand bias. Reference information of replicative strands and replication-timing regions were obtained from Repli-seq data of the ENCODE project (https://www.encodeproject.org/) (The E.P.C. et al., 2012). The transcriptional strand coordinates were inferred from the known footprints and transcriptional direction of protein coding genes. First, for a given mutational signature, one could calculate the ‘expected’ ratio of mutations between transcribed and non-transcribed strand, or between lagging and leading strands, according to the distribution of trinucleotide sequence context in these regions. Second, the ‘observed’ ratio of mutations between different strands can be identified through mapping mutations to the genomic coordinates of all gene footprints (for transcription) or leading/lagging regions (for replication). Third, all mutations were orientated towards pyrimidines as the mutated base (as this has become the convention in the field). This helped denote which strand the mutation was on. Fourth, the level of asymmetry between different strands was measured by calculating the odds ratio of mutations occurring on one strand (e.g., transcribed or leading strand) vs. on the other strand (e.g., non-transcribed or lagging strand).

Results

Knockouts of five genes involved in the mismatch repair (MMR) pathway (Gupta et al., 2012; Palombo et al., 1995, Warren et al., 2007), MSH2, MSH6, MLH1, PMS2, and PMS1, produced substitution and indel signatures (FIGS. 8A and 8B) but not double substitution signatures despite a previously reported association (Alexandrov et al., 2020). ΔMLH1, ΔMSH2, and ΔMSH6 produced identical qualitative substitution signatures (cossim: 0.99) characterized by a single strong peak at CCT>CAT/AGG>ATG, and multiple peaks of C>T and T>C (FIG. 8A). In contrast, ΔPMS2 generated a signature of predominantly T>C transitions with a slight predominance at ATA, ATG, and CIG (FIG. 8C). The single peak at CCT>CAT/AGG>ATG remains visible in the ΔPMS2 substitution signature, albeit markedly reduced (10% to 3%). In addition, ΔMSH2, ΔMSH6, and ΔMLH1 generated indel signatures dominated by A/T deletions at long repetitive sequences. In contrast, ΔPMS2 produced similar amounts of A/T insertions and A/T deletions at long repetitive sequences (FIGS. 8B, 8J, 8I). ΔPMS1 generated A/T deletions only at long poly[d(A-T)] (>=5 bp) and long deletions (>1 bp) at repetitive sequences (FIG. 8J).

In-depth analysis of these mutational signatures allowed us to determine putative sources of endogenous DNA damage (FIG. 8C) acted upon by MMR.

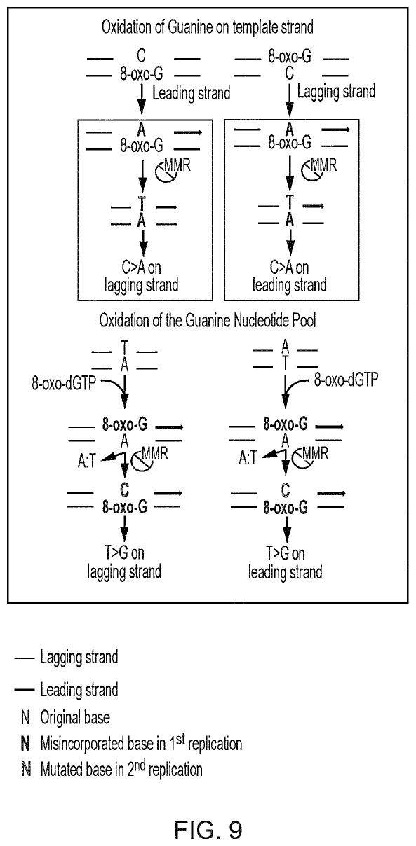

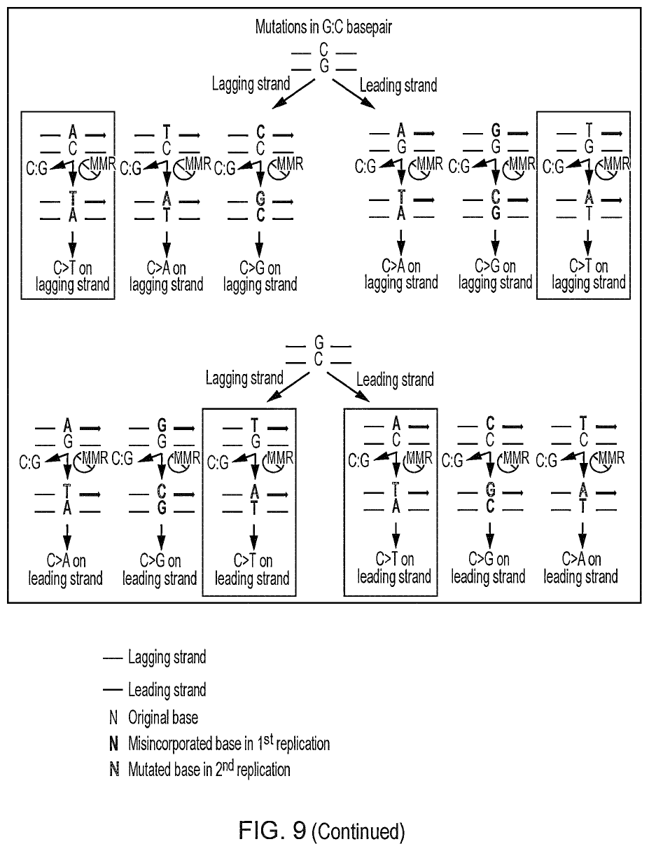

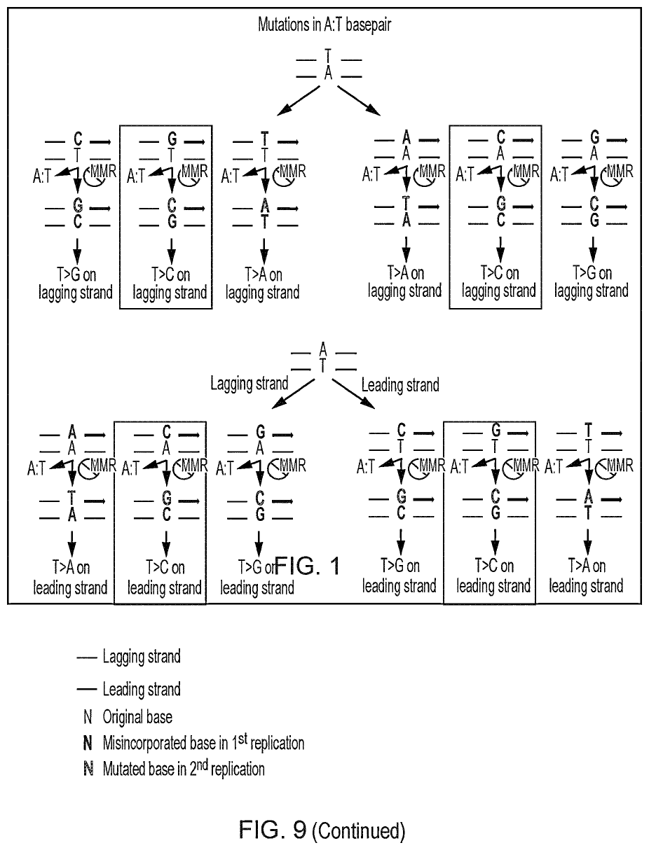

First, we consistently observed replication strand bias across ΔMLH1, ΔMSH2, ΔMSH6, and ΔPMS2: C>A on the lagging strand (equivalent to G>T leading strand bias), C>T on the leading strand (or G>A lagging) and T>C lagging (or A>G leading) (FIG. 8D). Under our experimental settings where exogenous DNA damage was not administered, mismatches may be generated by DNA polymerases α, δ or ε during replication. In the absence of MMR, these lesions become permanently etched as mutations. To understand which replicative polymerases could be causing these mutations, we analyzed putative progeny of all twelve possible base/base mismatches (FIG. 9). T/G mismatches are the most thermodynamically stable and represents the most frequent polymerase error (Aboul-ela et al., 1985). Our assessment suggests that the predominance of T>C transitions on the lagging-strand can only be explained by misincorporation of T by lagging strand polymerases, pol-α and/or pol-δ leading to G/T mismatches (FIG. 8C). Similarly, the observed bias for C>T transitions on the leading strand is likely to be predominantly caused by misincorporation of G on lagging strand by pol-α and/or pol-δ resulting in T/G mismatches (FIG. 8C).

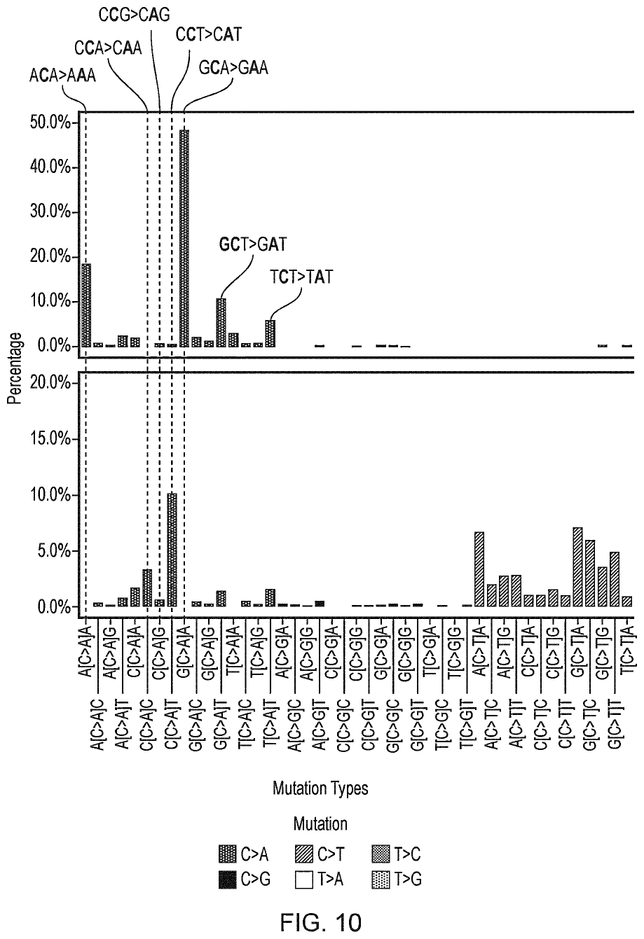

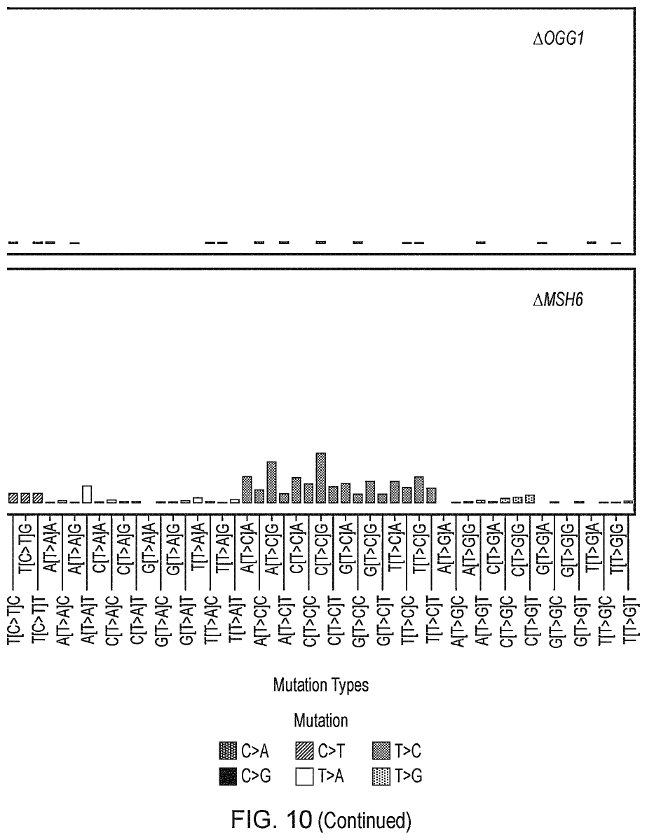

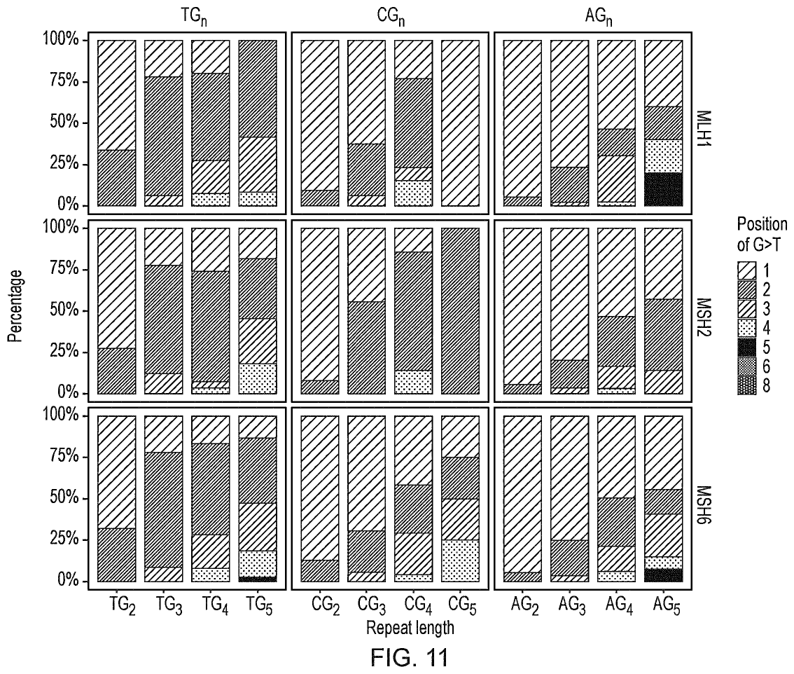

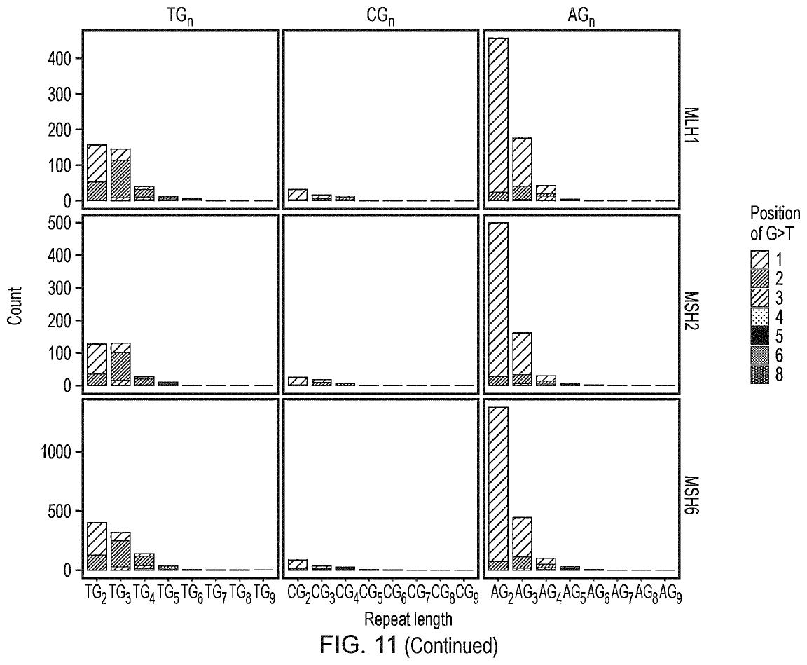

Second, the predominance of C>A transversions could be explained by differential processing of 8-oxo-dGs (FIG. 8C) (Patel et al., 1984; Matray & Kool, 1999). The predominant C>A/G>T peak in MMR-deficient cells occurs at CCT>CAT/AGG>ATG followed by CCC>CAT/GGG>GTG and is distinct from the C>A/G>T peaks observed in ΔOGG1 (FIG. 10). However, we previously showed that there is a depletion of mutations at CC/GG sequence motifs for ΔOGG1. Intriguingly, the experimental data suggest that the 8-oxo-G:A mismatches can be repaired by MMR, preventing C>A/G>T mutations⁵³. Furthermore, that G>T/C>A mutations of MMR-deficient cells occurred most frequently at the second G in 5′-TGₙ (n>=3) in ΔMLH1, ΔMSH2, and ΔMSH6 (FIGS. 8E and 11). This is consistent with previous reports (Morikawa et al., 2014) of the classical imprint of guanine oxidation at polyG tracts where site reactivity in double-stranded 5′-TG¹G²G³G⁴T sequence is reported as G²>G³>G¹>G⁴. These results implicate the activity of MMR in repairing 8-oxo-G:A mismatches at GG motifs that perhaps cannot be cleared by OGG1 in BER (base excision repair). As for G>T leading strand bias, studies in yeast have demonstrated that an excess of 8-oxo-dG-associated mutations occurs during leading strand synthesis (Pavlov et al., 2002). Furthermore, translesion synthesis polymerase η is also more error-prone when bypassing 8-oxo-dG on the leading strand (Mudrak et al., 2009), which would result in increased 8-oxoG/A mispairs on the leading strand.

Third, we found that T>A transversions at ATT were strikingly persistent in MMR knockout signatures, although with modest peak size (<3% normalized signature, FIG. 8A). Additional sequence context information revealed that T>A occurred most frequently at AATTT or TTTAA, which were junctions of polyA and polyT tracts (FIG. 8F) (Meier et al., 2018; Lang et al., 2013). Moreover, the length of 5′- and 3′-flanking homopolymers influenced the likelihood of mutation occurrence: T>A transversions were one to two orders of magnitude more likely to occur when flanked by homopolymers of 5′polyA/3′polyT (AₙTₘ) or 5′polyT/3′polyA (TₙAₘ), than when there were no flanking homopolymeric tracts (FIG. 8G).

Since polynucleotide repeat tracts predispose to indels due to replication slippage and are a known source of mutagenesis in MMR-deficient cells, we hypothesize that the T>A transversions observed at sites of abutting polyA and polyT tracts are the result of a ‘reverse template slippage’. In this scenario, the polymerase replicating across a mixed repeat sequence such as a repeat of 6 As followed by 4 Ts in which the template slipped at one of the As would incorporate five instead of six Ts opposite the A repeat (red arrow pathway in FIG. 8H). If at this point the template were to revert to its original correct alignment, this would give rise to an A/A mismatch that would result in a T>A transversion. If the slippage remained, this would give rise to a single nucleotide deletion, a characteristic feature of MMR-deficient cells known as microsatellite instability (MSI) (FIG. 8B, indel signatures).

### Example 4—Gene-Specific Characteristics of Mutational Signatures of MMR-Deficiency

In this example, the inventors compared and validated the mutational signatures identified in Example 2 associated with genes involved in the MMR pathway.

Methods

See Examples 1-3.

CMMRD patient sample collection. Four CMMRD patients were recruited at Doce de Octubre University Hospital, Spain, St George's Hospital in London and Great Ormond Street Hospital under the auspices of the Insignia project. This included two PMS2-mutant patients and two MSH6-mutant patients. Table 5 shows the genotypes of these four patients. A healthy donor was recruited as control.

TABLE 5
Genotypes of four CMMRD patients.

| Patient | Gene | Mutations |
|---|---|---|
| CMMRD3 | PMS2 | c.736_741delCCCCCTinsTGTGTGTGAAG - |
| | | stop gained |
| CMMRD77 | PMS2 | c.[2007-2A > G]; [2007-2A > G] - |
| | | splice acceptor variant |
| CMMRD89 | MSH6 | c.[2653A > T]; [2653A > T] - |
| | | nonsense |
| CMMRD94 | MSH6 | c.3932_3933insAGTT - frameshift |
| Patient | Gene | Mutations |

Generation of iPSCs from Constitutional Mismatch Repair Deficiency (CMMRD) Patients. Peripheral blood mononuclear cells (PBMCs) isolation, erythroblast expansion, and IPSC derivation were done by the Cellular Generation and Phenotyping facility at the Wellcome Sanger Institute, Hinxton, according to Agu et al 2015. Briefly, whole blood samples collected from consented CMMRD patients were diluted with PBS, and PBMCs were separated using standard Ficoll Paque density gradient centrifugation method. Following the PBMC separation, samples were cultured in media favouring expansion into erythroblasts for 9 days. Reprogramming of erythroblasts enriched fractions was done using non-integrating CytoTune-iPS Sendai Reprogramming kit (Invitrogen) based on the manufacturer's recommendations. The kit contains three Sendai virus-based reprogramming vectors encoding the four Yamanaka factors, Oct3/4, Sox2, Klf4, and c-Myc. Successful reprogramming was confirmed via genotyping array and expression array.

Results

There are uncertainties regarding which of the cancer-derived signatures (described in Alexandrov, L. B. et al. (2020) and Degasperi, A. et al. (2020)) are truly MMR-deficiency signatures. It was suggested that SBS6, SBS14, SBS15, SBS20, SBS21, SBS26, and SBS44 were MMR-deficiency related (Alexandrov, L. B. et al. (2020)). In an independent analytical exercise, only two MMR-associated signatures were identified (Degasperi, A. et al. (2020)), although variations of the signatures were seen in different tissue types (Degasperi, A. et al. (2020)). An experimental process would help to obtain clarity in this regard (Nik-Zainal, S. et al., 2015; Zou, X. et al., 2018; Christensen, S. et al., 2019; Kucab, J. E. et al., 2019).

As described above, substitution patterns of ΔMSH2, ΔMSH6, and ΔMLH1 showed enormous qualitative similarities to each other and were distinct from ΔPMS2 (FIG. 8A). We next expanded indel channels according to the length of polynucleotides, obtaining a higher resolution of MMR deficiency-associated indel signatures (FIG. 8B, see Methods in Example 2). ΔMSH2, ΔMSH6, and ΔMLH1 had very similar indel profiles, dominated by T deletions at increasing lengths of polyT tracts, with minor contributions of T insertions and C deletions. In contrast, ΔPMS2 had similar proportions but different profiles between T insertions and deletions (FIGS. 8B and 8I).

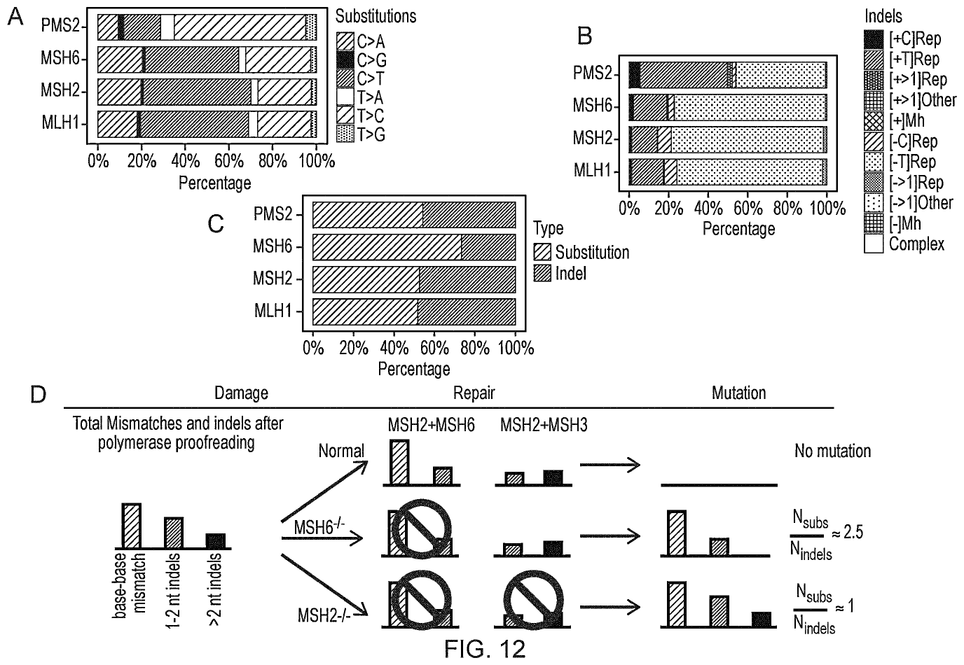

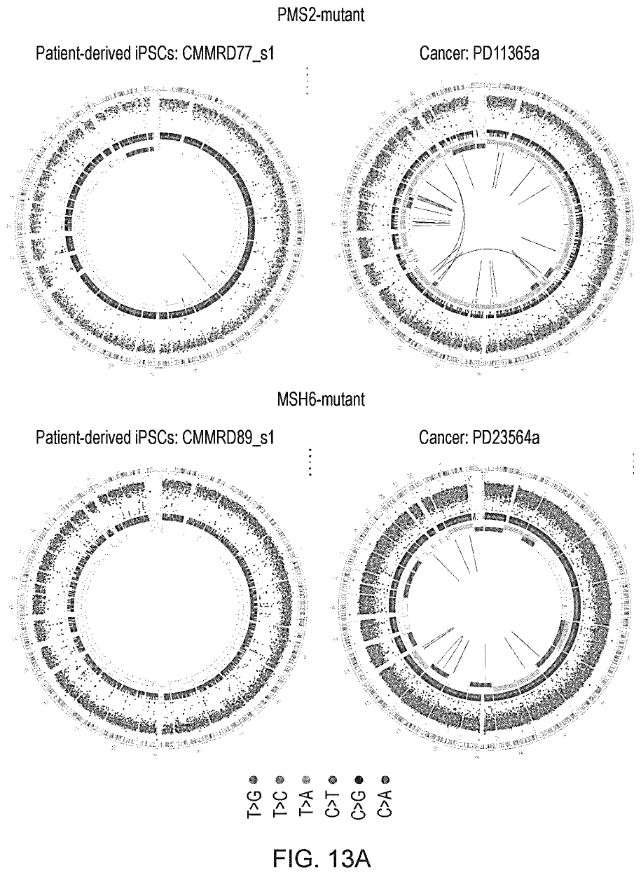

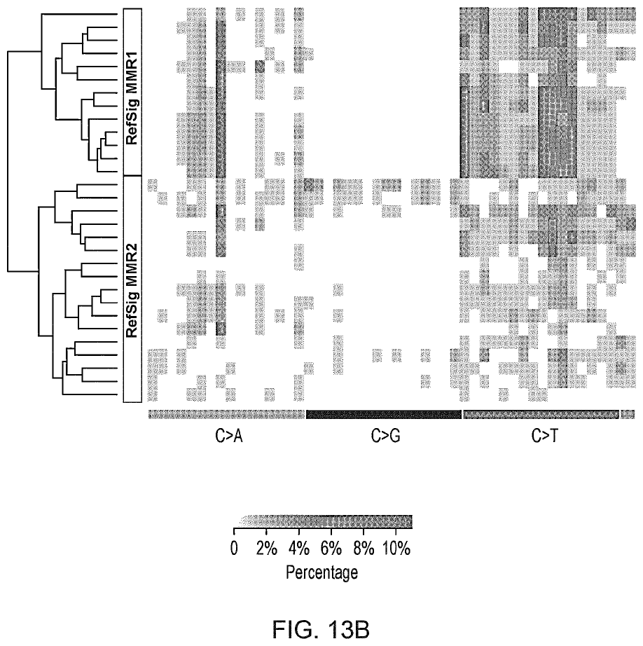

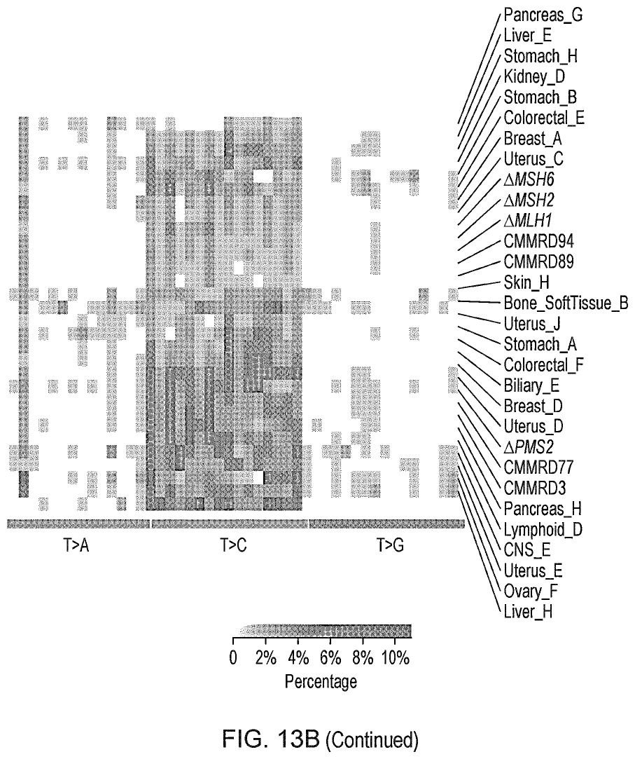

While the qualitative indel profiles of ΔMSH2, ΔMSH6, and ΔMLH1 were very similar, their quantitative burdens were rather different (FIGS. 4E and 12). ΔMLH1 and ΔMSH2 had high indel burdens, while ΔMSH6 had half the burden of indel mutagenesis. Substitution-to-indel ratios showed that ΔMSH2, ΔPMS2, and ΔMLH1 produced similar amounts of substitutions and indels, while ΔMSH6 generated nearly 2.5 times more substitutions than indels (FIG. 12). This result is in-keeping with known protein interactions and functions: MSH2 and MSH6 form the heterodimer MutSα that addresses primarily base-base mismatches and small (1-2 nt) indels (Palombo et al., 1995; Drummond et al., 1995). MSH2 can also heterodimerize with MSH3 to form the heterodimer MutSβ, which does not recognize base-base mismatches, but can address indels of 1-15 nt (Palombo et al., 1996). This functional redundancy in the repair of small indels between MSH6 and MSH3 explains the smaller number of indels observed in ΔMSH6 (FIG. 13E) compared to ΔMSH2 cells. This is consistent with the near-identical MSI phenotypes of Msh2^{-/-} and Msh3^{-/-}; Msh6^{-/-} mice (Wind et al., 1999).

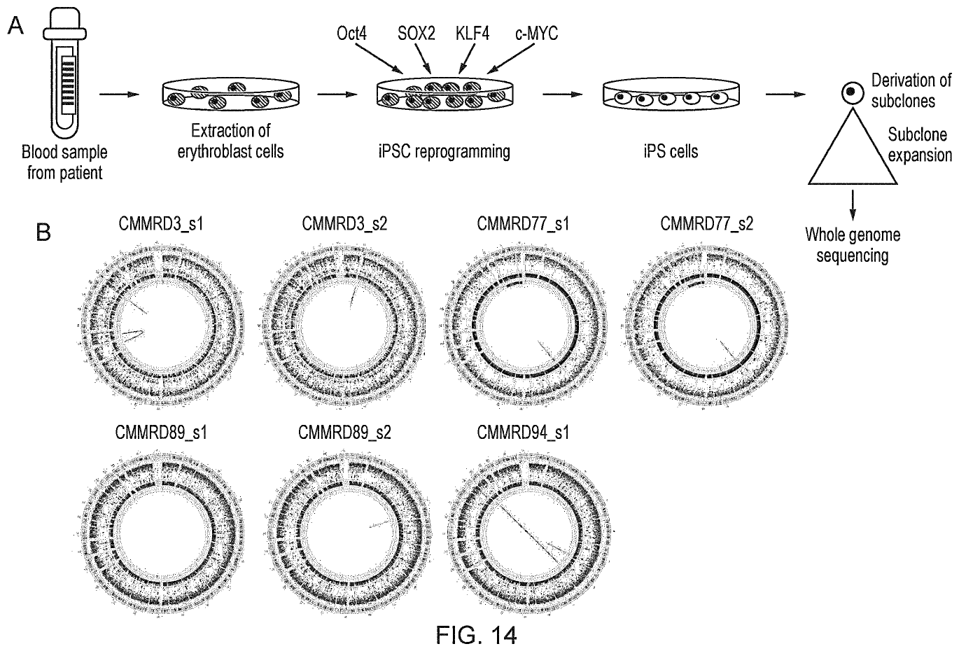

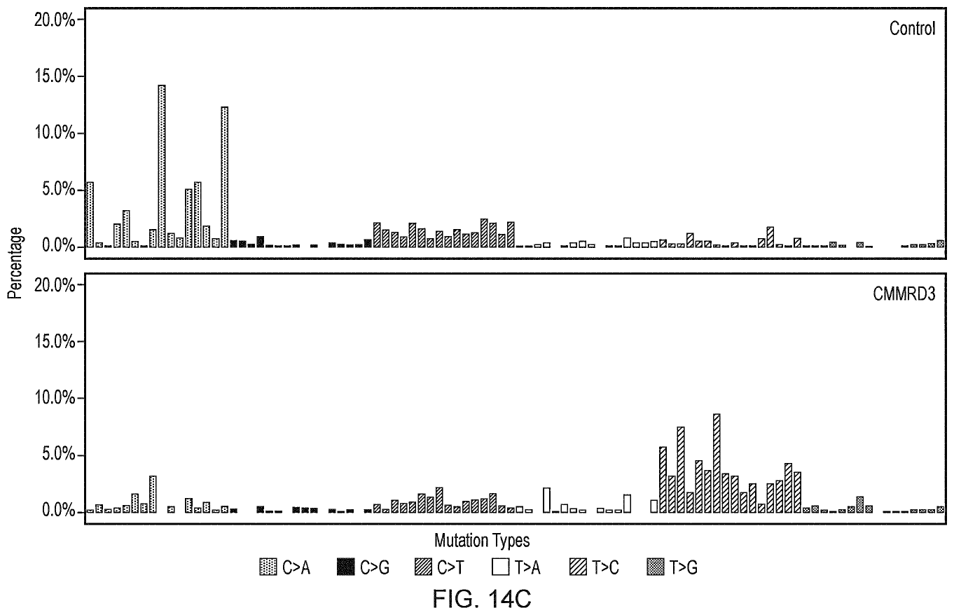

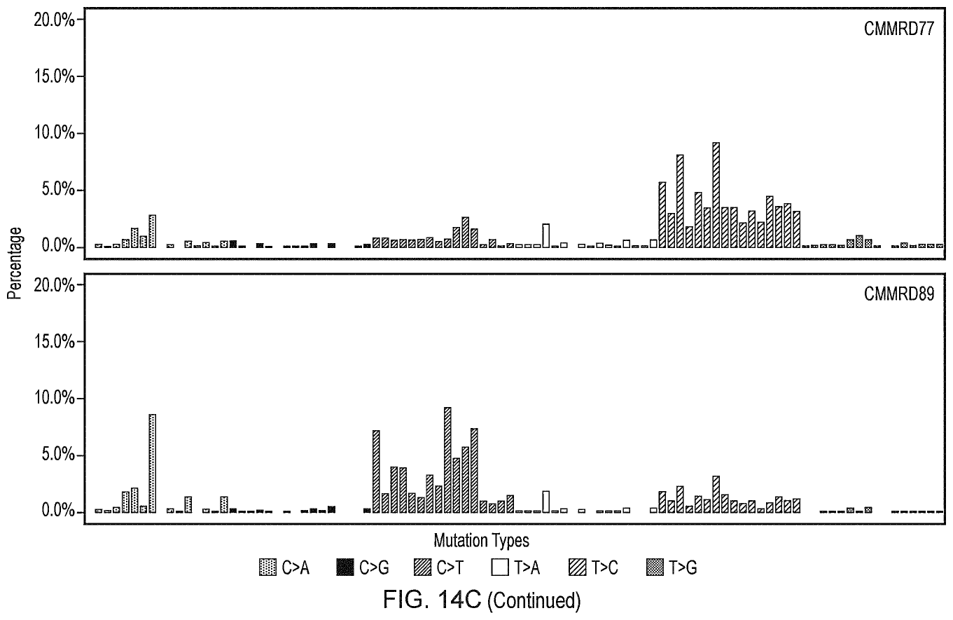

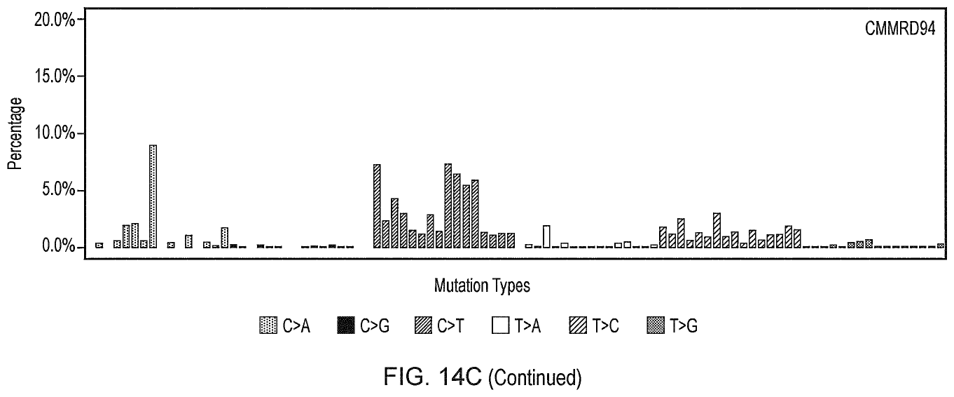

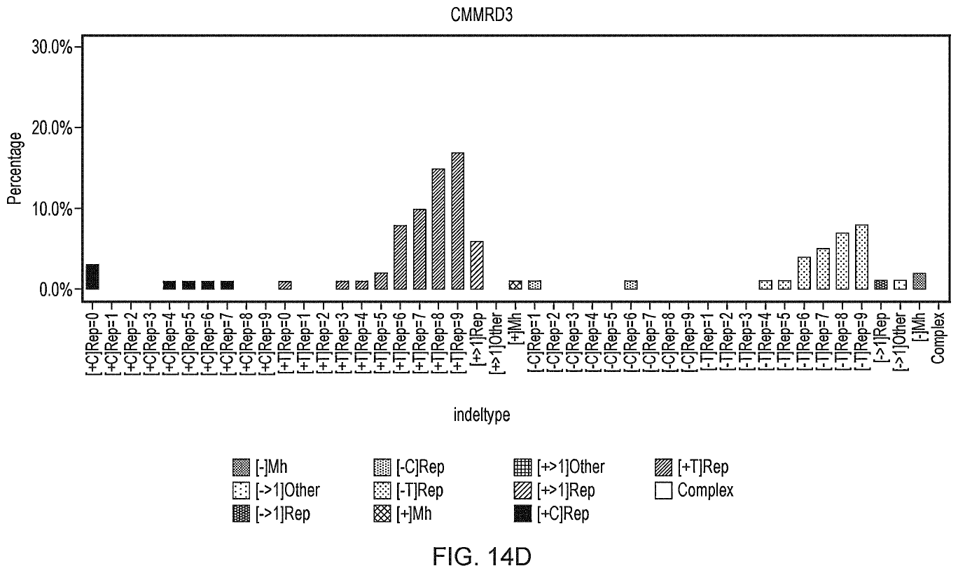

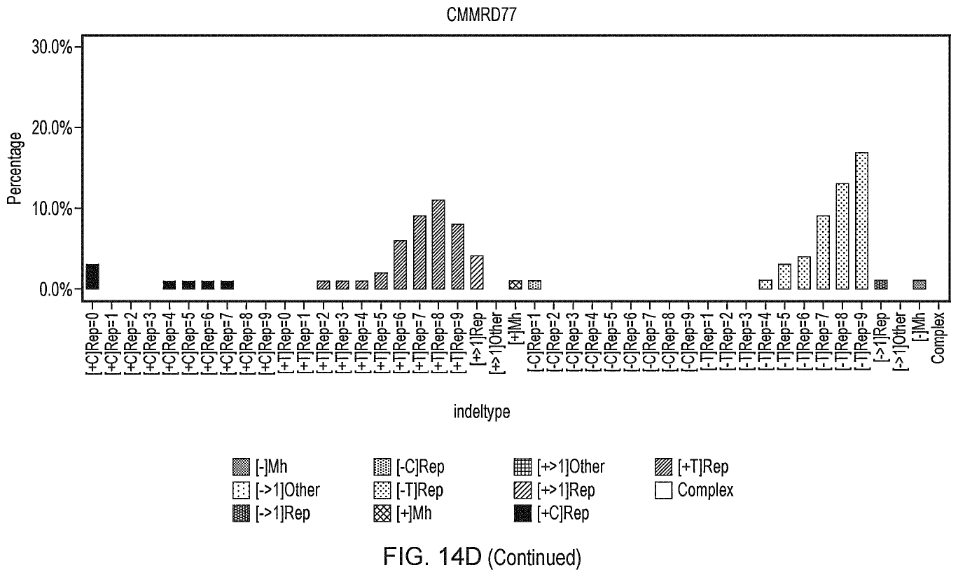

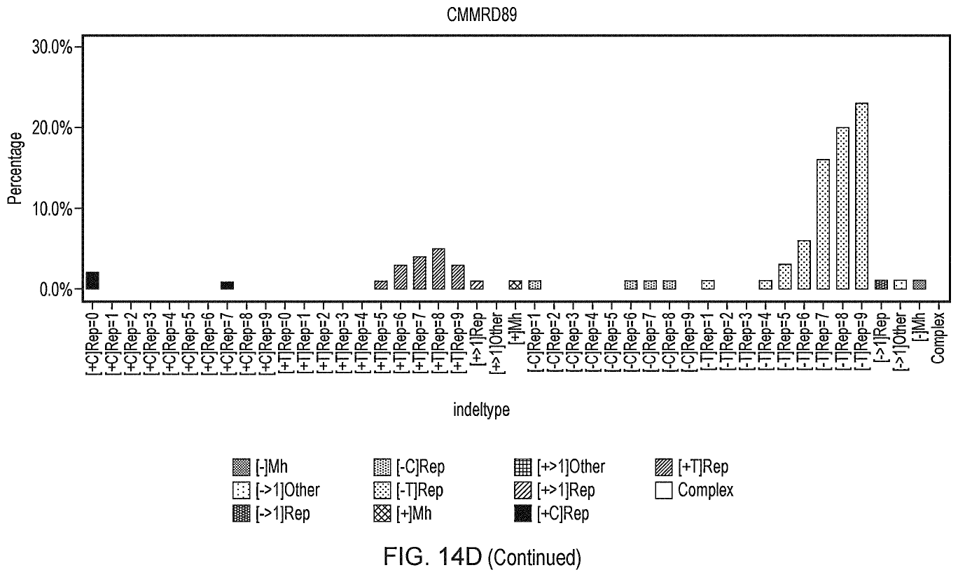

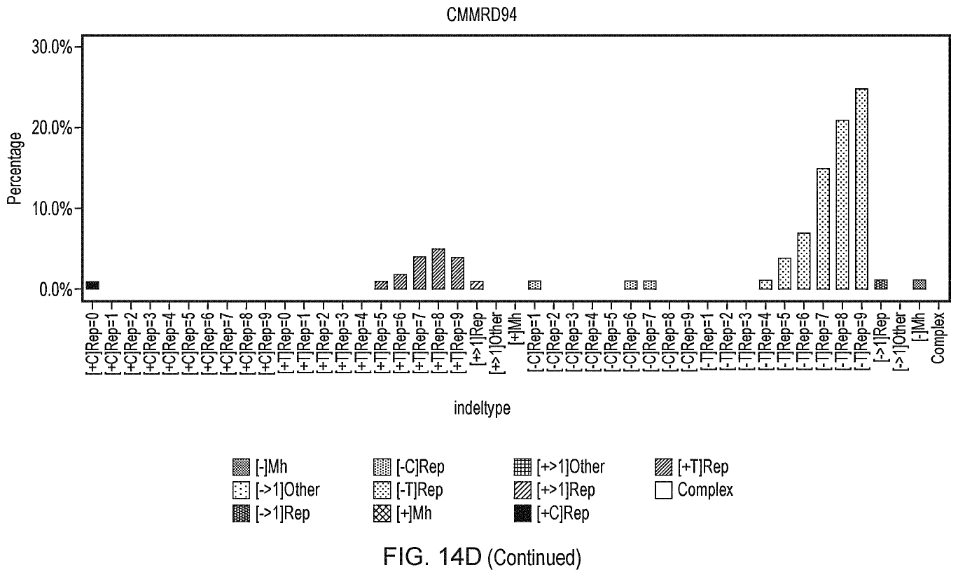

Thus, there are clear qualitative differences between substitution and indel profiles of ΔMSH2, ΔMSH6, and ΔMLH1 from ΔPMS2. To validate these two gene-specific experimentally-generated MMR knock-out signatures, we interrogated genomic profiles of normal cells derived from patients with inherited autosomal recessive defects in MMR genes resulting in Constitutional Mismatch Repair Deficiency (CMMRD), a severe, hereditary cancer predisposition syndrome characterized by an increased risk of early-onset (often pediatric) malignancies and cutaneous café-au-lait macules (Poulogiannis et al., 2010; Heinen et al., 2016). hiPSCs were generated from erythroblasts derived from blood samples of four CMMRD patients (two PMS2 homozygotes and two MSH6 homozygotes) and two healthy control⁶⁴. hiPSC clones obtained were genotyped (Agu et al., 2015). Expression arrays and cellomics-based immunohistochemistry were performed to ensure that pluripotent stem cells were generated (see Methods). Parental clones were grown out to allow mutation accumulation, single-cell subclones were derived, and whole-genome sequenced (FIG. 14A).

Gene-specificity of mutational signatures seen in CMMRD hiPSCs was virtually identical to those of the CRISPR-Cas9 knockouts and cancers (FIGS. 13A and 14B). The PMS2 CMMRD patterns carried the same propensity for T>C mutations, the small contribution of C>T mutations and the single peak of C>A/G>T at CCT/AGG, as seen in ΔPMS2, and the MSH6 CMMRD patterns carried the excess of C>T mutations with a very pronounced C>A/G>T at CCT/AGG similar to ΔMLH1, ΔMSH2 and ΔMSH6 clones (FIG. 14C). Indel propensities seen in the knockout MMR clones were also reflected in the patient-derived cells (FIG. 14D). Accordingly, gene-specificity of signatures generated in the experimental knockout system is well-recapitulated in an independent patient-derived cellular system of normal cells.

Furthermore, gene-specific MMR signatures were seen in the International Cancer Genome Consortium (ICGC) cohort of >2,500 primary WGS cancers (Degasperi, A. et al., 2020). Indeed, biallelic MSH2/MSH6/MLH1 mutant tumors carried the same signature (RefSig MMR1) as ΔMSH2/ΔMSH6/ΔMLH1 clones (FIG. 13B). We also identified biallelic PMS2 mutants in several cancers, including breast and ovarian cancers with mutation patterns (RefSig MMR2) that were indistinguishable from our experimentally-generated ΔPMS2 signatures (FIG. 13B).

### Example 5—Informing Classification of MMR-Deficient Tumors Using Experimental Data

In this example, the inventors developed an algorithm to classify tumours according to MMR-deficiency status using the insights generated in Examples 1-4.

Methods

See Examples 1-4.

MMRDetect algorithm. We trained a mismatch repair (MMR) deficiency logistic regression-based classifier, called MMRDetect, based on mutational signatures obtained from the experimental work. We obtained mutation data from 336 WGS colorectal cancers with accompanying immunohistochemistry (IHC) staining of the four MMR proteins (MSH2, MSH6, MLH1 and PMS2) from UK100,000 Genomes Project (UK100kGP). Within this cohort of 336 colorectal cancers, there were 79 (24%) cancers with abnormal IHC staining indicative of MMR deficiency. 336 cancers were randomly divided into a training set and a test set by using the R function sample( ). The training set had 180 MMR-proficient and 56 MMR-deficient samples. The test data set had 77 MMR-proficient and 23 MMR-deficient samples (Table 6).

TABLE 6
List of 336 colorectal cancer samples from GEL that were used
for training and test data set for building MMRDetect.

| ID | RIN | DRM | MMRs | MCS | MSIs | MMRD | MSIseq |
|---|---|---|---|---|---|---|---|
| col2348_3 | 69938 | 0.9881 | 14508.64 | 0.6615 | M | M | M |
| col2348_5 | 978 | 0.7606 | 0 | 0.5944 | nM | nM | nM |
| col2348_7 | 143398 | 0.9882 | 20620.06 | 0.6178 | M | M | M |
| col2348_9 | 1997 | 0.8043 | 0 | 0.558 | nM | nM | nM |
| col2348_10 | 192873 | 0.9868 | 111652.3 | 0.6037 | M | M | M |
| col2348_12 | 1702 | 0.945 | 0 | 0.5563 | nM | nM | nM |
| col2348_14 | 1293 | 0.8904 | 0 | 0.5695 | nM | nM | nM |
| col2348_15 | 2636 | 0.8669 | 0 | 0.5679 | nM | nM | nM |
| col2348_19 | 1177 | 0.8985 | 0 | 0.5627 | nM | nM | nM |
| col2348_22 | 1316 | 0.8836 | 0 | 0.5451 | nM | nM | nM |
| col2348_23 | 1606 | 0.9322 | 0 | 0.5394 | nM | nM | nM |
| col2348_27 | 1098 | 0.9395 | 0 | 0.5775 | nM | nM | nM |
| col2348_28 | 171762 | 0.9814 | 46131.17 | 0.7574 | M | M | M |
| col2348_29 | 1794 | 0.9927 | 0 | 0.5108 | nM | nM | nM |
| col2348_30 | 1898 | 0.9501 | 0 | 0.5811 | nM | nM | nM |
| col2348_33 | 891 | 0.5794 | 0 | 0.5545 | nM | nM | nM |
| col2348_41 | 1573 | 0.8883 | 757.87 | 0.5595 | nM | nM | nM |
| col2348_42 | 2659 | 0.9621 | 0 | 0.5236 | nM | nM | nM |
| col2348_43 | 1068 | 0.8337 | 0 | 0.5621 | nM | nM | nM |
| col2348_45 | 1759 | 0.9461 | 0 | 0.5398 | nM | nM | nM |
| col2348_46 | 1384 | 0.8899 | 0 | 0.5738 | nM | nM | nM |
| col2348_48 | 2014 | 0.919 | 0 | 0.5977 | nM | nM | nM |
| col2348_52 | 1650 | 0.839 | 0 | 0.5734 | nM | nM | nM |
| col2348_53 | 184848 | 0.995 | 98196.02 | 0.8483 | M | M | M |
| col2348_54 | 188004 | 0.9908 | 77402.58 | 0.8563 | M | M | M |
| col2348_55 | 1289 | 0.948 | 0 | 0.4299 | nM | nM | nM |
| col2348_57 | 2521 | 0.9523 | 0 | 0.5507 | nM | nM | nM |
| col2348_58 | 1589 | 0.9576 | 0 | 0.5783 | nM | nM | nM |
| col2348_60 | 1267 | 0.7884 | 0 | 0.5936 | nM | nM | nM |
| col2348_62 | 145318 | 0.981 | 46428.16 | 0.7629 | M | M | M |
| col2348_63 | 28430 | 0.883 | 0 | 0.2154 | nM | nM | M |
| col2348_64 | 121529 | 0.9875 | 42927.13 | 0.7142 | M | M | M |
| col2348_65 | 1098 | 0.859 | 718.45 | 0.5863 | nM | nM | nM |
| col2348_66 | 2165 | 0.9472 | 0 | 0.5764 | nM | nM | nM |
| col2348_69 | 1792 | 0.9735 | 0 | 0.5699 | nM | nM | nM |
| col2348_70 | 1730 | 0.8836 | 773.29 | 0.556 | nM | nM | nM |
| col2348_72 | 139956 | 0.9891 | 62917.98 | 0.7072 | M | M | M |
| col2348_76 | 1318 | 0.8093 | 0 | 0.5614 | nM | nM | nM |
| col2348_80 | 226322 | 0.9899 | 161862.8 | 0.7945 | M | M | M |
| col2348_81 | 1456 | 0.968 | 0 | 0.5623 | nM | nM | nM |
| col2348_82 | 1849 | 0.8108 | 0 | 0.5383 | nM | nM | nM |
| col2348_84 | 936 | 0.8694 | 0 | 0.5634 | nM | nM | nM |
| col2348_85 | 2514 | 0.9362 | 1653.08 | 0.4989 | nM | nM | nM |
| col2348_86 | 4094 | 0.9794 | 0 | 0.5224 | nM | nM | nM |
| col2348_89 | 861 | 0.9108 | 596.54 | 0.5798 | nM | nM | nM |
| col2348_90 | 1131 | 0.8699 | 0 | 0.5145 | nM | nM | nM |
| col2348_91 | 2087 | 0.9186 | 0 | 0.5856 | nM | nM | nM |
| col2348_93 | 756 | 0.5591 | 0 | 0.549 | nM | nM | nM |
| col2348_95 | 1359 | 0.805 | 0 | 0.5931 | nM | nM | nM |
| col2348_96 | 1409 | 0.9581 | 0 | 0.5907 | nM | nM | nM |
| col2348_97 | 205421 | 0.9892 | 102214.2 | 0.8032 | M | M | M |
| col2348_98 | 1001 | 0.9104 | 0 | 0.5789 | nM | nM | nM |
| col2348_104 | 1979 | 0.8887 | 1043.49 | 0.5582 | nM | nM | nM |
| col2348_105 | 2440 | 0.9384 | 0 | 0.5125 | nM | nM | nM |
| col2348_108 | 2356 | 0.9493 | 0 | 0.5805 | nM | nM | nM |
| col2348_109 | 1337 | 0.9074 | 0 | 0.5073 | nM | nM | nM |
| col2348_116 | 970 | 0.8773 | 0 | 0.5982 | nM | nM | nM |
| col2348_123 | 855 | 0.9409 | 0 | 0.5762 | nM | nM | nM |
| col2348_124 | 189521 | 0.9928 | 39434.05 | 0.6906 | nM | M | M |
| col2348_125 | 1659 | 0.978 | 0 | 0.5715 | nM | nM | nM |
| col2348_131 | 913 | 0.922 | 0 | 0.5734 | nM | nM | nM |
| col2348_136 | 1551 | 0.682 | 0 | 0.517 | nM | nM | nM |
| col2348_140 | 95682 | 0.9832 | 60116.02 | 0.8079 | M | M | M |
| col2348_144 | 281488 | 0.9885 | 184228.5 | 0.8949 | M | M | M |
| col2348_145 | 3053 | 0.7129 | 2196.38 | 0.5235 | nM | nM | nM |
| col2348_147 | 2241 | 0.8895 | 1785.35 | 0.587 | nM | nM | nM |
| col2348_148 | 1428 | 0.935 | 0 | 0.5783 | nM | nM | nM |
| col2348_150 | 938 | 0.9029 | 0 | 0.5034 | nM | nM | nM |
| col2348_151 | 3153 | 0.9737 | 0 | 0.5644 | nM | nM | nM |
| col2348_185 | 1500 | 0.9089 | 0 | 0.5655 | nM | nM | nM |
| col2348_194 | 1047 | 0.935 | 0 | 0.5544 | nM | nM | nM |
| col2348_196 | 1024 | 0.9388 | 0 | 0.549 | nM | nM | nM |
| col2348_197 | 3490 | 0.9734 | 2543.8 | 0.5975 | nM | nM | nM |
| col2348_199 | 1494 | 0.9197 | 0 | 0.6142 | nM | nM | nM |
| col2348_215 | 2667 | 0.9594 | 0 | 0.5603 | nM | nM | nM |
| col2348_216 | 1461 | 0.9349 | 0 | 0.5307 | nM | nM | nM |
| col2348_218 | 949 | 0.9013 | 0 | 0.6313 | nM | nM | nM |
| col2348_221 | 1315 | 0.9302 | 0 | 0.571 | nM | nM | nM |
| col2348_231 | 1218 | 0.9257 | 0 | 0.5574 | nM | nM | nM |
| col2348_244 | 2142 | 0.9739 | 0 | 0.2663 | nM | nM | nM |
| col2348_247 | 3118 | 0.9666 | 0 | 0.3322 | nM | nM | nM |
| col2348_256 | 2436 | 0.9739 | 0 | 0.2829 | nM | nM | nM |
| col2348_265 | 2919 | 0.9776 | 0 | 0.6147 | nM | nM | nM |
| col2348_270 | 1400 | 0.653 | 1868.34 | 0.6407 | nM | nM | nM |
| col2348_276 | 3126 | 0.9635 | 0 | 0.5209 | nM | nM | nM |
| col2348_277 | 147169 | 0.986 | 39930.95 | 0.7975 | M | M | M |
| col2348_297 | 765 | 0.8723 | 0 | 0.5617 | nM | nM | nM |
| col2348_300 | 547 | 0.897 | 0 | 0.5651 | nM | nM | nM |
| col2348_317 | 890 | 0.7693 | 0 | 0.5299 | nM | nM | nM |
| col2348_334 | 2037 | 0.9681 | 0 | 0.5358 | nM | nM | nM |
| col2348_335 | 782 | 0.8897 | 0 | 0.5559 | nM | nM | nM |
| col2348_337 | 1539 | 0.9476 | 0 | 0.5305 | nM | nM | nM |
| col2348_338 | 1297 | 0.9104 | 0 | 0.555 | nM | nM | nM |
| col2348_340 | 2823 | 0.9622 | 3240.14 | 0.5885 | nM | nM | nM |
| col2348_341 | 8105 | 0.9137 | 0 | 0.1822 | nM | nM | nM |
| col2348_342 | 3576 | 0.9547 | 0 | 0.5494 | nM | nM | nM |
| col2348_355 | 2868 | 0.9471 | 0 | 0.5759 | nM | nM | nM |
| col2348_356 | 644 | 0.8267 | 0 | 0.5681 | nM | nM | nM |
| col2348_357 | 2834 | 0.9841 | 0 | 0.5622 | nM | nM | nM |
| col2348_359 | 1543 | 0.9447 | 1295.58 | 0.5255 | nM | nM | nM |
| col2348_360 | 697 | 0.8052 | 0 | 0.5463 | nM | nM | nM |
| col2348_375 | 2816 | 0.9169 | 1841.28 | 0.5203 | nM | nM | nM |
| col2348_377 | 173727 | 0.9925 | 46508.52 | 0.7798 | M | M | M |
| col2348_385 | 1026 | 0.7842 | 811.08 | 0.5357 | nM | nM | nM |
| col2348_387 | 1340 | 0.9113 | 593.4 | 0.5895 | nM | nM | nM |
| col2348_388 | 116214 | 0.986 | 33438.5 | 0.8401 | M | M | M |
| col2348_390 | 2487 | 0.9102 | 0 | 0.5258 | nM | nM | nM |
| col2348_399 | 1211 | 0.9821 | 0 | 0.5487 | nM | nM | nM |
| col2348_403 | 145783 | 0.9945 | 45940.36 | 0.8548 | M | M | M |
| col2348_407 | 829 | 0.9184 | 0 | 0.5115 | nM | nM | nM |
| col2348_428 | 2444 | 0.9417 | 0 | 0.397 | nM | nM | nM |
| col2348_434 | 1447 | 0.9601 | 0 | 0.5802 | nM | nM | nM |
| col2348_444 | 1247 | 0.8918 | 1516.48 | 0.6203 | nM | nM | nM |
| col2348_446 | 122192 | 0.9903 | 57035.73 | 0.8554 | M | M | M |
| col2348_448 | 2366 | 0.9932 | 0 | 0.5017 | nM | nM | nM |
| col2348_449 | 1028 | 0.8298 | 0 | 0.5465 | nM | nM | nM |
| col2348_450 | 178506 | 0.9867 | 42244.26 | 0.728 | M | M | M |
| col2348_456 | 863 | 0.9046 | 0 | 0.5485 | nM | nM | nM |
| col2348_465 | 1113 | 0.9352 | 0 | 0.5333 | nM | nM | nM |
| col2348_466 | 1866 | 0.9484 | 5192.23 | 0.5578 | nM | nM | nM |
| col2348_467 | 1266 | 0.9441 | 0 | 0.504 | nM | nM | nM |
| col2348_469 | 1116 | 0.8863 | 0 | 0.5138 | nM | nM | nM |
| col2348_470 | 1686 | 0.9153 | 1407.74 | 0.5593 | nM | nM | nM |
| col2348_477 | 2090 | 0.9869 | 1395.03 | 0.4787 | nM | nM | nM |
| col2348_482 | 2551 | 0.9766 | 0 | 0.5219 | nM | nM | nM |
| col2348_484 | 3797 | 0.9698 | 3086.44 | 0.6282 | nM | nM | nM |
| col2348_486 | 1951 | 0.9533 | 0 | 0.5497 | nM | nM | nM |
| col2348_487 | 1054 | 0.8912 | 3675.69 | 0.5675 | nM | nM | nM |
| col2348_490 | 1900 | 0.9715 | 0 | 0.5959 | nM | nM | nM |
| col2348_491 | 165710 | 0.9952 | 76517.36 | 0.7402 | M | M | M |
| col2348_496 | 772 | 0.9055 | 0 | 0.5243 | nM | nM | nM |
| col2348_502 | 1690 | 0.9703 | 0 | 0.5526 | nM | nM | nM |
| col2348_518 | 1328 | 0.9313 | 0 | 0.5662 | nM | nM | nM |
| col2348_539 | 2179 | 0.9834 | 0 | 0.5445 | nM | nM | nM |
| col2348_541 | 188781 | 0.9842 | 56508.25 | 0.7416 | M | M | M |
| col2348_558 | 1336 | 0.9189 | 0 | 0.6065 | nM | nM | nM |
| col2348_577 | 788 | 0.755 | 905.63 | 0.6576 | nM | nM | nM |
| col2348_598 | 1264 | 0.9548 | 0 | 0.5638 | nM | nM | nM |
| col2348_601 | 773 | 0.8941 | 0 | 0.5725 | nM | nM | nM |
| col2348_619 | 646 | 0.9441 | 0 | 0.5531 | nM | nM | nM |
| col2348_630 | 291592 | 0.9951 | 229635.1 | 0.9039 | M | M | M |
| col2348_637 | 4815 | 0.6999 | 0 | 0.5693 | nM | nM | nM |
| col2348_639 | 2691 | 0.9756 | 0 | 0.511 | nM | nM | nM |
| col2348_641 | 1020 | 0.9131 | 0 | 0.5354 | nM | nM | nM |
| col2348_645 | 1431 | 0.961 | 0 | 0.5703 | nM | nM | nM |
| col2348_648 | 1077 | 0.8499 | 0 | 0.5557 | nM | nM | nM |
| col2348_654 | 869 | 0.8778 | 0 | 0.5607 | nM | nM | nM |
| col2348_659 | 913 | 0.9552 | 0 | 0.5698 | nM | nM | nM |
| col2348_665 | 831 | 0.9359 | 0 | 0.5682 | nM | nM | nM |
| col2348_671 | 1105 | 0.9395 | 0 | 0.5397 | nM | nM | nM |
| col2348_674 | 129937 | 0.9842 | 40362.99 | 0.8873 | M | M | M |
| col2348_677 | 142371 | 0.9929 | 25664.47 | 0.6567 | M | M | M |
| col2348_682 | 1011 | 0.9556 | 0 | 0.5673 | nM | nM | nM |
| col2348_683 | 667 | 0.8813 | 0 | 0.5052 | nM | nM | nM |
| col2348_684 | 583 | 0.7584 | 0 | 0.3614 | nM | nM | nM |
| col2348_686 | 988 | 0.9062 | 0 | 0.5861 | nM | nM | nM |
| col2348_689 | 107093 | 0.9839 | 59799.03 | 0.7499 | nM | M | M |
| col2348_696 | 1862 | 0.9657 | 0 | 0.5333 | nM | nM | nM |
| col2348_701 | 1586 | 0.9819 | 0 | 0.5727 | nM | nM | nM |
| col2348_705 | 1267 | 0.9594 | 1003.29 | 0.5903 | nM | nM | nM |
| col2348_719 | 1537 | 0.9656 | 0 | 0.589 | nM | nM | nM |
| col2348_722 | 3869 | 0.9846 | 0 | 0.5813 | nM | nM | nM |
| col2348_723 | 188493 | 0.9938 | 94883.59 | 0.8798 | M | M | M |
| col2348_736 | 122430 | 0.988 | 48561.83 | 0.8755 | M | M | M |
| col2348_749 | 2535 | 0.9785 | 0 | 0.5495 | nM | nM | nM |
| col2348_755 | 2919 | 0.885 | 0 | 0.3268 | nM | nM | nM |
| col2348_757 | 134407 | 0.981 | 72815.66 | 0.744 | M | M | M |
| col2348_764 | 1359 | 0.9464 | 0 | 0.5262 | nM | nM | nM |
| col2348_766 | 130145 | 0.9872 | 43629.83 | 0.845 | M | M | M |
| col2348_772 | 162058 | 0.9839 | 56487.18 | 0.6927 | M | M | M |
| col2348_781 | 1826 | 0.9558 | 0 | 0.5794 | M | nM | nM |
| col2348_790 | 117832 | 0.9953 | 77317.85 | 0.8781 | M | M | M |
| col2348_793 | 1789 | 0.9626 | 0 | 0.4923 | nM | nM | nM |
| col2348_794 | 1464 | 0.9658 | 0 | 0.5622 | nM | nM | nM |
| col2348_796 | 1735 | 0.9492 | 0 | 0.6075 | nM | nM | nM |
| col2348_798 | 1320 | 0.6093 | 0 | 0.5296 | nM | nM | nM |
| col2348_811 | 2896 | 0.9802 | 0 | 0.2508 | nM | nM | nM |
| col2348_813 | 1006 | 0.9131 | 0 | 0.5543 | nM | nM | nM |
| col2348_815 | 119501 | 0.9858 | 72886.43 | 0.7937 | M | M | M |
| col2348_820 | 121435 | 0.9892 | 50159.15 | 0.7263 | M | M | M |
| col2348_822 | 115142 | 0.9854 | 33865.39 | 0.8798 | M | M | M |
| col2348_826 | 1599 | 0.7748 | 0 | 0.4565 | nM | nM | nM |
| col2348_832 | 171131 | 0.9882 | 65084.74 | 0.8345 | M | M | M |
| col2348_834 | 916 | 0.9086 | 0 | 0.5615 | nM | nM | nM |
| col2348_836 | 112925 | 0.9852 | 39446.84 | 0.8976 | M | M | M |
| col2348_837 | 92724 | 0.9807 | 37213.17 | 0.8253 | M | M | M |
| col2348_838 | 129528 | 0.9809 | 52461.36 | 0.8921 | M | M | M |
| col2348_841 | 116871 | 0.9847 | 49143.34 | 0.8339 | M | M | M |
| col2348_842 | 161919 | 0.9706 | 83595.32 | 0.885 | M | M | M |
| col2348_845 | 810 | 0.8648 | 1224.89 | 0.4864 | nM | nM | nM |
| col2348_846 | 66220 | 0.977 | 24410.76 | 0.835 | M | M | M |
| col2348_850 | 2289 | 0.942 | 6830.34 | 0.5141 | nM | nM | nM |
| col2348_883 | 801 | 0.5578 | 2726.97 | 0.7541 | nM | nM | nM |
| col2348_886 | 1873 | 0.9776 | 0 | 0.5511 | nM | nM | nM |
| col2348_904 | 2279 | 0.9386 | 0 | 0.4514 | nM | nM | nM |
| col2348_907 | 1599 | 0.9383 | 0 | 0.476 | nM | nM | nM |
| col2348_913 | 2407 | 0.9782 | 1254.96 | 0.5654 | nM | nM | nM |
| col2348_914 | 742 | 0.9274 | 0 | 0.5879 | nM | nM | nM |
| col2348_915 | 33510 | 0.9873 | 34334.3 | 0.7776 | M | M | M |
| col2348_918 | 30964 | 0.9833 | 20901.87 | 0.7173 | M | M | M |
| col2348_922 | 1378 | 0.8737 | 0 | 0.4483 | nM | nM | nM |
| col2348_925 | 691 | 0.8383 | 0 | 0.5503 | nM | nM | nM |
| col2348_927 | 1173 | 0.8196 | 0 | 0.5627 | nM | nM | nM |
| col2348_928 | 1385 | 0.9453 | 0 | 0.5486 | nM | nM | nM |
| col2348_929 | 170608 | 0.9889 | 107439.2 | 0.8325 | M | M | M |
| col2348_932 | 762 | 0.9423 | 0 | 0.5614 | nM | nM | nM |
| col2348_937 | 839 | 0.8219 | 0 | 0.5326 | nM | nM | nM |
| col2348_938 | 1865 | 0.9594 | 0 | 0.4924 | nM | nM | nM |
| col2348_941 | 1684 | 0.9521 | 0 | 0.2988 | nM | nM | nM |
| col2348_946 | 2019 | 0.9665 | 0 | 0.5364 | nM | nM | nM |
| col2348_949 | 1086 | 0.8378 | 1051.8 | 0.5149 | nM | nM | nM |
| col2348_951 | 1699 | 0.6505 | 0 | 0.511 | nM | nM | nM |
| col2348_954 | 1167 | 0.904 | 0 | 0.5304 | nM | nM | nM |
| col2348_973 | 1430 | 0.9221 | 0 | 0.5735 | nM | nM | nM |
| col2348_974 | 930 | 0.9622 | 0 | 0.5763 | nM | nM | nM |
| col2348_976 | 781 | 0.7832 | 594.22 | 0.4886 | nM | nM | nM |
| col2348_978 | 1083 | 0.9359 | 0 | 0.5061 | nM | nM | nM |
| col2348_986 | 1167 | 0.8848 | 0 | 0.5112 | nM | nM | nM |
| col2348_987 | 905 | 0.8978 | 0 | 0.5042 | nM | nM | nM |
| col2348_992 | 875 | 0.9445 | 0 | 0.6411 | nM | nM | nM |
| col2348_1003 | 2226 | 0.9553 | 1194.51 | 0.5409 | nM | nM | nM |
| col2348_1011 | 2888 | 0.9276 | 0 | 0.5613 | nM | nM | nM |
| col2348_1012 | 2745 | 0.9042 | 768.14 | 0.5697 | nM | nM | nM |
| col2348_1013 | 155258 | 0.9857 | 44623.12 | 0.8649 | M | M | M |
| col2348_1014 | 1889 | 0.8543 | 0 | 0.5789 | nM | nM | nM |
| col2348_1015 | 1822 | 0.9901 | 0 | 0.521 | nM | nM | nM |
| col2348_1018 | 643 | 0.8996 | 0 | 0.5766 | nM | nM | nM |
| col2348_1021 | 1533 | 0.9749 | 0 | 0.4391 | M | nM | nM |
| col2348_1022 | 188633 | 0.9828 | 90996.97 | 0.8208 | M | M | M |
| col2348_1027 | 900 | 0.9506 | 0 | 0.5283 | nM | nM | nM |
| col2348_1032 | 504 | 0.9141 | 0 | 0.5815 | nM | nM | nM |
| col2348_1036 | 219938 | 0.9916 | 227001.7 | 0.6858 | M | M | M |
| col2348_1038 | 1475 | 0.9074 | 1245.06 | 0.5258 | nM | nM | nM |
| col2348_1039 | 644 | 0.7689 | 0 | 0.5823 | nM | nM | nM |
| col2348_1040 | 1173 | 0.8897 | 723.71 | 0.6034 | nM | nM | nM |
| col2348_1047 | 1151 | 0.9387 | 0 | 0.5691 | nM | nM | nM |
| col2348_1049 | 1265 | 0.9399 | 896.37 | 0.5681 | nM | nM | nM |
| col2348_1053 | 141060 | 0.9931 | 30015.83 | 0.8193 | M | M | M |
| col2348_1056 | 2479 | 0.9581 | 0 | 0.5414 | nM | nM | nM |
| col2348_1060 | 837 | 0.8914 | 0 | 0.5531 | nM | nM | nM |
| col2348_1064 | 1131 | 0.901 | 0 | 0.5783 | nM | nM | nM |
| col2348_1072 | 155766 | 0.9953 | 67003.74 | 0.8781 | M | M | M |
| col2348_1077 | 838 | 0.8126 | 0 | 0.5849 | nM | nM | nM |
| col2348_1083 | 1589 | 0.89 | 1608.7 | 0.5835 | nM | nM | nM |
| col2348_1085 | 3053 | 0.9705 | 0 | 0.5095 | nM | nM | nM |
| col2348_1094 | 25663 | 0.9649 | 35757.25 | 0.7695 | M | M | M |
| col2348_1104 | 1835 | 0.8852 | 0 | 0.4277 | nM | nM | nM |
| col2348_1105 | 3076 | 0.9044 | 1158.7 | 0.5433 | nM | nM | nM |
| col2348_1107 | 2072 | 0.9025 | 714.02 | 0.6149 | nM | nM | nM |
| col2348_1108 | 1883 | 0.8596 | 0 | 0.5036 | nM | nM | nM |
| col2348_1109 | 148484 | 0.9883 | 71870.47 | 0.8755 | M | M | M |
| col2348_1110 | 2167 | 0.9118 | 1208.42 | 0.5199 | nM | nM | nM |
| col2348_1111 | 2329 | 0.8547 | 978.79 | 0.6137 | nM | nM | nM |
| col2348_1112 | 934 | 0.7969 | 0 | 0.6066 | nM | nM | nM |
| col2348_1116 | 32849 | 0.9827 | 26524.89 | 0.7947 | M | M | M |
| col2348_1120 | 119470 | 0.9908 | 41842.74 | 0.8776 | M | M | M |
| col2348_1121 | 1115 | 0.9155 | 1049.84 | 0.5496 | nM | nM | nM |
| col2348_1123 | 1375 | 0.6977 | 0 | 0.4107 | nM | nM | nM |
| col2348_1124 | 1607 | 0.9466 | 0 | 0.5536 | nM | nM | nM |
| col2348_1127 | 162792 | 0.9884 | 42576.63 | 0.8255 | M | M | M |
| col2348_1130 | 709 | 0.954 | 0 | 0.5453 | nM | nM | nM |
| col2348_1131 | 108775 | 0.9903 | 47985.68 | 0.796 | M | M | M |
| col2348_1138 | 1045 | 0.9613 | 0 | 0.5739 | nM | nM | nM |
| col2348_1144 | 2258 | 0.9826 | 0 | 0.5414 | nM | nM | nM |
| col2348_1152 | 1593 | 0.9399 | 0 | 0.5831 | nM | nM | nM |
| col2348_1160 | 1551 | 0.9677 | 1138.83 | 0.5815 | nM | nM | nM |
| col2348_1161 | 1055 | 0.8305 | 812.78 | 0.5093 | nM | nM | nM |
| col2348_1163 | 1104 | 0.822 | 0 | 0.5062 | nM | nM | nM |
| col2348_1164 | 763 | 0.7531 | 0 | 0.4956 | nM | nM | nM |
| col2348_1165 | 1347 | 0.9724 | 0 | 0.5219 | nM | nM | nM |
| col2348_1168 | 996 | 0.9192 | 0 | 0.5735 | nM | nM | nM |
| col2348_1170 | 2264 | 0.9449 | 1370.73 | 0.5996 | nM | nM | nM |
| col2348_1171 | 732 | 0.9406 | 0 | 0.5534 | nM | nM | nM |
| col2348_1172 | 1788 | 0.9389 | 0 | 0.4028 | nM | nM | nM |
| col2348_1175 | 127595 | 0.9902 | 44835.68 | 0.824 | M | M | M |
| col2348_1177 | 73574 | 0.9898 | 1011838 | 0.9105 | M | M | M |
| col2348_1179 | 970 | 0.8399 | 0 | 0.5232 | nM | nM | nM |
| col2348_1181 | 138435 | 0.9897 | 70723.22 | 0.8404 | M | M | M |
| col2348_1183 | 1287 | 0.7577 | 0 | 0.5965 | nM | nM | nM |
| col2348_1190 | 944 | 0.9568 | 0 | 0.4485 | nM | nM | nM |
| col2348_1245 | 924 | 0.9247 | 0 | 0.5921 | nM | nM | nM |
| col2348_1293 | 196188 | 0.9891 | 100509.7 | 0.8003 | M | M | M |
| col2348_1301 | 660 | 0.9289 | 0 | 0.5245 | nM | nM | nM |
| col2348_1307 | 800 | 0.8729 | 583.7 | 0.5807 | nM | nM | nM |
| col2348_1308 | 1506 | 0.8927 | 0 | 0.6121 | nM | nM | nM |
| col2348_1309 | 1383 | 0.9586 | 0 | 0.4245 | nM | nM | nM |
| col2348_1338 | 1364 | 0.9482 | 0 | 0.5657 | nM | nM | nM |
| col2348_1341 | 2328 | 0.9852 | 0 | 0.5819 | nM | nM | nM |
| col2348_1344 | 1633 | 0.9593 | 1130.82 | 0.5648 | nM | nM | nM |
| col2348_1347 | 116509 | 0.9904 | 39107.43 | 0.8175 | M | M | M |
| col2348_1368 | 1124 | 0.9498 | 0 | 0.5693 | nM | nM | nM |
| col2348_1375 | 123338 | 0.9889 | 55438.88 | 0.9016 | M | M | M |
| col2348_1419 | 221914 | 0.9967 | 95059.8 | 0.8788 | M | M | M |
| col2348_1427 | 2501 | 0.8529 | 1286.69 | 0.453 | M | nM | nM |
| col2348_1428 | 155971 | 0.9915 | 61329.72 | 0.8344 | M | M | M |
| col2348_1429 | 672 | 0.8282 | 0 | 0.5835 | M | nM | nM |
| col2348_1446 | 1419 | 0.921 | 0 | 0.5322 | nM | nM | nM |
| col2348_1447 | 2489 | 0.9723 | 0 | 0.5664 | nM | nM | nM |
| col2348_1449 | 121686 | 0.9879 | 47975.16 | 0.8543 | M | M | M |
| col2348_1451 | 2536 | 0.9782 | 0 | 0.4692 | nM | nM | nM |
| col2348_1453 | 1121 | 0.9566 | 0 | 0.5993 | nM | nM | nM |
| col2348_1455 | 31797 | 0.9925 | 42519.1 | 0.7888 | M | M | M |
| col2348_1465 | 1212 | 0.8927 | 0 | 0.463 | nM | nM | nM |
| col2348_1471 | 950 | 0.952 | 0 | 0.5597 | nM | nM | nM |
| col2348_1473 | 152302 | 0.9885 | 69941.44 | 0.8728 | M | M | M |
| col2348_1475 | 130781 | 0.9858 | 45733.94 | 0.8141 | M | M | M |
| col2348_1476 | 1017 | 0.7655 | 0 | 0.4907 | nM | nM | nM |
| col2348_1481 | 22842 | 0.8331 | 0 | 0.2069 | nM | nM | M |
| col2348_1488 | 123217 | 0.989 | 36511.15 | 0.8268 | M | M | M |
| col2348_1492 | 1192 | 0.8594 | 0 | 0.4773 | nM | nM | nM |
| col2348_1509 | 1847 | 0.9708 | 0 | 0.5369 | nM | nM | nM |
| col2348_1511 | 1169 | 0.9131 | 0 | 0.5742 | nM | nM | nM |
| col2348_1514 | 1125 | 0.9263 | 0 | 0.4737 | nM | nM | nM |
| col2348_1516 | 183134 | 0.9867 | 80919.97 | 0.8403 | M | M | M |
| col2348_1518 | 70507 | 0.979 | 20834.6 | 0.8432 | M | M | M |
| col2348_1523 | 985 | 0.9031 | 0 | 0.591 | nM | nM | nM |
| col2348_1528 | 141156 | 0.9905 | 38317.08 | 0.8508 | M | M | M |
| col2348_1529 | 121822 | 0.9854 | 33140.28 | 0.8444 | M | M | M |
| col2348_1530 | 138351 | 0.99 | 43259.42 | 0.7648 | M | M | M |
| col2348_1545 | 1592 | 0.9411 | 796.96 | 0.596 | nM | nM | nM |
| col2348_1546 | 2329 | 0.9184 | 2324.03 | 0.5211 | nM | nM | nM |
| col2348_1586 | 1472 | 0.9593 | 0 | 0.581 | nM | nM | nM |
| col2348_1629 | 1445 | 0.9812 | 0 | 0.5665 | nM | nM | nM |
| col2348_1633 | 1494 | 0.978 | 0 | 0.5786 | nM | nM | nM |
| col2348_1649 | 1293 | 0.9266 | 0 | 0.5686 | nM | nM | nM |
| col2348_1682 | 1229 | 0.8813 | 915.69 | 0.5463 | nM | nM | nM |
| col2348_1684 | 191020 | 0.9955 | 53790.62 | 0.8158 | M | M | M |
| col2348_1692 | 179095 | 0.9941 | 70004.9 | 0.8241 | M | M | M |
| col2348_1820 | 3140 | 0.9714 | 1110.13 | 0.5887 | nM | nM | nM |
| col2348_1821 | 1023 | 0.7829 | 1294.75 | 0.5758 | nM | nM | nM |
| col2348_1826 | 1196 | 0.7998 | 2609.33 | 0.6097 | nM | nM | nM |
| col2348_1827 | 1508 | 0.8913 | 0 | 0.5743 | nM | nM | nM |
| col2348_1829 | 1457 | 0.959 | 0 | 0.5775 | nM | nM | nM |
| col2348_1830 | 130878 | 0.9888 | 28220.22 | 0.8308 | M | M | M |
| col2348_1846 | 2268 | 0.8739 | 0 | 0.5624 | nM | nM | nM |
| col2348_1858 | 3249 | 0.9706 | 0 | 0.421 | nM | nM | nM |

ID = patient identifier.
RIN = repetitive indel number, DRM = repetitive deletion mean, MMRs = MM signature sum of exposure, CS = max cos similarity, MSIs = MSI status, MMRD = status predicted by MMRDetect, MSIseq = status predicted by MSIse, nM = non-MSI (non-MMR deficient), M = MSI (MMR deficient).

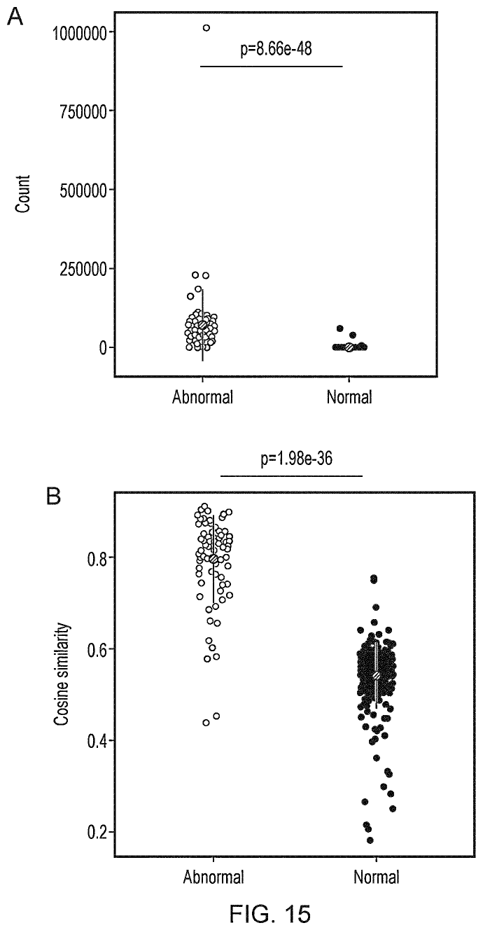

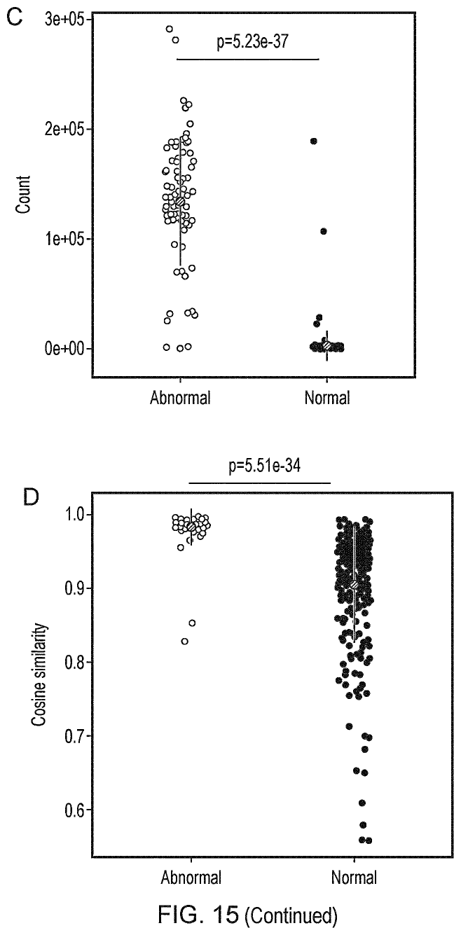

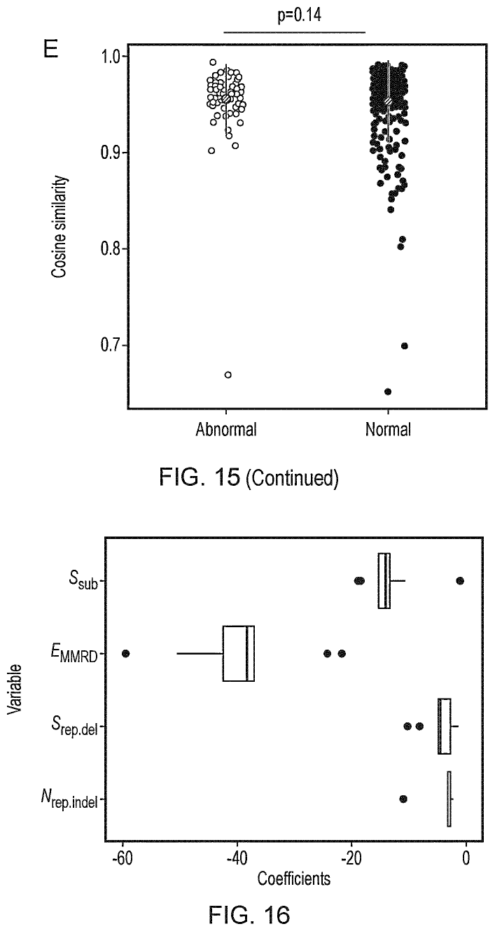

Based on the experimental data, we investigated four potential predictor variables in MMRDetect (FIG. 15):
1) The sum of exposures of MMR mutational signatures (E_{MMRD}). We fitted tissue-specific substitution signatures to each tumor using an R package (signature.tools.lib) published by Degasperi et al. (2020).
2) The maximum cosine similarities between the substitution profiles of cancer samples and those of MMR gene knockouts (S_{sub}), in particular the signatures of PMS2, MLH1, MSH2 and MSH6 knockouts (derived from the set of 4 knockouts for each gene, background adjusted as explained in the methods section in Example 2). For each cancer sample, we calculated the cosine similarity between the substitution profile and substitution signatures of the four MMR gene knockouts (i.e. S₁=Cossim(Profileₜᵤₘₒᵣ, Sig_{ΔPMS2}), mS₂=Cossim(Profileₜᵤₘₒᵣ, Sig_{ΔMLH1}), S₃=Cossim(Profileₜᵤₘₒᵣ, Sig_{ΔMSH2}) S4=Cossim(Profileₜᵤₘₒᵣ, Sig_{ΔMSH6})). The maximum value was used in fitting the model (i.e. S_{sub}=max(S₁, S₂, S₃, S₄)).
3) The number of repeat-mediated indels (N_{rep.indel}). We examined the sequence context of each indel. Only the indels occurring at repetitive regions were used. Repetitive regions were defined as any region of the human reference genome that as 2 or more repeats of the same sequence motif (e.g. AA, AAA, AAAA, AAAAA, ATAT, ATATAT, ATATATAT, CAGCAG, CAGCAGCAG, CAGCAGCAGCAGCAG are all repetitive regions).
4) The cosine similarities between the profiles of repeat-mediated deletions of cancer samples and those of MMR gene knockouts (S_{rep.indel}). For each cancer sample, we calculated the cosine similarity between the repeat-mediated deletion profile and those of the four MMR gene knockouts. The mean value was used for fitting the model.

The values of different variables were transformed to between 0 and 1 using formula x′=x/max(x) for comparability. This is performed for all training samples and for all samples that are subsequently evaluated for testing purposes or in use to identify MMR deficiency in a subject. Table 6 shows calculated parameters of 336 tumors for MSIseq and MMRDetect. The logistic regression algorithm (function glm( )) provided in R package glmnet was employed as the framework of MMRDetect. Table 7 provides the weight (coefficients) of the four variables obtained from training the model using the training data set, and the value of the intercept weight. A ten-fold cross validation was performed for the training data to evaluate the stability of the weights (FIG. 16).

TABLE 7
Weights for the variables used in MMRDetect. These weights
were obtained by training the classifier using 180 MMR-
proficient and 56 MMR-deficient colorectal cancers.

| Variables | Weight |
|---|---|
| E_{MMRD} | −42.95 |
| S_{sub} | −14.53 |
| N_{rep.indel} | −2.96 |
| S_{rep.del} | −4.62 |
| β₀ (intercept) | 16.043 |

Additional four datasets were used to compare the performance of MMRDetect and MSIseq:
1) 2610 tumors from three different studies (Nik-Zainal et al., 2016; Campbell et al., 2020; Staaf et al., 2019);
2) 2024 Hartwig metastatic cancers (Priestley et al., 2019);
3) additional 2012 colorectal cancers from the UK100kGP;
4) 713 uterine samples from UK100kGP.

The characteristics of each of these cohorts are shown in Tables 8-11 below.

TABLE 8
Characteristics of 2012 colorectal cancers from the UK100 kGP.

| | nonMMRd | MMRd |
|---|---|---|
| MMRDetect | 1697 samples | 315 samples |
| MSIseq | 1694 samples | 318 samples |

| | |
|---|---|
| MSIseq − MMRDetect | Concordance = 2005 samples |
| | Non-concordance = 7 |

| | | |
|---|---|---|
| E_{MMRD} (Min./1st | 0.0 0.0 0.0 333.4 0.0 | 1644 40535 54445 69018 |
| Qu./Median/Mean/3rd | 18751.4 | 79269 554958 |

| | |
|---|---|
| Qu./Max.) | 0 0 0 11087 1130 554958 |

| | | |
|---|---|---|
| N_{rep.indel} (Min./1st | 56 965 1284 1661 | 608 111836 138746 |
| Qu./Median/Mean/3rd | 1741 124928 | 140954 165227 349255 |

| | |
|---|---|
| Qu./Max.) | 56 1019 1427 23469 2293 349255 |

| | | |
|---|---|---|
| S_{rep.del} (Min./1st | 0.05241 0.86033 0.91447 | 0.8966 0.9855 0.9883 |
| Qu./Median/Mean/3rd | 0.88232 0.95269 0.99206 | 0.9877 0.9914 0.9974 |

| | |
|---|---|
| Qu./Max.) | 0.05241 0.87366 0.93004 0.89881 0.96869 0.99737 |

| | | |
|---|---|---|
| Ins rep mean (Min./1st | 0.2613 0.9461 0.9656 | 0.8256 0.9521 0.9645 |
| Qu./Median/Mean/3rd | 0.9476 0.9765 0.9951 | 0.9611 0.9743 0.9942 |

| | |
|---|---|
| Qu./Max.) | 0.2613 0.9480 0.9654 0.9497 0.9760 0.9951 |

| | | |
|---|---|---|
| S_{sub} (Min./1st | 0.1475 0.5358 0.5595 | 0.6048 0.7803 0.8218 |
| Qu./Median/Mean/3rd | 0.5474 0.5797 0.7322 | 0.8155 0.8627 0.9489 |

| | |
|---|---|
| Qu./Max.) | 0.1475 0.5403 0.5665 0.5894 0.5957 0.9489 |

nonMMRd = not MMR deficient.
MMRd = MMR deficient.
Ins rep mean = mean cosine similarities between the profiles of repeat-mediated insertions of cancer samples and those of MMR gene knockouts.
Min = minimum, 1st Qu = first quartile, 3^{rd} Qu. = third quartile, Max = maximum.

TABLE 9
Characteristics of 713 uterine samples from UK100 kGP.

| | nonMMRd | MMRd |
|---|---|---|
| MMRDetect | 489 samples | 224 samples |
| MSIseq | 498 samples | 215 samples |
| MSIseq − MMRDetect | Concordance = 692 samples | |
| | Non-concordance = 21 | |
| E_{MMRD} (Min./1st | 0.0 0.0 407.5 1710.2 | 1848 20420 31246 97495 |
| Qu./Median/Mean/3rd | 608.2 134987.9 | 46381 1190029 |
| Qu./Max.) | 0.0 318.8 584.2 31802.5 20247.7 1190029.1 | |
| N_{rep.indel} (Min./1st | 80 367 499 1583 715 | 5710 35081 50716 56462 |
| Qu./Median/Mean/3rd | 44776 | 71401 226004 |
| Qu./Max.) | 80 429 680 18824 32467 226004 | |
| S_{rep.del} (Min./1st | 0.1035 0.5096 0.7056 | 0.5104 0.9732 0.9790 |
| Qu./Median/Mean/3rd | 0.6611 0.8294 0.9915 | 0.9720 0.9848 0.9974 |
| Qu./Max.) | 0.1035 0.5967 0.8198 0.7588 0.9719 0.9974 | |
| Ins rep mean (Min./1st | 0.0765 0.8799 0.9317 | 0.7695 0.9350 0.9506 |
| Qu./Median/Mean/3rd | 0.9001 0.9558 0.9882 | 0.9462 0.9678 0.9942 |
| Qu./Max.) | 0.0765 0.8979 0.9403 0.9146 0.9595 0.9942 | |
| S_{sub} (Min./1st | 0.1295 0.5768 0.6097 | 0.2074 0.8083 0.8659 |
| Qu./Median/Mean/3rd | 0.5650 0.6296 0.7181 | 0.8296 0.9072 0.9658 |

| | |
|---|---|
| Qu./Max.) | 0.1295 0.5941 0.6257 0.6482 0.7939 0.9658 |

nonMMRd = not MMR deficient.
MMRd = MMR deficient.
Ins rep mean = mean cosine similarities between the profiles of repeat-mediated insertions of cancer samples and those of MMR gene knockouts.
Min = minimum, 1st Qu = first quartile, 3^{rd} Qu. = third quartile, Max = maximum.

TABLE 10
Characteristics of 2024 Hartwig metastatic cancer samples.

| | nonMMRd | MMRd |
|---|---|---|
| primary tumour | Biliary: 53 Bone/Soft tissue: 104 Breast: 434 Choroid: 1 | |
| location | CNS: 51 Colon/Rectum: 378 CUP: 4 Esophagus: 95 | |
| | Head and neck: 43 Kidney: 56 Liver: 29 Lung: 169 | |
| | Lymphoid: 1 NET: 1 Other: 5 Ovary: 97 Pancreas: 54 | |
| | Prostate: 210 Skin: 168 Stomach: 27 Urinary tract: 1 | |
| | Uterus: 43 | |
| MMRDetect | 1972 samples | 52 samples |
| MSIseq | 1965 samples | 59 samples |
| MSIseq − MMRDetect | Concordance = 2017 samples | |
| | Non-concordance = 7 samples | |
| E_{MMRD} (Min./1st | 0.0 0.0 0.0 338.6 199.8 | 4736 21767 44776 |
| Qu./Median/Mean/3rd | 26012.6 | 58289 67259 407659 |
| Qu./Max.) | 0 0 0 1827 316 407659 | |
| N_{rep.indel} (Min./1st | 22.0 313.0 546.5 890.1 | 13445 33531 68134 |
| Qu./Median/Mean/3rd | 1114.5 36238.0 | 80981 122775 200687 |
| Qu./Max.) | 22 319 561 2948 1196 200687 | |
| S_{rep.del} (Min./1st | 0.06106 0.30939 0.51178 | 0.9201 0.9811 0.9843 |
| Qu./Median/Mean/3rd | 0.55439 0.84854 0.98828 | 0.9818 0.9887 0.9969 |
| Qu./Max.) | 0.06106 0.31556 0.53266 0.56537 0.86793 0.99693 | |
| Ins rep mean (Min./1st | 0.2770 0.8308 0.9322 | 0.9105 0.9582 0.9695 |
| Qu./Median/Mean/3rd | 0.8733 0.9670 0.9926 | 0.9648 0.9775 0.9866 |
| Qu./Max.) | 0.2770 0.8345 0.9345 0.8757 0.9674 0.9926 | |
| S_{sub} (Min./1st | 0.08825 0.43990 0.56446 | 0.5774 0.7630 0.8047 |
| Qu./Median/Mean/3rd | 0.50861 0.62215 0.76327 | 0.8021 0.8502 0.9166 |

| | |
|---|---|
| Qu./Max.) | 0.08825 0.44598 0.56747 0.51615 0.62498 0.91662 |

nonMMRd = not MMR deficient.
MMRd = MMR deficient.
Ins rep mean = mean cosine similarities between the profiles of repeat-mediated insertions of cancer samples and those of MMR gene knockouts.
Min = minimum, 1st Qu = first quartile, 3^{rd} Qu. = third quartile, Max = maximum.

TABLE 11
Characteristics of 2610 tumour samples from three studies (PCAWG).

| | nonMMRd | MMRd |
|---|---|---|
| MMRDetect | 2580 samples | 30 samples |
| MSIseq | 2595 samples | 15 samples |

| | |
|---|---|
| MSIseq − MMRDetect | Concordance = 2591samples |
| | Non-concordance = 19 |

| | | |
|---|---|---|
| E_{MMRD} (Min./1st | 0.0 0.0 0.0 233.5 253.2 | 7600 14479 23573 40330 |
| Qu./Median/Mean/3rd | 47358.8 | 48445 144739 |

| | |
|---|---|
| Qu./Max.) | 0.0 0.0 0.0 694.3 284.1 144738.5 |

| | | |
|---|---|---|
| N_{rep.indel} (Min./1st | 25.0 148.0 273.0 451.9 | 2885 9878 20019 35915 |
| Qu./Median/Mean/3rd | 462.0 24000.0 | 57856 124093 |

| | |
|---|---|
| Qu./Max.) | 25.0 149.0 275.0 859.5 472.8 124093.0 |

| | | |
|---|---|---|
| S_{rep.del} (Min./1st | 0.04524 0.20507 0.33183 | 0.7838 0.9849 0.9904 |
| Qu./Median/Mean/3rd | 0.41704 0.59078 0.99471 | 0.9799 0.9950 0.9973 |

| | |
|---|---|
| Qu./Max.) | 0.04524 0.20607 0.33687 0.42351 0.60522 0.99731 |

| | | |
|---|---|---|
| Ins rep mean (Min./1st | 0.0000 0.8121 0.9123 | 0.7336 0.9762 0.9812 |
| Qu./Median/Mean/3rd | 0.8480 0.9560 0.9938 | 0.9685 0.9872 0.9928 |

| | |
|---|---|
| Qu./Max.) | 0.0000 0.8154 0.9135 0.8494 0.9570 0.9938 |

| | | |
|---|---|---|
| S_{sub} (Min./1st | 0.08704 0.53110 0.60298 | 0.7386 0.8237 0.8554 |
| Qu./Median/Mean/3rd | 0.56690 0.64891 0.85256 | 0.8546 0.9045 0.9518 |

| | |
|---|---|
| Qu./Max.) | 0.08704 0.53210 0.60379 0.57021 0.65032 0.95177 |

nonMMRd = not MMR deficient.
MMRd = MMR deficient.
Ins rep mean = mean cosine similarities between the profiles of repeat-mediated insertions of cancer samples and those of MMR gene knockouts.
Min = minimum, 1st Qu = first quartile, 3^{rd} Qu. = third quartile, Max = maximum.

Results

Algorithms to classify MMR-deficiency tumors have been developed using massively-parallel sequencing data (Ni Huang et al., 2013; Wang & Liang, 2018; Cortes-Ciriano, 2017; Salipante et al., 2014; Hause et al., 2016). These classifiers depend on detecting elevated tumor mutational burdens (TMB) or microsatellite instability (MSI). New knowledge from our experimental data and awareness of tissue-specific signature variation (FIG. 13B) led us to derive an MMR-deficiency classifier.

We obtained WGS data on 336 colorectal cancers from patients recruited via the National Health Service-based UK 100,000 Genomes Project (UK100kGP) run by Genomics England (GEL). These samples critically had accompanying immunohistochemistry (IHC) validation of MMR-deficiency status based on protein staining of MSH2, MSH6, MLH1 and PMS2. 79 out of 336 cases were identified as MMR-deficient (˜24%). This cohort of 336 samples were randomly assigned into a training set (comprising 180 MMR-proficient and 56 MMR-deficient samples) or a test set (comprising 77 MMR-proficient and 23 MMR-deficient samples). We developed a logistic regression classifier, called MMRDetect, using new mutational-signatures-based parameters derived from the experimental insights gained from our studies above: 1) the exposure of MMR-deficient substitution signatures (E_{MMRD}); 2) the cosine similarity between substitution profile of the tumor and that of MMR knockouts (S_{sub}); 3) the mutation burden of indels in repetitive regions (N_{rep.indel}), and 4) the cosine similarity between repeat-mediated deletion profile of the tumor and that of MMR knockouts (S_{rep.indel}) (further details in Methods, FIGS. 15-17, Table 6, Table 7). A ten-fold cross-validation in the training set was conducted. As a comparator, we applied another widely-used MSI classifier MSIseq (Ni Huang et al., 2013) to the same cohort of 336 colorectal cancers.

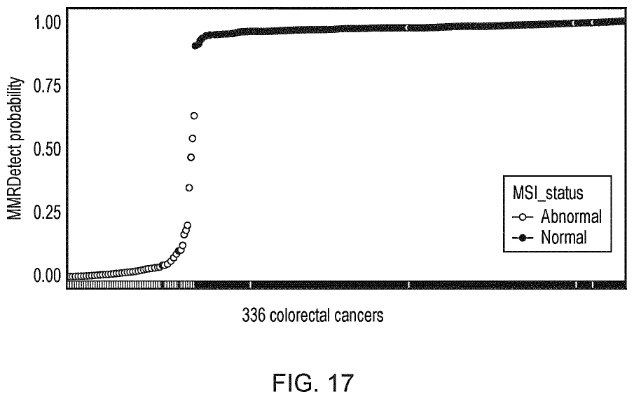

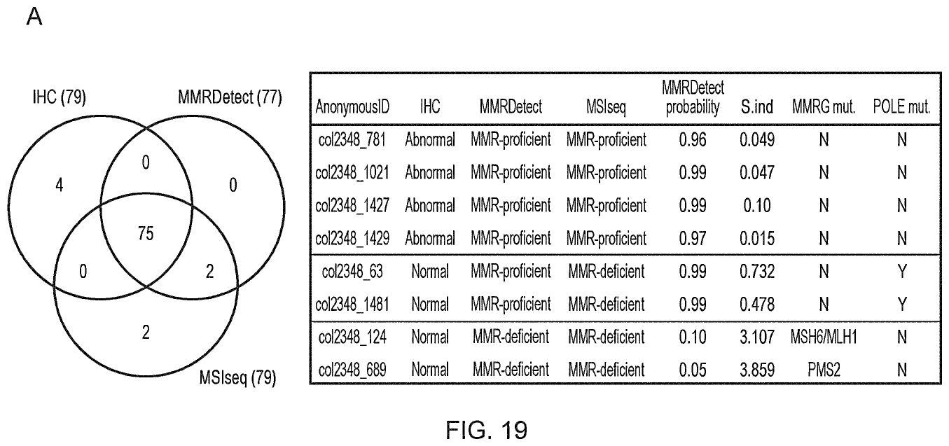

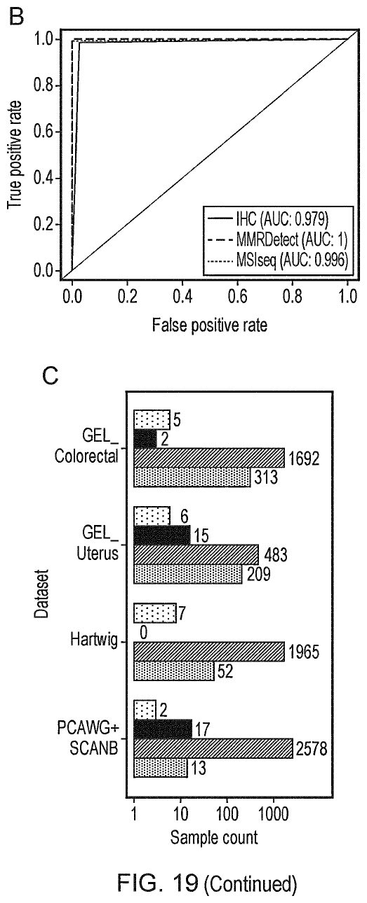

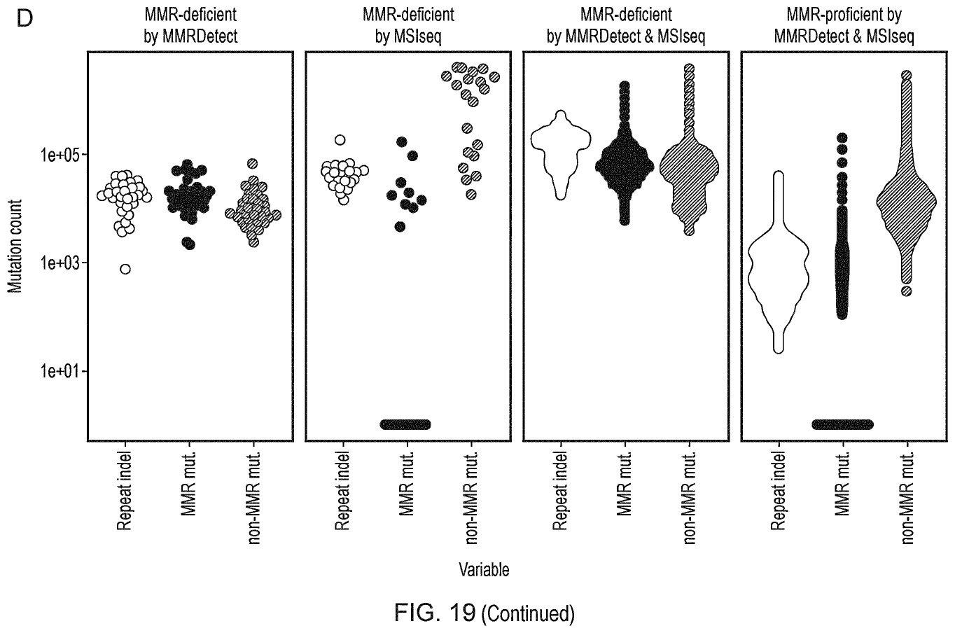

Samples with MMRDetect-calculated probability <0.7 are defined as MMR-deficient by MMRDetect (FIG. 17). In all, 75 of 336 samples were concordantly defined as MMR-deficient by MMRDetect, MSIseq and IHC (FIG. 19A, Table 6). Eight samples had discordant statuses, including 4 samples with MMR-deficiency only by IHC, 2 samples by MSIseq and MMRDetect and not IHC, and 2 samples uniquely called by MSIseq. To understand these discordances, we sought driver mutations. Among these 8 samples, the 2 samples (col2348_124 and col2348_689) which were missed by IHC, had confirmed loss-of-function mutations in MMR genes. Additionally, the two cases uniquely called by MSIseq were misclassified, and were in fact POLE mutant cases and not MMR-deficient (col2348_1481 and col2348_63) (FIG. 19A). While receiver operating characteristic (ROC) curves generated by these three methods show generally excellent performance across the board, MMRDetect had the highest AUC of 1 (FIG. 19B).

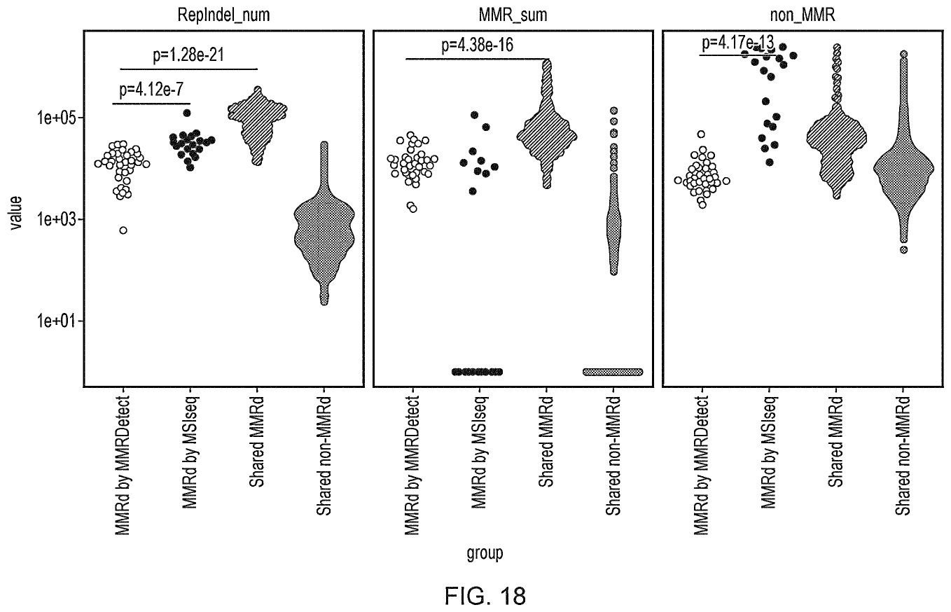

We next directly compared MMRDetect and MSIseq on another 2012 colorectal and 713 uterine samples from UK100kGP, 2,610 published WGS primary cancers (Nik-Zainal et al., 2016; Campbell et al., 2020; Staaf eta I., 2019) and 2024 WGS metastatic cancers (Priestley et al., 2019) (Tables 8-11, Methods). There was very high concordance between MMRDetect and MSISeq for classifying tumors (0.97 to 0.997 (FIG. 19C)). To understand the discrepancies between the two algorithms, we compared variables that were used by the two classifiers (FIG. 19D) and found that samples uniquely identified as MMR-deficient by MSIseq had a significantly higher number of repeat-mediated indels (N_{rep.indel}) and non-MMR-deficiency signatures (E_{non-MMRD}) than the ones identified as MMR-deficient by only MMRDetect (p<0.001, Mann-Whitney test, FIG. 18). This was indicative of a higher likelihood of misclassifying samples with high indel loads caused by non-MMR-deficient mutational processes (i.e. false positives) for MSIseq, a known generic problem reported for NGS indel-based classifiers (Fujimoto et al., 2020). Indeed, many of these samples showed mutational signatures associated with being proofreading POLE mutants. This demonstrates that MMRDetect has an improved specificity over MSIseq. It is also notable that samples identified as MMR-deficient by only MMRDetect had significantly lower numbers of repeat-mediated indels (N_{rep.indel}) and MMR-related substitution signatures (E_{MMRD}), than samples concordantly identified as MMR-deficient by both MSIseq and MMRDetect (p<0.001, Mann-Whitney test, FIG. 18), suggesting that MMRDetect may have improved sensitivity for MMR-deficient cancers with lower overall MMR-related mutation counts (E_{MMRD}). Indeed, of 15 bona fide MMR-deficient breast cancers, a tumor-type that is not as proliferative as colon/uterine cancer and has lower mutation numbers in general, MMRDetect identified 13 cases (87%), whilst MSIseq identified five (˜33%) of the fifteen samples, as the remaining ten samples had lower repeat-mediated indel loads (2885-18863). The two cases missed by MMRDetect had very low levels of MMR-related signatures and were complicated by high levels of APOBEC-related mutagenesis. Thus, MMRDetect has enhanced sensitivity particularly at detecting MMR-deficient samples with lower mutation burdens (FIG. 19D), although could miss cases where MMR-deficiency is present at a very low level. We note that the current version of MMRDetect classifier has been trained on highly-proliferative colorectal cancers. More sequencing data would likely improve MMRDetect further in terms of sensitivity of detection in other tumor types. This may in particular result in slightly different weights of the predictive variables in the trained models, although at least the relative importance of these variables is no expected to change dramatically.

### DISCUSSION

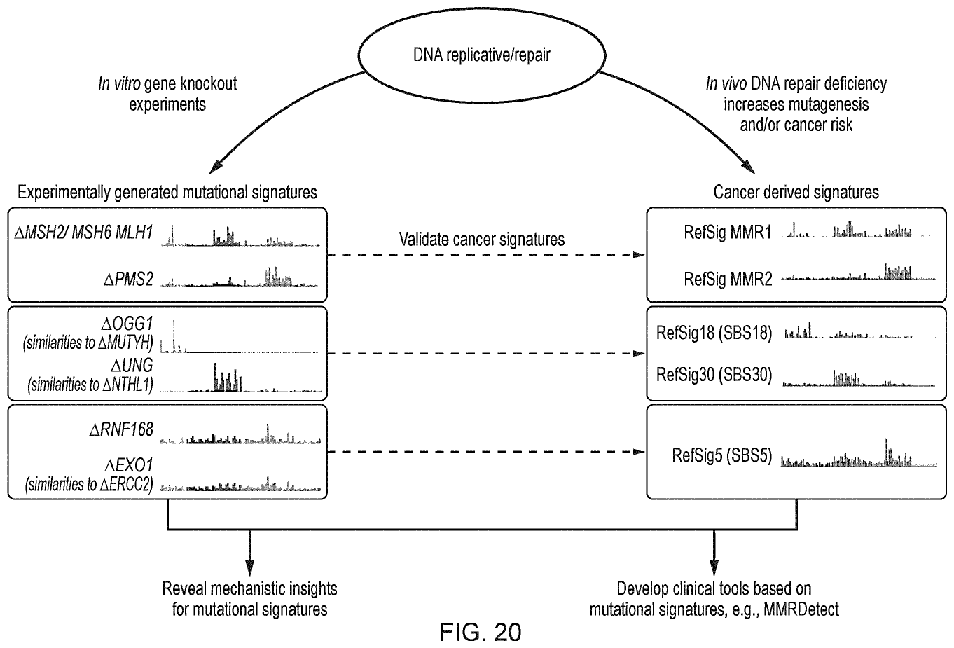

Unlike signatures of environmental mutagens that are historic, signatures of repair pathway defects are likely to be on-going in human cancer cells, and could serve as biomarkers of targetable abnormalities for precision medicine (Mardis, 2019; Berger & Mardis, 2018; Wood et al., 2001) (FIG. 20). This is important for pathways where there are selective therapeutic strategies available. These experiments led us to develop a more sensitive and specific mutational-signature-based assay to detect MMR deficiency, MMRDetect. Current TMB-based assays have reduced sensitivity to detect MMR deficiency because many tissues do not have high proliferative rates and may not meet the detection criteria of such assays. They may also falsely call MMR-deficient cases as MMR-proficient, because single components were used for measurement (e.g., indel burden or substitution count only). High mutational burdens can be due to different biological processes (Campbell et al., 2017). Consequently, assays based on burden alone are unlikely to be adequately specific. As a community, we are at the early stages of seeking experimental validation of mutational signatures. However, we hope that our approach, which leans on experimental data, provides a template for improving biological understanding of how mutational patterns arise, and that this, in turn, could help us propose improved tools for tumour characterization going forward.

### REFERENCES

Haradhvala, N. J. et al. Distinct mutational signatures characterize concurrent loss of polymerase proofreading and mismatch repair. Nature Communications 9, 1746 (2018).
Alexandrov, L. B. et al. The repertoire of mutational signatures in human cancer. Nature 578, 94-101 (2020).
Kim, J. et al. Somatic ERCC2 mutations are associated with a distinct genomic signature in urothelial tumors. Nature Genetics 48, 600-606 (2016).
Nik-Zainal, S. et al. The genome as a record of environmental exposure. Mutagenesis 30, 763-770 (2015).
Zou, X. et al. Validating the concept of mutational signatures with isogenic cell models. Nature Communications 9, 1744 (2018).
Christensen, S. et al. 5-Fluorouracil treatment induces characteristic T>G mutations in human cancer. Nature Communications 10, 4571 (2019).
Kucab, J. E. et al. A Compendium of Mutational Signatures of Environmental Agents. Cell 177, 821-836.e16 (2019).
Mardis, E. R. The Impact of Next-Generation Sequencing on Cancer Genomics: From Discovery to Clinic. Cold Spring Harbor Perspectives in Medicine 9(2019).
Berger, M. F. & Mardis, E. R. The emerging clinical relevance of genomics in cancer medicine. Nature Reviews Clinical Oncology 15, 353-365 (2018).
Wood, R. D., Mitchell, M., Sgouros, J. & Lindahl, T. Human DNA Repair Genes. Science 291, 1284-1289 (2001).
Abid, A., Zhang, M. J., Bagaria, V. K. & Zou, J. Exploring patterns enriched in a dataset with contrastive principal component analysis. Nature Communications 9, 2134 (2018).
van der Maaten, L. & Hinton, G. Visualizing Data using t-SNE. Journal of Machine Learning Research 9, 2579-2605 (2008).
Gupta, S., Gellert, M. & Yang, W. Mechanism of mismatch recognition revealed by human MutSßbound to unpaired DNA loops. Nat Struct Mol Biol 19, 72-78 (2012).
Palombo, F. et al. GTBP, a 160-kilodalton protein essential for mismatch-binding activity in human cells. Science 268, 1912 (1995).
Warren, J. J. et al. Structure of the Human MutSα\DNA\Lesion Recognition Complex. Molecular Cell 26, 579-592 (2007).
Aboul-ela, F., Koh, D., Tinoco, I., Jr. & Martin, F. H. Base-base mismatches. Thermodynamics of double helix formation for dCA3XA3G+dCT3YT3G (X, Y=A,C,G,T). Nucleic acids research 13, 4811-4824 (1985).
Patel, D. J., Kozlowski, S. A., Ikuta, S. & Itakura, K. Dynamics of DNA duplexes containing internal G.T, G.A, A.C, and T.C pairs: hydrogen exchange at and adjacent to mismatch sites. Fed Proc 43, 2663-70 (1984).
Matray, T. J. & Kool, E. T. A specific partner for abasic damage in DNA. Nature 399, 704-708 (1999).
Morikawa, M. et al. Analysis of guanine oxidation products in double-stranded DNA and proposed guanine oxidation pathways in single-stranded, double-stranded or quadruplex DNA. Biomolecules 4, 140-159 (2014).
Pavlov, Y. I., Newlon, C. S. & Kunkel, T. A. Yeast Origins Establish a Strand Bias for Replicational Mutagenesis. Molecular Cell 10, 207-213 (2002).
Mudrak, S. V., Welz-Voegele, C. & Jinks-Robertson, S. The Polymerase η Translesion Synthesis DNA Polymerase Acts Independently of the Mismatch Repair System To Limit Mutagenesis Caused by 7,8-Dihydro-8-Oxoguanine in Yeast. Molecular and Cellular Biology 29, 5316 (2009).
Meier, B. et al. Mutational signatures of DNA mismatch repair deficiency in C. elegans and human cancers. Genome Research 28, 666-675 (2018).
Lang, G. I., Parsons, L. & Gammie, A. E. Mutation Rates, Spectra, and Genome-Wide Distribution of Spontaneous Mutations in Mismatch Repair Deficient Yeast. G3: Genes, Genomes, Genetics 3, 1453 (2013).
Drummond, J. T., Li, G. M., Longley, M. J. & Modrich, P. Isolation of an hMSH2-p160 heterodimer that restores DNA mismatch repair to tumor cells. Science 268, 1909 (1995).
Palombo, F. et al. hMutSβ, a heterodimer of hMSH2 and hMSH3, binds to insertion/deletion loops in DNA. Current Biology 6, 1181-1184 (1996).
Wind, N. d. et al. HNPCC-like cancer predisposition in mice through simultaneous loss of Msh3 and Msh6 mismatch-repair protein functions. Nature Genetics 23, 359-362 (1999).
Poulogiannis, G., Frayling, I. M. & Arends, M. J. DNA mismatch repair deficiency in sporadic colorectal cancer and Lynch syndrome. Histopathology 56, 167-179 (2010).
Heinen, C. D. Mismatch repair defects and Lynch syndrome: The role of the basic scientist in the battle against cancer. DNA Repair 38, 127-134 (2016).
Agu, Chukwuma A. et al. Successful Generation of Human Induced Pluripotent Stem Cell Lines from Blood Samples Held at Room Temperature for up to 48 hr. Stem Cell Reports 5, 660-671 (2015).
Ni Huang, M. et al. MSIseq: Software for Assessing Microsatellite Instability from Catalogs of Somatic Mutations. Scientific Reports 5, 13321 (2015).
Niu, B. et al. MSIsensor: microsatellite instability detection using paired tumor-normal sequence data. Bioinformatics 30, 1015-1016 (2013).
Wang, C. & Liang, C. MSIpred: a python package for tumor microsatellite instability classification from tumor mutation annotation data using a support vector machine. Scientific Reports 8, 17546 (2018).
Cortes-Ciriano, I., Lee, S., Park, W. Y., Kim, T. M. & Park, P. J. A molecular portrait of microsatellite instability across multiple cancers. Nature Communications 8, 15180 (2017).
Salipante, S. J., Scroggins, S. M., Hampel, H. L., Turner, E. H. & Pritchard, C. C. Microsatellite Instability Detection by Next Generation Sequencing. Clinical Chemistry 60, 1192-1199 (2014).
Hause, R. J., Pritchard, C. C., Shendure, J. & Salipante, S. J. Classification and characterization of microsatellite instability across 18 cancer types. Nature Medicine 22, 1342 (2016).
Nik-Zainal, S. et al. Landscape of somatic mutations in 560 breast cancer whole-genome sequences. Nature 534, 47-54 (2016).
Campbell, P. J. et al. Pan-cancer analysis of whole genomes. Nature 578, 82-93 (2020).
Staaf, J. et al. Whole-genome sequencing of triple-negative breast cancers in a population-based clinical study. Nature Medicine 25, 1526-1533 (2019).
Priestley, P. et al. Pan-cancer whole-genome analyses of metastatic solid tumours. Nature 575, 210-216 (2019).
Fujimoto, A. et al. Comprehensive analysis of indels in whole-genome microsatellite regions and microsatellite instability across 21 cancer types. Genome Research 30, 334-346 (2020).
Campbell, B. B. et al. Comprehensive Analysis of Hypermutation in Human Cancer. Cell 171, 1042-1056.e10 (2017).
Bressan, R. B. et al. Efficient CRISPR/Cas9-assisted gene targeting enables rapid and precise genetic manipulation of mammalian neural stem cells. Development 144, 635 (2017).
Tate, P. H. & Skarnes, W. C. Bi-allelic gene targeting in mouse embryonic stem cells. Methods 53, 331-8 (2011).
Hodgkins, A. et al. WGE: a CRISPR database for genome engineering. Bioinformatics 31, 3078-80 (2015).
Li, H. Aligning sequence reads, clone sequences and assembly contigs with BWA-MEM. arXiv, 1303.3997 (2013).
Jones, D. et al. cgpCaVEManWrapper: Simple execution of CaVEMan in order to detect somatic single nucleotide variants in NGS data. in Current protocols in bioinformatics Vol. 56 15.10.1-15.10.18 (2016).
Raine, K. M. et al. cgpPindel: Identifying Somatically Acquired Insertion and Deletion Events from Paired End Sequencing. Current protocols in bioinformatics 52, 15.7.1-15.7.12 (2015).
Cradick, T. J., Qiu, P., Lee, C. M., Fine, E. J. & Bao, G. COSMID: A Web-based Tool for Identifying and Validating CRISPR/Cas Off-target Sites. Molecular therapy. Nucleic acids 3, e214-e214 (2014).
The, E. P. C. et al. An integrated encyclopedia of DNA elements in the human genome. Nature 489, 57 (2012).
Quinlan, A. R. & Hall, I. M. BEDTools: a flexible suite of utilities for comparing genomic features. Bioinformatics 26, 841-842 (2010).
Team, R. C. R: A language and environment for statistical computing, (R Foundation for Statistical Computing, Vienna, Austria, 2017).
Wickham, H. ggplot2: elegant graphics for data analysis, (Springer New York, 2009).
Jover, R. et al. The efficacy of adjuvant chemotherapy with 5-fluorouracil in colorectal cancer depends on the mismatch repair status. Eur J Cancer. 2009 February; 45(3):365-73.
Devaud N, Gallinger S. Chemotherapy of MMR-deficient colorectal cancer. Fam Cancer. 2013 Jun; 12(2):301-6.
Zhao, P., Li, L., Jiang, X. et al. Mismatch repair deficiency/microsatellite instability-high as a predictor for anti-PD-1/P D-L1 immunotherapy efficacy. J Hematol Oncol 12, 54 (2019).
Sinicrope F A. DNA mismatch repair and adjuvant chemotherapy in sporadic colon cancer. Nat Rev Clin Oncol. 2010 March; 7(3):174-7.
Li, G M. Mechanisms and functions of DNA mismatch repair. Cell Res 18, 85-98 (2008).
Popat S, Hubner R, Houlston R S. Systematic review of microsatellite instability and colorectal cancer prognosis. J Clin Oncol. 2005 Jan. 20; 23(3):609-18.
Lindahl, T. & Nyberg, B. Rate of depurination of native deoxyribonucleic acid. Biochemistry 11, 3610-8 (1972).
Helleday, T., Eshtad, S. & Nik-Zainal, S. Mechanisms underlying mutational signatures in human cancers. Nat Rev Genet 15, 585-598 (2014).
Alexandrov, L. B. et al. Signatures of mutational processes in human cancer. Nature 500, 415-421 (2013).
Nik-Zainal, S. et al. The life history of 21 breast cancers. Cell 149, 994-1007 (2012).
Nik-Zainal, S. et al. Mutational processes molding the genomes of 21 breast cancers. Cell 149, 979-993 (2012).
Julian S. Gehring, Bernd Fischer, Michael Lawrence, Wolfgang Huber. SomaticSignatures: inferring mutational signatures from single-nucleotide variants. Bioinformatics, Volume 31, Issue 22, 15 Nov. 2015, Pages 3673-3675.
Damiano Fantini, Vania Vidimar, Yanni Yu, Salvatore Condello & Joshua J. Meeks. MutSignatures: an R package for extraction and analysis of cancer mutational signatures. Scientific Reports volume 10, Article number: 18217 (2020).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A method of characterising a DNA sample obtained from a tumour, the method including the steps of:
determining the value of one or more mutational signature metrics for the sample, wherein the mutational signature metrics are selected from: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts;
based on said values of said one or more mutational signature metrics, determining whether said sample has a high or low likelihood of being mismatch repair (MMR)-deficient.

2. The method of claim 1, wherein determining the value of one or more mutational signature metrics for the sample comprises determining the similarity between the substitution profile of the sample and that of one or more MMR gene knockouts.

3. The method of claim 1 or claim 2, wherein determining the value of one or more mutational signature metrics for the sample comprises determining the exposure of one or more mutational signatures of MMR.

4. The method of claim 2 or claim 3, wherein determining the value of one or more mutational signature metrics for the sample further comprises determining the number of repeat mediated indels in the mutational profile of the sample, and/or determining the similarity between the repeat mediated deletion profile of the sample and that of one or more MMR gene knockouts.

5. The method of any preceding claim, wherein determining the value of one or more mutational signature metrics for the sample comprises determining the value of all of: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts.

6. The method of any preceding claim, wherein determining whether said sample has a high or low likelihood of being MMR-deficient comprises using said values of said one or more mutational signature metrics to classify said sample between a class associated with a high likelihood of being mismatch repair (MMR)-deficient and a class associated with a low likelihood of being MMR-deficient.

7. The method of any preceding claim, wherein determining whether said sample has a high or low likelihood of being MMR-deficient comprises:
generating, using said values of said one or more mutational signature metrics, a probabilistic score; and
based on said probabilistic score, determining whether said sample has a high or low likelihood of being MMR-deficient.

8. The method of claim 7, wherein determining, based on said probabilistic score, whether said sample has a high or low likelihood of being MMR-deficient comprises comparing said probabilistic score with one or more predetermined thresholds, and determining that the sample has a high likelihood of being MMR-deficient if the probabilistic score is below a first predetermined threshold, and a low likelihood of being MMR-deficient if the probabilistic score is at or above a second predetermined threshold, optionally wherein the first and second predetermined threshold are the same.

9. The method of claim 7 or claim 8, wherein the probabilistic score is obtained using a logistic regression model, optionally wherein the probabilistic score is generated using the formula:
$$log ( \frac{p}{1 - p} ) = {β}_{0} + {\sum }_{i = 1}^{k} {β}_{i} {x}_{i}$$
where p is the probability that a sample has a particular MMR deficiency status, β₀ is an intercept weight, β is a vector of weights for each of k variables, and x is a vector of variables associated with the sample, wherein the variables comprise said one or more mutational signature metrics or variables derived therefrom.

10. The method of any preceding claim, wherein determining the value of one or more mutational signature metrics for the sample comprises scaling the value of each mutational signature metric.

11. The method of any preceding claim, wherein determining whether said sample has a high or low likelihood of being mismatch repair (MMR)-deficient based on the value of said mutational signature metrics for the sample comprises weighting each of said values by a predetermined weighting factor.

12. The method of claim 11, wherein the predetermined weighting factors are such that:
the exposure of one or more mutational signatures of mismatch repair (MMR) has a higher weight than any of: the similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts; and/or
the similarity between the substitution profile of the sample and that of one or more MMR gene knockouts has a higher weight than any of: the number of repeat mediated indels in the mutational profile of the sample, and the similarity between the repeat mediated deletion profile of the sample and that of one or more MMR gene knockouts; and/or
the exposure of one or more mutational signatures of mismatch repair (MMR) and the similarity between the substitution profile of the sample and that of one or more MMR gene knockouts both have a higher respective weight than any of: the number of repeat mediated indels in the mutational profile of the sample, and the similarity between the repeat mediated deletion profile of the sample and that of one or more MMR gene knockouts; and/or
the exposure of one or more mutational signatures of mismatch repair (MMR) has a higher weight than the similarity between the substitution profile of the sample and that of one or more MMR gene knockouts, the similarity between the substitution profile of the sample and that of one or more MMR gene knockouts has a higher weight than the similarity between the repeat mediated deletion profile of the sample and that of one or more MMR gene knockouts, and the similarity between the repeat mediated deletion profile of the sample and that of one or more MMR gene knockouts has a higher weight than the number of repeat mediated indels in the mutational profile of the sample.

13. The method of any preceding claim, determining whether said sample has a high or low likelihood of being mismatch repair (MMR)-deficient based on said values of said one or more mutational signature metrics comprises using a machine learning model that has been trained using training data comprising the values of said mutational signature metrics for a plurality of samples that have a known MMR deficiency status.

14. The method of any preceding claims, wherein determining the value of one or more mutational signature metrics for the sample comprises cataloguing the somatic mutations in said sample to produce a mutational catalogue for that sample, wherein the value of said mutational signature metrics is derived from said mutational catalogue.

15. The method of claim 14, wherein cataloguing the somatic mutations in said sample comprises determining the number of mutations in the mutational catalogue which are attributable to each of a plurality of base substitution classes and/or indel classes which are determined to be present, optionally wherein the base substitution classes include all possible trinucleotide substitution classes and/or wherein the indel classes include classes for multiple combinations of indel type, e.g. selected from insertion, deletion and complex, indel size, e.g. selected from 1-bp or longer, and flanking sequence, such as e.g. repeat-mediated, microhomology-mediated or other.

16. The method of any preceding claim, wherein:
determining the value of the exposure of one or more mutational signatures of MMR for the sample comprises determining the value of the exposure to a plurality of mutational signatures of MMR and summing the values of the exposure to each of the plurality of mutational signatures of MMR; and/or
determining the value of the exposure of one or more mutational signatures of MMR for the sample is performed as described in Degasperi et al.; and/or
determining the value of the exposure of one or more mutational signatures of MMR for the sample is performed by identifying the matrix E that satisfies C≈PE where C is a mutational catalogue for the sample, P is a signature matrix comprising the one or more mutational signatures of MMR, and E is an exposure matrix; and/or
the one or more mutational signatures of MMR are selected from RefSig MMR1 and RefSig MMR2; and/or
the one or more mutational signatures of MMR are selected from known mutational signatures that have been derived from mutational catalogues associated with a plurality of cancer samples.

17. The method of any preceding claim, wherein:
determining the value of the similarity between a substitution or repeat mediated deletion profile of the sample and that of one or more MMR gene knockouts comprises determine the cosine similarity between pairs of profiles;
determining the value of similarity between a substitution or repeat mediated deletion profile of the sample and that of one or more MMR gene knockouts comprises determining the value of similarity between a substitution or repeat mediated deletion profile of the sample and that of each of a plurality of MMR gene knockouts to obtain a plurality of similarity values, and obtaining a summarised similarity value for the plurality of similarity values, optionally wherein the summarised similarity value is the maximum or the mean similarity value; and/or
determining the value of similarity between a substitution profile of the sample and that of one or more MMR gene knockouts comprises determining the value of similarity between a substitution profile of the sample and that of each of a plurality of MMR gene knockouts to obtain a plurality of similarity values, and obtaining a summarised similarity value for the plurality of similarity values, wherein the summarised similarity value is the maximum similarity value; and/or
determining the value of similarity between a repeat mediated deletion profile of the sample and that of one or more MMR gene knockouts comprises determining the value of similarity between a repeat mediated deletion profile of the sample and that of each of a plurality of MMR gene knockouts to obtain a plurality of similarity values, and obtaining a summarised similarity value for the plurality of similarity values, wherein the summarised similarity value is the mean similarity value; and/or
wherein the one or more MMR gene knockouts are selected from: MSH2, MSH3, MSH6, MLH1, PMS2, and PMS1.

18. The method of any preceding claim, wherein:
determining the number of repeat mediated indels in the mutational profile of the sample comprises obtaining a mutational catalogue for the sample and determining the number of insertions and deletions in the mutational profile that occur within repetitive regions, and/or
wherein repetitive regions are regions comprising multiple repeats of the same sequence motif, optionally wherein a sequence motif is a sequence of between 1 and 9 bases in length.

19. The method of any preceding claim, further comprising obtaining the sample from a tumour of a subject and/or obtaining sequence data from a sample from a tumour, and/or providing to a user one or more of: the value of the one or more mutational signature metrics, a value derived therefrom (such as e.g. a probabilistic score), and a determination of whether the sample has a high likelihood or a low likelihood of being MMR-deficient.

20. A method of predicting whether a subject with cancer is likely to respond to an immunotherapy, the method comprising characterising a sample obtained from a tumour in the subject as having a high or low likelihood of being MMR-deficient using a method of any preceding claim, wherein if the sample is characterised as having a high likelihood of being MMR-deficient, the subject is likely to respond to immunotherapy.

21. An immunotherapy for use in a method of treatment of cancer in a subject, the method comprising:
(i) determining whether a DNA sample obtained from said subject has a high or low likelihood of being MMR-deficient using a method according to any one of claims 1 to 19; and
(ii) administering the immunotherapy to said subject if the DNA sample is determined to have a high likelihood of being MMR-deficient.

22. A method of providing a tool for characterising a DNA sample obtained from a tumour, the method including the steps of:
obtaining mutational signature profiles for a plurality of training samples associated with known MMR-deficiency status;
determining the value of one or more mutational signature metrics for the training samples, wherein the mutational signature metrics are selected from: exposure of one or more mutational signatures of mismatch repair (MMR), similarity between the substitution profile of the sample and that of one or more MMR gene knockouts, the number of repeat mediated indels in the mutational profile of the sample, and the similarity between the repeat mediated deletion profile of the sample and that of one or more MMR gene knockouts; and
training a machine learning model to predict, based on said values of said one or more mutational signature metrics, whether each training sample has a high or low likelihood of being mismatch repair (MMR)-deficient.

23. A system comprising:
a processor; and
a computer readable medium comprising instructions that, when executed by the processor, cause the processor to perform the steps of the method of any of claims 1 to 20 or 22.


SEQUENCE LISTING (85 sequences)

SEQ ID NO: 1 - DNA, 15 bp, Artificial Sequence
  Sequence motif
cagcagcagcagcag

SEQ ID NO: 2 - DNA, 26 bp, Artificial Sequence
  Casette specific primer
tgatatcgtggtatcgttatgcgcct

SEQ ID NO: 3 - DNA, 30 bp, Artificial Sequence
  Casette specific primer
catgtctggatccgggggtaccgcgtcgag

SEQ ID NO: 4 - DNA, 23 bp, Artificial Sequence
  UNG gRNA1
attgctaatagcagagttgctgg

SEQ ID NO: 5 - DNA, 23 bp, Artificial Sequence
  OGG1 gRNA1
cgtggactcccacttccaagagg

SEQ ID NO: 6 - DNA, 23 bp, Artificial Sequence
  POLB gRNA1
tcagcccaattcgctgatgatgg

SEQ ID NO: 7 - DNA, 23 bp, Artificial Sequence
  TDG gRNA1
ttgtaagcagccattagtcccgg

SEQ ID NO: 8 - DNA, 23 bp, Artificial Sequence
  PARP1 gRNA1
ctttatcctctgtagcaaggagg

SEQ ID NO: 9 - DNA, 23 bp, Artificial Sequence
  PARP2 gRNA1
gtgtacagccaaggtggggaagg

SEQ ID NO: 10 - DNA, 23 bp, Artificial Sequence
  MDC1 gRNA1
aaaatctgtcaagaacagaaagg

SEQ ID NO: 11 - DNA, 23 bp, Artificial Sequence
  RNF168 gRNA1
gagtccacgacgatacccggcgg

SEQ ID NO: 12 - DNA, 23 bp, Artificial Sequence
  RNF8 gRNA1
tgagggccaatggacaattatgg

SEQ ID NO: 13 - DNA, 23 bp, Artificial Sequence
  TP53 gRNA1
gatggccatggcgcggacgcggg

SEQ ID NO: 14 - DNA, 23 bp, Artificial Sequence
  ATM gRNA1
cgaattcgagtgtgtgaattagg

SEQ ID NO: 15 - DNA, 23 bp, Artificial Sequence
  NBN gRNA1
caagaagagcatgcaaccaaagg

SEQ ID NO: 16 - DNA, 23 bp, Artificial Sequence
  TP53BP1 gRNA1
gttgtctgcacaagaacttatgg

SEQ ID NO: 17 - DNA, 23 bp, Artificial Sequence
  ATP2B4 gRNA1
ggcttccgtatgtacagcaaggg

SEQ ID NO: 18 - DNA, 23 bp, Artificial Sequence
  POLE3 gRNA1
gctaacaactttgcaatgaaagg

SEQ ID NO: 19 - DNA, 23 bp, Artificial Sequence
  POLE4 gRNA1
tgcctactgttgcgctcagcagg

SEQ ID NO: 20 - DNA, 23 bp, Artificial Sequence
  PIAS1 gRNA1
attccacaactcacttacgatgg

SEQ ID NO: 21 - DNA, 23 bp, Artificial Sequence
  PIAS4 gRNA1
agtacttaaacggactgggacgg

SEQ ID NO: 22 - DNA, 23 bp, Artificial Sequence
  C1orf86 gRNA1
agtgggctccgggccgcacctgg

SEQ ID NO: 23 - DNA, 23 bp, Artificial Sequence
  DCLRE1A gRNA1
cgtcctgttttgcagataactgg

SEQ ID NO: 24 - DNA, 23 bp, Artificial Sequence
  FAN1 gRNA1
ggtggacgcctttctcaaattgg

SEQ ID NO: 25 - DNA, 23 bp, Artificial Sequence
  FANCM gRNA1
gatgaagctcataaagctctcgg

SEQ ID NO: 26 - DNA, 23 bp, Artificial Sequence
  PIF1 gRNA1
gacttccctgttcctggacaggg

SEQ ID NO: 27 - DNA, 23 bp, Artificial Sequence
  SETX gRNA1
tgttgaagcactttgtcggatgg

SEQ ID NO: 28 - DNA, 23 bp, Artificial Sequence
  RECQL5 gRNA1
atgcctttggccaacagagcagg

SEQ ID NO: 29 - DNA, 23 bp, Artificial Sequence
  WRN gRNA1
ttaaaaatggaaagaaatctggg

SEQ ID NO: 30 - DNA, 23 bp, Artificial Sequence
  EXO1 gRNA1
ttggcttgtcgtcttctgcaagg

SEQ ID NO: 31 - DNA, 23 bp, Artificial Sequence
  POLN gRNA1
acagaagaaacgggggtctgtgg

SEQ ID NO: 32 - DNA, 23 bp, Artificial Sequence
  MSH6 gRNA1
ggaacattcatccgcgagaaagg

SEQ ID NO: 33 - DNA, 23 bp, Artificial Sequence
  MLH1 gRNA1
ggctgcatacactgtttctatgg

SEQ ID NO: 34 - DNA, 23 bp, Artificial Sequence
  MSH2 gRNA1
tcaaactgagagagattgccagg

SEQ ID NO: 35 - DNA, 23 bp, Artificial Sequence
  PMS1 gRNA1
gtatccttaaacctgacttaagg

SEQ ID NO: 36 - DNA, 23 bp, Artificial Sequence
  PMS2 gRNA1
atgctgtcttctagcacttcagg

SEQ ID NO: 37 - DNA, 23 bp, Artificial Sequence
  C9orf142 gRNA1
aggctgcgggcgctgacactggg

SEQ ID NO: 38 - DNA, 23 bp, Artificial Sequence
  NHEJ1 gRNA1
tcaccaatgctgcatgcctctgg

SEQ ID NO: 39 - DNA, 23 bp, Artificial Sequence
  POLM gRNA1
aacatgtgtcgcttcggagctgg

SEQ ID NO: 40 - DNA, 23 bp, Artificial Sequence
  POLQ gRNA1
aataaaagtagacggttatatgg

SEQ ID NO: 41 - DNA, 23 bp, Artificial Sequence
  PRKDC gRNA1
cctggaatcctttctgaaacagg

SEQ ID NO: 42 - DNA, 23 bp, Artificial Sequence
  XRCC4 gRNA1
agaacttatttgttattgcttgg

SEQ ID NO: 43 - DNA, 23 bp, Artificial Sequence
  POLI gRNA1
tacttgctagtcttttaaacagg

SEQ ID NO: 44 - DNA, 23 bp, Artificial Sequence
  PRIMPOL gRNA1
tttaacaaacctgccaacccagg

SEQ ID NO: 45 - DNA, 23 bp, Artificial Sequence
  RAD18 gRNA1
cgcttagcctctgagggatctgg

SEQ ID NO: 46 - DNA, 23 bp, Artificial Sequence
  REV1 gRNA1
ccatttgcttgcgcagaatctgg

SEQ ID NO: 47 - DNA, 23 bp, Artificial Sequence
  OGG1 gRNA2
gagccagggtaacatctagctgg

SEQ ID NO: 48 - DNA, 23 bp, Artificial Sequence
  POLB gRNA2
tgaaccatcatcagcgaattggg

SEQ ID NO: 49 - DNA, 23 bp, Artificial Sequence
  PARP1 gRNA2
tcccaggagtcaagagtgaaggg

SEQ ID NO: 50 - DNA, 23 bp, Artificial Sequence
  PARP2  gRNA2
agctttgccctttaacagcaagg

SEQ ID NO: 51 - DNA, 23 bp, Artificial Sequence
  MDC1 gRNA2
ggcgtatggtaaaaaaatcaagg

SEQ ID NO: 52 - DNA, 23 bp, Artificial Sequence
  RNF168 gRNA2
ctctcgtcaacgtggaactgtgg

SEQ ID NO: 53 - DNA, 23 bp, Artificial Sequence
  RNF8 gRNA1
agtggtttcgagaaatcatcagg

SEQ ID NO: 54 - DNA, 23 bp, Artificial Sequence
  TP53 gRNA2
gagcgctgctcagatagcgatgg

SEQ ID NO: 55 - DNA, 23 bp, Artificial Sequence
  ATM gRNA2
agttgacagccaaagtcttgagg

SEQ ID NO: 56 - DNA, 23 bp, Artificial Sequence
  NBN gRNA2
aatcaagctatattgcaacttgg

SEQ ID NO: 57 - DNA, 23 bp, Artificial Sequence
  TP53BP1 gRNA2
catagcagcaacagatgctttgg

SEQ ID NO: 58 - DNA, 23 bp, Artificial Sequence
  ATP2B4 gRNA2
accgttgggattcctgatgacgg

SEQ ID NO: 59 - DNA, 23 bp, Artificial Sequence
  POLE3 gRNA2
tcaatggggtaacgaaccgctgg

SEQ ID NO: 60 - DNA, 23 bp, Artificial Sequence
  POLE4 gRNA2
gcctactgttgcgctcagcaggg

SEQ ID NO: 61 - DNA, 23 bp, Artificial Sequence
  PIAS1 gRNA2
cataggacttgaatgtacgttgg

SEQ ID NO: 62 - DNA, 23 bp, Artificial Sequence
  PIAS4 gRNA2
tcaatattggggcctgccagcgg

SEQ ID NO: 63 - DNA, 23 bp, Artificial Sequence
  C1orf86 gRNA2
tcggacccaagaccttttcctgg

SEQ ID NO: 64 - DNA, 23 bp, Artificial Sequence
  DCLRE1A gRNA2
atatattcacccattgccactgg

SEQ ID NO: 65 - DNA, 23 bp, Artificial Sequence
  FAN1 gRNA2
attgggattcaccaagtggaagg

SEQ ID NO: 66 - DNA, 23 bp, Artificial Sequence
  FANCM gRNA2
cgggacaagctcctctagaaagg

SEQ ID NO: 67 - DNA, 23 bp, Artificial Sequence
  PIF1 gRNA2
ctgggctcactgccccccacagg

SEQ ID NO: 68 - DNA, 23 bp, Artificial Sequence
  SETX gRNA2
tgttgaagcactttgtcggatgg

SEQ ID NO: 69 - DNA, 23 bp, Artificial Sequence
  RECQL5 gRNA2
tcattgctttgattcaggtgagg

SEQ ID NO: 70 - DNA, 23 bp, Artificial Sequence
  WRN gRNA2
gttctaccgtgccactattgagg

SEQ ID NO: 71 - DNA, 23 bp, Artificial Sequence
  EXO1 gRNA2
tcttcgtgaggggaaagtctcgg

SEQ ID NO: 72 - DNA, 23 bp, Artificial Sequence
  POLN gRNA2
gtaaaacgccaagcagagggtgg

SEQ ID NO: 73 - DNA, 23 bp, Artificial Sequence
  MSH6 gRNA2
aaaccagacaaggccaccagggg

SEQ ID NO: 74 - DNA, 23 bp, Artificial Sequence
  MSH2 gRNA2
aatgatatgtcagcttccattgg

SEQ ID NO: 75 - DNA, 23 bp, Artificial Sequence
  PMS1 gRNA2
gcagttacagttgtacctgttgg

SEQ ID NO: 76 - DNA, 23 bp, Artificial Sequence
  C9orf142 gRNA2
gggcccccctgaaagcgtcaggg

SEQ ID NO: 77 - DNA, 23 bp, Artificial Sequence
  NHEJ1 gRNA2
gaatctgcaggatctgtatatgg

SEQ ID NO: 78 - DNA, 23 bp, Artificial Sequence
  POLM gRNA2
agaggaggccgtcagctggcagg

SEQ ID NO: 79 - DNA, 23 bp, Artificial Sequence
  POLQ gRNA2
tctgatcaatcgcctcatagagg

SEQ ID NO: 80 - DNA, 23 bp, Artificial Sequence
  PRKDC gRNA2
ttttcaattctacatttgtgtgg

SEQ ID NO: 81 - DNA, 23 bp, Artificial Sequence
  XRCC4 gRNA2
ttcaactttctctaggttgaagg

SEQ ID NO: 82 - DNA, 23 bp, Artificial Sequence
  PRIMPOL gRNA2
agcttgcacacagcattttcagg

SEQ ID NO: 83 - DNA, 23 bp, Artificial Sequence
  RAD18 gRNA2
ctccagacagtctttaaagcagg

SEQ ID NO: 84 - DNA, 23 bp, Artificial Sequence
  REV1 gRNA2
aattgcatcttgtagttatgagg

SEQ ID NO: 85 - DNA, 11 bp, Artificial Sequence
  PMS2 mutation
tgtgtgtgaag